(19) United States
(12) Patent Application Publication
(10) Pub. No.: US 2025/0090648 A1
(43) Pub. Date: Mar. 20, 2025

(54) NEXT GENERATION MRNA VACCINES

(71) Applicant: FuTr Bio Ltda., Floriano´polis (BR)

(72) Inventors: Daniel Santos MANSUR, Florianópolis, SC (BR); André BÁFICA, Florianópolis, SC (BR)

(21) Appl. No.: 18/810,225

(22) Filed: Aug. 20, 2024

Related U.S. Application Data
(63) Continuation of application No. PCT/IB2023/000094, filed on Feb. 21, 2023.
(60) Provisional application No. 63/312,745, filed on Feb. 22, 2022.
(60) Provisional application No. 63/479,974, filed on Jan. 13, 2023.

(51) Int. Cl. A61K 39/12; A61K 39/00; A61P 37/04

(52) U.S. Cl. CPC A61K 39/12; A61P 37/04; A61K 2039/53; A61K 2039/55516; A61K 2039/6075

(57) ABSTRACT

Described herein are next generation vaccine compositions, including mRNA vaccines having flavivirus untranslated regions and vaccines comprising a (major histocompatibility complex) MHC binding peptide.

### CROSS-REFERENCE TO RELATED APPLICATIONS

This application is a continuation of International Application No. PCT/IB2023/000094, filed Feb. 21, 2023, which claims the benefit of U.S. Provisional Application No. 63/312,745, filed on Feb. 22, 2022, and U.S. Provisional Application No. 63/479,974, filed on Jan. 13, 2023, each of which are incorporated herein by reference in their entirety.

### REFERENCE TO A SEQUENCE LISTING

This application contains a Sequence Listing which has been submitted electronically in XML format and is hereby incorporated by reference in its entirety. Said XML copy, created on Aug. 19, 2024, is named FUTR62558_701_301.xml and is 263,220 bytes in size.

### BACKGROUND

mRNA vaccines are gene-based vaccines that use mRNA as a vehicle to deliver a gene sequence encoding an antigen to induce an immune response in a subject. Several mRNA vaccine platforms have been developed in recent years, especially to respond to the COVID-19 pandemic. However, such first generation mRNA vaccines have several downsides, including production with modified nucleotides, requiring numerous doses for efficacy, and requiring healthy cellular systems to translate mRNA in vivo. Accordingly, there is a need for mRNA vaccines with improved efficacy, stability, and safety.

### SUMMARY

In certain aspects, provided herein are second generation mRNA vaccines that overcome one or more of the downsides of first generation mRNA vaccines. In some cases, mRNA vaccines herein comprise one or more untranslated regions of a flavivirus. In some cases, mRNA vaccines herein are capable of translation during cellular stress responses.

Further provided are non-mRNA vaccines that employ one or more features of the second generation vaccines herein. For instance, in some cases mRNA and non-mRNA vaccines comprise a MHC (major histocompatibility complex) binding peptide as a molecular booster.

Certain embodiments herein include a nucleic acid composition comprising a 5′ untranslated region (5′ UTR) of a first flavivirus, a 3′ untranslated region (3′ UTR) of a second flavivirus, a first polynucleotide encoding a first peptide that is exogenous to the first flavivirus and/or the second flavivirus, and a polynucleotide encoding a major histocompatibility complex (MHC) binding peptide. Certain embodiments herein include a method of expressing a first peptide in a cell, the method comprising delivering to the cell a nucleic acid composition comprising a 5′ untranslated region (5′ UTR) of a first flavivirus, a 3′ untranslated region (3′ UTR) of a second flavivirus, a first polynucleotide encoding the first peptide, wherein the first peptide is exogenous to the first flavivirus and/or the second flavivirus, and a polynucleotide encoding a major histocompatibility complex (MHC) binding peptide. Certain embodiments herein include a method of inducing an immune response in a subject, the method comprising administering to the subject a nucleic acid composition comprising a 5′ untranslated region (5′ UTR) of a first flavivirus, a 3′ untranslated region (3′ UTR) of a second flavivirus, a first polynucleotide encoding a first peptide that is exogenous to the first flavivirus and/or the second flavivirus, and a polynucleotide encoding a major histocompatibility complex (MHC) binding peptide.

In some embodiments, the 5′ UTR is a 5′ UTR of a dengue virus (DENV), West Nile virus (WNV), Japanese encephalitis virus (JEV), yellow fever virus (YFV), Zika virus (ZIKV), or tick-born encephalitis virus (TBEV); and the 3′ UTR is a 3′ UTR of a dengue virus (DENV), West Nile virus (WNV), Japanese encephalitis virus (JEV), yellow fever virus (YFV), Zika virus (ZIKV), or tick-born encephalitis virus (TBEV); and/or wherein the first flavivirus is the same as the second flavivirus. In some embodiments, the 5′ UTR is a 5′ UTR of a DENV, and the 3′ UTR is a 3′ UTR of a DENV. In some embodiments, the 5′ UTR is homologous or at least 80% identical to a sequence of Table 1, the 3′ UTR is homologous or at least 80% identical to a sequence of Table 2. In some embodiments, the MHC binding peptide comprises a sequence homologous or at least 80% identical to any one of SEQ ID NOS: 136-163. In some embodiments, the MHC binding peptide comprises a sequence at least 80% identical to 10 or more nucleobases of a pathogen. In some embodiments, the polynucleotide encoding a MHC binding peptide encodes a plurality of MHC binding peptides, optionally wherein each of the plurality of MHC binding peptides is the same or different from another of the plurality of MHC binding peptides. In some embodiments, the plurality of MHC binding peptides is about 2, 3, 4, 5, 6, 7, 8, 9, or 10 MHC binding peptides. In some embodiments, the nucleic acid composition comprises a polynucleotide linker between two polynucleotides encoding two of the plurality of MHC binding peptides. In some embodiments, the polynucleotide linker encodes a cleavage site. In some embodiments, the nucleic acid composition is more resistant to RNAse degradation as compared to a control composition comprising a non-flavivirus 5′ UTR, a non-flavivirus 3′ UTR, and the polynucleotide encoding the first peptide. In some embodiments, the nucleic acid composition comprises a polynucleotide encoding a signal peptide. In some embodiments, the nucleic acid composition comprises a polynucleotide encoding a cleavage site. In some embodiments, the nucleic acid composition does not comprise a sequence encoding 10 or more contiguous amino acids of a structural protein of the first flavivirus or the second flavivirus. In some embodiments, the nucleic acid composition does not comprise a sequence encoding 10 or more contiguous amino acids of a non-structural protein of the first flavivirus or the second flavivirus. In some embodiments, the first peptide is a pathogen-associated antigen.

Certain embodiments herein include a method of expressing a peptide in a cell, the method comprising delivering to the cell a nucleic acid composition comprising a 5′ untranslated region (5′ UTR) of a first flavivirus, a 3′ untranslated region (3′ UTR) of a second flavivirus, and a polynucleotide encoding the peptide, wherein the polynucleotide encoding the peptide is exogenous to the first flavivirus and/or the second flavivirus. Certain embodiments herein include a method of inducing an immune response in a subject, the method comprising administering to the subject a nucleic acid composition comprising a 5′ untranslated region (5′ UTR) of a first flavivirus, a 3′ untranslated region (3′ UTR) of a second flavivirus, and a polynucleotide encoding a peptide, wherein the polynucleotide encoding the peptide is exogenous to the first flavivirus and/or the second flavivirus. In some embodiments, the polynucleotide is translated into the peptide during cellular stress. In some embodiments, the peptide is expressed from the nucleic acid composition more than the peptide expressed from a control composition comprising a non-flavivirus 5′ UTR, a non-flavivirus 3′ UTR, and the polynucleotide encoding the peptide.

Certain embodiments herein include a nucleic acid composition comprising a 5′ untranslated region (5′ UTR) of a first flavivirus, a 3′ untranslated region (3′ UTR) of a second flavivirus, and a polynucleotide encoding a peptide, wherein the polynucleotide is exogenous to the first flavivirus and/or the second flavivirus.

In some embodiments, the nucleic acid composition is more resistant to RNAse degradation as compared to a control composition comprising a non-flavivirus 5′ UTR, a non-flavivirus 3′ UTR, and the polynucleotide encoding the peptide. In some embodiments, the nucleic acid comprises a polynucleotide encoding a signal peptide. In some embodiments, the nucleic acid composition comprises a polynucleotide encoding a cleavage site. In some embodiments, the 5′ UTR is a 5′ UTR of a dengue virus (DENV), West Nile virus (WNV), Japanese encephalitis virus (JEV), yellow fever virus (YFV), Zika virus (ZIKV), or tick-born encephalitis virus (TBEV). In some embodiments, the 3′ UTR is a 3′ UTR of a dengue virus (DENV), West Nile virus (WNV), Japanese encephalitis virus (JEV), yellow fever virus (YFV), Zika virus (ZIKV), or tick-born encephalitis virus (TBEV). In some embodiments, the 5′ UTR is a 5′ UTR of a DENV, and the 3′ UTR is a 3′ UTR of a DENV. In some embodiments, the 5′ UTR is homologous or at least 80% identical to a sequence of Table 1, the 3′ UTR is homologous or at least 80% identical to a sequence of Table 2. In some embodiments, the nucleic acid composition does not comprise a sequence encoding 10 or more contiguous amino acids of a structural protein of the first flavivirus or the second flavivirus. In some embodiments, the nucleic acid composition does not comprise a sequence encoding 10 or more contiguous amino acids of a non-structural protein of the first flavivirus or the second flavivirus. In some embodiments, the nucleic acid composition of any one of claims 23-32, wherein the peptide is a pathogen-associated antigen.

Certain embodiments herein include a method of inducing an immune response in a subject, the method comprising administering to the subject a nucleic acid composition comprising a polynucleotide encoding a first peptide and a polynucleotide encoding a major histocompatibility complex (MHC) binding peptide. Certain embodiments herein include a nucleic acid composition comprising a polynucleotide encoding a first peptide and a polynucleotide encoding a MHC binding peptide. In some embodiments, the polynucleotide encoding a MHC binding peptide encodes a plurality of MHC binding peptides, optionally wherein each of the plurality of MHC binding peptides is the same or different from another of the plurality of MHC binding peptides. In some embodiments, the plurality of MHC binding peptides is about 2, 3, 4, 5, 6, 7, 8, 9, or 10 MHC binding peptides. In some embodiments, the nucleic acid composition comprises a polynucleotide linker between two polynucleotides encoding two of the plurality of MHC binding peptides. In some embodiments, the polynucleotide linker encodes a cleavage site. In some embodiments, the MHC binding peptide comprises a sequence homologous or at least 80% identical to any one of SEQ ID NOS: 136-163. In some embodiments, the MHC binding peptide comprises a sequence at least 80% identical to 10 or more nucleobases of a pathogen. In some embodiments, the first peptide is a pathogen-associated antigen. In some embodiments, provided is a method of expressing the first peptide in a cell, the method comprising delivering to the cell the nucleic acid composition.

In one aspect, provided herein is a nucleic acid comprising (i) a first exogenous polynucleotide, and (ii) a 5′ untranslated region (5′ UTR) of a first flavivirus and/or a 3′ untranslated region (3′ UTR) of a second flavivirus. In some embodiments, the first flavivirus is a tick-borne flavivirus (TBFV), a mosquito-borne flavivirus (MBFV), an insect-specific flavivirus (ISFV), no-known vector flavivirus (NKFV), or a non-classified flavivirus (NCFV). In some embodiments, the first flavivirus is a dengue virus (DENV), West Nile virus (WNV), Japanese encephalitis virus (JEV), yellow fever virus (YFV), Zika virus (ZIKV), tick-born encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV). In some embodiments, the first flavivirus is a dengue virus (DENV). In some embodiments, the dengue virus is a dengue virus serotype 4 (DENV-4). In some embodiments, the second flavivirus is a tick-borne flavivirus (TBFV), a mosquito-borne flavivirus (MBFV), an insect-specific flavivirus (ISFV), no-known vector flavivirus (NKFV), or a non-classified flavivirus (NCFV). In some embodiments, the second flavivirus is a dengue virus (DENV), West Nile virus (WNV), Japanese encephalitis virus (JEV), yellow fever virus (YFV), Zika virus (ZIKV), tick-born encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV). In some embodiments, the second flavivirus is a dengue virus (DENV). In some embodiments, the dengue virus is a dengue virus serotype 4 (DENV-4). In some embodiments, the first flavivirus and the second flavivirus are the same flavivirus.

In some embodiments, the 5′ UTR comprises a sequence at least about 80% identical to any one of SEQ ID NOS: 1-36, or comprises a sequence at least 80% identical to at least 50, 60, 70, 80, 90, or 100 contiguous bases of a virus of Table 1. In some embodiments, the 5′ UTR comprises a sequence derived from any one of SEQ ID NOS: 1-36, or of a virus of Table 1. In some embodiments, the 5′ UTR is at least 80% identical to SEQ ID NO: 5 or 36.

In some embodiments, the 3′ UTR comprises a sequence at least about 80% identical to any one of SEQ ID NOS: 37-70, or comprises a sequence at least 80% identical to at least 50, 60, 70, 80, 90, or 100 contiguous bases of a virus of Table 2. In some embodiments, the 3′ UTR comprises a sequence derived from any one of SEQ ID NOS: 37-70, or of a virus of Table 2. In some embodiments, the 3′ UTR is at least 80% identical to SEQ ID NO: 40.

In some embodiments, the 5′ UTR comprises the stem loop A of the 5′ UTR of the first flavivirus. In some embodiments, the 5′ UTR comprises the stem loop B of the 5′ UTR of the first flavivirus. In some embodiments, the 5′ UTR comprises the 5′ ATG of the first flavivirus. In some embodiments, the 5′ UTR comprises the capsid-coding region hairpin element (cHP) of the first flavivirus. In some embodiments, the 5′ UTR comprises the 5′ conserved sequence of the first flavivirus. In some embodiments, the 3′ UTR comprises at least one endonuclease resistance sequence of the second flavivirus. In some embodiments, the 3′ UTR comprises the short hairpin structure of the second flavivirus. In some embodiments, the 3′ UTR comprises the 3′ cyclization sequence of the second flavivirus. In some embodiments, the 3′ UTR comprises the 3′ TAG, TAA, or TGA of the second flavivirus. In some embodiments, the 5′ UTR does not comprise a 5′ cap modification. In some embodiments, the 5′ UTR comprises a 5′ cap modification. In some embodiments, the 5′ UTR has a length of about 80 bases to about 200 bases. In some embodiments, the 3′ UTR has a length of about 200 to about 700 bases.

In some embodiments, the nucleic acid does not comprise a sequence encoding 10 or more contiguous amino acids of a structural protein of the first flavivirus or the second flavivirus. In some embodiments, the nucleic acid does not comprise a sequence encoding 10 or more contiguous amino acids of any structural protein of the first flavivirus or the second flavivirus. In some embodiments, the structural protein is a capsid, membrane, or envelope protein of the first flavivirus or the second flavivirus. In some embodiments, the nucleic acid does not comprise a sequence encoding 10 or more contiguous amino acids of a non-structural protein of the first flavivirus or the second flavivirus. In some embodiments, the nucleic acid does not comprise a sequence encoding 10 or more contiguous amino acids of any non-structural protein of the first flavivirus or the second flavivirus. In some embodiments, the nucleic acid does not comprise a sequence 3′ to the exogenous nucleotide sequence comprising at least 10 bases having at least 80% adenosine residues. In some embodiments, the exogenous polynucleotide encodes a polypeptide. In some embodiments, the exogenous polynucleotide is translated into the polypeptide in healthy cells or during cellular stress responses.

In some embodiments, the nucleic acid is resistant to degradation by a RNAse. In some embodiments, the RNAse is XRN-1. In some embodiments, the RNAse comprises one or more of the extracellular RNAses selected from the group consisting of hRNAse1, hRNAse2, hRNAse3, hRNAse 4, hRNAse5, hRNAse6, hRNAse7, hRNAse8, hRNAse9, hRNAse10, hRNAse1 1, hRNAse12, hRNAse13, bovine seminal RNAse, bovine milk RNAse, rodent RNAse, frog RNAse, RNAseT2, plant self-incompatibility RNAse, or bacterial RNAse.

In some embodiments, the nucleic acid has no or fewer than 10 base modifications. In some embodiments, the nucleic acid has no or fewer than 10 backbone modifications. In some embodiments, the nucleic acid has no or fewer than 10 sugar modifications. In some embodiments, the nucleic acid is a deoxyribonucleic acid (DNA).

Also provided herein is a ribonucleic acid (RNA) transcribed from DNA described herein. In some embodiments, the RNA is transcribed in vitro or in vivo.

In some embodiments, the nucleic acid is a ribonucleic acid (RNA). In some embodiments, the RNA is a messenger RNA. In some embodiments, the nucleic acid comprises a self-cleavage site. In some embodiments, the nucleic acid comprises an internal ribosome entry site. In some embodiments, the nucleic acid comprises a sequence encoding a peptide that induces ribosomal skipping during translation. In some embodiments, the nucleic acid comprises a sequence encoding a peptide motif of DxExNPGP, where x is any amino acid. In some embodiments, the nucleic acid comprises a sequence at least 80% identical to SEQ ID NO: 71. In some embodiments, the nucleic acid comprises a sequence encoding a signal peptide. In some embodiments, the signal peptide is Gaussia luciferase, human albumin, human chymotrypsinogen, human interleukin-2, or human trypsinogen-2. In some embodiments, the signal peptide is at least 80% identical to any one of SEQ ID NOS: 107-112. In some embodiments, the signal peptide is at least 80% identical to SEQ ID NO: 107. In some embodiments, the nucleic acid comprises a sequence encoding a cleavage site positioned between the 5′ UTR and the exogenous polynucleotide. In some embodiments, the cleavage site comprises an exopeptidase, endopeptidase and/or exopeptidase cleavage site. In some embodiments, the cleavage site is a proteasome cleavage site, a cysteine protease cleavage site, an aspartate protease cleavage site, a serine protease cleavage site, or a combination thereof. In some embodiments, the sequence encoding the cleavage site comprises a sequence at least 80% identical to any one of SEQ ID NOS: 73-82. In some embodiments, the sequence encoding the cleavage site comprises a sequence at least 80% identical to SEQ ID NO: 81. In some embodiments, the cleavage site comprises a sequence at least 80% identical to any one of SEQ ID NOS: 83-92. In some embodiments, the cleavage site comprises a sequence at least 80% identical to SEQ ID NO: 91.

In some embodiments, the exogenous polynucleotide encodes a pathogen-associated antigen. In some embodiments, the pathogen is a virus, bacteria, fungus, protozoa, or helminth. In some embodiments, the exogenous polynucleotide encodes a viral structural protein, a viral envelope protein, a viral capsid protein, or a viral nonstructural protein, or any combination thereof. In some embodiments, the exogenous polynucleotide encodes an antigen from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus. In some embodiments, the exogenous polynucleotide encodes an antigen from a bacteria selected from Helicobacter pylori, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii. In some embodiments, the exogenous polynucleotide encodes an antigen from a fungi selected from Cryptococcus neoformans, Histoplasma capsulatum, Coccidioides immitis, Blastomyces dermatitidis, Chlamydia trachomatis, and Candida albicans. In some embodiments, the exogenous polynucleotide encodes an antigen from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major. In some embodiments, the exogenous polynucleotide comprises a sequence at least 80% identical to any one of SEQ ID NOS: 93-96. In some embodiments, the exogenous polynucleotide encodes an antigen having a sequence at least 80% identical to any one of SEQ ID NOS: 97-100.

In one aspect, provided herein is a method of inducing an immune response in a subject, the method comprising administering to the subject the nucleic acid.

In another aspect, provided herein is a nucleic acid composition comprising a first sequence encoding a first antigen, and a second sequence encoding a MHC binding peptide. In some embodiments, the MHC binding peptide is a MHC class I and/or a MHC class II peptide. In some embodiments, the second sequence comprises a sequence at least 80% identical to any one of SEQ ID NOS: 113-135. In some embodiments, the second sequence comprises a sequence at least 80% identical to SEQ ID NO: 113. In some embodiments, the MHC binding peptide comprises a sequence at least 80% identical to any one of SEQ ID NOS: 136-163. In some embodiments, the MHC binding peptide comprises a sequence at least 80% identical to SEQ ID NO: 136. In some embodiments, the second sequence comprises a pathogen-associated sequence.

In some embodiments, the pathogen is a virus, bacteria, fungus, protozoa, or helminth. In some embodiments, the second sequence is at least 80% identical to 10 or more nucleobases from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Bimaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus.

In some embodiments, the second sequence is at least 80% identical to 10 or more nucleobases from a bacteria selected from Helicobacter pylori, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii. In some embodiments, the second sequence is at least 80% identical to 10 or more nucleobases from a fungi selected from Cryptococcus neoformans, Histoplasma capsulatum, Coccidioides immitis, Blastomyces dermatitidis, Chlamydia trachomatis, and Candida albicans. In some embodiments, the second sequence is at least 80% identical to 10 or more nucleobases from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major.

In some embodiments, the MHC binding peptide has a length of 7-20 peptides. In some embodiments, the nucleic acid comprises two or more sequences encoding a MHC binding peptide.

In some embodiments, the first sequence is at least 80% identical to 10 or more nucleobases from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Bimaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus. In some embodiments, the first sequence is at least 80% identical to 10 or more nucleobases from a bacteria selected from Helicobacter pyloris, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii. In some embodiments, the first sequence is at least 80% identical to 10 or more nucleobases from a fungi selected from Cryptococcus neoformans, Histoplasma capsulatum, Coccidioides immitis, Blastomyces dermatitidis, Chlamydia trachomatis, and Candida albicans. In some embodiments, the first sequence is at least 80% identical to 10 or more nucleobases from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania major.

In some embodiments, the first antigen has a sequence at least 80% identical to any one of SEQ ID NOS: 97-100. In some embodiments, the first sequence comprises a sequence at least 80% identical to any one of SEQ ID NOS: 93-96. In some embodiments, the first sequence and the second sequence are present on two separate nucleic acid strands. In some embodiments, the first sequence and the second sequence are connected.

In some embodiments, the nucleic acid comprises a sequence encoding a cleavage site. In some embodiments, the cleavage site comprises an exopeptidase, endopeptidase and/or exopeptidase cleavage site. In some embodiments, the cleavage site is a proteasome cleavage site, a cysteine protease cleavage site, an aspartate protease cleavage site, or a serine protease cleavage site. In some embodiments, the sequence encoding the cleavage site comprises a sequence at least 80% identical to any one of SEQ ID NOS: 73-82. In some embodiments, the sequence encoding the cleavage site comprises a sequence at least 80% identical to SEQ ID NO: 81. In some embodiments, the cleavage site comprises a sequence at least 80% identical to any one of SEQ ID NOS: 83-92. In some embodiments, the cleavage site comprises a sequence at least 80% identical to SEQ ID NO: 91.

In some embodiments, the nucleic acid comprises a sequence encoding a signal peptide. In some embodiments, the signal peptide is Gaussia luciferase, human albumin, human chymotrypsinogen, human interleukin-2, or human trypsinogen-2. In some embodiments, the signal peptide is at least 80% identical to any one of SEQ ID NOS: 107-112. In some embodiments, the signal peptide is at least 80% identical to SEQ ID NO: 107.

In some embodiments, the nucleic acid is a deoxyribonucleic acid (DNA).

Further provided herein is a ribonucleic acid (RNA) transcribed from the DNA. In some embodiments, the RNA is transcribed in vitro or in vivo.

In some embodiments, the nucleic acid is a ribonucleic acid (RNA). In some embodiments, the RNA is a messenger RNA.

Also provided herein is a peptide translated from the nucleic acid.

In another aspect, provided herein is a method of inducing an immune response in a subject, the method comprising administering to the subject the nucleic acid or the peptide. In some embodiments, the nucleic acid is delivered via a lipid nanoparticle, virus-like particle, or naked.

In yet another aspect, provided herein is a nucleic acid comprising (i) a first exogenous polynucleotide, (ii) a 5′ untranslated region (5′ UTR) of a first flavivirus and/or a 3′ untranslated region (3′ UTR) of a second flavivirus, and (iii) a polynucleotide encoding a MHC binding peptide. In some embodiments, the first flavivirus is a tick-borne flavivirus (TBFV), a mosquito-borne flavivirus (MBFV), an insect-specific flavivirus (ISFV), no-known vector flavivirus (NKFV), or a non-classified flavivirus (NCFV). In some embodiments, the first flavivirus is a dengue virus (DENV), West Nile virus (WNV), Japanese encephalitis virus (JEV), yellow fever virus (YFV), Zika virus (ZIKV), tick-born encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV). In some embodiments, the first flavivirus is a dengue virus (DENV). In some embodiments, the dengue virus is a dengue virus serotype 4 (DENV-4). In some embodiments, the second flavivirus is a tick-borne flavivirus (TBFV), a mosquito-borne flavivirus (MBFV), an insect-specific flavivirus (ISFV), no-known vector flavivirus (NKFV), or a non-classified flavivirus (NCFV). In some embodiments, the second flavivirus is a dengue virus (DENV), West Nile virus (WNV), Japanese encephalitis virus (JEV), yellow fever virus (YFV), Zika virus (ZIKV), tick-born encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV). In some embodiments, the second flavivirus is a dengue virus (DENV). In some embodiments, the dengue virus is a dengue virus serotype 4 (DENV-4). In some embodiments, the first flavivirus and the second flavivirus are the same flavivirus.

In some embodiments, the 5′ UTR comprises a sequence at least about 80% identical to any one of SEQ ID NOS: 1-36 or comprises a sequence at least 80% identical to at least 50, 60, 70, 80, 90, or 100 contiguous bases of a virus of Table 1. In some embodiments, the 5′ UTR comprises a sequence derived from any one of SEQ ID NOS: 1-36, or of a virus of Table 1. In some embodiments, the 5′ UTR is at least 80% identical to SEQ ID NO: 5 or 36. In some embodiments, the 3′ UTR comprises a sequence at least about 80% identical to any one of SEQ ID NOS: 37-70, or comprises a sequence at least 80% identical to at least 50, 60, 70, 80, 90, or 100 contiguous bases of a virus of Table 2. In some embodiments, the 3′ UTR comprises a sequence derived from any one of SEQ ID NOS: 37-70, or of a virus of Table 2. In some embodiments, the 3′ UTR is at least 80% identical to SEQ ID NO: 40. In some embodiments, the 5′ UTR comprises the stem loop A of the 5′ UTR of the first flavivirus. In some embodiments, the 5′ UTR comprises the stem loop B of the 5′ UTR of the first flavivirus. In some embodiments, the 5′ UTR comprises the 5′ ATG of the first flavivirus. In some embodiments, the 5′ UTR comprises the capsid-coding region hairpin element (cHP) of the first flavivirus. In some embodiments, the 5′ UTR comprises the 5′ conserved sequence of the first flavivirus.

In some embodiments, the 3′ UTR comprises at least one endonuclease resistance sequence of the second flavivirus. In some embodiments, the 3′ UTR comprises the short hairpin structure of the second flavivirus. In some embodiments, the 3′ UTR comprises the 3′ cyclization sequence of the second flavivirus. In some embodiments, the 3′ UTR comprises the 3′ TAG, TAA, or TGA of the second flavivirus.

In some embodiments, the 5′ UTR does not comprise a 5′ cap modification. In some embodiments, the 5′ UTR comprises a 5′ cap modification. In some embodiments, the 5′ UTR has a length of about 80 bases to about 200 bases. In some embodiments, the 3′ UTR has a length of about 200 to about 700 bases.

In some embodiments, the nucleic acid does not comprise a sequence encoding 10 or more contiguous amino acids of a structural protein of the first flavivirus or the second flavivirus. In some embodiments, the nucleic acid does not comprise a sequence encoding 10 or more contiguous amino acids of any structural protein of the first flavivirus or the second flavivirus. In some embodiments, the structural protein is a capsid, membrane, or envelope protein of the first flavivirus or the second flavivirus. In some embodiments, the nucleic acid does not comprise a sequence encoding 10 or more contiguous amino acids of a non-structural protein of the first flavivirus or the second flavivirus. In some embodiments, the nucleic acid does not comprise a sequence encoding 10 or more contiguous amino acids of any non-structural protein of the first flavivirus or the second flavivirus. In some embodiments, the nucleic acid does not comprise a sequence 3′ to the exogenous nucleotide sequence comprising at least 10 bases having at least 80% adenosine residues. In some embodiments, the exogenous polynucleotide encodes a polypeptide. In some embodiments, the exogenous polynucleotide is translated into the polypeptide in healthy cells or during cellular stress responses.

In some embodiments, the nucleic acid is resistant to degradation by a RNAse. In some embodiments, the RNAse is XRN-1. In some embodiments, the RNAse comprises one or more of the extracellular RNAses selected from the group consisting of hRNAse1, hRNAse2, hRNAse3, hRNAse 4, hRNAse5, hRNAse6, hRNAse7, hRNAse8, hRNAse9, hRNAse10, hRNAse1 1, hRNAse12, hRNAse13, bovine seminal RNAse, bovine milk RNAse, rodent RNAse, frog RNAse, RNAseT2, plant self-incompatibility RNAse, or bacterial RNAse.

In some embodiments, the nucleic acid has no or fewer than 10 base modifications. In some embodiments, the nucleic acid has no or fewer than 10 backbone modifications. In some embodiments, the nucleic acid has no or fewer than 10 sugar modifications.

In some embodiments, the nucleic acid is a deoxyribonucleic acid (DNA).

Further provided herein is a ribonucleic acid (RNA) transcribed from the DNA. In some embodiments, the RNA is transcribed in vitro or in vivo.

In some embodiments, the nucleic acid is a ribonucleic acid (RNA). In some embodiments, the RNA is a messenger RNA. In some embodiments, the nucleic acid comprises a self-cleavage site. In some embodiments, the nucleic acid comprises an internal ribosome entry site. In some embodiments, the nucleic acid comprises a sequence encoding a peptide that induces ribosomal skipping during translation. In some embodiments, the nucleic acid comprises a sequence encoding a peptide motif of DxExNPGP, where x is any amino acid. In some embodiments, the nucleic acid comprises a sequence at least 80% identical to SEQ ID NO: 71.

In some embodiments, the nucleic acid comprises a sequence encoding a signal peptide. In some embodiments, the signal peptide is Gaussia luciferase, human albumin, human chymotrypsinogen, human interleukin-2, or human trypsinogen-2. In some embodiments, the signal peptide is at least 80% identical to any one of SEQ ID NOS: 107-112. In some embodiments, the signal peptide is at least 80% identical to SEQ ID NO: 107.

In some embodiments, the nucleic acid comprises a sequence encoding a cleavage site. In some embodiments, the sequence encoding the cleavage site is positioned between the 5′ UTR and the exogenous polynucleotide. In some embodiments, the cleavage site comprises an exopeptidase, endopeptidase and/or exopeptidase cleavage site. In some embodiments, the cleavage site is a proteasome cleavage site, a cysteine protease cleavage site, an aspartate protease cleavage site, a serine protease cleavage site, or a combination thereof. In some embodiments, the sequence encoding the cleavage site comprises a sequence at least 80% identical to any one of SEQ ID NOS: 73-82. In some embodiments, the sequence encoding the cleavage site comprises a sequence at least 80% identical to SEQ ID NO: 81. In some embodiments, the cleavage site comprises a sequence at least 80% identical to any one of SEQ ID NOS: 83-92. In some embodiments, the cleavage site comprises a sequence at least 80% identical to SEQ ID NO: 91.

In some embodiments, the exogenous polynucleotide encodes a pathogen-associated antigen. In some embodiments, the pathogen is a virus, bacteria, fungus, protozoa, or helminth.

In some embodiments, the exogenous polynucleotide encodes a viral structural protein, a viral envelope protein, a viral capsid protein, or a viral nonstructural protein, or any combination thereof. In some embodiments, the exogenous polynucleotide encodes an antigen from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Bimaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); Iridoviridae (e.g., African swine fever virus); Hepatitis C virus; Norwalk virus; and Astrovirus; optionally wherein the exogenous polynucleotide comprises a sequence at least 80% identical to 10 or more nucleobases from the virus. In some embodiments, the exogenous polynucleotide encodes an antigen from a bacteria selected from Helicobacter pylori, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii; optionally wherein the exogenous polynucleotide comprises a sequence at least 80% identical to 10 or more nucleobases from the bacteria. In some embodiments, the exogenous polynucleotide encodes an antigen from a fungi selected from Cryptococcus neoformans, Histoplasma capsulatum, Coccidioides immitis, Blastomyces dermatitidis, Chlamydia trachomatis, and Candida albicans; optionally wherein the exogenous polynucleotide comprises a sequence at least 80% identical to 10 or more nucleobases from the fungi. In some embodiments, the exogenous polynucleotide encodes an antigen from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major; optionally wherein the exogenous polynucleotide comprises a sequence at least 80% identical to 10 or more nucleobases from the protozoa.

In some embodiments, the exogenous polynucleotide comprises a sequence at least 80% identical to any one of SEQ ID NOS: 93-96. In some embodiments, the exogenous polynucleotide encodes an antigen having a sequence at least 80% identical to any one of SEQ ID NOS: 97-100.

In some embodiments, the first exogenous polynucleotide and the polynucleotide encoding the MHC binding peptide are present on two separate nucleic acid strands. In some embodiments, the first exogenous polynucleotide and the polynucleotide encoding the MHC binding peptide are connected.

In some embodiments, the MHC binding peptide is a MHC class I and/or a MHC class II peptide. In some embodiments, the polynucleotide encoding the MHC binding peptide comprises a sequence at least 80% identical to any one of SEQ ID NOS: 113-135. In some embodiments, the polynucleotide encoding the MHC binding peptide comprises a sequence at least 80% identical to SEQ ID NO: 113. In some embodiments, the MHC binding peptide comprises a sequence at least 80% identical to any one of SEQ ID NOS: 136-163. In some embodiments, the MHC binding peptide comprises a sequence at least 80% identical to SEQ ID NO: 136. In some embodiments, the polynucleotide encoding the MHC binding peptide comprises a pathogen-associated sequence.

In some embodiments, the pathogen is a virus, bacteria, fungus, protozoa, or helminth. In some embodiments, the polynucleotide encoding the MHC binding peptide is at least 80% identical to 10 or more nucleobases from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picomaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus. In some embodiments, the polynucleotide encoding the MHC binding peptide is at least 80% identical to 10 or more nucleobases from a bacteria selected from Helicobacter pylori, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii. In some embodiments, the polynucleotide encoding the MHC binding peptide is at least 80% identical to 10 or more nucleobases from a fungi selected from Cryptococcus neoformans, Histoplasma capsulatum, Coccidioides immitis, Blastomyces dermatitidis, Chlamydia trachomatis, and Candida albicans. In some embodiments, the polynucleotide encoding the MHC binding peptide is at least 80% identical to 10 or more nucleobases from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major.

In some embodiments, the MHC binding peptide has a length of 7-20 peptides. In some embodiments, the nucleic acid comprises two or more sequences encoding a MHC binding peptide.

Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 1. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 2. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 3. Any of the nucleic acids may comprise a sequence encoding a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 3. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 4. Any of the nucleic acids may comprise a sequence encoding a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 4. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 5. Any of the nucleic acids may comprise a sequence encoding a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 5. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 6. Any of the nucleic acids may comprise a sequence encoding a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 7. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8. Any of the nucleic acids may comprise a sequence homologous to a sequence of Table 1. Any of the nucleic acids may comprise a sequence homologous to a sequence of Table 2. Any of the nucleic acids may comprise a sequence homologous to a sequence of Table 3. Any of the nucleic acids may comprise a sequence encoding a sequence homologous to a sequence of Table 3. Any of the nucleic acids may comprise a sequence homologous to a sequence of Table 4. Any of the nucleic acids may comprise a sequence encoding a sequence homologous to a sequence of Table 4. Any of the nucleic acids may comprise a sequence homologous to a sequence of Table 5. Any of the nucleic acids may comprise a sequence encoding a sequence homologous to a sequence of Table 5. Any of the nucleic acids may comprise a sequence homologous to a sequence of Table 6. Any of the nucleic acids may comprise a sequence encoding a sequence homologous to a sequence of Table 7. Any of the nucleic acids may comprise a sequence homologous to a sequence of Table 8.

Also provided herein is a peptide translated from a nucleic acid described herein. Also provided herein is a method of expressing the peptide translated from a nucleic acid described herein.

Also provided herein is a method of inducing an immune response in a subject, the method comprising administering to the subject the nucleic acid or the peptide. In some embodiments, the nucleic acid is delivered via a lipid nanoparticle, virus-like particle, or naked.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 is a schematic view of an example mRNA vaccine described herein.

FIG. 2A is a schematic view of an example mRNA vaccine having a booster positioned at the 5′ end of the antigen sequence (*indicates that the signal peptide mRNA sequence is optional for this particular construct).

FIG. 2B is a schematic view of an example mRNA vaccine having a booster positioned at the 3′ end of the antigen sequence (*indicates that the signal peptide mRNA sequence is optional for this particular construct).

FIG. 2C is a schematic view of an example mRNA vaccine comprising multiple antigens and boosters (*indicates that the signal peptide mRNA sequence is optional for this particular construct).

FIG. 3 shows that an embodiment of a mRNA vaccine having flavivirus UTRs for canonical and non-canonical translation of the antigen.

FIGS. 4A-4D are schematic views of example mRNA vaccine constructs.

FIG. 5 shows in vitro transcription of RNA from FIGS. 4A-4D.

FIGS. 6A-6C show that example UTRs described herein promote protein expression of exogenous polynucleotides in cell free and mammalian cell systems.

FIG. 7 shows that example mRNA constructs described herein are resistant to cellular stress.

FIG. 8 shows that example mRNA constructs described herein having flavivirus UTRs are resistant to XRN1 degradation as compared to mRNA constructs having commercial UTRs.

FIGS. 9A-9B show that example UTRs described herein promote protein expression of exogenous polynucleotides in mammalian cells.

FIG. 10 shows that example UTRs described herein promote RBD translation in a mammalian cell system.

FIGS. 11A-11B show that an example mRNA vaccine described herein induces IFN-gamma by antigen-primed CD4+ T cells in vitro.

FIG. 12 shows that example UTRs described herein promote protein translation in vivo.

### DESCRIPTION OF THE INVENTION

In certain aspects, described herein are nucleic acid compositions comprising one or more flavivirus untranslated regions and an exogenous polynucleotide. In certain embodiments, the nucleic acid compositions are mRNA vaccines and the exogenous polynucleotide encodes an antigen. In some cases the exogenous polynucleotide is translated in both healthy and stressed cells, the nucleic acid composition is resistant to RNAse, and/or the nucleic acid is produced in fewer steps than traditional mRNA vaccines.

In certain aspects, described herein are nucleic acid compositions comprising a first sequence encoding an antigen, and a second sequence encoding a MHC binding peptide. In some cases, the nucleic acid composition comprises one or more flavivirus untranslated regions. Further provided are peptide compositions comprising the first antigen and the MHC binding peptide. In some cases, the nucleic acid and/or peptide compositions are vaccine compositions.

### Nucleic Acid Compositions

In one aspect, provided herein are nucleic acid compositions comprising (i) a first exogenous polynucleotide, and (ii) a 5′ untranslated region (5′ UTR) of a first flavivirus and/or a 3′ untranslated region (3′ UTR) of a second flavivirus. Certain exogeneous polynucleotides encode for a first antigen. Non-limiting examples of exogenous polynucleotides and UTRs are described herein.

In another aspect, provided herein are nucleic acid compositions comprising a first sequence encoding a first antigen, and a second sequence encoding a MHC binding peptide.

Further provided are nucleic acid compositions comprising a polynucleotide encoding a first antigen, a 5′ UTR of a first flavivirus and/or a 3′ UTR of a second flavivirus, and a polynucleotide encoding a MHC binding peptide.

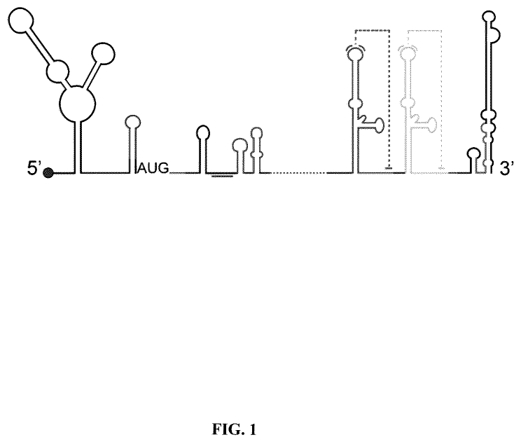

FIG. 1 provides a schematic view of an example nucleic acid composition comprising a flavivirus UTR as described herein. The composition of FIG. 1 comprises a 5′ flavivirus UTR (single line), polynucleotide encoding an antigen (dotted line), and a 3′ flavivirus UTR (single line). In this example, the 5′ UTR provides for canonical and/or alternative translation of the antigen, there is no polyadenylation, and the 3′ UTR is endonuclease resistant (e.g., to an RNAse such as XRN-1).

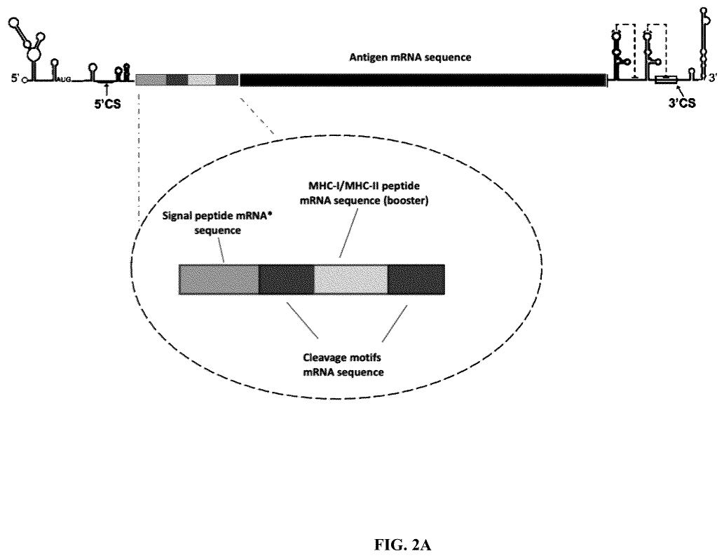

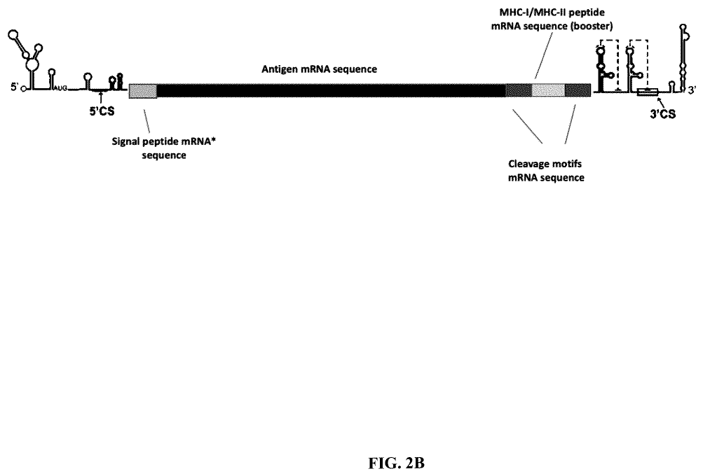

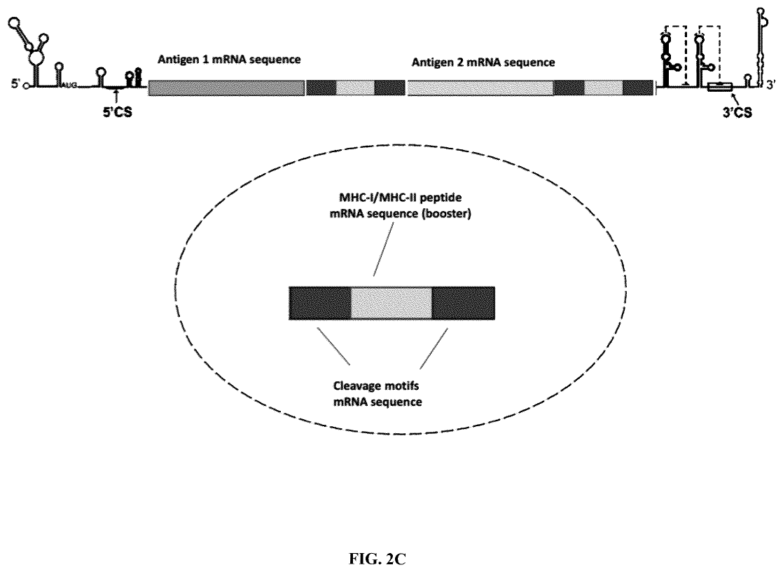

FIG. 2A provides a schematic view of an example nucleic acid composition comprising a booster positioned at the 5′ end of the polynucleotide encoding the antigen. The composition of FIG. 2A comprises a 5′ flavivirus UTR, a polynucleotide encoding a signal peptide, a polynucleotide encoding a MHC-I/MHC-II binding peptide (sometimes referred to as a booster), polynucleotides encoding cleavage sites (cleavage motifs), a polynucleotide encoding an antigen (antigen mRNA sequence), and a 3′ flavivirus UTR. In this example, the signal peptide is optional.

FIG. 2B provides a schematic view of an example mRNA vaccine having a booster positioned at the 3′ end of the polynucleotide encoding the antigen. The composition of FIG. 2B comprises a 5′ flavivirus UTR, a polynucleotide encoding a signal peptide, a polynucleotide encoding an antigen (antigen mRNA sequence), a polynucleotide encoding a MHC-I/MHC-II binding peptide (sometimes referred to as a booster), polynucleotides encoding cleavage sites (cleavage motifs), and a 3′ flavivirus UTR. In this example, the signal peptide is optional.

FIG. 2C provides a schematic view of an example mRNA vaccine having multiple sequences encoding antigens and boosters. The composition of FIG. 2C comprises a 5′ flavivirus UTR, a polynucleotide encoding a first antigen (antigen 1 mRNA sequence), polynucleotides encoding cleavage sites (cleavage motifs), a polynucleotide encoding a MHC-I/MHC-II binding peptide 1 (booster 1), a polynucleotide encoding a second antigen (antigen 2 mRNA sequence), a polynucleotide encoding a MHC-1/MHC-II binding peptide 2 (booster 2), and a 3′ flavivirus UTR. In this example, the signal peptide is optional. The antigens can be the same or different. The MHC-I/MHC-II binding peptides (boosters) can be the same or different.

In some embodiments, mRNA vaccines having flavivirus UTRs are capable of canonical (Cap-1 dependent) and non-canonical (Cap-1 independent) translation of the antigen. For instance, as determined via a method provided in Example 2.

Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 1. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 2. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 3. Any of the nucleic acids may comprise a sequence encoding a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 3. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 4. Any of the nucleic acids may comprise a sequence encoding a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 4. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 5. Any of the nucleic acids may comprise a sequence encoding a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 5. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 6. Any of the nucleic acids may comprise a sequence encoding a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 7. Any of the nucleic acids may comprise a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence of Table 8. Any of the nucleic acids may comprise a sequence homologous to a sequence of Table 1. Any of the nucleic acids may comprise a sequence homologous to a sequence of Table 2. Any of the nucleic acids may comprise a sequence homologous to a sequence of Table 3. Any of the nucleic acids may comprise a sequence encoding a sequence homologous to a sequence of Table 3. Any of the nucleic acids may comprise a sequence homologous to a sequence of Table 4. Any of the nucleic acids may comprise a sequence encoding a sequence homologous to a sequence of Table 4. Any of the nucleic acids may comprise a sequence homologous to a sequence of Table 5. Any of the nucleic acids may comprise a sequence encoding a sequence homologous to a sequence of Table 5. Any of the nucleic acids may comprise a sequence homologous to a sequence of Table 6. Any of the nucleic acids may comprise a sequence encoding a sequence homologous to a sequence of Table 7. Any of the nucleic acids may comprise a sequence homologous to a sequence of Table 8.

### Untranslated Region

Certain nucleic acid compositions herein comprise an untranslated region (UTR) of a flavivirus. In certain aspects, a UTR refers to an untranslated terminal mRNA region surrounding the protein coding region of the mRNA molecule. In some embodiments, a UTR may be located upstream (5′) from the start codon of an expression sequence described herein. In some embodiments, a UTR may be located downstream (3′) from the stop codon of an expression sequence described herein. UTRs play an important role in the stability and translation of mRNA molecules in mammalian cells. The use of a UTR of a flavivirus described herein provides several beneficial features for mRNA vaccine applications. In some aspects, nucleic acid compositions comprising a UTR of a flavivirus can initiate canonical and non-canonical protein synthesis in healthy cells as well as during cellular stress responses. Cells undergo a wide range of molecular changes in response to environmental stressors, including but not limited to, extreme temperature, exposure to toxins or microorganisms, mechanical damages, tumors, and/or nutrient starvation. In some aspects, by using a UTR of a flavivirus, a nucleic acid composition herein can initiate the mRNA translation process even under the condition of stress. In some aspects, nucleic acid compositions comprising a UTR of a flavivirus described herein are resistant to degradation by RNAses at the 3′ UTR, therefore the stability of mRNA vaccines can be significantly increased. Moreover, in some aspects, nucleic acid compositions comprising a UTR of a flavivirus described herein do not require polyadenylation at the 3′ UTR, therefore production time and costs can be reduced.

Provided herein, in certain embodiments, are nucleic acid compositions comprising a 5′ UTR of a first flavivirus and/or a 3′ UTR of a second flavivirus. In some embodiments, the nucleic acid compositions comprises the 5′ UTR or the first flavivirus and the 3′ UTR of the second flavivirus. In some embodiments, the first flavivirus and the second flavivirus are the same flavivirus. In other embodiments, the first flavivirus and the second flavivirus are different flaviviruses.

Provided herein, in certain embodiments, are nucleic acid compositions comprising a 5′ UTR of a first flavivirus. In some embodiments, the first flavivirus is a tick-borne flavivirus (TBFV), a mosquito-borne flavivirus (MBFV), an insect-specific flavivirus (ISFV), no-known vector flavivirus (NKFV), or a non-classified flavivirus (NCFV). In some embodiments, the first flavivirus is a dengue virus (DENV), West Nile virus (WNV), Japanese encephalitis virus (JEV), yellow fever virus (YFV), Zika virus (ZIKV), tick-born encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV).

In some embodiments, the first flavivirus is a dengue virus (DENV). Examples of the dengue virus (DENV) include, without limitation, a dengue virus serotype 1 (DENV-1), a dengue virus serotype 2 (DENV-2), a dengue virus serotype 3 (DENV-3), and a dengue virus serotype 4 (DENV-4).

In some embodiments, the 5′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 1-36. In some embodiments, a 5′ UTR comprises a sequence at least 80% identical to at least 50, 60, 70, 80, 90, or 100 contiguous bases of a virus of Table 1.

In some embodiments, the 5′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to SEQ ID NO: 36. In some embodiments, a 5′ UTR comprises a sequence at least 80% identical to at least 50, 60, 70, 80, 90, or 100 contiguous bases of a Dengue virus 4.

In some embodiments, the 5′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the 5′ UTR of SEQ ID NO: 164. In some embodiments, a 5′ UTR comprises a sequence at least 80% identical to at least 50, 60, 70, 80, 90, or 100 contiguous bases of the 5′ UTR of SEQ ID NO: 164. In some embodiments, the 5′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the 5′ UTR of SEQ ID NO: 166. In some embodiments, a 5′ UTR comprises a sequence at least 80% identical to at least 50, 60, 70, 80, 90, or 100 contiguous bases of the 5′ UTR of SEQ ID NO: 166. In some embodiments, the 5′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the 5′ UTR of SEQ ID NO: 175. In some embodiments, a 5′ UTR comprises a sequence at least 80% identical to at least 30, 40, or 50 contiguous bases of the 5′ UTR of SEQ ID NO: 175.

In some embodiments, the 5′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the first 161 bases of SEQ ID NO: 164. In some embodiments, a 5′ UTR comprises a sequence at least 80% identical to at least 50, 60, 70, 80, 90, or 100 contiguous bases of the first 161 bases of SEQ ID NO: 164. In some embodiments, the 5′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the first 161 bases of SEQ ID NO: 166. In some embodiments, a 5′ UTR comprises a sequence at least 80% identical to at least 50, 60, 70, 80, 90, or 100 contiguous bases of the first 161 bases of SEQ ID NO: 166. In some embodiments, the 5′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the first 54 bases of SEQ ID NO: 175. In some embodiments, a 5′ UTR comprises a sequence at least 80% identical to at least 30, 40, or 50 contiguous bases of the first 54 bases of SEQ ID NO: 175.

TABLE 1
EXAMPLE 5′ UTR SEQUENCES

| | SEQ | |
|---|---|---|
| | ID | |
| Flavivirus | NO | Sequence |
| Dengue virus 1 | 1 | AGTTGTTAGTCTACGTGGACCGACAAGAACAGTTTCGAATCGGAAGC |
| (GenBank: | | TTGCTTAACGTAGTTCTAACAGTTTTTTATTAGAGAGCAGATCTCTG |
| KC692498.1) | | |
| Dengue virus 2 | 2 | AGTTGTTAGTCTACGTGGACCGACAAAGACAGATTCTTTGAGGGAGC |
| (GenBank: | | TAAGCTCAACGTAGTTCTAACAGTTTTTTAATTAGAGAGCAGATCTCT |
| MW577822.1) | | G |
| Dengue virus 3 | 3 | AGTTGTTTATCTACGTGGACCGACAAGAACAGTTTCGACTCGGAAGC |
| (GenBank: | | TTGCTTAACGTAGTGCTGACAGTTTTTTATTAGAGAGCAGATCTCTG |
| MN018383.1) | | |
| Dengue virus 4 | 4 | AGTTGTTAGTCTGTGTGGACCGACAAGGACAGTTCCAAATCGGAAGC |
| (GenBank: | | TTGCTTAACACAGTTCTAACAGTTTATTTAGATAGAGAGCAGATCTCT |
| MN018390.1) | | GGAAAA |
| Dengue virus 4 | 5 | AGTTGTTAGTCTGTGTGGACCGACAAGGACAGTTCTAAATCGGAAGC |
| | | TTGCTTAACGCAGTTCTAACAGTTTGTTTAGATAGAGAGCAGATCTCT |
| | | GGAAAA |
| West Nile virus | 6 | AGTAGTTCGCCTGTGTGAGCTGACAAACTTAGTAGTGTTTGTGAGGAT |
| (GenBank: | | TAACAACAATTAACACAGTGCGAGCTGTTTCTTAGCACGAAGATCTC |
| LC318700.1) | | G |
| Japanese | 7 | AGAAGTTTATCTGTGTGAACTTCTTGGCTTAGTATTGTTGAGAAGAAT |
| encephalitis | | CGAGAGATTAGTGCAGTTTAAACAGTTTTTTAGAACGGAAGATAACC |
| virus (GenBank: | | |
| AF080251.1) | | |
| Yellow fever | 8 | AGTAAATCCTGTGTGCTAATTGAGGTGCATTGGTCTGCAAATCGAGTT |
| virus (GenBank: | | GCTAGGCAATAAACACATTTGGATTAATTTTAATCGTTCGTTGAGCGA |
| MT107250.1) | | TTAGCAGAGAACTGACCAGAAC |
| Yellow fever | 9 | GTGCTAATTGAGGTGCATTGGTCTGCAAATCGAGTTGCTAGGCAATA |
| virus (GenBank: | | AACACATTTGGATTAATTTTAATCGTTCGTTGAGCGATTAGCAGAGAA |
| MT956629.1) | | CTGACCAGAAC |
| Zika virus | 10 | GTGTGAATCAGACTGCGACAGTTCGAGTTTGAAGCGAAAGCTAGCAA |
| (GenBank | | CAGTATCAACAGGTTTTATTTTGGATTTGGAAACGAGAGTTTCTGGTC |
| MH882538.1) | | |
| Tick-borne | 11 | AGATTTTCTTGCACGTGCATGCGTTTGCTTCGGATAGCATTAGCAGCG |
| encephalitis | | GCAGGTTCGGAAGAGACATTGTCTCGTTTCTACTAGTCGTGAACGTGT |
| virus (GenBank: | | TGAGAAAAAGACAGCTTAGGAGAACAAGAGCTGGGG |
| MH645619.1) | | |
| Usutu virus | 12 | AGTCGTTCGTCTGCGTGAGCTCTACTACTTAGTATTGTTTTTGGAGGA |
| (GenBank: | | TCGTGAGATTAACACAGTGCCGGCAGTTTCTTTGAGCGTTGATTTTCA |
| AY453411.1) | | |
| Border disease | 13 | GTATACGGGAGTAGCTCATGCCCGTATACAAAATTGGATATTCCAAA |
| virus (NCBI | | ACTCGATTGGGTTAGGGAGCCCTCCTAGCGACGGCCGAACCGTGTTA |
| Reference | | ACCATACACGTAGTAGGACTAGCAGACGGGAGGACTAGCCATCGTGG |
| Sequence: | | TGAGATCCCTGAGCAGTCTAAATCCTGAGTACAGGATAGTCGTCAGT |
| NC_003679.1) | | AGTTCAACGCAGGCACGGTTCTGCCTTGAGATGCTACGTGGACGAGG |
| | | GCATGCCCAAGACTTGCTTTAATCTCGGCGGGGGTCGCCGAGGTGAA |
| | | AACACCTAACGGTGTTGGGGTTACAGCCTGATAGGGTGCTGCAGAGG |
| | | CCCACGAATAGGCTAGTATAAAAATCTCTGCTGTACATGGCAC |
| Bovine viral | 14 | GTATACGAGAATTAGAAAAGGCACTCGTATACGTATTGGGCAATTAA |
| diarrhea virus | | AAATAATAATTAGGCCTAGGGAACAAATCCCTCTCAGCGAAGGCCGA |
| (NCBI | | AAAGAGGCTAGCCATGCCCTTAGTAGGACTAGCATAATGAGGGGGGT |
| Reference | | AGCAACAGTGGTGAGTTCGTTGGATGGCTTAAGCCCTGAGTACAGGG |
| Sequence: | | TAGTCGTCAGTGGTTCGACGCCTTGGAATAAAGGTCTCGAGATGCCA |
| NC_001461.1) | | CGTGGACGAGGGCATGCCCAAAGCACATCTTAACCTGAGCGGGGGTC |
| | | GCCCAGGTAAAAGCAGTTTTAACCGACTGTTACGAATACAGCCTGAT |
| | | AGGGTGCTGCAGAGGCCCACTGTATTGCTACTAAAAATCTCTGCTGTA |
| | | CATGGCAC |
| Bussuquara | 15 | AGTATTTCTTCTGCGTGAGACCATTGCGACAGTTCGTACCGGTGAGTT |
| virus (NCBI | | TTGACTTAACGCAGTGAGAAAAGTTTTCGAGGAAAGACGAGAAGCGA |
| Reference | | ATTCTCTGA |
| Sequence: | | |
| NC_009026.2) | | |
| Cell fusing | 16 | ACTTCGGCTTAGCTACACCACAGTTTTGGTTACGCTTATATTTTCAAA |
| agent virus | | GCTTAAGTTGTTTTTAATTTTTGCCGAGAGACCGTGAGGTTGAACCCG |
| (NCBI | | GCAAGGA |
| Reference | | |
| Sequence: | | |
| NC_001564.2) | | |
| Classical swine | 17 | GTATACGAGGTTAGTTCATTCTCGTATGCATGATTGGACAAATCAAAA |
| fever virus | | TTTCAATTTGGTTCAGGGCCTCCCTCCAGCGACGGCCGAACTGGGCTA |
| (NCBI | | GCCATGCCCACAGTAGGACTAGCAAACGGAGGGACTAGCCGTAGTGG |
| Reference | | CGAGCTCCCTGGGTGGTCTAAGTCCTGAGTACAGGACAGTCGTCAGT |
| Sequence: | | AGTTCGACGTGAGCAGAAGCCCACCTCGAGATGCTATGTGGACGAGG |
| NC_002657.1) | | GCATGCCCAAGACACACCTTAACCCTAGCGGGGGTCGCTAGGGTGAA |
| | | ATCACACCACGTGATGGGAGTACGACCTGATAGGGCGCTGCAGAGGC |
| | | CCACTATTAGGCTAGTATAAAAATCTCTGCTGTACATGGCAC |
| Culex flavivirus | 18 | AGTTTTTAAAAACTTCGGCTTGGTTACACCGCAGATTGGTTACACCTA |
| (NCBI | | CACAAGGCTTGAGTTGTTTATAATAGTCGTTTTTCTCGCAGAA |
| Reference | | |
| Sequence: | | |
| NC_008604.2) | | |
| Entebbe bat | 19 | AGTAAATTTTGCGTGCTAGTCGCTTGGCGTTAGTCCGTGAAGTGAGTT |
| virus (NCBI | | TTTGGATACATTGTACCAGAGATTAACACGTTGAAATTATTTCTGAAA |
| Reference | | ACAGAAAATCAGAATCAGACGCG |
| Sequence: | | |
| NC_008718.1) | | |
| Pestivirus | 20 | GTATACGAGTTTAGCTCAATCCTCGTATACAATATTGGGCGTCACCAA |
| giraffe-1 (NCBI | | ATATAGATTTGGCATAGGCAACACCCCGATGCGAAGGCCGAAAAGGG |
| Reference | | CTAACCATGCCCTTAGTAGGACTAGCAAAAAATCGGGGACTAGCCCA |
| Sequence: | | GGTGGTGAGCTTCCTGGATGACCGAAGCCCTGAGTACAGGGCAGTCG |
| NC_003678.1) | | TCAACAGTTCAACACGCAGAATAGGTTTGCGTCTTGATATGCTGTGTG |
| | | GACGAGGGCATGCCCACGGTACATCTTAACCTATCCGGGGGTCGGAT |
| | | AGGCGAAAGTCCAGTATTGGACTGGGAGTACAGCCTGATAGGGTGTT |
| | | GCAGAGACCCATCTGATAGGCTAGTATAAAAAACTCTGCTGTACATG |
| | | GCAC |
| Hepatitis C virus | 21 | GCCAGCCCCCTGATGGGGGCGACACTCCACCATGAATCACTCCCCTG |
| (GenBank: | | TGAGGAACTACTGTCTTCACGCAGAAAGCGTCTAGCCATGGCGTTAG |
| AF009606.1) | | TATGAGTGTCGTGCAGCCTCCAGGACCCCCCCTCCCGGGAGAGCCAT |
| | | AGTGGTCTGCGGAACCGGTGAGTACACCGGAATTGCCAGGACGACCG |
| | | GGTCCTTTCTTGGATAAACCCGCTCAATGCCTGGAGATTTGGGCGTGC |
| | | CCCCGCAAGACTGCTAGCCGAGTAGTGTTGGGTCGCGAAAGGCCTTG |
| | | TGGTACTGCCTGATAGGGTGCTTGCGAGTGCCCCGGGAGGTCTCGTA |
| | | GACCGTGCACC |
| Hepatitis GB | 22 | ACCACAAACACTCCAGTTTGTTACACTCCGCTAGGAATGCTCCTGGAG |
| virus B (NCBI | | CACCCCCCCTAGCAGGGCGTGGGGGATTTCCCCTGCCCGTCTGCAGA |
| Reference | | AGGGTGGAGCCAACCACCTTAGTATGTAGGCGGCGGGACTCATGACG |
| Sequence: | | CTCGCGTGATGACAAGCGCCAAGCTTGACTTGGATGGCCCTGATGGG |
| NC_001655.1) | | CGTTCATGGGTTCGGTGGTGGTGGCGCTTTAGGCAGCCTCCACGCCCA |
| | | CCACCTCCCAGATAGAGCGGCGGCACTGTAGGGAAGACCGGGGACC |
| | | GGTCACTACCAAGGACGCAGACCTCTTTTTGAGTATCACGCCTCCGGA |
| | | AGTAGTTGGGCAAGCCCACCTATATGTGTTGGGATGGTTGGGGTTAG |
| | | CCATCCATACCGTACTGCCTGATAGGGTCCTTGCGAGGGGATCTGGG |
| | | AGTCTCGTAGACCGTAGCAC |
| GB virus | 23 | ACGTGGGGGAGTTGATCCCCCCCCCCCGGCACTGGGTGCAAGCCCCA |
| C/Hepatitis G | | GAAACCGACGCCTATCTAAGTAGACGCAATGACTCGGCGCCGACTCG |
| virus (NCBI | | GCGACCGGCCAAAAGGTGGTGGATGGGTGATGACAGGGTTGGTAGGT |
| Reference | | CGTAAATCCCGGTCACCTTGGTAGCCACTATAGGTGGGTCTTAAGAG |
| Sequence: | | AAGGTTAAGATTCCTCTTGTGCCTGCGGCGAGACCGCGCACGGTCCA |
| NC_001710.1) | | CAGGTGTTGGCCCTACCGGTGGGAATAAGGGCCCGACGTCAGGCTCG |
| | | TCGTTAAACCGAGCCCGTTACCCACCTGGGCAAACGACGCCCACGTA |
| | | CGGTCCACGTCGCCCTTCAATGTCTCTCTTGACCAATAGGCGTAGCCG |
| | | GCGAGTTGACAAGGACCAGTGGGGGCCGGGGGCTTGGAGAGGGACT |
| | | CCAAGTCCCGCCCTTCCCGGTGGGCCGGGAAATGC |
| Ilheus virus | 24 | AGAAATTCACCTGTGTGAATTTCACTAACCGTTTTAGTGGAGAGAACT |
| (NCBI | | TTTGTTTAACACAGTCTGAATAGTTTTTTAGCAAGGGATTTCCC |
| Reference | | |
| Sequence: | | |
| NC_009028.2) | | |
| Kamiti River | 25 | AGTTTTTGAAAACTTCTGTGAATGTTTATATCCTTAGTCGGATCGAGC |
| virus (NCBI | | TAAATTTTAAATCAAAGGAGTTGTTCGGAAAAGTGACCTTGGTTCGTT |
| Reference | | |
| Sequence: | | |
| NC_005064.1) | | |
| Kokobera virus | 26 | AGATGTTCACCTGTGTGAACTAACCAGACAGATCGAAGTTAGGTGAT |
| (NCBI | | TACATAACACAGTGTGAACAAGTTTTTTGAACAGCA |
| Reference | | |
| Sequence: | | |
| NC_009029.2) | | |
| Langat virus | 27 | AGATTTTCTTGCGCGTGCATGCGTGTGCTTCAGACAGCCCAGGCAGCG |
| (NCBI | | ACTGTGATTGTGGATATTCTTTCTGCAAGTTTTGTCGTGAACGTGTTG |
| Reference | | AGAAAAAGACAGCTTAGGAGAACAAGAGCTGGGA |
| Sequence: | | |
| NC_003690.1) | | |
| Louping ill virus | 28 | AGATTTTCTTGCACGTGCGATAGCTTCGGACAGCTTTGGCAGCGGCAG |
| (NCBI | | GTTTGAAAGAGACATTTTTTTTTCTTTCATCAGCCGTGAACGTGTTGA |
| Reference | | GAAAAAGACAGCTTAGGAGAACAAGAGCTGGGG |
| Sequence: | | |
| NC_001809.1) | | |
| Modoc virus | 29 | AGTTGATCCTGCCAGCGGTGGGTCGCTACTGTTTCGCGAACCAGTCGT |
| (NCBI | | TTTGACAGTTGGTTGGGATCAAATTTGTTCTGTGCGCGTCACGCCACT |
| Reference | | TTTTGTGGCGGGA |
| Sequence: | | |
| NC_003635.1) | | |
| Montana myotis | 30 | AGTTGGTTTTGCCGGCTACAACGATCCTCCGTAGGAAGCGTTGGTGTC |
| leukoencephalitis | | TTGGACATTGCCGAGTTGAAACCTTGGTTTCCGGCTGGAAACCACGTC |
| virus (NCBI | | GCTCTTCGTCAA |
| Reference | | |
| Sequence: | | |
| NC_004119.1 | | |
| Murray Valley | 31 | AGACGTTCATCTGCGTGAGCTTCCGATCTCAGTATTGTTTGGAAGGAT |
| encephalitis | | CATTGATTAACGCGGTTTGAACAGTTTTTTGGAGCTTTTGATTTCAA |
| virus (NCBI | | |
| Reference | | |
| Sequence: | | |
| NC_000943.1) | | |
| Omsk | 32 | AGATTTTCTTGCACGTGCGTGCGCTTGCTTCAGACAGCAATAGCAGCG |
| hemorrhagic | | GCAGGGTTGGTGGAAGGAATTGCCCGCATCAGCCAGTCGTGAACGTG |
| fever virus | | TTGAGAAAAAGACAGCTTAGGAGAACAAGAGCTGGGG |
| (NCBI | | |
| Reference | | |
| Sequence: | | |
| NC_005062.1) | | |
| Powassan virus | 33 | AGATTTTCTTGCACGTGTGTGCGGGTGCTTTAGTCAGTGTCCGCAGCG |
| (NCBI | | TTCTGTTGAACGTGAGTGTGTTGAGAAAAAGACAGCTTAGGAGAACA |
| Reference | | AGAGCTGGGAGTGGTT |
| Sequence: | | |
| NC_003687.1) | | |
| Sepik virus | 34 | AGTATATTCTGCGTGCTAATCGTTCAACGTTAGTCCGTGGAGTGAGCT |
| (NCBI | | TCTGTTAAGTTGTTAACACGTTTGAATAATTTCTACTGAAAGGGTAGA |
| Reference | | GAAAAGGAGTTTTGCTTCTC |
| Sequence: | | |
| NC_008719.1) | | |
| Yokose virus | 35 | AGTAAATTTTGCGTGCTAGTCGCTGAGCGTCAGACCGCAAAGTGAGT |
| (NCBI | | TTTTAGTGATCTAAAGTGAGGAGTTATTCTTACTGTCATCAAACACTA |
| Reference | | CAAATAAACACGTTGAAATTATTTCCGGAAGAACAACTGTCCGGAAT |
| Sequence: | | CAAAGACG |
| NC_005039.1) | | |
| Dengue virus 4 | 36 | AGTTGTTAGTCTGTGTGGACCGACAAGGACAGTTCTAAATCGGAAGC |
| | | TTGCTTAACGCAGTTCTAACAGTTTGTTTAGATAGAGAGCAGATCTCT |
| | | GGAAAAATGAACCAACGAAAAAGGGTGGTTAGACCACCTTTCAATAT |
| | | GCTGAAACGCGAGAGAAAC |

In some embodiments, a 5′ UTR is provided as a flanking region to nucleic acids (e.g., mRNAs). In some embodiments, a 5′ UTR is homologous or heterologous to the coding region found in nucleic acids. In some embodiments, multiple 5′ UTRs are included in the flanking region. In some embodiments, the multiple 5′ UTRs are present from the same or different sequences. In some embodiments, any portion of the flanking regions, including none, are codon optimized. In some embodiments, codon optimization is a method to match codon frequencies in target and host organisms to ensure proper folding, customize transcriptional and translational control regions, insert or remove protein trafficking sequences, remove/add post translational modification sites in encoded protein (e.g. glycosylation sites), add, remove or shuffle protein domains, bias GC content to increase mRNA stability or reduce secondary structures, minimize tandem repeat codons or base runs that may impair gene construction or expression, insert or delete restriction sites, or modify ribosome binding sites and mRNA degradation sites. Examples of codon optimization tools, algorithms and services including, but not limited to, services from GeneArt (Life Technologies), DNA2.0 (Menlo Park Calif) and/or proprietary methods.

In some embodiments, a 5′ UTR sequence includes at least one translation enhancer element. In some embodiments, the translational enhancer element is a sequence that increases the amount of polypeptide or protein produced from a polynucleotide. In some embodiments, the translation enhancer element is located between the transcription promoter and the start codon. In some embodiments, a translation enhancer element is located in the 5′ UTR of a nucleic acid (e.g., mRNA) undergoing cap-dependent or cap-independent translation.

In some embodiments, a 5′ UTR comprises the stem loop A of the 5′ UTR of the first flavivirus. In some embodiments, a 5′ UTR comprises the stem loop B of the 5′ UTR of the first flavivirus. In some embodiments, a 5′ UTR comprises the 5′ ATG of the first flavivirus. In some embodiments, a 5′ UTR comprises the capsid-coding region hairpin element (cHP) of the first flavivirus. As a non-limiting example, SEQ ID NO: 36 comprises a cHP. In some embodiments, a 5′ UTR comprises the 5′ conserved sequence of the first flavivirus. In some embodiments, a 5′ UTR does not comprise a 5′ cap modification. In other embodiments, a 5′ UTR comprises a 5′ cap modification.

In some embodiments, a 5′ UTR has a length of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more than 500 bases. In some embodiments, a 5′ UTR has a length of about 80-200, 80-180, 80-160, 80-140, 80-120, 80-100, 100-200, 100-180, 100-160, 100-140, 100-120, 120-200, 120-180, 120-160, 120-140, 140-200, 160-180, or 180-200 bases.

In some embodiments, a 5′ UTR is a 5′ UTR of a flavivirus, wherein the flavivirus is not a tick-borne flavivirus (TBFV), a mosquito-borne flavivirus (MBFV), an insect-specific flavivirus (ISFV), no-known vector flavivirus (NKFV), or a non-classified flavivirus (NCFV). In some embodiments, a 5′ UTR is a 5′ UTR of a flavivirus, wherein the flavivirus is not a dengue virus (DENV), West Nile virus (WNV), Japanese encephalitis virus (JEV), yellow fever virus (YFV), Zika virus (ZIKV), tick-born encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV). In some cases, the flavivirus is not a West Nile virus (WNV). In some cases, the flavivirus is not a Japanese encephalitis virus (JEV). In some cases, the flavivirus is not a yellow fever virus (YFV). In some cases, the flavivirus is not a Zika virus (ZIKV). In some cases, the flavivirus is not a tick-born encephalitis virus (TBEV). In some cases, the flavivirus is not a Usutu virus (USUV). In some cases, the flavivirus is not a Apoi virus (APOIV). In some cases, the flavivirus is not a border disease virus (BDV). In some cases, the flavivirus is not a bovine viral diarrhea virus (BVDV). In some cases, the flavivirus is not a Bussuquara virus (BSQV). In some cases, the flavivirus is not a cell fusing agent virus (CFAV). In some cases, the flavivirus is not a classical swine fever virus (CSFV). In some cases, the flavivirus is not a Culex flavivirus (CxFV). In some cases, the flavivirus is not a Entebbe bat virus (ENTV). In some cases, the flavivirus is not a pestivirus giraffe-1. In some cases, the flavivirus is not a hepatitis C virus (HCV). In some cases, the flavivirus is not a hepatitis GB virus B (GBV-B). In some cases, the flavivirus is not a GB virus C/hepatitis G virus (GBV-C). In some cases, the flavivirus is not a Ilheus virus (ILHV). In some cases, the flavivirus is not a Kamiti river virus (KRV). In some cases, the flavivirus is not a Kokobera virus (KOKV). In some cases, the flavivirus is not a Langat virus (LGTV). In some cases, the flavivirus is not a Louping ill virus (LIV). In some cases, the flavivirus is not a Modoc virus (MODV). In some cases, the flavivirus is not a Montana myotis leukoencephalitis virus (MMLV). In some cases, the flavivirus is not a Murray Valley encephalitis virus (MVEV). In some cases, the flavivirus is not a Omsk hemorrhagic fever virus (OHFV). In some cases, the flavivirus is not a Powassan virus (POWV). In some cases, the flavivirus is not a Rio Bravo virus (RBV). In some cases, the flavivirus is not a Sepik virus (SEPV). In some cases, the flavivirus is not a Tamana bat virus (TABV). In some cases, the flavivirus is not a Yokose virus (YOKV).

Provided herein, in certain embodiments, are nucleic acid compositions comprising a 3′ UTR of a second flavivirus. In some embodiments, the second flavivirus is a tick-borne flavivirus (TBFV), a mosquito-borne flavivirus (MBFV), an insect-specific flavivirus (ISFV), no-known vector flavivirus (NKFV), or a non-classified flavivirus (NCFV). In some embodiments, the second flavivirus is a dengue virus (DENV), West Nile virus (WNV), Japanese encephalitis virus (JEV), yellow fever virus (YFV), Zika virus (ZIKV), tick-bom encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV).

In some embodiments, the second flavivirus is a dengue virus (DENV). Examples of the dengue virus (DENV) include, without limitation, a dengue virus serotype 1 (DENV-1), a dengue virus serotype 2 (DENV-2), a dengue virus serotype 3 (DENV-3), and a dengue virus serotype 4 (DENV-4).

In some embodiments, a 3′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 37-70. In some embodiments, a 3′ UTR comprises a sequence at least 80% identical to at least 50, 60, 70, 80, 90, or 100 contiguous bases of a virus of Table 2.

In some embodiments, the 3′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to SEQ ID NO: 40. In some embodiments, a 3′ UTR comprises a sequence at least 80% identical to at least 50, 60, 70, 80, 90, or 100 contiguous bases of a Dengue virus 4.

In some embodiments, the 3′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the 3′ UTR of SEQ ID NO: 164. In some embodiments, a 3′ UTR comprises a sequence at least 80% identical to at least 50, 60, 70, 80, 90, or 100 contiguous bases of the 3′ UTR of SEQ ID NO: 164. In some embodiments, the 3′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the last 384 bases of SEQ ID NO: 164. In some embodiments, a 3′ UTR comprises a sequence at least 80% identical to at least 50, 60, 70, 80, 90, or 100 contiguous bases of the last 384 bases of SEQ ID NO: 164. In some embodiments, the 3′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the 3′ UTR of SEQ ID NO: 175. In some embodiments, a 3′ UTR comprises a sequence at least 80% identical to at least 50, 60, 70, 80, 90, or 100 contiguous bases of the 3′ UTR of SEQ ID NO: 175. In some embodiments, the 3′ UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the last 296 underlined bases of SEQ ID NO: 175. In some embodiments, a 3′ UTR comprises a sequence at least 80% identical to at least 50, 60, 70, 80, 90, or 100 contiguous bases of the last 296 underlined bases of SEQ ID NO: 175.

TABLE 2
Example 3′ UTR sequences

| | SEQ | |
|---|---|---|
| | ID | |
| Flavivirus | NO | Sequence |
| Dengue virus 1 | 37 | GTCAACACACTCATGAAATAAAGGAAAATAGAAGATCAAACAAAGT |
| (GenBank: | | GAGAAGTCAGGCCAGATTAAGCCATAGTACGGAAAGAGCTATGCTG |
| KC692498.1) | | CCTGTGAGCCCCGTCCAAGGACGTAAAATGAAGTCAGGCCGAAAGC |
| | | CACGGATTGAGCAAGCCGTGCTGCCTGTGGCTCCATCGTGGGGATGT |
| | | AAAAACCCGGGAGGCTGCAACCCATGGAAGCTGTACGCATGGGGTA |
| | | GCAGACTAGTGGTTAGAGGAGACCCCTCCCTAGACATAACGCAGCA |
| | | GGGGGCCCAACACCAGGGGAAGCTGTACCTTGGTGGTAAGGACTA |
| | | GAGGTTAGAGGAGACCCCCCGCACAACAACAAACAGCATATTGACG |
| | | CTGGGAGAGACCAGAGATCCTGCTGTCTCTACAGCATCATTCCAGGC |
| | | ACAGAACGCCAGAAAATGGAATGGTGCTGTTGAATCAACAGGTTCT |
| Dengue virus 2 | 38 | AAGGCGAAACTAACATGAAACAAGGCTGAAAGTCAGGTCGGATTAA |
| (GenBank: | | GCCATAGTACGGGAAAAACTATGCTACCTGTGAGCCCCGTCCAAGG |
| KC692498.1) | | ACGTAAAAAGAAGTCAGGCCATCACAAAAATGCCACAGCTTGAGCA |
| | | AACTGTGCAGCCTGTAGCTCCACCTGAGGAGGTGTAAAAAACCCGG |
| | | GAGGCCACAAACCATGGAAGCTGTACGCATGGCGTAGTGGACTAGC |
| | | GGTTAGAGGAGACCCCTCCCTTACAAATCGCAGCAACAACGGGGGC |
| | | CCAAGGTGAGATGAAGCTGTAGTCTCACTGGAAGGACTAGAGGTTA |
| | | GAGGAGACCCCCCCAAAACAAAAAACAGCATATTGACGCTGGGAAA |
| | | GACCAGAGATCCTGCTGTCTCCTCAGCATCATTCCAGGCACAGAACG |
| | | CCAGAAAATGGAATGGTGCTGTTGAATCAACAGGTTCT |
| Dengue virus 3 | 39 | ACACAGGAAGTGAAAAAGAGGCAAACTGTCAGGCCACTTTAAGCCA |
| (GenBank: | | CAGTACGGAAGAAGCTGTGCAGCCTGTGAGCCCCGTCCAAGGACGT |
| MN018383.1) | | TAAAAGAAGAAGTCAGGCCCAAAAGCCACGGTTTGAGCAAACCGTG |
| | | CTGCCTGTAGCTCCGTCGTGGGGACGTAAAAACCTGGGAGGCTGCA |
| | | AACTGTGGAAGCTGTACGCACGGTGTAGCAGACTAGCGGTTAGAGG |
| | | AGACCCCTCCCATGACACAACGCAGCAGCGGGGCCCGAGCACTGAG |
| | | GGAAGCTGTACCTCTTTGCAAAGGACTAGAGGTTAGAGGAGACCCC |
| | | CCGCAAACAAAAACAGCATATTGACGCTGGGAGAGACCAGAGATCC |
| | | TGCTGTCTCCTCAGCATCATTCCAGGCACAGAACGCCAGAAAATGGA |
| | | ATGGTGCTGTTGAATCAACAGGTTCT |
| Dengue virus 4 | 40 | TTACCAACAACAAACACCAAAGGCTATTGAAGTCAGGCCACTTGTGC |
| (GenBank: | | CACGGCTGGAGCAAACCGTGCTGCCTGTAGCTCCGCCAATAACGGG |
| MN018390.1) | | AGGCGTTATAATTCCCAGGGAGGCCATGCGCCACGGAAGCTGTACG |
| | | CGTGGCATATTGGACTAGCGGTTAGAGGAGACCCCTCCCATCACCAA |
| | | CAAAACGCAGCAAAAGGGGGCCCGAAGCCAGGAGGAAGCTGTACTC |
| | | CTGGTGGAAGGACTAGAGGTTAGAGGAGACCCCCCCAACACAAAAA |
| | | CAGCATATTGACGCTGGGAAAGACCAGAGATCCTGCTGTCTCTACAA |
| | | CATCAATCCAGGCACAGAGCGCCGCAAGATGGATTGGTGTTGTTGAT |
| | | CCAACAGGTTCT |
| West Nile virus | 41 | ATAACAAAGCTGTATTGAGTAGTTGTATAGTTGTAGTGTTTTTAGTA |
| (GenBank: | | ATTTGAATTATGATTAATTATTTAGGCTTAAGATAGTATTATAGTTAG |
| LC318700.1) | | TTTAGTGTAAATAGGATTTATTGAGAATGGAAGTCAGGCCAGATTAA |
| | | TGCTGCCACCGGAAGTTGAGTAGACGGTGCTGCCTGCGGCTCAACCC |
| | | CAGGAGGACTGGGTGACCAAAGCTGCGAGGTGATCCACGTAAGCCC |
| | | TCAGAACCGTCTCGGAAGGAGGACCCCACGTGCTTTAGCCTCAAAGC |
| | | CCAGTGTCAGACCACACTTTAGTGTGCCACTCTGCGGAGGGTGCAGT |
| | | CTGCGATAGTGCCCCAGGTGGACTGGGTTAACAAAGGCAAAACATC |
| | | GCCCCACGCGGCCATAACCCTGGCTATGGTGTTAACCAGGGAGAAG |
| | | GGACTAGAGGTTAGAGGAGACCCCGCGTCAAAAAGTGCACGGCCCA |
| | | ACTTGGCTAAAGCTGTAAGCCAAGGGAAGGACTAGAGGTTAGAGGA |
| | | GACCCCGTGCCAAAAACACCAAAAGAAACAGCATATTGACACCTGG |
| | | GATAGACTAGGGGATCTTCTGCTCTGCACAACCAGCCACACGGCACA |
| | | GTGCGCCGATATAGGTGGCTGGTGGTGCTAGAACACAGGATCT |
| Japanese | 42 | TTTGATTTAAGGTAGAAAAATAAACCATGTAAATAATGTAAATGAG |
| encephalitis | | AAAATGTATGTATATGGAGTCAGGCCAGCAAAAGCTGCCACCGGAT |
| virus (GenBank: | | ACTGGGTAGACGGTGCTGCCTGCGTCTCAGTCCCAGGAGGACTGGGT |
| AF080251.1) | | TAACAAATCTGACAACAGAAAGTGAGAAAGCCCTCGGAACCGTCTC |
| | | GGAAGTAGGTCCCTGCTCACCGGAAGTTGAAAGACCAACGTCAGGC |
| | | CACAAGTTTGTGCCACTCCGCTTGGGAGTGCGGCCTGCGCAGCCCCA |
| | | GGAGGACTGGGTTACCAAAGCCGTTGAGGCCCCCACGGCCCAAGCC |
| | | TTGTCTAGGATGCAATAGACGAGGTGTAAGGACTAGAGGTTAGAGG |
| | | AGACCCCGTGGAAACAACAACATGCGGCCCAAGCCCCCTCGAAGCT |
| | | GTAGAGGAGGTGGAAGGACTAGAGGTTAGAGGAGACCCCGCATTTG |
| | | CATCAAACAGCATATTGACACCTGGGAATAGACTGGGAGATCTTCTG |
| | | CTCTATCTCAACATCAGCTACTAGGCACAGAGCGCCGAAGTATGTAG |
| | | CTGGTGGTGAGGAAGAACACAGGATCT |
| Yellow fever | 43 | AACACCATCTAACAGGAATAACCGGGATACAAACCACGGGTGGAGA |
| virus (GenBank: | | ACCGGACTCCCCACAACCTGAAACCGGGATATAAACCACGGCTGGA |
| MT107250.1) | | GAACCGGACTCCGCACTTAAAATGAAACAGAAACCGGGATAAAAAC |
| | | TACGGATGGAGAACCGGACTCCACACATTGAGACAGAAGAAGTTGT |
| | | CAGCCCAGAACCCCACACGAGTTTTGCCACTGCTAAGCTGTGAGGCA |
| | | GTGCAGGCTGGGACAGCCGACCTCCAGGTTGCGAAAAACCTGGTTTC |
| | | TGGGACCTCCCACCCCAGAGTAAAAAGAACGGAGCCTCCGCTACCA |
| | | CCCTCCCACGTGGTGGTAGAAAGACGGGGTCTAGAGGTTAGAGGAG |
| | | ACCCTCCAGGGAACAAATAGTGGGACCATATTGACGCCAGGGAAAG |
| | | ACCGGAGTGGTTCTCTGCTTTTCCTCCAGAGGTCTGTGAGCACAGTTT |
| | | GCTCAAGAATAAGCAGACCTTTGGATGACAAACACAAAACCACT |
| Yellow fever | 44 | AACACCATCTAATAGGAATAACCGGGATACAAACCACGGGTGGAGA |
| virus (GenBank: | | ACCGGACTCCCCACAACTTGAAACCGGGATATAAACCACGGCTGGA |
| MT956629.1) | | GAACCGGACTCCGCACTTAAAATGAAACAGAAACCGGGATAAAAAC |
| | | TACGGATGGAGAACCGGACTCCACACATTGAGACAGAAGAAGTTGT |
| | | CAGCCCAGAACTCCACACGAGTTTTGCCACTGCTAAGCTGTGAGGCA |
| | | GTGCAGGCTGGGACAGCCGACCTCCAGGTTGCGAAAAACCTGGTTTC |
| | | TGGGACCTCCCACCCCAGAGTAAAAAGAACGGAGCCTCCGCTACCA |
| | | CCCTCCCACGTGGTGGTAGAAAGACGGGGTCTAGAGGTTAGAGGAG |
| | | ACCCTCCAGGGAACAAATAGTGGGACCATATTGACGCCAGGGAAAG |
| | | ACCGGAGTGGTTCTCTGCTTTTCCTCCAGGGGTCTGTGAGCACAGTTT |
| | | GCTCAAGAATAAGCAG |
| Zika virus | 45 | GCACCAATCTTAATGTTGTCAGGCCTGCTAGTCAGCCACAGCTTGGG |
| (GenBank | | GAAAGCTGTGCAGCCTGTGACCCCCCCAGGAGAAGCTGGGAAACCA |
| MH882538.1) | | AGCCTATAGTCAGGCCGGGAACGCCATGGCACGGAAGAAGCCATGC |
| | | TGCCTGTGAGCCCCTCAGAGGACACTGAGTCAAAAAACCCCACGCG |
| | | CTTGGAGGCGCAGGATGGGAAAAGAAGGTGGCGACCTTCCCCACCC |
| | | TTCAATCTGGGGCCTGAACTGGAGATCAGCTGTGGATCTCCAGAAGA |
| | | GGGACTAGTGGTTAGAGGAGACCCCCTGGAAAACGCAAAACAGCAT |
| | | ATTGACGCTGGGAAAGACCAGAGACTCCATGAGTTTCCACCACGCTG |
| | | GCCGCCAGGCACAGATCGCCGAATAGCGGCGGCCGGTGTGGGGAAA |
| Tick-borne | 46 | AACCAAAGTGTGACAGAGCAAAACCTGGAGGGCTCGTAAAATATTG |
| encephalitis | | TCCAGAATCAAAAACCACAGCAAGCAAAACACAGAAACAGAGCTCG |
| virus (GenBank: | | GACTGGAGAGCTCTTAAAACAAAAAAGCCAGAATTGAGCTGAACCT |
| MH645619.1) | | GGAGGGCTCATTAAACATTGTCCAGACAAAACAAAACAGACATGAT |
| | | CACAAGCAAAGGAAAGAGGCTGAGCAAAGGTCCTGAATGACCAGAC |
| | | CGGTCTTACCGCGGGCTGGGAAGGGGGGCCAGAATGCGAGGCCACA |
| | | GACCATGGAATGCTGCGGCAGCGCGCGAGAGCGACGGGGAAATGGT |
| | | CGCACCCGACGCACCATCCATGAAGCAACACTTCGTGAGACCCCCCC |
| | | GGCCAGTGGAGGGGGAAGCTGGTCAGGGGTGAAAGCACCCCCAGAG |
| | | TGCACTATGGCAACACGCCAGTGAGAGTGGCGACGGGAAAATGGTC |
| | | GATCCCGACGTAGGGCACTCTGTAAAACTTTGTGAGACCCCCTGCAT |
| | | CATGACAAGGCCTAACATGATGCACGAAAGGGAGGCCCCCGGAAGC |
| | | GAGCTTCCGGGAGGAGGGAAGGGAGAAATTGGCAGCTCCCTTCAGG |
| | | ATTTTTCCTCCTCCTATACTAAATTCCCCCTCAATAGAGGGGGGGG |
| | | GTTCTTGTTCTCCCTGAGCCACCATCACCCAGACACAGATAGTCTGA |
| | | CAAGGAGGTGATGTGTGACTCGGAAAAACACCCGCT |
| Usutu virus | 47 | ATAAGTGTTTAGGGTTTTGCAATTTAATTAAATATGCAATGTAATTTA |
| (GenBank: | | GTTGTAAATATTTGATTGTGTAGCTTTATTTAGCATTGTTTTAGGATA |
| AY453411.1) | | GTAGAAGTTAAGGTTTTATTTAGTTATTTTATTTAATTGAATTTGATA |
| | | GTCAGGCCAGGGCAACCTGCCACCGGAAGTTGAGTAGACGGTGCTG |
| | | CCTGCGACTCAACCCCAGGCGGACTGGGTTAACAAAGCTGACCGCT |
| | | GATGATGGGAAAGCCCCTCAGAACCGTTTCGGAGAGGGACCCTGCC |
| | | TATTGGAAGCGTCCAGCCCGTGTCAGGCCGCAAAGCGCCACTTCGCC |
| | | AAGGAGTGCAGCCTGTACGGCCCCAGGAGGACTGGGTTACCAAAGC |
| | | CGAAAGGCCCCCACGGCCCAAGCGAACAGACGGTGATGCGAACTGT |
| | | TCGTGGAAGGACTAGAGGTTAGAGGAGACCCCGTGGAACTTAGGTG |
| | | CGGCCCAAGCCGTTTCCGAAGCTGTAGGAACGGTGGAAGGACTAGA |
| | | GGTTAGAGGAGACCCCGCATCATAAGCATCAAAAAAACAGCATATT |
| | | GACACCTGGGAATTAGACTAGGAGATCTTCTGCTCTATTCCAACATC |
| | | AACCACAAGGCACAGAGCGCCGAAAATTGTGGCTGGTGGGGAACTA |
| | | GACCACAGGATCT |
| Border disease | 48 | ACCATAGCTGAGCATTTCATGACAACACGCCAAGGGCCACTAAATTG |
| virus (NCBI | | TATATATAACTGTGTAAATATTTACCTATTTATTTACTGTTATTTATTT |
| Reference | | AATAGAGACAGTGATATTTATTTAATAGCTTATCTATTTATTTATTTG |
| Sequence: | | ATGGGATGTAGATGGCAACTAACTACCTCATAGGACCACACTACACT |
| NC_003679.1) | | CATTTTTAAAACTACAGCACTTTAGCTGGAAGGGAAAAGCCTGAAGT |
| | | CCAGAGTTGGATTAAGGAAAAACCCTAACAGCCCC |
| Bovine viral | 49 | GACAAAATGTATATATTGTAAATAAATTAATCCATGTACATAGTGTA |
| diarrhea virus | | TATAAATATAGTTGGGACCGTCCACCTCAAGAAGACGACACGCCCA |
| (NCBI | | ACACGCACAGCTAAACAGTAGTCAAGATTATCTACCTCAAGATAAC |
| Reference | | ACTACATTTAATGCACACAGCACTTTAGCTGTATGAGGATACGCCCG |
| Sequence: | | ACGTCTATAGTTGGACTAGGGAAGACCTCTAACAG |
| NC_001461.1) | | |
| Bussuquara | 50 | GCTAAGATAAAAGAGAAAAAGAGGGTTTGAGTCAGGCCAGAAATGC |
| virus (NCBI | | CACCGGATAAAGGTAGACGGTGCTGCCTGCAACCTTTCTGCGGAAG |
| Reference | | GAATAACCGCAGTCAATAAAACCAAAAAGAGGGAGTTGAGAACCCT |
| Sequence: | | TTGGGCCGCCCAGGCCTGGGATTGAACCGTTGATCCCAGGCGAAGG |
| NC_009026.2) | | GACTAGAGGTTAGAGGAGACCCAGCCTTTCTCACCAACCCAAGGCC |
| | | CAACCTTGCTGAACCTTTAGGCAGGTAAAAGGACTAGAGGTTAGAG |
| | | GAGACCCCTTGGCAAAACAGTTAACGCACCAAAAGAAACAGCATAT |
| | | TGACACCTGGGATAGACCGGAGAATTTGCTGCCTCGCAACACCTCCC |
| | | ACCCGGCACAGAACGCCGACATGGTGGGAGGGGTCGTAAGACACCA |
| | | GATTCT |
| Cell fusing | 51 | ACGAAATCGAATAGAGCCGTGAGGAACCAGCATCCTCCCGGCCACA |
| agent virus | | GGAGCAGGGCATGAAAATGTCGGGCATGACGAACCCGCTCCCCCGA |
| (NCBI | | GTCCCCTGGCAACAGGGTGTGTTCCCTTATGGAGCACGTTCGAGCAG |
| Reference | | GGCACATTAGTGTCGGGCGTGACGCACCCGCTCCCCTCAGTCCCCTG |
| Sequence: | | TGCAACAGGGAGGGCACTTGTAACCCCCGTAGGAGGGTGCCCGCTT |
| NC_001564.2) | | CCGTCCTACAAAAACCTCTGATCATAGGTACCTGATCTAAGATGGTG |
| | | GTGGCGGCCCATCTTATCATTTAGCTAGCTGATGGTCTTAAGCATCC |
| | | CTCCCATGGAATGGGTAAGAGAAGCCTGCAAACAAAACTGGATGGC |
| | | ACCAGTGCTCTTACAAAATGGCAGCCAAAGCGATCCAGAGCTTTCAA |
| | | AACTGGACGGGGCAACAGGGAGAAATCCCGGGGTAGCGAACCTCCT |
| | | CCGTTAATGTGAAAAAGTATGGGGAAAGAACTCATCTTAACCTCCCA |
| | | CCGTTAGGGAGTTTTGATTATCTTTTCTATACCATAGATGC |
| Classical swine | 52 | GCGCGGGTAACCCGGGATCTGGACCCGCCAGTAGAACCCTGTTGTA |
| fever virus | | GATAACACTAATTTTTTTTTATTTATTTAGATATTACTATTTATTTATT |
| (NCBI | | TATTTATTTATTGAATGAGTAAGAACTGGTACAAACTACCTCAAGTT |
| Reference | | ACCACACTACACTCATTTTTAACAGCACTTTAGCTGGAAGGAAAATT |
| Sequence: | | CCTGACGTCCACAGTTGGACTAAGGTAATTTCCTAACGGCCC |
| NC_002657.1) | | |
| Culex flavivirus | 53 | GAATCACGCGAATCGTAGAGAACCACATCTCTAGAAAAGGTTAACG |
| (NCBI | | TTGCGAAGCAACGGGAACCCCGTAAGGAAGGACAAGGCTGTCCTTG |
| Reference | | AGTACTAACGACACTCCGGCCCCAGTTCCCAGAGCCAGGGTTTTAGC |
| Sequence: | | TCCACGGTGCTGGAAGTCACCCTCGCAGCCATGGCTGCACGACGCGC |
| NC_008604.2) | | GCAAGGAAGGACATGGCTGTCCTTGGGTACGAACGACACCCCGCCC |
| | | CCAGTTCTCAAGGTTAGAGTTATAACCTCAGGGTGTTGGAAGACATC |
| | | CAGGCCATAGTAGGGCCATCGCAAGGGAGGATTTTCCTCGGGTACTG |
| | | ACCATACCCCGACCCCAGTCCGATAGGTCATGGAATGACCCCATGGT |
| | | GCTGAGAGGGCATCCAAACAAGCTGAGCATCTTGGATTCTGCTCCCG |
| | | TAAGGAAAGCGCAAGCTTTGAGCATTGACAACGCTCCGGCCCCAGT |
| | | CCCCCAGGTTATGGGAGAATAACCCCGACGTGCTGGAAGGGCACGA |
| | | ATCACCGCAAGGTGAGGGCGCACAGGATAGAATCCAGGTGACTGAC |
| | | GCCACCTCCCGAAATGTGTATAGTAACAGAGCATGCCTGCAGCAGC |
| | | AGGTCTCCACCGTTAGGAGACTTGTTGCGGGCAAGCTCTTGTTCACG |
| | | TCT |
| Entebbe bat | 54 | ATGAAAATCTTGGAATAAAGTCAGGCCGCAGCGTCTAAAACCGGAG |
| virus (NCBI | | CCTCCGCTGGGAAACCAGTCGACGGGGACTAGAGGTTAGAGGAGAC |
| Reference | | CCCCCGCGCCCATAACCAACATAAAACAGCATATTGACACCTGGGA |
| Sequence: | | AAAGACCGGAGACTCTG |
| NC_008718.1) | | |
| Pestivirus | 55 | GCAGTAAGCAGCTCCCAATGTAACATAATGTAAATAAATGTGACTTT |
| giraffe-1 (NCBI | | ATGTAAATGCAAGGCAGTAAGCAGCTCCCAATGTAACATAATGTAA |
| Reference | | ATAAATGTAACTTTATGTAAATGCAAGTAGAGTAGTTAGAGTTCTAA |
| Sequence: | | GGACATACTACATAGAGACAACAACTACCTCATTTTTAAAAACAGCA |
| NC_003678.1) | | CTTTAGCTGGAAGGGGATATTCCGACGTCCACTGTTGGTCTAGGAAA |
| | | AAACCCTGAAGGCCCC |
| Hepatitis C virus | 56 | AGGTTGGGGTAAACACTCCGGCCTCTTAGGCCATTTCCTGTTTTTTTT |
| (GenBank: | | TTTTTTTTTTTTTTTTTTTTTTTTTTTTTTTTTTTTTTTTTTTCTTTTTTTT |
| AF009606.1) | | TTTTTTTTTCCTTTTTTTTTTTTTTTTTTTTCTTTCCTTCTTTTTTCCTTT |
| | | CTTTTCCTTCCTTCTTTAATGGTGGCTCCATCTTAGCCCTAGTCACGG |
| | | CTAGCTGTGAAAGGTCCGTGAGCCGCATGACTGCAGAGAGTGCTGA |
| | | TACTGGCCTCTCTGCAGATCATGT |
| Hepatitis GB | 57 | ACCCCCAAATTCAAAATTAACTAACAGTTTTTTTTTTTTTTTTTTTTTT |
| virus B (NCBI | | TAGGGCAGCGGCAACAGGGGAGACCCCGGGCTTAACGACCCCGCCG |
| Reference | | ATGTGAGTTTGGCGACCATGGTGGATCAGAACCGTTTCGGGTGAAGC |
| Sequence: | | CATGGTCTGAAGGGGATGACGTCCCTTCTGGCTCATCCACAAAAACC |
| NC_001655.1) | | GTCTCGGGTGGGTGAGGAGTCCTGGCTGTGTGGGAAGCAGTCAGTAT |
| | | AATTCCCGTCGTGTGTGGTGACGCCTCACGACGTATTTGTCCGCTGT |
| | | GCAGAGCGTAGTACCAAGGGCTGCACCCCGGTTTTTGTTCCAAGCGG |
| | | AGGGCAACCCCCGCTTGGAATTAAAAACT |
| GB virus | 58 | ACTAAATTCATCTGTTGCGGCAAGGTCTGGTGACTGATCATCACCGG |
| C/Hepatitis G | | AGGAGGTTCCCGCCCTCCCCGCCCCAGGGGTCTCCCCGCTGGGTAAA |
| virus (NCBI | | AAGGGCCCGGCCTTGGGAGGCATGGTGGTTACTAACCCCCTGGCAG |
| Reference | | GGTCAAAGCCTGATGGTGCTAATGCACTGCCACTTCGGTGGCGGGTC |
| Sequence: | | GCTACCTTATAGCGTAATCCGTGACTACGGGCTGCTCGCAGAGCCCT |
| NC_001710.1) | | CCCCGGATGGGGCACAGTGCACTGTGATCTGAAGGGGTGCACCCCG |
| | | GGAAGAGCTCGGCCCGAAGGCCGGSTTCTACT |
| Ilheus virus | 59 | ACCCAAAAGACCAAAAAAGGACAATTGTGTCAGGCCATGGAAACAT |
| (NCBI | | GCCACCCAAAGCTTGTAGAGGGTGCAGCCTGCGCCAAGCCCCAGGA |
| Reference | | GGACTGGGTTACCAAAGCCGTTAGGCCCCCACGGCCCATTTCAGGAG |
| Sequence: | | ACAGCGCGACTCCTGGAGGAAGGACTAGAGGTTAGAGGAGACCCGT |
| NC_009028.2) | | GGAACATCGCTGAGGCCCAAACCAGCCCGAAGCTGTAGGACTGGTG |
| | | GAAGGACTAGAGGTTAGTGGAGACCCCTCAGCACCAAGCGCGAAAC |
| | | AAACAGCATATTGACGCCTGGGAAAGACCGGGAGATCCTCTGCTTTC |
| | | CATCACCAGCCACTAGGCACAGATCGCCGCAAGTAGTGGCTGGTGG |
| | | TGAAAAACACATGGATCT |
| Kamiti River | 60 | TGAGACAAAGGTCCTTGAGTCCAAGTTCCTATCCAAGAAGGAACAC |
| virus (NCBI | | CCTCCCCCTAACCCCCCCCTCCAAAAGTCCCCATCCCTTCCCCCTCTC |
| Reference | | CTTTCTGGAGTTTGCATCTGTCTCTATCCCAAGCCCTCAGTGGTTTAA |
| Sequence: | | GACAGGGGGTATTTGGAACTGATTTCCATAACCCCTCATGCGCGACT |
| NC_005064.1) | | TTTAGAGCAGGGCACGAAAGTGTCGGGCATGACGCACCCGCTCCCC |
| | | CGAGTCCCCTGAAAATAGGGTGGGCAATGCACTCCTGAGTAGGACG |
| | | GGAGCCCAGAATCCTACAAAACCCTCGCCATGGGAACTGGCATGAC |
| | | ACAGGAGTGGTGACCTGTCTCATACATGACACCTTGAAACCCCACCC |
| | | GTGACAGCATGGGCTGGCCTCTAACCCTCTGGGTAATGCTCGTACAT |
| | | GGCAGCAATCCTGGTTCTCGCAACTCCAGTCGAATCTTCGAGTACAC |
| | | GGGAACAAGGATCAGCAATGTTTTTACGACATCACCAAGACGGGTG |
| | | GAATGTCCAACCCCCCGGTAGCATCCGTGCCAAAATGGTGGCTCTCG |
| | | CAACTCCGGTGGAATCTTCGATCCCATCGGAGTGAGAGTCAGTAATT |
| | | TTTCGCGGTGCCTCCCGGACCGTGGAATGCCGGCCCGGACGTCTAGG |
| | | TAGGAACGTAGGCGTTTCGGATTGTGGTTGACCGCTGGGTGGTGCTC |
| | | ATATTTGAAGCATCTCTCAGAGTCTCTTACCACAACCTGAAATGTCT |
| | | GAGATAGAAGTGGCGGCCTATCTCATTGAAAACGCCATTTGAGCAG |
| | | GGCACGAAAGTGTCGGGCCTGACGCACCCGCTCCCCCGAGTCCCCTG |
| | | GAAACAGGGTGGGCCTCGAAAAATCCACCGTAGGAAGGAGCCCAAT |
| | | CCTACAAGAACCCTCTGGTCATAGGCACCTGACCTGGGATAAGAGTG |
| | | GCGCCTTATCTCATATTTAGCTAGCTGGTGGACTCAAGCACCCCCCC |
| | | CCATGGAATGGGGTAAGAGAGGCCTGTAAACATCGCTGGATGGCTC |
| | | CAGCACTCTTATAAATTGGCCGCCAAGCGATCCGGAGCTTTCAAAAC |
| | | CGGACGGAGCAACAGGGAATTTCCCGGGGACGCGTACCCCCTCCGT |
| | | AATGTGAAAAAGTATGGGGAAAAGAACCCAGCTAAATCTCCCACCG |
| | | ATAGGGAGTTTGGACTATCTTTTCTATACCATAAATGCGCT |
| Kokobera virus | 61 | ATGAAGAGAATGAAGTGAGTTATTTTGTTGTGATAGTCAGGCCTGAA |
| (NCBI | | AAGCCACCTGATCCGGTGAAGGTGCTGCCTGCATCCGGCCTGGAGTG |
| Reference | | ATGCTCCAGTGTCGTGGAACAACAACCGATGGAGCCAAGCCCGGAG |
| Sequence: | | GGGATCCGGCCCCCGACTTCCGGAGGTTGCCACACCTTGTAAATATG |
| NC_009029.2) | | TACATACAGAGTCAGATCCGAAAGGCCACCAGTTTGGTGCAGAACT |
| | | GGTGCTATCTGTGAACACTCCCAGGAGGACTGGGTAAACAAAGCCA |
| | | TTAGGGACCATCACGGCCCGAGGGGGAGAAGAACGCGAACTCCCCC |
| | | AAAGGACTAGAGGTTAGAGGAGACCCGTGATTAGGGAGATGAGGGA |
| | | GCCCATCTCAGGGAAAGCTGTAACCCTGGGGGAAGGACTAGAGGTT |
| | | AGAGGAGACCCTCCCACAAAGAAGCGCAAACACAAAACAGCATATT |
| | | GACACCTGGGAAAGACTAGGGGATTTGCTGCTCTGGACTTCCGGCTC |
| | | TCGGCACAGAACGCCGTTGAGGAGCCGGAGGCCCAAAACACCAGAT |
| | | CT |
| Langat virus | 62 | AGCCAGACACAAGGAGTCCAACCTGGAGGGCTCTTGAAAAACTCGT |
| (NCBI | | CCAGAAACCAAACAAATGAGCAAGTCAACAGGAGATGATAACTCGT |
| Reference | | ACGAGCTGATCTCCAACACACAAGAAAAATGGTGGGATGCGGCAAC |
| Sequence: | | GCACGAGGCTCGTGACGGGGAAATGATCGCTCCCGACGCACCCCTC |
| NC_003690.1) | | CATTGGAGACAACTTCGTGAGATCCCCCAGGTGTTTAGGGGCACACG |
| | | CCTGAGGTAAGCAAGCCCCAGGGCGCATTCCGGCAGCACACCAGTG |
| | | AGAGTGGTGACGGGAAACTGGTCACTCCCGACGGAGCTGCGCCTTG |
| | | TGAAACTTTGTGAGACCCCTTGCGTCCAGAGAAGGCCGAACTGGGC |
| | | GTTATAAGGAGGCCCCCAGGGGGAAACCCCTGGGAGGAGGGAAGA |
| | | GAGAAATTGGCAACTCTCTTCAGGATATTTCCTCCTCCTATACCAAA |
| | | TTCCCCCTCGTCAGAGGGGGGGCGGTTCTTGTTCTCCCTGAGCCACC |
| | | ATCACCTAGACACAGATAGTCTGAAAAGGAGGTGATGCGTGTCTCG |
| | | GAAAAACACCCGCT |
| Louping ill virus | 63 | GCCTAGCTTGTGACAGAGCAAAACTTGAAGAGCTCGCAAGGAAACC |
| (NCBI | | ATGGAATGATGCGGCACGGCGCGACAGCGACGGGGAAATGGTCGCA |
| Reference | | CCCGACGCACCATCCATGAGGCAGCAATTCGTGAGACCCCCCTGGCC |
| Sequence: | | AGGAAAGGGGAAAACAGGCCAGGGGTGAAAACACCCCCAGAGTGC |
| NC_001809.1) | | ACCACGGCAACACGCCAGTGAGAGTGGCGACGGGGAGATGGTCGAT |
| | | CCCGACGTAGGGCACTCTGCAAGATTTTGCGAGACCCCCCGCCCCAT |
| | | GACAAGGCCGAACATGGAGCATTAAAGGGAGGCCCCCGGAAGCATG |
| | | CTTCCGGGAGGAGGGAAGAGAGAAATTGGCAGCTCTCTTCAGGGTT |
| | | TTTCCTCCTCCTATACCAAATTTCCCCCTCGACAGAGGGGGGGGGT |
| | | TCTTGTTCTCCCTGAGCCACCATCACCCAGACACAGATAGTCTGACA |
| | | AGGAGGTGATGTGTGACTCGGAAAAACACCCGCT |
| Modoc virus | 64 | ACAATGAAATAATTAAATGAAAGAGTGTTGAGGGCAACCAGTGGGC |
| (NCBI | | TAGCCACATGGGTATGACGCACCCACCCTCTGCATTCTTGTAAATAC |
| Reference | | TTTGGCCAGTCATTGTAAATAGGTTAGGGAGCCGGGCCCAACCCAGC |
| Sequence: | | TAGGGATAGCCTTTCTGGGGTAAGGACTAGAGGTTAGTGGAGACCC |
| NC_003635.1) | | CCGGCTTTTGAAGTTAGGGCAACACAGGGAGTGGTTCAATTGGCCAG |
| | | AACCGCTCTGGCGTTTGCCTCCTGTTATTTTCCAAATTCCCGTTACCG |
| | | GGGGTGGGGTGATTAGCCATGGTCGCACAGATCAAGCTCAGATTGCT |
| | | TACATGTAATCTGTGTGGTCATGAATATGACCTCCGCT |
| Montana myotis | 65 | TAGATCCAGCAACACCTAAAATGTACATAGAAAACAACTAATGGAA |
| leukoencephalitis | | AAAATGCGAGTGAGGGCAACTCTGGGATTAGCTCAATGGGTGTGAC |
| virus (NCBI | | GACCCTACCCTTCCGCATTTGTAAATAATTGAGCCAGTCATTTCCGTA |
| Reference | | GGGAAGAGAGTTATTCGCTCCTCTCGAGATTGAGCGGCCTGCTCCTT |
| Sequence: | | GGAGCATGAGATGGGAGGCCCGAAGCAAAGCTGAAAGGACTAGCG |
| NC_004119.1) | | GTTAGAGGAGACCCCTTCCATCTCTGGTATCAAATTTCATGGAGTTT |
| | | ACTCCATGGTGGCTAGAACCCATAGCGGGGGTGAACCACATTGGCT |
| | | AAGGTTCACCAGCTTTTGCTCCCGCGTTTTTCAAATTGCCTCATCTTG |
| | | AATGGGGGGCGGCGTGGATATATACTCCAGCCAGAAAAGACTCAGA |
| | | TTGTCTCATGACTTTCTGACTGGCGTACATAGCCATCCGCT |
| Murray Valley | 66 | ATAACATTGATAGAAAATTTTGTAAATATTTAATGTAATATAGTATA |
| encephalitis | | GGTAAAATTTTTTGAAATTAAGTAAAATTAAGTAGCAAGACTTGATA |
| virus (NCBI | | GTCAGGCCAGCCGGTTAGGCTGCCACCGAAGGTTGGTAGACGGTGC |
| Reference | | TGCCTGCGACCAACCCCAGGAGGACTGGGTTACCAAAGCTGATTCTC |
| Sequence: | | CACGGTTGGAAAGCCTCCCAGAACCGTCTCGGAAGAGGAGTCCCTG |
| NC_000943.1) | | CCAACAATGGAGATGAAGCCCGTGTCAGATCGCGAAAGCGCCACTT |
| | | CGCCGAGGAGTGCAATCTGTGAGGCCCCAGGAGGACTGGGTAAACA |
| | | AAGCCGTAAGGCCCCCGCAGCCCGGGCCGGGAGGAGGTGATGCAAA |
| | | CCCCGGCGAAGGACTAGAGGTTAGAGGAGACCCTGCGGAAGAAATG |
| | | AGTGGCCCAAGCTCGCCGAAGCTGTAAGGCGGGTGGACGGACTAGA |
| | | GGTTAGAGGAGACCCCACTCTCAAAAGCATCAAACAACAGCATATT |
| | | GACACCTGGGAAAAGACTAGGAGATCTTCTGCTCTATTCCAACATCA |
| | | GTCACAAGGCACCGAGCGCCGAACACTGTGACTGATGGGGGAGAAG |
| | | ACCACAGGATCT |
| Omsk | 67 | CCACAGACAACCATAGAGCAAAAGCACCATTTCGTGAGACCCCCCT |
| hemorrhagic | | GCCAGTTGAAGGGGGAAGCTGGCCGGTGGTAGAAAACCCCCCAACA |
| fever virus | | GGGTGCCAAACGGCAACACGCCAGTGAGAGTGGCGACGGGAACATG |
| (NCBI | | GTCGCTCCCGACGTAGGGCACTCTATCCAATTTTGTGAGACCCCCCG |
| Reference | | CACCATGGAAGGCCAAACATGGTGCATGAAGGGAAAGGCCCCCGGA |
| Sequence: | | AGCTTGCTTCCGGGAGGAGGGAAGAGAGAAATTGGCAGCTCTCTTC |
| NC_005062.1) | | AGGAAATTTCCTCCTCCTATACCAAATTCCCCCTCATCTGAGGGGGG |
| | | GCGGTTCTTGTTCTCCCTGAGCCACCATCACCCAGACACAGGCAGTC |
| | | TAACAAGGAGGTGATGTGTGACTCGGAACAACACCCGCT |
| Powassan virus | 68 | ACTAGCATGACTGAACAGTCAAAAGAACCCTAACACAGGGGATGGT |
| (NCBI | | GTGGCAGCGCACAACGACATCGTGACGGGAGTGGGTCGCCCCCGAC |
| Reference | | GCACCATCCTCTTGGGAAAAATTTTCGTGAGACCCTCACGGCTGGCA |
| Sequence: | | AAGGGCACCAGTCGTGTAGTAAGAAGGCCCTGGCCCAGTGCGGCAG |
| NC_003687.1) | | CACACTCAGTGACGGGAAAGTGGTCGCTCCCGACGTAACTGGGTAA |
| | | AAACGAACTTTGTGAGACCAAAAGGCCTCCTGGAAGGCTCACCAGG |
| | | AGTTAGGCCGTTTAGGAGCCCCCGAGCATAACTCGGGAGGAGGGAG |
| | | GAAGAAAATTGGCAATCTTCCTCGGGATTTTTCCGCCTCCTATACTA |
| | | AATTTCCCCCAGGAAACTGGGGGGGCGGTTCTTGTTCTCCCTGAGCC |
| | | ACCACCATCCAGGCACAGATAGCCTGACAAGGAGATGGTGTGTGAC |
| | | TCGGAAAAACACCCGCT |
| Sepik virus | 69 | ACAGACTGACACAAAATAAGTGACCAGAATGGGACTAAACCACCTA |
| | | CTATATGTAAAACCGGGATAAAAACCACGGAGAGGACCGGACCTCT |
| | | CACTATGTAAAACCGGTATACAAACCAAAACAGACAGGACCGGACC |
| | | TGCCTGATGTCAGCCCGTCATAATGACGCCATGGCTAAGCTGTGAGG |
| | | CCATGCTGGCTGGGATAGCCGCGACCACCCGCGTAATGGGGTTCCTG |
| | | GATTGCTCGATCCGGGGTAAAAAATTTTTAGGGAGCCTCCGCCTGCT |
| | | GCGTCCGCGCGCAGCAGGAAAGAAGGGGTCTAGAGGTTAGAGGAGA |
| | | CCCTCCCGAGCACTATAGCGGACCATATTGACGCCTGGGAAAGACC |
| | | GGAGACACTCCTTGATTCTCACCTTTCTCACCCTTAAGCACAGATTGC |
| | | TTGAATGCAGGGTGGGGAAGTTGGGAACCAACTAGTGTCT |
| Yokose virus | 70 | GAGCAATAAAAAATTTTAAAGACAAAAGTGTCAGGCCAAGATTGAG |
| (NCBI | | AAAATCTTGCCACAGCTTGGCAGACTGTGCAGCCTGCAGCCCTAGAG |
| Reference | | GGAGACTGACCAACTCCCTTTAGTAGAAAAGGTCAGGGAAGAACTT |
| Sequence: | | GAGGATGGGTGTGGCCTCAAGATCTCTTCTCAAAAAACGGACTGAA |
| NC_005039.1) | | CACCACACCTAGATGAAGATAGTAGGGGAGCCTCCGCCAATGGTGG |
| | | CTTTACATATTGAGCTACTGCATTGGTCGATGGGGACTAGCGGTTAG |
| | | AGGAGACCCTCTCCTACGCATGGATTTTGCAATATGTTGACATCAGG |
| | | GAAAGACCGGGTGTTTGTCGGTTCCGGAGAGCTCCGGAGGCCAGGG |
| | | CGCCGTTTGCCCGTAGTTTATAACTGGCCTTCGGGGATCGAAGGAGT |
| | | TGCCAAACACT |

In some embodiments, a 3′ UTR comprises adenylate-uridylate-rich elements (AREs). In some embodiments, ARE is a region with frequent adenine and uridine bases in a mRNA. In some embodiments, AREs include class I AREs that have dispersed AUUUA motifs within or near-rich regions; class II AREs that have overlapping AUUUA motifs within or near U-rich regions; and class III AREs that have a U-rich region but no AUUUA repeats. In some embodiments, AREs contribute to the stability of RNA stability in mammalian cells. Proteins binding to AREs to stabilize the mRNA include, but not limited to, HuA, HuB, HuC, HuD, and HuR. Proteins binding to AREs to destabilize mRNA include, but not limited to, AUF1, TTP, BRF1, TIA-1, TIAR, and KSRP. In some embodiments, AREs are removed or mutated to increase the intracellular stability of the RNA and thus increase translation and production of the resultant protein.

In some embodiments, a 3′ UTR comprises at least one endonuclease resistance sequence of the second flavivirus. In some embodiments, a 3′ UTR comprises the short hairpin structure of the second flavivirus. In some embodiments, a 3′ UTR comprises the 3′ cyclization sequence of the second flavivirus. In some embodiments, a 3′ UTR comprises a termination codon of the second flavivirus. For instance, the termination codon of the second flavivirus is TAG, TAA, or TGA.

In some embodiments, a 3′ UTR has a length of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more than 1000 bases. In some embodiments, a 3′ UTR has a length of about 200-700, 200-650, 200-600, 200-550, 200-500, 200-450, 200-400, 200-350, 200-300, 200-250, 250-700, 250-650, 250-600, 250-550, 250-500, 250-450, 250-400, 250-350, 250-300, 300-700, 300-650, 300-600, 300-550, 300-500, 300-450, 300-400, 300-350, 350-700, 350-650, 350-600, 350-550, 350-500, 350-450, 350-400, 400-700, 400-650, 400-600, 400-550, 400-500, 400-450, 450-700, 450-650, 450-600, 450-550, 450-500, 500-700, 500-650, 500-600, 500-550, 550-700, 550-650, 550-600, 600-700, 600-650, or 650-700 bases.

In some embodiments, a 3′ UTR is a 3′ UTR of a flavivirus, wherein the flavivirus is not a tick-borne flavivirus (TBFV), a mosquito-borne flavivirus (MBFV), an insect-specific flavivirus (ISFV), no-known vector flavivirus (NKFV), or a non-classified flavivirus (NCFV). In some embodiments, a 5′ UTR is a 5′ UTR of a flavivirus, wherein the flavivirus is not a dengue virus (DENV), West Nile virus (WNV), Japanese encephalitis virus (JEV), yellow fever virus (YFV), Zika virus (ZIKV), tick-born encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV). In some cases, the flavivirus is not a West Nile virus (WNV). In some cases, the flavivirus is not a Japanese encephalitis virus (JEV). In some cases, the flavivirus is not a yellow fever virus (YFV). In some cases, the flavivirus is not a Zika virus (ZIKV). In some cases, the flavivirus is not a tick-born encephalitis virus (TBEV). In some cases, the flavivirus is not a Usutu virus (USUV). In some cases, the flavivirus is not a Apoi virus (APOIV). In some cases, the flavivirus is not a border disease virus (BDV). In some cases, the flavivirus is not a bovine viral diarrhea virus (BVDV). In some cases, the flavivirus is not a Bussuquara virus (BSQV). In some cases, the flavivirus is not a cell fusing agent virus (CFAV). In some cases, the flavivirus is not a classical swine fever virus (CSFV). In some cases, the flavivirus is not a Culex flavivirus (CxFV). In some cases, the flavivirus is not a Entebbe bat virus (ENTV). In some cases, the flavivirus is not a pestivirus giraffe-1. In some cases, the flavivirus is not a hepatitis C virus (HCV). In some cases, the flavivirus is not a hepatitis GB virus B (GBV-B). In some cases, the flavivirus is not a GB virus C/hepatitis G virus (GBV-C). In some cases, the flavivirus is not a Ilheus virus (ILHV). In some cases, the flavivirus is not a Kamiti river virus (KRV). In some cases, the flavivirus is not a Kokobera virus (KOKV). In some cases, the flavivirus is not a Langat virus (LGTV). In some cases, the flavivirus is not a Louping ill virus (LIV). In some cases, the flavivirus is not a Modoc virus (MODV). In some cases, the flavivirus is not a Montana myotis leukoencephalitis virus (MMLV). In some cases, the flavivirus is not a Murray Valley encephalitis virus (MVEV). In some cases, the flavivirus is not a Omsk hemorrhagic fever virus (OHFV). In some cases, the flavivirus is not a Powassan virus (POWV). In some cases, the flavivirus is not a Rio Bravo virus (RBV). In some cases, the flavivirus is not a Sepik virus (SEPV). In some cases, the flavivirus is not a Tamana bat virus (TABV). In some cases, the flavivirus is not a Yokose virus (YOKV).

### Exogenous Polynucleotide

Certain nucleic acid compositions herein comprise an exogenous polynucleotide. In some embodiments, an exogenous polynucleotide is a polynucleotide that is not present in a subject, e.g., a mammalian subject. In some embodiments, an exogenous polynucleotide is a polynucleotide that encodes for an antigen. In some embodiments, an exogenous polynucleotide is not a flavivirus polynucleotide.

In some embodiments, as used herein, a subject refers to any animal, including, but not limited to, humans, non-human primates, rodents, and domestic and game animals. Primates include chimpanzees, cynomolgus monkeys, spider monkeys, and macaques, e.g., Rhesus. Rodents include mice, rats, woodchucks, ferrets, rabbits, and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish, and salmon. In certain embodiments, the subject is a human.

In some embodiments, the exogenous polynucleotide encodes a polypeptide. In some embodiments, the exogenous polynucleotide is translated into the polypeptide in healthy or during cellular stress responses. In some embodiments, the cellular stress response encompasses a wide range of molecular changes that cells undergo in response to environmental stressors, including but not limited to, extreme temperature, exposure to toxins or microorganisms, mechanical damages, tumors, and/or nutrient starvation. In absence of the stress responses, cells may be considered healthy.

Non-limiting example exogenous polynucleotides are described elsewhere herein, including, but not limited to, those encoding viral antigens, bacterial antigens, fungal antigens, protozoal antigens, and helminth antigens, and the polynucleotides and peptides of Table 4.

### Nuclease Resistance

Provided herein, in some embodiments, are nucleic acid compositions that are resistant to degradation by RNAse. In some embodiments, the nucleic acid composition is resistant to degradation by XRN-1 (Gene ID 54464). In some embodiments, the nucleic acid composition is resistant to degradation by one or more of the extracellular RNAses. The extracellular RNAses include, but not limited to, mammalian, amphibian, and bacterial RNases. In some embodiments, the extracellular RNAse is a member of the vertebrate-specific gene superfamily. In some embodiments, the vertebrate-specific gene superfamily is the RNAseA superfamily. Non-limiting example RNAseA superfamily members include hRNAse1, hRNAse2, hRNAse3, hRNAse 4, hRNAse5, hRNAse6, hRNAse7, hRNAse8, hRNAse9, hRNAse10, hRNAse11, hRNAse12, and hRNAse13. Other vertebrate RNAseA family members include, but not limited to, bovine seminal RNAses, bovine milk RNAses, rodent RNAses, and frog RNAses. Other extracellular RNAses include, but not limited to, RNAsesT2, plant self-incompatibility RNAses (S-RNases), and bacterial RNAses.

### 5′ Cap Sequence

Provided herein, in some embodiments, are nucleic acid compositions that do not comprise a 5′ cap sequence. In other embodiments, the nucleic acid compositions described herein comprise a 5′ cap sequence. In certain aspects, a 5′ cap sequence is a modified nucleotide on the 5′ end of an mRNA molecule that comprises a guanine (G) nucleotide connected to mRNA via 5′ to 5′ triphosphate linkage. This guanosine is methylated on the 7 position directly after capping in vivo by a methyltransferase. This process is called 5′ capping. In some embodiments, the nucleic acid compositions do not require the 5′ capping process. In some embodiments, the nucleic acid compositions that do not comprise a 5′ cap sequence can maintain the stability and efficiency of vaccines (e.g., mRNA vaccines) by using a 5′ flavivirus UTR and/or a 3′ flavivirus UTR. Since the nucleic acid compositions do not require a 5′ cap, production time and cost may be significantly reduced.

polyA Sequence

Provided herein, in some embodiments, are nucleic acid compositions that do not comprise a polyA sequence. In other embodiments, the nucleic acid compositions described herein comprise a polyA sequence. A polyA sequence is a region of mRNA that is located downstream from the 3′ UTR that protects mRNA from enzymatic degradation and allows the mature mRNA molecule to be exported from the nucleus and translated into a protein by ribosomes in the cytoplasm. In some cases, a polyA sequence is a long chain of adenine nucleotides. For instance, a polyA sequence contains 10 to 300 adenosine nucleotides. In some cases, a polyA sequence comprises at least 10 bases having at least 80% adenosine residues. In some embodiments, the nucleic acid compositions do not require a polyA sequence. In some embodiments, the nucleic acid compositions that do not comprise a polyA sequence can maintain the stability and efficiency of vaccines (e.g., mRNA vaccines) by using a 5′ flavivirus UTR and/or a 3′ flavivirus UTR. In some cases where the nucleic acid compositions do not require a polyA sequence, production methods and costs may be reduced by eliminating an enzymatic step.

### Cleavage Sites

Provided herein, in some embodiments, are nucleic acid compositions that comprise a polynucleotide encoding a cleavage site. In some cases, the nucleic acid composition comprises one or more polynucleotides encoding one or more cleavage sites. For example, the nucleic acid comprises 2, 3, 4, 5, 6, 7, or 8 polynucleotides, where each polynucleotide encodes a cleavage site. In some such cases, one or more of the polynucleotides may be the same or different. In some embodiments, the cleavage site is positioned between the 5′ UTR and the exogenous polynucleotide. In some embodiments, the cleavage site comprises an exopeptidase, endopeptidase and/or exopeptidase cleavage site. In some embodiments, the cleavage site is a proteasome cleavage site, a cysteine protease cleavage site (cathepsin B, F, H, L, S, Z, and AEP, for asparaginylendopeptidase), an aspartate protease cleavage site (cathepsin D, E), a serine protease cleavage site (cathepsin A, G), or a combination thereof. In some embodiments, the cleavage site comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to SEQ ID NO: 81.

In some embodiments, the nucleic acid composition comprises a self-cleavage site. In some embodiments, the nucleic acid composition comprises an internal ribosome entry site. In some embodiments, the nucleic acid composition comprises a sequence encoding a peptide that induces ribosomal skipping during translation. In some embodiments, the sequence encoding a peptide that induces ribosomal skipping during translation is a peptide motif of DxExNPGP (SEQ ID NO: 165), where x is any amino acid. In some embodiments, the peptide motif of DxExNPGP is encoded by a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to SEQ ID NO: 71 (GCCACCAACTTCAGCCTGCTGAAGCAGGCCGGCGACGTGGAGGAGAACCCCGGCC CC). In some embodiments, the peptide motif of DxExNPGP comprises at least 80%, 81%, 82%, 83%, 84%, 85%, 86%, 87%, 88%, 89%, 90%, 91%, 92%, 93%, 94%, 95%, 96%, 97%, 98%, 99%, or is 100% identity to SEQ ID NO: 72 (ATNFSLLKQAGDVEENPGP).

In some embodiments, the nucleic acid composition comprises a cleavage site comprising a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 73-82. In some embodiments, the nucleic acid composition comprises a polynucleotide encoding a cleavage site comprising a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 83-92.

TABLE 3
Example linkers and cleavage sites

| | SEQ ID NO | | | |
|---|---|---|---|---|
| Linker/ | (nucleic | | SEQ ID NO | Peptide |
| cleavage site | acid) | Nucleic acid sequence | (peptide) | sequence |
| Cathepsin A | 73 | GACAGGGTGTACATCCA | 83 | DRVYIHPFHL |
| AH002594.2 | | CCCCTTCCACCTG | | |
| Cathepsin B | 74 | ATCCTGGCCCAGGTGGT | 84 | ILAQVVGD |
| AC277835.1 | | GGGCGAC | | |
| Cathepsin D | 75 | GAGAGGAACCTGCTGAG | 85 | ERNLLSVA |
| NM_001374086.1 | | CGTGGCC | | |
| Cathepsin E | 76 | ATCAGGAGCTTCGTGGA | 86 | IRSFVETK |
| AH013565.2 | | GACCAAG | | |
| Cathepsin F | 77 | AGCGCCAAGCCCGTGAG | 87 | SAKPVSQM |
| AB202096.1 | | CCAGATG | | |
| Cathepsin G | 78 | CAGGAGGCCTTCGACAT | 88 | QEAFDISKK |
| NM_006142.5 | | CAGCAAGAAG | | |
| Cathepsin H | 79 | AACCAGGGCAGGATCGA | 89 | NQGRIEPD |
| AC279654.1 | | GCCCGAC | | |
| Cathepsin L | 80 | GTGCTGGTGGAGAGGAG | 90 | VLVERSAA |
| EF445028.1 | | CGCCGCC | | |
| Cathepsin S | 81 | GGCAGGTGGCACAAGGT | 91 | GRWHKVSVR |
| CP068261.2 | | GAGCGTGAGGTGGGAG | | WE |
| AEP | 82 | GCCTACAAGAACGTGGT | 92 | AYKNVVGA |
| M93010.1 | | GGGCGCC | | |

### Signal Peptides

Provided herein, in some embodiments, are nucleic acid compositions that comprise a polynucleotide encoding a signal peptide. Non-limiting example signal peptides include Gaussia luciferase, human albumin, human chymotrypsinogen, human interleukin-2, and human trypsinogen-2. Further non-limiting example exogenous polynucleotides are described elsewhere herein, including, but not limited to, those described in Tables 5 and 8. In some embodiments, a signal peptide is encoded by the signal peptide sequence in SEQ ID NO: 164, 172, 173, 178, or 179. In some embodiments, a signal peptide is the signal peptide in SEQ ID NO: 171, 174, or 180.

### Nucleic Acid Modifications

In some embodiments, the nucleic acid composition has 0, 1, 2, 3, 4, 5, 6, 7, 8, 9, 10, 11, 12, 13, 14, 15, 16, 17, 18, 19, or 20 base modifications. In some embodiments, the nucleic acid composition has 0, 1, 2, 3, 4, 5, 6, 7, 8, 9, 10, 11, 12, 13, 14, 15, 16, 17, 18, 19, or 20 backbone modifications. In some embodiments, the nucleic acid composition has 0, 1, 2, 3, 4, 5, 6, 7, 8, 9, 10, 11, 12, 13, 14, 15, 16, 17, 18, 19, or 20 sugar modifications. In some cases, the nucleic acid composition has no base modifications. In some cases, the nucleic acid composition has no backbone modifications. In some cases, the nucleic acid composition has no sugar modifications. In a non-limiting example, the nucleic acid composition has no base modifications, no backbone modifications, and no sugar modifications.

### RNA Compositions

In some embodiments, the nucleic acid composition is a ribonucleic acid (RNA). In some embodiments, the RNA is a messenger RNA (mRNA). mRNA refers to any polynucleotide that encodes one or more polypeptides and can be translated to produce the encoded polypeptide in vitro, in vivo, in situ, or ex vivo. The skilled artisan will appreciate that nucleic acid sequences described herein will recite “T”s in a DNA sequence but where the sequence represents RNA (e.g., mRNA), the “T”s would be substituted with “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

### Flavivirus Structural and Non-Structural Proteins

In some embodiments, the nucleic acid does not comprise a sequence encoding 10 or more contiguous amino acids of a structural protein of the first flavivirus or the second flavivirus. Non-limiting example structural proteins include a capsid, membrane, and envelope protein of the first flavivirus or the second flavivirus. In some embodiments, the nucleic acid does not comprise a sequence encoding 10 or more contiguous amino acids of any structural protein of the first flavivirus or the second flavivirus.

In some embodiments, the nucleic acid does not comprise a sequence encoding 10 or more contiguous amino acids of a non-structural protein of the first flavivirus or the second flavivirus. In some embodiments, the nucleic acid does not comprise a sequence encoding 10 or more contiguous amino acids of any non-structural protein of the first flavivirus or the second flavivirus.

### MHC Binding Peptides

Provided herein, in some embodiments, are nucleic acid compositions comprising a polynucleotide encoding a MHC binding peptide, sometimes referred to herein as a “booster”. Non-limiting example MHC binding peptides are described elsewhere herein, including, but not limited to, viral peptides, bacterial peptides, fungal peptides, protozoal peptides, synthetic peptides, mammalian peptides and helminth peptides, and those disclosed in Table 6 and Table 7. In some embodiments, compositions herein comprise one or a plurality of boosters, for example, 1, 2, 3, 4, 5, 6, 7, 8, 9, or 10 boosters or MHC binding peptides.

### Peptide Compositions

In one aspect, provided herein are peptide compositions comprising a peptide translated from an exogenous polynucleotide described herein. In another aspect, provided herein are peptide compositions comprising an antigen peptide. Non-limiting example peptides translated from exogenous polynucleotides, and antigen peptides, are described elsewhere herein. For example, without limitation, viral peptides, bacterial peptides, fungal peptides, protozoal peptides, helminth peptides, viral antigens, bacterial antigens, fungal antigens, protozoal antigens, helminth antigens, and the peptides of Table 4. In some embodiments, a translated peptide and/or antigen peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 97-100.

In another aspect, provided herein are peptide compositions comprising a MHC binding peptide. Non-limiting example MHC binding peptides are described elsewhere herein, including, but limited to, viral peptides, bacterial peptides, fungal peptides, protozoal peptides, and helminth peptides, and those disclosed in Table 6 and Table 7. In some embodiments, a MHC peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 136-163. In some embodiments, a MHC peptide is encoded by a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 113-135.

In yet another aspect, provided herein are peptide compositions comprising a peptide translated from an exogenous polynucleotide described herein and a MHC binding peptide. In yet another aspect, provided herein are peptide compositions comprising an antigen peptide described herein and a MHC binding peptide. The MHC peptide may be connected to the translated peptide or antigen, or separate.

In some embodiments, peptide compositions herein are peptide vaccines. The peptides may be translated in vitro or in vivo.

### Vaccines

Various embodiments of the nucleic acid compositions and peptide compositions described herein are vaccines. A vaccine is a composition that induces the immune response to a particular pathogen or disease. Conventional protein-based vaccines typically contain an agent that resembles a disease-causing microorganism and is often made from weakened or dead forms of the microbe, its toxins, or one of its surface proteins. The agent induces an immune response to recognize the agent as a threat and eliminate it from a subject's body. If the subject is exposed to the same infectious agent in the future, any microorganisms and proteins associated with that agent will be quickly recognized and destroyed. Gene-based vaccines use a different approach that takes advantage of the process that cells use to make proteins. The gene-based vaccines involve a DNA or RNA vector to deliver a gene sequence encoding an antigen into host cells. The host cells then use the genetic information to produce the antigen that triggers an immune response in a subject. There are two types of the gene-based vaccines—DNA vaccines and mRNA vaccines. mRNA vaccines have several advantages over conventional protein-based vaccines as well as DNA vaccines. First, mRNA vaccines can respond to infectious diseases more rapidly and effectively because they can synthesize antigens via translation from the mRNA immediately after its transfection. Second, mRNA vaccines can be produced easily and less expensively in the laboratory using a DNA template with readily available materials. Third, mRNA vaccines are as safe as conventional protein-based vaccines because mRNA is a non-infectious platform, thus there is no potential risk of infection. Fourth, mRNA vaccine is a safer platform than a DNA vaccine because mRNA carries a short sequence to be translated and does not interact with the host genome. Since the translation of antigens takes place in the cytoplasm rather than the nucleus, mRNA is less likely to integrate itself into the host genome than DNA vaccines and the RNA strand in the vaccine is degraded once the protein is made. Any gene-based vaccine or therapy can benefit from the disclosure described herein. A gene-based vaccine includes, but not limited to, a DNA vaccine and an mRNA vaccine. Additionally, protein-based molecules (e.g., vaccines, therapies, tools) generated with mRNA design can also benefit from the disclosure described herein.

In certain aspects, provided herein are vaccines (e.g., mRNA vaccines) that produce prophylactically- and/or therapeutically-efficacious levels, concentrations and/or titers of antigen-specific antibodies in the blood or serum of a vaccinated subject. In certain aspects, the term “antibody titer” refers to the amount of antigen-specific antibody produces in a subject. In some embodiments, antibody titer is determined or measured by enzyme-linked immunosorbent assay (ELISA). In other embodiments, antibody titer is determined or measured by neutralization assay (e.g., by microneutralization assay). In certain aspects, an antibody titer measurement is expressed as a ratio, such as 1:40, 1:100, etc. Further provided herein are vaccines (e.g., mRNA vaccines) that produce a high antibody titer. For instance, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In some embodiments, the antibody titer is produced or reached by 1 day, 2 days, 3 days, 4 days, 5 days, 6 days, 7 days, 8 days, 9 days, 10 days, 20 days, 30 days, 40 days, 50 days, 60 days, 70 days, 80 days, 90 days, 100 days, 110 days, 120 days, 130 days, 140 days, 150 days, 160 days, 170 days, 180 days, or more days following vaccination. In some embodiments, the titer is produced or reached following a single dose of vaccine administered to the subject. In other embodiments, the titer is produced or reached following multiple doses, e.g., following a first and a second dose (e.g., a booster dose). In certain aspects, antigen-specific antibodies are measured in units of μg/ml or are measured in units of IU/L (International Units per liter) or mIU/ml (milli International Units per ml). In some embodiments, an efficacious vaccine produces >0.05 μg/ml, >0.1 μg/ml, >0.2 μg/ml, >0.3 μg/ml, >0.4 μg/ml, >0.5 μg/ml, >1 μg/ml, >2 μg/ml, >3 μg/ml, 4 μg/ml, >5 μg/ml, >6 μg/ml, >7 μg/ml, >8 μg/ml, >9 μg/ml, or >10 μg/ml. In some embodiments, an efficacious vaccine produces >10 mIU/ml, >20 mIU/ml, >30 mIU/ml, >40 mIU/ml, >50 mIU/ml, >60 mIU/ml, >70 mIU/ml, >80 mIU/ml, >90 mIU/ml, >100 mIU/ml, >200 mIU/ml, >500 mIU/ml or >1000 mIU/ml. In some embodiments, the antibody level or concentration is produced or reached by 1 day, 2 days, 3 days, 4 days, 5 days, 6 days, 7 days, 8 days, 9 days, 10 days, 20 days, 30 days, 40 days, 50 days, 60 days, 70 days, 80 days, 90 days, 100 days, 110 days, 120 days, 130 days, 140 days, 150 days, 160 days, 170 days, 180 days, or more days following vaccination. In some embodiments, the level or concentration is produced or reached following a single dose of vaccine administered to the subject. In other embodiments, the level or concentration is produced or reached following multiple doses, e.g., following a first and a second dose (e.g., a booster dose). In some embodiments, antibody level or concentration is determined or measured by enzyme-linked immunosorbent assay (ELISA). In other embodiments, antibody level or concentration is determined or measured by neutralization assay, e.g., by microneutralization assay.

In certain aspects, vaccines (e.g., mRNA vaccines) described herein may be administered by any route which results in a therapeutically effective outcome. Non-limiting examples of administration methods include intradermal, intramuscular, intravenous, and/or subcutaneous administration. The present disclosure provides methods comprising administering vaccines (e.g., mRNA vaccines) to a subject in need thereof. The exact amount required will vary from subject to subject, depending on the age, general condition, and immunization status of the subject, the severity of the disease, the particular composition, its mode of administration, its mode of activity, and the like. Vaccine (e.g., mRNA vaccine) compositions are typically formulated in dosage unit form for ease of administration and uniformity of dosage. The total daily usage of vaccine (e.g., mRNA) compositions may be decided by the attending physician within the scope of sound medical judgment. The specific therapeutically effective, prophylactically effective, or appropriate imaging dose level for any particular patient will depend upon a variety of factors including, but not limited to, the disease being treated and the severity of the disease; the activity of the specific compound administered; the specific composition administered; the age, body weight, general health, sex and diet of the patient; the time of administration, route of administration, and rate of excretion of the specific compound administered; the duration of the treatment; drugs used in combination or coincidental with the specific compound administered; and like factors well known in the medical arts.

### Exogenous Polynucleotides and Antigens

In one aspect, provided herein are nucleic acid compositions comprising an exogenous polynucleotide. In another aspect, provided herein are nucleic acid compositions comprising a polypeptide that encodes an antigen. In another aspect, provided herein are peptide compositions comprising an antigen. In some embodiments, an exogenous polynucleotide encodes an antigen.

In some embodiments, the nucleic acid composition comprises an exogenous polynucleotide encoding a pathogen-associated antigen. In some embodiments, the peptide composition comprises a pathogen-associated antigen. Pathogens include, without limitation, virus, bacteria, fungus, protozoa, and helminth.

### Viral Antigens

In some embodiments, the pathogen-associated antigen is a viral antigen. Non-limiting example viral antigens include antigens from viruses selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picomaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus.

### Bacterial Antigens

In some embodiments, the pathogen-associated antigen is a bacterial antigen. Non-limiting example bacterial antigens include antigens from viruses selected from Helicobacter pyloris, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii.

### Fungal Antigens

In some embodiments, the pathogen-associated antigen is a fungal antigen. Non-limiting example fungal antigens include antigens from viruses selected from Cryptococcus neoformans, Histoplasma capsulatum, Coccidioides immitis, Blastomyces dermatitidis, Chlamydia trachomatis, and Candida albicans.

### Protozoal Antigens

In some embodiments, the pathogen-associated antigen is a protozoal antigen. Non-limiting example protozoal antigens include antigens from viruses selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major.

### Helminth Antigens

In some embodiments, the pathogen-associated antigen is a helminth antigen. Non-limiting example helminth antigens include antigens from viruses selected from hookworm, Onchocerca volvulus, Brugia malayi, and Ascaris lumbricoides, Ancylostoma caninum excretory/secretory products (AcES), and Schistosoma mansoni.

### Non-Limiting Example Antigen Sequences

In some embodiments, the exogenous polynucleotide encodes a viral structural protein, a viral envelope protein, a viral capsid protein, or a viral nonstructural protein, or any combination thereof.

In some embodiments, the exogenous polynucleotide encodes an antigen. Non-limiting examples of the antigen include Spike SARS-Cov-2, hepatitis B surface antigen, L1 major capsid protein of human papillomavirus (HPV), HA hemagglutinin [Influenza A virus (A/goose/Guangdong/1/1996(H5N1)], and derivatives thereof.

In some embodiments, an antigen comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 97-100. In some embodiments, a polynucleotide encoding an antigen comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 93-96. In some embodiments, an exogenous polynucleotide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 93-96. In some embodiments, an exogenous polynucleotide encodes an antigen comprising a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 97-100. In some embodiments, an exogenous polynucleotide encodes an antigen of SEQ ID NO: 97, wherein the antigen is the antigen RBD as disclosed in Table 8, or a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to the antigen RBD as disclosed in Table 8.

In some embodiments, a polynucleotide encoding an antigen is codon optimized. In some embodiments, codon optimization is a method to match codon frequencies in target and host organisms to ensure proper folding, customize transcriptional and translational control regions, insert or remove protein trafficking sequences, remove/add post translational modification sites in encoded protein (e.g. glycosylation sites), add, remove or shuffle protein domains, bias GC content to increase mRNA stability or reduce secondary structures, minimize tandem repeat codons or base runs that may impair gene construction or expression, insert or delete restriction sites, or modify ribosome binding sites and mRNA degradation sites. As a non-limiting example, a polynucleotide encoding an antigen is optimized for a human subject. For instance, SEQ ID NO: 93 is codon optimized for humans. As another non-limiting example, an antigen comprises one or more amino acid substitutions (e.g., up to 10% or up to 5% of the total amino acid sequence). The one or more amino acid substitutions may render the antigen more stable (e.g., less prone to aggregation), as compared to the antigen that does not have the one or more amino acid substitutions. For instance, SEQ ID NO: 97 comprises the following substitutions: K986P, V987P, K417T, E484K, and N501Y.

TABLE 4
Example antigen sequences

| | SEQ | |
|---|---|---|
| | ID | |
| Antigen | NO | Nucleic acid sequence |
| COVID-19 | 93 | GTGAACCTGACCACCAGAACACAGCTGCCTCCAGCCTACACCAA |
| Spike | | CAGCTTTACCAGAGGCGTGTACTACCCTGACAAGGTGTTCAGAT |
| stabilized | | CCAGTGTGCTGCACTCTACCCAGGACCTGTTCCTGCCTTTCTTCA |
| (K986P and | | GCAACGTGACCTGGTTCCACGCCATCCACGTGTCCGGCACCAAT |
| V987P), | | GGCACCAAGAGATTCGACAACCCCGTGCTGCCCTTCAACGACGG |
| K417T, | | GGTGTACTTTGCCAGCACCGAGAAGTCCAACATCATCAGAGGCT |
| E484K, | | GGATCTTCGGCACCACACTGGACAGCAAGACCCAGAGCCTGCTG |
| N501Y | | ATCGTGAACAACGCCACCAACGTGGTCATCAAAGTGTGCGAGTT |
| | | CCAGTTCTGCAACGACCCCTTCCTGGGCGTCTACTACCACAAGAA |
| | | CAACAAGAGCTGGATGGAAAGCGAGTTCCGGGTGTACAGCAGC |
| | | GCCAACAACTGCACCTTCGAGTACGTGTCCCAGCCTTTCCTGATG |
| | | GACCTGGAAGGCAAGCAGGGCAACTTCAAGAACCTGCGCGAGTT |
| | | CGTGTTCAAGAACATCGACGGCTACTTCAAGATCTACAGCAAGC |
| | | ACACCCCTATCAACCTCGTGCGGGATCTGCCTCAGGGCTTCTCTG |
| | | CTCTGGAACCCCTGGTGGATCTGCCCATCGGCATCAACATCACCC |
| | | GGTTTCAGACACTGCTGGCCCTGCACAGAAGCTACCTGACACCT |
| | | GGCGATAGCAGCAGCGGATGGACAGCTGGTGCCGCCGCTTACTA |
| | | TGTGGGCTACCTGCAGCCTAGAACCTTCCTGCTGAAGTACAACG |
| | | AGAACGGCACCATCACCGACGCCGTGGATTGTGCTCTGGCTCCT |
| | | CTGAGCGAGACAAAGTGCACCCTGAAGTCCTTCACCGTGGAAAA |
| | | GGGCATCTACCAGACCAGCAACTTCCGGGTGCAGCCCACCGAGT |
| | | CCATCGTGCGGTTCCCCAATATCACCAATCTGTGCCCCTTCGGCG |
| | | AGGTGTTCAATGCCACCAGATTCGCCTCTGTGTACGCCTGGAACC |
| | | GGAAGCGGATCAGCAATTGCGTGGCCGACTACTCCGTGCTGTAC |
| | | AACTCCGCCAGCTTCAGCACCTTCAAGTGCTACGGCGTGTCCCCT |
| | | ACCAAGCTGAACGACCTGTGCTTCACAAACGTGTACGCCGACAG |
| | | CTTCGTGATCCGGGGAGATGAAGTGCGGCAGATTGCCCCTGGAC |
| | | AGACAGGCACTATCGCCGACTACAACTACAAGCTGCCCGACGAC |
| | | TTCACCGGCTGTGTGATTGCCTGGAACAGCAACAACCTGGACTC |
| | | CAAAGTCGGCGGCAACTACAATTACCTGTACCGGCTGTTCCGGA |
| | | AGTCCAATCTGAAGCCCTTCGAGCGGGACATCTCCACCGAGATC |
| | | TATCAGGCCGGCAGCACCCCTTGTAACGGCGTGAAAGGCTTCAA |
| | | CTGCTACTTCCCACTGCAGTCCTACGGCTTTCAGCCCACGTATGG |
| | | CGTGGGCTATCAGCCCTACAGAGTGGTGGTGCTGAGCTTCGAAC |
| | | TGCTGCATGCCCCTGCCACAGTGTGCGGCCCTAAGAAAAGCACC |
| | | AATCTCGTGAAGAACAAATGCGTGAACTTCAACTTCAACGGCCT |
| | | GACCGGCACCGGCGTGCTGACAGAGAGCAACAAGAAGTTCCTGC |
| | | CATTCCAGCAGTTTGGCCGGGACATCGCCGATACCACAGACGCC |
| | | GTTAGAGATCCCCAGACACTGGAAATCCTGGACATCACCCCTTG |
| | | CAGCTTCGGCGGAGTGTCTGTGATCACCCCTGGCACCAACACCA |
| | | GCAATCAGGTGGCAGTGCTGTACCAGGACGTGAACTGTACCGAA |
| | | GTGCCCGTGGCCATTCACGCCGATCAGCTGACACCTACATGGCG |
| | | GGTGTACTCCACCGGCAGCAATGTGTTTCAGACCAGAGCCGGCT |
| | | GTCTGATCGGAGCCGAGCACGTGAACAATAGCTACGAGTGCGAC |
| | | ATCCCCATCGGCGCTGGCATCTGTGCCAGCTACCAGACACAGAC |
| | | AAACAGCCCCAGACGGGCCAGATCTGTGGCCAGCCAGAGCATCA |
| | | TTGCCTACACAATGTCTCTGGGCGCCGAGAACAGCGTGGCCTAC |
| | | TCCAACAACTCTATCGCTATCCCCACCAACTTCACCATCAGCGTG |
| | | ACCACAGAGATCCTGCCTGTGTCCATGACCAAGACCAGCGTGGA |
| | | CTGCACCATGTACATCTGCGGCGATTCCACCGAGTGCTCCAACCT |
| | | GCTGCTGCAGTACGGCAGCTTCTGCACCCAGCTGAATAGAGCCC |
| | | TGACAGGGATCGCCGTGGAACAGGACAAGAACACCCAAGAGGT |
| | | GTTCGCCCAAGTGAAGCAGATCTACAAGACCCCTCCTATCAAGG |
| | | ACTTCGGCGGCTTCAATTTCAGCCAGATTCTGCCCGATCCTAGCA |
| | | AGCCCAGCAAGCGGAGCTTCATCGAGGACCTGCTGTTCAACAAA |
| | | GTGACACTGGCCGACGCCGGCTTCATCAAGCAGTATGGCGATTG |
| | | TCTGGGCGACATTGCCGCCAGGGATCTGATTTGCGCCCAGAAGT |
| | | TTAACGGACTGACAGTGCTGCCTCCTCTGCTGACCGATGAGATG |
| | | ATCGCCCAGTACACATCTGCCCTGCTGGCCGGCACAATCACAAG |
| | | CGGCTGGACATTTGGAGCTGGCGCCGCTCTGCAGATCCCCTTTGC |
| | | TATGCAGATGGCCTACCGGTTCAACGGCATCGGAGTGACCCAGA |
| | | ATGTGCTGTACGAGAACCAGAAGCTGATCGCCAACCAGTTCAAC |
| | | AGCGCCATCGGCAAGATCCAGGACAGCCTGAGCAGCACAGCAA |
| | | GCGCCCTGGGAAAGCTGCAGGACGTGGTCAACCAGAATGCCCAG |
| | | GCACTGAACACCCTGGTCAAGCAGCTGTCCTCCAACTTCGGCGC |
| | | CATCAGCTCTGTGCTGAACGACATCCTGAGCAGACTGGACCCGC |
| | | CGGAAGCCGAGGTGCAGATCGACAGACTGATCACCGGAAGGCT |
| | | GCAGTCCCTGCAGACCTACGTTACCCAGCAGCTGATCAGAGCCG |
| | | CCGAGATTAGAGCCTCTGCCAATCTGGCCGCCACCAAGATGTCT |
| | | GAGTGTGTGCTGGGCCAGAGCAAGAGAGTGGACTTTTGCGGCAA |
| | | GGGCTACCACCTGATGAGCTTCCCTCAGTCTGCCCCTCACGGCGT |
| | | GGTGTTTCTGCACGTGACATACGTGCCCGCTCAAGAGAAGAATT |
| | | TCACCACCGCTCCAGCCATCTGCCACGACGGCAAAGCCCACTTTC |
| | | CTAGAGAAGGCGTGTTCGTGTCCAACGGCACCCATTGGTTCGTG |
| | | ACCCAGCGGAACTTCTACGAGCCCCAGATCATCACCACCGACAA |
| | | CACCTTCGTGTCTGGCAACTGCGACGTCGTGATCGGCATTGTGAA |
| | | CAATACCGTGTACGACCCTCTGCAGCCCGAGCTGGACAGCTTCA |
| | | AAGAGGAACTGGATAAGTACTTTAAGAACCACACAAGCCCCGAT |
| | | GTGGACCTGGGCGACATCAGCGGAATCAATGCCAGCGTCGTGAA |
| | | CATCCAGAAAGAGATCGACCGGCTGAACGAGGTGGCCAAGAAT |
| | | CTGAACGAGAGCCTGATCGACCTGCAAGAACTGGGGAAGTACGA |
| | | GCAGTACATCAAGTGGCCCTGGTACATCTGGCTGGGCTTTATCGC |
| | | CGGACTGATTGCCATCGTGATGGTCACAATCATGCTGTGTTGCAT |
| | | GACCAGCTGCTGTAGCTGCCTGAAGGGCTGTTGTAGCTGTGGCA |
| | | GCTGCTGCTAA |
| Hepatitis B | 94 | ATGCCCCTATCCTATCAACACTTCCGGAGACTACTGTTGTTAGAC |
| Surface | | GACGAGGCAGGTCCCCTAGAAGAAGAACTCCCTCGCCTCGCAGA |
| Antigen | | CGAAGGTCTCAATCGCCGCGTCGCAGAAGATCTCAATCTCGGGA |
| | | ATCTCAATGTTAGTATTCCTTGGACTCATAAGGTGGGGAACTTTA |
| | | CTGGGCTTTATTCTTCTACTGTACCTGTCTTTAATCCTCATTGGAA |
| | | AACACCATCTTTTCCTAATATACATTTACACCAAGACATTATCAA |
| | | AAAATGTGAACAGTTTGTAGGCCCACTCACAGTTAATGAGAAAA |
| | | GAAGATTGCAATTGATTATGCCTGCCAGGTTTTATCCAAAGGTTA |
| | | CCAAATATTTACCATTGGATAAGGGTATTAAACCTTATTATCCAG |
| | | AACATCTAGTTAATCATTACTTCCAAACTAGACACTATTTACACA |
| | | CTCTATGGAAGGCGGGTATATTATATAAGAGAGAAACAACACAT |
| | | AGCGCCTCATTTTGTGGGTCACCATATTCTTGGGAACAAGATCTA |
| | | CAGCATGGGGCAGAATCTTTCCACCAGCAATCCTCTGGGATTCTT |
| | | TCCCGACCACCAGTTGGATCCAGCCTTCAGAGCAAACACCGCAA |
| | | ATCCAGATTGGGACTTCAATCCCAACAAGGACACCTGGCCAGAC |
| | | GCCAACAAGGTAGGAGCTGGAGCATTCGGGCTGGGTTTCACCCC |
| | | ACCGCACGGAGGCCTTTTGGGGTGGAGCCCTCAGGCTCAGGGCA |
| | | TACTACAAACTTTGCCAGCAAATCCGCCTCCTGCCTCCACCAATC |
| | | GCCAGTCAGGAAGGCAGCCTACCCCGCTGTCTCCACCTTTGAGA |
| | | AACACTCATCCTCAGGCCATGCAGTGGAATTCCACAACCTTCCAC |
| | | CAAACTCTGCAAGATCCCAGAGTGAGAGGCCTGTATTTCCCTGCT |
| | | GGTGGCTCCAGTTCAGGAACAGTAAACCCTGTTCTGACTACTGCC |
| | | TCTCCCTTATCGTCAATCTTCTCGAGGATTGGGGACCCTGCGCTG |
| | | AACATGGAGAACATCACATCAGGATTCCTAGGACCCCTTCTCGT |
| | | GTTACAGGCGGGGTTTTTCTTGTTGACAAGAATCCTCACAATACC |
| | | GCAGAGTCTAGACTCGTGGTGGACTTCTCTCAATTTTCTAGGGGG |
| | | AACTACCGTGTGTCTTGGCCAAAATTCGCAGTCCCCAACCTCCAA |
| | | TCACTCACCAACCTCTTGTCCTCCAACTTGTCCTGGTTATCGCTG |
| | | GATGTGTCTGCGGCGTTTTATCATCTTCCTCTTCATCCTGCTGCTA |
| | | TGCCTCATCTTCTTGTTGGTTCTTCTGGACTATCAAGGTATGTTGC |
| | | CCGTTTGTCCTCTAATTCCAGGATCCTCAACAACCAGCACGGGAC |
| | | CATGCCGGACCTGCATGACTACTGCTCAAGGAACCTCTATGTATC |
| | | CCTCCTGTTGCTGTACCAAACCTTCGGACGGAAATTGCACCTGTA |
| | | TTCCCATCCCATCATCCTGGGCTTTCGGAAAATTCCTATGGGAGT |
| | | GGGCCTCAGCCCGTTTCTCCTGGCTCAGTTTACTAGTGCCATTTG |
| | | TTCAGTGGTTCGTAGGGCTTTCCCCCACTGTTTGGCTTTCAGTTAT |
| | | ATGGATGATGTGGTATTGGGGGCCAAGTCTGTACAGCATCTTGA |
| | | GTCCCTTTTTACCGCTGTTACCAATTTTCTTTTGTCTTTGGGTATA |
| | | CATTTAAACCCTAACAAAACAAAGAGATGGGGTTACTCTCTAAA |
| | | TTTTATGGGTTATGTCATTGGATGTTATGGGTCCTTGCCACAAGA |
| | | ACACATCATACAAAAAATCAAAGAATGTTTTAGAAAACTTCCTA |
| | | TTAACAGGCCTATTGATTGGAAAGTATGTCAACGAATTGTGGGT |
| | | CTTTTGGGTTTTGCTGCCCCTTTTACACAATGTGGTTATCCTGCGT |
| | | TGATGCCTTTGTATGCATGTATTCAATCTAAGCAGGCTTTCACTT |
| | | TCTCGCCAACTTACAAGGCCTTTCTGTGTAAACAATACCTGAACC |
| | | TTTACCCCGTTGCCCGGCAACGGCCAGGTCTGTGCCAAGTGTTTG |
| | | CTGACGCAACCCCCACTGGCTGGGGCTTGGTCATGGGCCATCAG |
| | | CGCATGCGTGGAACCTTTTCGGCTCCTCTGCCGATCCATACTGCG |
| | | GAACTCCTAGCCGCTTGTTTTGCTCGCAGCAGGTCTGGAGCAAAC |
| | | ATTATCGGGACTGATAACTCTGTTGTCCTATCCCGCAAATATACA |
| | | TCGTTTCCATGGCTGCTAGGCTGTGCTGCCAACTGGATCCTGCGC |
| | | GGGACGTCCTTTGTTTACGTCCCGTCGGCGCTGAATCCTGCGGAC |
| | | GACCCTTCTCGGGGTCGCTTGGGACTCTCTCGTCCCCTTCTCCGT |
| | | CTGCCGTTCCGACCGACCACGGGGCGCACCTCTCTTTACGCGGAC |
| | | TCCCCGTCTGTGCCTTCTCATCTGCCGGACCGTGTGCACTTCGCTT |
| | | CACCTCTGCACGTCGCATGGAGACCACCGTGA |
| L1 major | 95 | ATGTCTCTTTGGCTGCCTAGTGAGGCCACTGTCTACTTGCCTCCT |
| capsid | | GTCCCAGTATCTAAGGTTGTAAGCACGGATGAATATGTTGCACG |
| protein HPV | | CACAAACATATATTATCATGCAGGAACATCCAGACTACTTGCAG |
| | | TTGGACATCCCTATTTTCCTATTAAAAAACCTAACAATAACAAAA |
| | | TATTAGTTCCTAAAGTATCAGGATTACAATACAGGGTATTTAGAA |
| | | TACATTTACCTGACCCCAATAAGTTTGGTTTTCCTGACACCTCAT |
| | | TTTATAATCCAGATACACAGCGGCTGGTTTGGGCCTGTGTAGGTG |
| | | TTGAGGTAGGTCGTGGTCAGCCATTAGGTGTGGGCATTAGTGGC |
| | | CATCCTTTATTAAATAAATTGGATGACACAGAAAATGCTAGTGCT |
| | | TATGCAGCAAATGCAGGTGTGGATAATAGAGAATGTATATCTAT |
| | | GGATTACAAACAAACACAATTGTGTTTAATTGGTTGCAAACCAC |
| | | CTATAGGGGAACACTGGGGCAAAGGATCCCCATGTACCAATGTT |
| | | GCAGTAAATCCAGGTGATTGTCCACCATTAGAGTTAATAAACAC |
| | | AGTTATTCAGGATGGTGATATGGTTGATACTGGCTTTGGTGCTAT |
| | | GGACTTTACTACATTACAGGCTAACAAAAGTGAAGTTCCACTGG |
| | | ATATTTGTACATCTATTTGCAAATATCCAGATTATATTAAAATGG |
| | | TGTCAGAACCATATGGCGACAGCTTATTTTTTTATTTACGAAGGG |
| | | AACAAATGTTTGTTAGACATTTATTTAATAGGGCTGGTACTGTTG |
| | | GTGAAAATGTACCAGACGATTTATACATTAAAGGCTCTGGGTCT |
| | | ACTGCAAATTTAGCCAGTTCAAATTATTTTCCTACACCTAGTGGT |
| | | TCTATGGTTACCTCTGATGCCCAAATATTCAATAAACCTTATTGG |
| | | TTACAACGAGCACAGGGCCACAATAATGGCATTTGTTGGGGTAA |
| | | CCAACTATTTGTTACTGTTGTTGATACTACACGCAGTACAAATAT |
| | | GTCATTATGTGCTGCCATATCTACTTCAGAAACTACATATAAAAA |
| | | TACTAACTTTAAGGAGTACCTACGACATGGGGAGGAATATGATT |
| | | TACAGTTTATTTTTCAACTGTGCAAAATAACCTTAACTGCAGACG |
| | | TTATGACATACATACATTCTATGAATTCCACTATTTTGGAGGACT |
| | | GGAATTTTGGTCTACAACCTCCCCCAGGAGGCACACTAGAAGAT |
| | | ACTTATAGGTTTGTAACATCCCAGGCAATTGCTTGTCAAAAACAT |
| | | ACACCTCCAGCACCTAAAGAAGATCCCCTTAAAAAATACACTTT |
| | | TTGGGAAGTAAATTTAAAGGAAAAGTTTTCTGCAGACCTAGATC |
| | | AGTTTCCTTTAGGACGCAAATTTTTACTACAAGCAGGATTGAAGG |
| | | CCAAACCAAAATTTACATTAGGAAAACGAAAAGCTACACCCACC |
| | | ACCTCATCTACCTCTACAACTGCTAAACGCAAAAAACGTAAGCT |
| | | GTAA |
| HA | 96 | ATGGAGAAAATAGTGCTTCTTCTTGCAATAGTCAGTCTTGTCAAA |
| hemagglutinin | | AGTGATCAGATTTGCATTGGTTACCATGCAAACAACTCGACAGA |
| [Influenza A | | GCAGGTTGACACAATAATGGAAAAGAACGTTACTGTTACACATG |
| virus | | CCCAAGACATACTGGAAAAGACACACAATGGGAAGCTCTGCGAT |
| (A/goose/ | | CTAAATGGAGTGAAGCCTCTCATTTTGAGAGATTGTAGTGTAGCT |
| Guangdong/1/ | | GGATGGCTCCTCGGAAACCCTATGTGTGACGAATTCATCAATGT |
| 1996(H5N1)] | | GCCGGAATGGTCTTACATAGTGGAGAAGGCCAGTCCAGCCAATG |
| | | ACCTCTGTTACCCAGGGGATTTCAACGACTATGAAGAACTGAAA |
| | | CACCTATTGAGCAGAACAAACCATTTTGAGAAAATTCAGATCAT |
| | | CCCCAAAAGTTCTTGGTCCAATCATGATGCCTCATCAGGGGTGA |
| | | GCTCAGCATGTCCATACCATGGGAGGTCCTCCTTTTTCAGAAATG |
| | | TGGTATGGCTTATCAAAAAGAACAGTGCATACCCAACAATAAAG |
| | | AGGAGCTACAATAATACCAACCAAGAAGATCTTTTAGTACTGTG |
| | | GGGGATTCACCATCCTAATGATGCGGCAGAGCAGACAAAGCTCT |
| | | ATCAAAACCCAACCACTTACATTTCCGTTGGAACATCAACACTG |
| | | AACCAGAGATTGGTTCCAGAAATAGCTACTAGACCCAAAGTAAA |
| | | CGGGCAAAGTGGAAGAATGGAGTTCTTCTGGACAATTTTAAAGC |
| | | CGAATGATGCCATCAATTTCGAGAGTAATGGAAATTTCATTGCTC |
| | | CAGAATATGCATACAAAATTGTCAAGAAAGGGGACTCAGCAATT |
| | | ATGAAAAGTGAATTGGAATATGGTAACTGCAACACCAAGTGTCA |
| | | AACTCCAATGGGGGCGATAAACTCTAGTATGCCATTCCACAACA |
| | | TACACCCCCTCACCATCGGGGAATGCCCCAAATATGTGAAATCA |
| | | AACAGATTAGTCCTTGCGACTGGACTCAGAAATACCCCTCAGAG |
| | | AGAGAGAAGAAGAAAAAAGAGAGGACTATTTGGAGCTATAGCA |
| | | GGTTTTATAGAGGGAGGATGGCAGGGAATGGTAGATGGTTGGTA |
| | | TGGGTACCACCATAGCAATGAGCAGGGGAGTGGATACGCTGCAG |
| | | ACAAAGAATCCACTCAAAAGGCAATAGATGGAGTCACCAATAA |
| | | GGTCAACTCGATCATTGACAAAATGAACACTCAGTTTGAGGCCG |
| | | TTGGAAGGGAATTTAATAACTTGGAAAGGAGGATAGAGAATTTA |
| | | AACAAGCAGATGGAAGACGGATTCCTAGATGTCTGGACTTATAA |
| | | TGCTGAACTTCTGGTTCTCATGGAAAATGAGAGAACTCTAGACTT |
| | | TCATGACTCAAATGTCAAGAACCTTTATGACAAGGTCCGACTAC |
| | | AGCTTAGGGATAATGCAAAGGAGCTGGGTAATGGTTGTTTCGAG |
| | | TTCTATCACAAATGTGATAATGAATGTATGGAAAGTGTAAAAAA |
| | | CGGAACGTATGACTACCCGCAGTATTCAGAAGAAGCAAGACTAA |
| | | ACAGAGAGGAAATAAGTGGAGTAAAATTGGAATCAATGGGAAC |
| | | TTACCAAATACTGTCAATTTATTCAACAGTGGCGAGTTCCCTAGC |
| | | ACTGGCAATCATGGTAGCTGGTCTATCTTTATGGATGTGCTCCAA |
| | | TGGATCGTTACAATGCAGAATTTGCATTTAA |
| Antigen | SEQ | Amino acid sequence |
| COVID-19 | 97 | VNLTTRTQLPPAYTNSFTRGVYYPDKVFRSSVLHSTQDLFLPFFSNV |
| Spike | | TWFHAIHVSGTNGTKRFDNPVLPFNDGVYFASTEKSNIIRGWIFGTT |
| stabilized | | LDSKTQSLLIVNNATNVVIKVCEFQFCNDPFLGVYYHKNNKSWMES |
| (K986P and | | EFRVYSSANNCTFEYVSQPFLMDLEGKQGNFKNLREFVFKNIDGYF |
| V987P), | | KIYSKHTPINLVRDLPQGFSALEPLVDLPIGINITRFQTLLALHRSYLT |
| K417T, | | PGDSSSGWTAGAAAYYVGYLQPRTFLLKYNENGTITDAVDCALAP |
| E484K, | | LSETKCTLKSFTVEKGIYQTSNFRVQPTESIVRFPNITNLCPFGEVFNA |
| N501Y | | TRFASVYAWNRKRISNCVADYSVLYNSASFSTFKCYGVSPTKLNDL |
| | | CFTNVYADSFVIRGDEVRQIAPGQTGTIADYNYKLPDDFTGCVIAW |
| | | NSNNLDSKVGGNYNYLYRLFRKSNLKPFERDISTEIYQAGSTPCNGV |
| | | KGFNCYFPLQSYGFQPTYGVGYQPYRVVVLSFELLHAPATVCGPKK |
| | | STNLVKNKCVNFNFNGLTGTGVLTESNKKFLPFQQFGRDIADTTDA |
| | | VRDPQTLEILDITPCSFGGVSVITPGTNTSNQVAVLYQDVNCTEVPV |
| | | AIHADQLTPTWRVYSTGSNVFQTRAGCLIGAEHVNNSYECDIPIGAG |
| | | ICASYQTQTNSPRRARSVASQSIIAYTMSLGAENSVAYSNNSIAIPTN |
| | | FTISVTTEILPVSMTKTSVDCTMYICGDSTECSNLLLQYGSFCTQLNR |
| | | ALTGIAVEQDKNTQEVFAQVKQIYKTPPIKDFGGFNFSQILPDPSKPS |
| | | KRSFIEDLLFNKVTLADAGFIKQYGDCLGDIAARDLICAQKFNGLTV |
| | | LPPLLTDEMIAQYTSALLAGTITSGWTFGAGAALQIPFAMQMAYRF |
| | | NGIGVTQNVLYENQKLIANQFNSAIGKIQDSLSSTASALGKLQDVVN |
| | | QNAQALNTLVKQLSSNFGAISSVLNDILSRLDPPEAEVQIDRLITGRL |
| | | QSLQTYVTQQLIRAAEIRASANLAATKMSECVLGQSKRVDFCGKGY |
| | | HLMSFPQSAPHGVVFLHVTYVPAQEKNFTTAPAICHDGKAHFPREG |
| | | VFVSNGTHWFVTQRNFYEPQIITTDNTFVSGNCDVVIGIVNNTVYDP |
| | | LQPELDSFKEELDKYFKNHTSPDVDLGDISGINASVVNIQKEIDRLNE |
| | | VAKNLNESLIDLQELGKYEQYIKWPWYIWLGFIAGLIAIVMVTIMLC |
| | | CMTSCCSCLKGCCSCGSCC |
| Hepatitis B | 98 | MPLSYQHFRRLLLLDDEAGPLEEELPRLADEGLNRRVAEDLNLGNL |
| Surface | | NVSIPWTHKVGNFTGLYSSTVPVFNPHWKTPSFPNIHLHQDIIKKCE |
| Antigen | | QFVGPLTVNEKRRLQLIMPARFYPKVTKYLPLDKGIKPYYPEHLVN |
| | | HYFQTRHYLHTLWKAGILYKRETTHSASFCGSPYSWEQDLQHGAES |
| | | FHQQSSGILSRPPVGSSLQSKHRKSRLGLQSQQGHLARRQQGRSWSI |
| | | RAGFHPTARRPFGVEPSGSGHTTNFASKSASCLHQSPVRKAAYPAV |
| | | STFEKHSSSGHAVEFHNLPPNSARSQSERPVFPCWWLQFRNSKPCSD |
| | | YCLSLIVNLLEDWGPCAEHGEHHIRIPRTPSRVTGGVFLVDKNPHNT |
| | | AESRLVVDFSQFSRGNYRVSWPKFAVPNLQSLTNLLSSNLSWLSLD |
| | | VSAAFYHLPLHPAAMPHLLVGSSGLSRYVARLSSNSRILNNQHGTM |
| | | PDLHDYCSRNLYVSLLLLYQTFGRKLHLYSHPIILGFRKIPMGVGLSP |
| | | FLLAQFTSAICSVVRRAFPHCLAFSYMDDVVLGAKSVQHLESLFTA |
| | | VTNFLLSLGIHLNPNKTKRWGYSLNFMGYVIGCYGSLPQEHIIQKIK |
| | | ECFRKLPINRPIDWKVCQRIVGLLGFAAPFTQCGYPALMPLYACIQS |
| | | KQAFTFSPTYKAFLCKQYLNLYPVARQRPGLCQVFADATPTGWGL |
| | | VMGHQRMRGTFSAPLPIHTAELLAACFARSRSGANIIGTDNSVVLSR |
| | | KYTSFPWLLGCAANWILRGTSFVYVPSALNPADDPSRGRLGLSRPL |
| | | LRLPFRPTTGRTSLYADSPSVPSHLPDRVHFASPLHVAWRPP |
| L1 major | 99 | MSLWLPSEATVYLPPVPVSKVVSTDEYVARTNIYYHAGTSRLLAVG |
| capsid | | HPYFPIKKPNNNKILVPKVSGLQYRVFRIHLPDPNKFGFPDTSFYNPD |
| protein HPV | | TQRLVWACVGVEVGRGQPLGVGISGHPLLNKLDDTENASAYAANA |
| | | GVDNRECISMDYKQTQLCLIGCKPPIGEHWGKGSPCTNVAVNPGDC |
| | | PPLELINTVIQDGDMVDTGFGAMDFTTLQANKSEVPLDICTSICKYP |
| | | DYIKMVSEPYGDSLFFYLRREQMFVRHLFNRAGTVGENVPDDLYIK |
| | | GSGSTANLASSNYFPTPSGSMVTSDAQIFNKPYWLQRAQGHNNGIC |
| | | WGNQLFVTVVDTTRSTNMSLCAAISTSETTYKNTNFKEYLRHGEEY |
| | | DLQFIFQLCKITLTADVMTYIHSMNSTILEDWNFGLQPPPGGTLEDT |
| | | YRFVTSQAIACQKHTPPAPKEDPLKKYTFWEVNLKEKFSADLDQFP |
| | | LGRKFLLQAGLKAKPKFTLGKRKATPTTSSTSTTAKRKKRKL |
| HA | 100 | MEKIVLLLAIVSLVKSDQICIGYHANNSTEQVDTIMEKNVTVTHAQD |
| hemagglutinin | | ILEKTHNGKLCDLNGVKPLILRDCSVAGWLLGNPMCDEFINVPEWS |
| [Influenza A | | YIVEKASPANDLCYPGDFNDYEELKHLLSRTNHFEKIQIIPKSSWSNH |
| virus | | DASSGVSSACPYHGRSSFFRNVVWLIKKNSAYPTIKRSYNNTNQED |
| (A/goose/ | | LLVLWGIHHPNDAAEQTKLYQNPTTYISVGTSTLNQRLVPEIATRPK |
| Guangdong/1/ | | VNGQSGRMEFFWTILKPNDAINFESNGNFIAPEYAYKIVKKGDSAIM |
| 1996(H5N1)] | | KSELEYGNCNTKCQTPMGAINSSMPFHNIHPLTIGECPKYVKSNRLV |
| | | LATGLRNTPQRERRRKKRGLFGAIAGFIEGGWQGMVDGWYGYHHS |
| | | NEQGSGYAADKESTQKAIDGVTNKVNSIIDKMNTQFEAVGREFNNL |
| | | ERRIENLNKQMEDGFLDVWTYNAELLVLMENERTLDFHDSNVKNL |
| | | YDKVRLQLRDNAKELGNGCFEFYHKCDNECMESVKNGTYDYPQY |
| | | SEEARLNREEISGVKLESMGTYQILSIYSTVASSLALAIMVAGLSLW |
| | | MCSNGSLQCRICI |

### Signal Peptides

Provided herein, in some embodiments, are nucleic acid compositions comprising a polynucleotide encoding a signal peptide. Further provided in some embodiments are peptide compositions comprising a signal peptide. In some embodiments, a signal peptide refers to a short polypeptide, which is from about 3 to 60 amino acids in length, present at the 5′ (or N-terminus) of newly synthesized proteins. Signal peptides function to prompt a cell to translocate the protein to the cellular membrane through a secretory pathway. Signal peptides generally contain an N-terminal region comprising positively charged amino acids, a hydrophobic region, and a short carboxy-terminal peptide region. In eukaryotes, the signal peptide directs the ribosome to the endoplasmic reticulum (ER) membrane and initiates the transpose of the newly synthesized protein for processing. Some signal peptides are cleaved from the protein by signal peptidase after the proteins are transported. Others remain uncleaved and function as a membrane anchor.

In some embodiments, the signal peptide is a native signal peptide or a non-native signal peptide. In some embodiments, the signal peptide is Gaussia luciferase, Human albumin, Human chymotrypsinogen, Human interleukin-2, or Human trypsinogen-2. In some embodiments, the signal peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 107-112. In some embodiments, the polynucleotide encoding the signal peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 101-106.

TABLE 5
Example signal peptide sequences

| | SEQ ID | | | |
|---|---|---|---|---|
| | NO | | SEQ ID | |
| | (nucleic | | NO | |
| Signal | acid) | Nucleic acid sequence | (peptide) | Peptide sequence |
| Spike signal | 101 | ATGTTCGTGTTTCTGGTG | 107 | MFVFLVLLPLVSSQC |
| peptide | | CTGCTGCCTCTGGTGTCC | | |
| | | AGCCAGTGT | | |
| Gaussia | 102 | ATGGGCGTGAAGGTGCTG | 108 | MGVKVLFALICIAVA |
| luciferase | | TTCGCCCTGATCTGCATC | | EA |
| | | GCCGTGGCCGAGGCC | | |
| Human | 103 | ATGAAGTGGGTGACCTTC | 109 | MKWVTFISLLFLFSS |
| albumin | | ATCAGCCTGCTGTTCCTG | | AYS |
| | | TTCAGCAGCGCCTACAGC | | |
| Human | 104 | ATGGCCTTCCTGTGGCTG | 110 | MAFLWLLSCWALLG |
| chymo- | | CTGAGCTGCTGGGCCCTG | | TTFG |
| trypsinogen | | CTGGGCACCACCTTCGGC | | |
| Human | 105 | ATGCAGCTGCTGAGCTGC | 111 | MQLLSCIALILALV |
| interleukin- | | ATCGCCCTGATCCTGGCC | | |
| 2 | | CTGGTG | | |
| Human | 106 | ATGAACCTGCTGCTGATC | 112 | MNLLLILTFVAAAVA |
| trypsinogen- | | CTGACCTTCGTGGCCGCC | | |
| 2 | | GCCGTGGCC | | |

### MHC Binding Peptides

In one aspect, provided herein are nucleic acid compositions comprising a sequence encoding a MHC binding peptide. In some embodiments, the nucleic acid composition comprises a first sequence encoding an antigen, and a second sequence encoding a MHC binding peptide, wherein the first and second sequence are located on the same or separate nucleic acid sequences. As a non-limiting example where the first and second sequences are on separate nucleic acid sequences, the first sequence is administered before, during, or after administration of the second sequence.

In another aspect, provided herein are peptide compositions comprising a MHC binding peptide. In some embodiments, the peptide composition comprises a MHC binding peptide and a peptide antigen, where the MHC binding peptide and the peptide antigen are on separate or connected polypeptides. As a non-limiting example where the MHC binding peptide and peptide antigen are located on separate polypeptides, the MHC binding peptide is administered to a subject before, during, or after administration of the peptide antigen. Example peptide compositions include vaccines, for instance, vaccines against a pathogen such as Hepatitis B, SARS-Cov2, Ebola, Pertussis, tetanus, HPV, and Diphtheria.

In some embodiments, the nucleic acid compositions comprising a sequence encoding a MHC binding peptide further comprise a flavivirus 5′ UTR and/or a flavivirus 3′ UTR, e.g., as disclosed herein. In some embodiments, the nucleic acid compositions comprising a sequence encoding a MHC binding peptide do not comprise a flavivirus 5′ UTR. In some embodiments, the nucleic acid compositions comprising a sequence encoding a MHC binding peptide do not comprise a flavivirus 3′ UTR.

In some embodiments, a MHC binding peptide refers to a peptide that binds to a major histocompatibility complex (MHC). A major histocompatibility complex (MHC) is a complex of genes that code for proteins found on the surfaces of cells that are important for signaling between lymphocytes and antigen presenting cells or diseased cells in immune system, wherein the MHC molecules bind peptides and present them for recognition by T cell receptors. There are two types of MHC molecules—MHC class I molecules and MHC class II molecules. MHC class I molecules are expressed in the membrane of almost every cell in an organism, while MHC class II molecules are restricted to macrophages and lymphocytes. In some embodiments, a MHC class I molecule has a length of about 5, 10, 15, or 20 amino acids. For instance, a MHC class I molecule has length of about 5, 6, 7, 8, 9, 10, 11, 12, 13, 14, 15, 16, 17, 18, 19, or 20 amino acids. In some embodiments, a MHC class II molecule has a length of about 5, 10, 15, 20, 25, 30, 35, or 40 amino acids. For instance, a MHC class I molecule has length of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids.

In some embodiments, provided herein are MHC binding peptides that bind to a major histocompatibility complex (MHC) at sufficient affinity to allow the peptide/MHC complex to interact with a T-cell receptor on T-cells. The binding affinity of the peptide/MHC complex with T-cell receptor on T-cells can be measured by cytokine production and/or T-cell proliferation. In embodiments, MHC binding peptides have an affinity IC50 value of 5000 nM or less, 500 nM or less, and 50 nM or less for binding to an MHC molecule. For instance, MHC I binding peptides have an affinity IC50 value of 5000 nM or less, 500 nM or less, or 50 nM or less for binding to an MHC class I molecule. For instance, MHC II binding peptides have an affinity IC50 value of 5000 nM or less, 500 nM or less, or 50 nM or less for binding to an MHC class II molecule.

In some embodiments, T cell antigen refers to a CD4+ T-cell antigen or a CD+ T-cell antigen. In some embodiments, a CD4+ T-cell antigen refers to any antigen that is recognized by a T-cell receptor on a CD4+ T cell via presentation of the antigen or portion thereof bound to a MHC class II molecule. In other embodiments, a CD8+ T-cell antigen refers to any antigen that is recognized by a T-cell receptor on a CD8+ T cell via presentation of the antigen or portion thereof bound to a MHC class I molecule. In some embodiments, T cell antigens are antigens that stimulate a CD4+ T cell response or a CD8+ T cell response. In some embodiments, T cell antigens are proteins or peptides, but may be other molecules such as lipids and glycolipids. In some embodiments, an antigen that is a T cell antigen is also a B cell antigen. In other embodiments, the T cell antigen is not also a B cell antigen.

In some embodiments, a MHC binding peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to about 7 or more amino acids of a pathogen protein. Pathogens include, without limitation, virus, bacteria, fungus, protozoa, and helminth. In some cases, 7 or more amino acids of a pathogen protein is about 7 to about 20 amino acids of a pathogen protein. For instance, about 7, 8, 9, 10, 11, 12, 13, 14, 15, 16, 17, 18, 19, or 20 amino acids of a pathogen protein.

### Viral Proteins

In some embodiments, a MHC binding peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to about 7 or more amino acids of a viral protein. Non-limiting example viruses include Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Bimaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus.

### Bacterial Proteins

In some embodiments, a MHC binding peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to about 7 or more amino acids of a bacterial protein. Non-limiting example bacteria include Helicobacter pyloris, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii.

### Fungal Proteins

In some embodiments, a MHC binding peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to about 7 or more amino acids of a fungal protein. Non-limiting example fungi include Cryptococcus neoformans, Histoplasma capsulatum, Coccidioides immitis, Blastomyces dermatitidis, Chlamydia trachomatis, and Candida albicans.

### Protozoal Proteins

In some embodiments, a MHC binding peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to about 7 or more amino acids of a protozoal protein. Non-limiting example protozoa include Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major.

### Helminth Proteins

In some embodiments, a MHC binding peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to about 7 or more amino acids of a helminth protein. Non-limiting example helminth include hookworm, Onchocerca volvulus, Brugia malayi, and Ascaris lumbricoides, Ancylostoma caninum excretory/secretory products (AcES), and Schistosoma mansoni.

### Non-Limiting Example MHC Binding Sequences

In some embodiments, a sequence encoding a MHC binding peptide comprises a sequence at least 80%, 81%, 82%, 83%, 84%, 85%, 86%, 87%, 88%, 89%, 90%, 91%, 92%, 93% 94%, 95%, 96%, 97%, 98%, 99%, or is 100% identical to any one of SEQ ID NOS: 113-135.

TABLE 6
Example nucleic acid sequences encoding MHC binding peptides

| | SEQ | |
|---|---|---|
| Antigen | ID NO | Nucleic acid sequence |
| Mycobacterium | 113 | TTCCAGGACGCCTACAACGCCGCCGGCGGCCACAACGCCGTGTTC |
| p25 | | |
| M. | 114 | ATGGCAGAGATGAAGACCGATGCCGCTACCCTCGCGCAGGAGGCAG |
| tuberculosis | | GTAATTTCGAGCGGATCTCCGGCGACCTGAAAACCCAGATCGACCAG |
| CFP-10 | | GTGGAGTCGACGGCAGGTTCGTTGCAGGGCCAGTGGCGCGGCGCGGC |
| | | GGGGACGGCCGCCCAGGCCGCGGTGGTGCGCTTCCAAGAAGCAGCCA |
| | | ATAAGCAGAAGCAGGAACTCGACGAGATCTCGACGAATATTCGTCAG |
| | | GCCGGCGTCCAATACTCGAGGGCCGACGAGGAGCAGCAGCAGGCGC |
| | | TGTCCTCGCAAATGGGCTTCTGA |
| SARS-CoV- | 115 | ATGTTCGTGTTCCTGGTGCTGCTGCCCCTGGTGAGCAGCCAGTGCGTG |
| 2 Spike | | AACCTGACCACCAGGACCCAGCTGCCCCCCGCCTACACCAACAGCTT |
| | | CACCAGGGGCGTGTACTACCCCGACAAGGTGTTCAGGAGCAGCGTGC |
| | | TGCACAGCACCCAGGACCTGTTCCTGCCCTTCTTCAGCAACGTGACCT |
| | | GGTTCCACGCCATCCACGTGAGCGGCACCAACGGCACCAAGAGGTTC |
| | | GACAACCCCGTGCTGCCCTTCAACGACGGCGTGTACTTCGCCAGCAC |
| | | CGAGAAGAGCAACATCATCAGGGGCTGGATCTTCGGCACCACCCTGG |
| | | ACAGCAAGACCCAGAGCCTGCTGATCGTGAACAACGCCACCAACGTG |
| | | GTGATCAAGGTGTGCGAGTTCCAGTTCTGCAACGACCCCTTCCTGGGC |
| | | GTGTACTACCACAAGAACAACAAGAGCTGGATGGAGAGCGAGTTCA |
| | | GGGTGTACAGCAGCGCCAACAACTGCACCTTCGAGTACGTGAGCCAG |
| | | CCCTTCCTGATGGACCTGGAGGGCAAGCAGGGCAACTTCAAGAACCT |
| | | GAGGGAGTTCGTGTTCAAGAACATCGACGGCTACTTCAAGATCTACA |
| | | GCAAGCACACCCCCATCAACCTGGTGAGGGACCTGCCCCAGGGCTTC |
| | | AGCGCCCTGGAGCCCCTGGTGGACCTGCCCATCGGCATCAACATCAC |
| | | CAGGTTCCAGACCCTGCTGGCCCTGCACAGGAGCTACCTGACCCCCG |
| | | GCGACAGCAGCAGCGGCTGGACCGCCGGCGCCGCCGCCTACTACGTG |
| | | GGCTACCTGCAGCCCAGGACCTTCCTGCTGAAGTACAACGAGAACGG |
| | | CACCATCACCGACGCCGTGGACTGCGCCCTGGACCCCCTGAGCGAGA |
| | | CCAAGTGCACCCTGAAGAGCTTCACCGTGGAGAAGGGCATCTACCAG |
| | | ACCAGCAACTTCAGGGTGCAGCCCACCGAGAGCATCGTGAGGTTCCC |
| | | CAACATCACCAACCTGTGCCCCTTCGGCGAGGTGTTCAACGCCACCA |
| | | GGTTCGCCAGCGTGTACGCCTGGAACAGGAAGAGGATCAGCAACTGC |
| | | GTGGCCGACTACAGCGTGCTGTACAACAGCGCCAGCTTCAGCACCTT |
| | | CAAGTGCTACGGCGTGAGCCCCACCAAGCTGAACGACCTGTGCTTCA |
| | | CCAACGTGTACGCCGACAGCTTCGTGATCAGGGGCGACGAGGTGAGG |
| | | CAGATCGCCCCCGGCCAGACCGGCAAGATCGCCGACTACAACTACAA |
| | | GCTGCCCGACGACTTCACCGGCTGCGTGATCGCCTGGAACAGCAACA |
| | | ACCTGGACAGCAAGGTGGGCGGCAACTACAACTACCTGTACAGGCTG |
| | | TTCAGGAAGAGCAACCTGAAGCCCTTCGAGAGGGACATCAGCACCGA |
| | | GATCTACCAGGCCGGCAGCACCCCCTGCAACGGCGTGGAGGGCTTCA |
| | | ACTGCTACTTCCCCCTGCAGAGCTACGGCTTCCAGCCCACCAACGGCG |
| | | TGGGCTACCAGCCCTACAGGGTGGTGGTGCTGAGCTTCGAGCTGCTG |
| | | CACGCCCCCGCCACCGTGTGCGGCCCCAAGAAGAGCACCAACCTGGT |
| | | GAAGAACAAGTGCGTGAACTTCAACTTCAACGGCCTGACCGGCACCG |
| | | GCGTGCTGACCGAGAGCAACAAGAAGTTCCTGCCCTTCCAGCAGTTC |
| | | GGCAGGGACATCGCCGACACCACCGACGCCGTGAGGGACCCCCAGA |
| | | CCCTGGAGATCCTGGACATCACCCCCTGCAGCTTCGGCGGCGTGAGC |
| | | GTGATCACCCCCGGCACCAACACCAGCAACCAGGTGGCCGTGCTGTA |
| | | CCAGGACGTGAACTGCACCGAGGTGCCCGTGGCCATCCACGCCGACC |
| | | AGCTGACCCCCACCTGGAGGGTGTACAGCACCGGCAGCAACGTGTTC |
| | | CAGACCAGGGCCGGCTGCCTGATCGGCGCCGAGCACGTGAACAACAG |
| | | CTACGAGTGCGACATCCCCATCGGCGCCGGCATCTGCGCCAGCTACC |
| | | AGACCCAGACCAACAGCCCCAGGAGGGCCAGGAGCGTGGCCAGCCA |
| | | GAGCATCATCGCCTACACCATGAGCCTGGGCGCCGAGAACAGCGTGG |
| | | CCTACAGCAACAACAGCATCGCCATCCCCACCAACTTCACCATCAGC |
| | | GTGACCACCGAGATCCTGCCCGTGAGCATGACCAAGACCAGCGTGGA |
| | | CTGCACCATGTACATCTGCGGCGACAGCACCGAGTGCAGCAACCTGC |
| | | TGCTGCAGTACGGCAGCTTCTGCACCCAGCTGAACAGGGCCCTGACC |
| | | GGCATCGCCGTGGAGCAGGACAAGAACACCCAGGAGGTGTTCGCCCA |
| | | GGTGAAGCAGATCTACAAGACCCCCCCCATCAAGGACTTCGGCGGCT |
| | | TCAACTTCAGCCAGATCCTGCCCGACCCCAGCAAGCCCAGCAAGAGG |
| | | AGCTTCATCGAGGACCTGCTGTTCAACAAGGTGACCCTGGCCGACGC |
| | | CGGCTTCATCAAGCAGTACGGCGACTGCCTGGGCGACATCGCCGCCA |
| | | GGGACCTGATCTGCGCCCAGAAGTTCAACGGCCTGACCGTGCTGCCC |
| | | CCCCTGCTGACCGACGAGATGATCGCCCAGTACACCAGCGCCCTGCT |
| | | GGCCGGCACCATCACCAGCGGCTGGACCTTCGGCGCCGCGCCGCCCT |
| | | GCAGATCCCCTTCGCCATGCAGATGGCCTACAGGTTCAACGGCATCG |
| | | GCGTGACCCAGAACGTGCTGTACGAGAACCAGAAGCTGATCGCCAAC |
| | | CAGTTCAACAGCGCCATCGGCAAGATCCAGGACAGCCTGAGCAGCAC |
| | | CGCCAGCGCCCTGGGCAAGCTGCAGGACGTGGTGAACCAGAACGCCC |
| | | AGGCCCTGAACACCCTGGTGAAGCAGCTGAGCAGCAACTTCGGCGCC |
| | | ATCAGCAGCGTGCTGAACGACATCCTGAGCAGGCTGGACAAGGTGGA |
| | | GGCCGAGGTGCAGATCGACAGGCTGATCACCGGCAGGCTGCAGAGCC |
| | | TGCAGACCTACGTGACCCAGCAGCTGATCAGGGCCGCCGAGATCAGG |
| | | GCCAGCGCCAACCTGGCCGCCACCAAGATGAGCGAGTGCGTGCTGGG |
| | | CCAGAGCAAGAGGGTGGACTTCTGCGGCAAGGGCTACCACCTGATGA |
| | | GCTTCCCCCAGAGCGCCCCCCACGGCGTGGTGTTCCTGCACGTGACCT |
| | | ACGTGCCCGCCCAGGAGAAGAACTTCACCACCGCCCCCGCCATCTGC |
| | | CACGACGGCAAGGCCCACTTCCCCAGGGAGGGCGTGTTCGTGAGCAA |
| | | CGGCACCCACTGGTTCGTGACCCAGAGGAACTTCTACGAGCCCCAGA |
| | | TCATCACCACCGACAACACCTTCGTGAGCGGCAACTGCGACGTGGTG |
| | | ATCGGCATCGTGAACAACACCGTGTACGACCCCCTGCAGCCCGAGCT |
| | | GGACAGCTTCAAGGAGGAGCTGGACAAGTACTTCAAGAACCACACCA |
| | | GCCCCGACGTGGACCTGGGCGACATCAGCGGCATCAACGCCAGCGTG |
| | | GTGAACATCCAGAAGGAGATCGACAGGCTGAACGAGGTGGCCAAGA |
| | | ACCTGAACGAGAGCCTGATCGACCTGCAGGAGCTGGGCAAGTACGAG |
| | | CAGTACATCAAGTGGCCCTGGTACATCTGGCTGGGCTTCATCGCCGGC |
| | | CTGATCGCCATCGTGATGGTGACCATCATGCTGTGCTGCATGACCAGC |
| | | TGCTGCAGCTGCCTGAAGGGCTGCTGCAGCTGCGGCAGCTGCTGCAA |
| | | GTTCGACGAGGACGACAGCGAGCCCGTGCTGAAGGGCGTGAAGCTGC |
| | | ACTACACC |
| Influenza A | 116 | ATGGAGAAAATAGTGCTTCTTCTTGCAATAGTCAGTCTTGTCAAAAGT |
| HA | | GATCAGATTTGCATTGGTTACCATGCAAACAACTCGACAGAGCAGGT |
| | | TGACACAATAATGGAAAAGAACGTTACTGTTACACATGCCCAAGACA |
| | | TACTGGAAAAGACACACAATGGGAAGCTCTGCGATCTAAATGGAGTG |
| | | AAGCCTCTCATTTTGAGAGATTGTAGTGTAGCTGGATGGCTCCTCGGA |
| | | AACCCTATGTGTGACGAATTCATCAATGTGCCGGAATGGTCTTACATA |
| | | GTGGAGAAGGCCAGTCCAGCCAATGACCTCTGTTACCCAGGGGATTT |
| | | CAACGACTATGAAGAACTGAAACACCTATTGAGCAGAACAAACCATT |
| | | TTGAGAAAATTCAGATCATCCCCAAAAGTTCTTGGTCCAATCATGATG |
| | | CCTCATCAGGGGTGAGCTCAGCATGTCCATACCATGGGAGGTCCTCCT |
| | | TTTTCAGAAATGTGGTATGGCTTATCAAAAAGAACAGTGCATACCCA |
| | | ACAATAAAGAGGAGCTACAATAATACCAACCAAGAAGATCTTTTAGT |
| | | ACTGTGGGGGATTCACCATCCTAATGATGCGGCAGAGCAGACAAAGC |
| | | TCTATCAAAACCCAACCACTTACATTTCCGTTGGAACATCAACACTGA |
| | | ACCAGAGATTGGTTCCAGAAATAGCTACTAGACCCAAAGTAAACGGG |
| | | CAAAGTGGAAGAATGGAGTTCTTCTGGACAATTTTAAAGCCGAATGA |
| | | TGCCATCAATTTCGAGAGTAATGGAAATTTCATTGCTCCAGAATATGC |
| | | ATACAAAATTGTCAAGAAAGGGGACTCAGCAATTATGAAAAGTGAAT |
| | | TGGAATATGGTAACTGCAACACCAAGTGTCAAACTCCAATGGGGGCG |
| | | ATAAACTCTAGTATGCCATTCCACAACATACACCCCCTCACCATCGGG |
| | | GAATGCCCCAAATATGTGAAATCAAACAGATTAGTCCTTGCGACTGG |
| | | ACTCAGAAATACCCCTCAGAGAGAGAGAAGAAGAAAAAAGAGAGGA |
| | | CTATTTGGAGCTATAGCAGGTTTTATAGAGGGAGGATGGCAGGGAAT |
| | | GGTAGATGGTTGGTATGGGTACCACCATAGCAATGAGCAGGGGAGTG |
| | | GATACGCTGCAGACAAAGAATCCACTCAAAAGGCAATAGATGGAGTC |
| | | ACCAATAAGGTCAACTCGATCATTGACAAAATGAACACTCAGTTTGA |
| | | GGCCGTTGGAAGGGAATTTAATAACTTGGAAAGGAGGATAGAGAATT |
| | | TAAACAAGCAGATGGAAGACGGATTCCTAGATGTCTGGACTTATAAT |
| | | GCTGAACTTCTGGTTCTCATGGAAAATGAGAGAACTCTAGACTTTCAT |
| | | GACTCAAATGTCAAGAACCTTTATGACAAGGTCCGACTACAGCTTAG |
| | | GGATAATGCAAAGGAGCTGGGTAATGGTTGTTTCGAGTTCTATCACA |
| | | AATGTGATAATGAATGTATGGAAAGTGTAAAAAACGGAACGTATGAC |
| | | TACCCGCAGTATTCAGAAGAAGCAAGACTAAACAGAGAGGAAATAA |
| | | GTGGAGTAAAATTGGAATCAATGGGAACTTACCAAATACTGTCAATT |
| | | TATTCAACAGTGGCGAGTTCCCTAGCACTGGCAATCATGGTAGCTGGT |
| | | CTATCTTTATGGATGTGCTCCAATGGATCGTTACAATGCAGAATTTGC |
| | | ATTTAA |
| Mtb ESAT- | 117 | ATGACAGAGCAGCAGTGGAATTTCGCGGGTATCGAGGCCGCGGCAAG |
| 6 | | CGCAATCCAGGGAAATGTCACGTCCATTCATTCCCTCCTTGACGAGGG |
| | | GAAGCAGTCCCTGACCAAGCTCGCAGCGGCCTGGGGCGGTAGCGGTT |
| | | CGGAGGCGTACCAGGGTGTCCAGCAAAAATGGGACGCCACGGCTACC |
| | | GAGCTGAACAACGCGCTGCAGAACCTGGCGCGGACGATCAGCGAAG |
| | | CCGGTCAGGCAATGGCTTCGACCGAAGGCAACGTCACTGGGATGTTC |
| | | GCATAG |
| Aspergillus | 118 | ATGTATTTCAAGTACACAGCAGCAGCCCTAGCTGCGGTGCTCCCTCTT |
| fumigatus | | TGCTCTGCACAGACTTGGTCAAAGTGCAATCCCCTTGAGAGTGAGTGT |
| Crf1/p41 | | TTTCATACCGACATATGATATACATCAGCTTATCTAACGATTGTTTTG |
| | | CAGAGACCTGCCCGCCCAACAAGGGTCTTGCTGCATCCACTTACACC |
| | | GCCGACTTCACCTCAGCTTCAGCTTTGGATCAATGGGAAGTCACTGCA |
| | | GGCAAAGTTCCCGTTGGCCCACAGGGCGCCGAGTTCACTGTCGCTAA |
| | | GCAAGGCGACGCACCTACCATTGACACCGACTTCTACTTCTTCTTCGG |
| | | AAAGGCCGAAGTGGTGATGAAGGCCGCTCCTGGCACAGGTGTTGTTA |
| | | GCAGCATCGTCCTGGAGTCGGATGATCTGGATGAGGTTGACTGGGTA |
| | | AGCCTGCTTGTCTATCATGTGTTCGTCTTGAGCCGGACTTAACGAAAG |
| | | CGCAGGAAGTATTGGGCGGTGACACCACTCAGGTTCAGACAAACTAC |
| | | TTTGGCAAAGGAGACACCACCACATATGACCGAGGCACTTACGTGCC |
| | | CGTTGCCACTCCTCAGGAGACTTTCCACACCTACACCATCGACTGGAC |
| | | CAAGGATGCCGTTACCTGGTCTATTGACGGTGCGGTCGTGCGTACGCT |
| | | CACGTACAACGATGCCAAGGGTGGCACTCGCTTCCCTCAGACTCCTAT |
| | | GCGCCTGAGACTTGGCAGCTGGGCCGGCGGCGACCCCAGCAACCCCA |
| | | AGGGCACCATCGAGTGGGCCGGTGGCTTGACCGACTACAGCGCGGGA |
| | | CCGTACACCATGTACGTCAAGTCCGTCCGTATCGAGAACGCCAACCC |
| | | CGCCGAGTCCTACACCTACTCGGACAACTCTGGCTCTTGGCAGAGCAT |
| | | CAAGTTCGACGGCTCCGTCGATATCTCCTCCAGCTCTTCCGTGACCTC |
| | | CTCCACCACCAGCACCGCCAGCTCCGCCAGCTCTACCTCGAGCAAGA |
| | | CCCCTTCCACCTCCACCCTGGCCACTTCCACCAAGGCGACTCCCACCC |
| | | CGTCTGGAACCAGCTCCGGCTCTAACTCGAGCTCCAGCGCGGAACCT |
| | | ACTACCACCGGCGGCACCGGCAGCAGCAACACCGGCTCTGGCTCCGG |
| | | CTCCGGCTCTGGCTCTGGCTCTAGCTCTAGCACGGGCTCCTCCACTAG |
| | | CGCCGGAGCCTCCGCCACCCCCGAGCTCTCCCAGGGCGCCGCCGGCT |
| | | CCATCAAGGGCTCGGTCACCGCCTGCGCTCTGGTGTTCGGCGCCGTCG |
| | | CTGCCGTGTTGGCATTCTAA |
| Pertussis | 119 | ATGCCGATCGACCGCAAGACGCTCTGCCATCTCCTGTCCGTTCTGCCG |
| toxin | | TTGGCCCTCCTCGGATCTCACGTGGCGCGGGCCTCCACGCCAGGCATC |
| subunit 2 | | GTCATTCCGCCGCAGGAACAGATTACCCAGCACGGCGGCCCCTATGG |
| | | ACGCTGCGCGAACAAGACCCGTGCCCTGACCGTGGCGGAATTGCGCG |
| | | GCAGCGGCGATCTGCAGGAGTACCTGCGTCATGTGACGCGCGGCTGG |
| | | TCAATATTTGCGCTCTACGATGGCACCTATCTCGGCGGCGAATATGGC |
| | | GGCGTGATCAAGGACGGAACACCCGGCGGCGCATTCGACCTGAAAAC |
| | | GACGTTCTGCATCATGACCACGCGCAATACGGGTCAACCCGCAACGG |
| | | ATCACTTCTACAGCAACGTCACCGCCACTCGCCTGCTCTCCAGCACCA |
| | | ACAGCAGGCTATGCGCGGTCTTCGTCAGAAGCGGGCAACCGGTCATT |
| | | GGCGCCTGCACCAGCCCGTATGACGGCAAGTACTGGAGCATGTACAG |
| | | CCGGCTGCGGAAAATGCTTTACCTGATCTACGTGGCCGGCATCTCCGT |
| | | ACGCGTCCATGTCAGCAAGGAAGAACAGTATTACGACTACGAAGACG |
| | | CAACGTTCGAGACTTACGCCCTTACCGGCATCTCCATCTGCAATCCGG |
| | | GATCATCCTTATGCTGA |
| HBV | 120 | AATTCCACAACCTTCCACCAAACTCTGCAAGATCCCAGAGTGAGAGG |
| envelope | | CCTGTATTTCCCTGCTGGTGGCTCCAGTTCAGGAACAGTAAACCCTGT |
| | | TCTGACTACTGCCTCTCCCTTATCGTCAATCTTCTCGAGGATTGGGGA |
| | | CCCTGCGCTGAACATGGAGAACATCACATCAGGATTCCTAGGACCCC |
| | | TTCTCGTGTTACAGGCGGGGTTTTTCTTGTTGACAAGAATCCTCACAA |
| | | TACCGCAGAGTCTAGACTCGTGGTGGACTTCTCTCAATTTTCTAGGGG |
| | | GAACTACCGTGTGTCTTGGCCAAAATTCGCAGTCCCCAACCTCCAATC |
| | | ACTCACCAACCTCTTGTCCTCCAACTTGTCCTGGTTATCGCTGGATGT |
| | | GTCTGCGGCGTTTTATCATCTTCCTCTTCATCCTGCTGCTATGCCTCAT |
| | | CTTCTTGTTGGTTCTTCTGGACTATCAAGGTATGTTGCCCGTTTGTCCT |
| | | CTAATTCCAGGATCCTCAACAACCAGCACGGGACCATGCCGGACCTG |
| | | CATGACTACTGCTCAAGGAACCTCTATGTATCCCTCCTGTTGCTGTAC |
| | | CAAACCTTCGGACGGAAATTGCACCTGTATTCCCATCCCATCATCCTG |
| | | GGCTTTCGGAAAATTCCTATGGGAGTGGGCCTCAGCCCGTTTCTCCTG |
| | | GCTCAGTTTACTAGTGCCATTTGTTCAGTGGTTCGTAGGGCTTTCCCC |
| | | CACTGTTTGGCTTTCAGTTATATGGATGATGTGGTATTGGGGGCCAAG |
| | | TCTGTACAGCATCTTGAGTCCCTTTTTACCGCTGTTACCAATTTTCTTT |
| | | TGTCTTTGGGTATACATTTAAACCCTAACAAAACAAAGAGATGGGGT |
| | | TACTCTCTAAATTTTATGGGTTATGTCATTGGATGTTATGGGTCCTTGC |
| | | CACAAGAACACATCATACAAAAAATCAAAGAATGTTTTAGAAAACTT |
| | | CCTATTAACAGGCCTATTGATTGGAAAGTATGTCAACGAATTGTGGGT |
| | | CTTTTGGGTTTTGCTGCCCCTTTTACACAATGTGGTTATCCTGCGTTGA |
| | | TGCCTTTGTATGCATGTATTCAATCTAAGCAGGCTTTCACTTTCTCGCC |
| | | AACTTACAAGGCCTTTCTGTGTAAACAATACCTGAACCTTTACCCCGT |
| | | TGCCCGGCAACGGCCAGGTCTGTGCCAAGTGTTTGCTGACGCAACCC |
| | | CCACTGGCTGGGGCTTGGTCATGGGCCATCAGCGCATGCGTGGAACC |
| | | TTTTCGGCTCCTCTGCCGATCCATACTGCGGAACTCCTAGCCGCTTGT |
| | | TTTGCTCGCAGCAGGTCTGGAGCAAACATTATCGGGACTGATAACTCT |
| | | GTTGTCCTATCCCGCAAATATACATCGTTTCCATGGCTGCTAGGCTGT |
| | | GCTGCCAACTGGATCCTGCGCGGGACGTCCTTTGTTTACGTCCCGTCG |
| | | GCGCTGAATCCTGCGGACGACCCTTCTCGGGGTCGCTTGGGACTCTCT |
| | | CGTCCCCTTCTCCGTCTGCCGTTCCGACCGACCACGGGGCGCACCTCT |
| | | CTTTACGCGGACTCCCCGTCTGTGCCTTCTCATCTGCCGGACCGTGTG |
| | | CACTTCGCTTCACCTCTGCACGTCGCATGGAGACCACCGTGAACGCCC |
| | | ACCAAATATTGCCCAAGGTCTTACATAAGAGGACTCTTGGACTCTCA |
| | | GCAATGTCAACGACCGACCTTGAGGCATACTTCAAAGACTGTTTGTTT |
| | | AAAGACTGGGAGGAGTTGGGGGAGGAGATTAGGTTAAAGGTCTTTGT |
| | | ACTAGGAGGCTGTAGGCATAAATTGGTCTGCGCACCAGCACCATGCA |
| | | ACTTTTTCACCTCTGCCTAATCATCTCTTGTTCATGTCCTACTGTTCAA |
| | | GCCTCCAAGCTGTGCCTTGGGTGGCTTTGGGGCATGGACATCGACCCT |
| | | TATAAAGAATTTGGAGCTACTGTGGAGTTACTCTCGTTTTTGCCTTCT |
| | | GACTTCTTTCCTTCAGTACGAGATCTTCTAGATACCGCCTCAGCTCTG |
| | | TATCGGGAAGCCTTAGAGTCTCCTGAGCATTGTTCACCTCACCATACT |
| | | GCACTCAGGCAAGCAATTCTTTGCTGGGGGGAACTAATGACTCTAGC |
| | | TACCTGGGTGGGTGTTAATTTGGAAGATCCAGCGTCTAGAGACCTAG |
| | | TAGTCAGTTATGTCAACACTAATATGGGCCTAAAGTTCAGGCAACTCT |
| | | TGTGGTTTCACATTTCTTGTCTCACTTTTGGAAGAGAAACAGTTATAG |
| | | AGTATTTGGTGTCTTTCGGAGTGTGGATTCGCACTCCTCCAGCTTATA |
| | | GACCACCAAATGCCCCTATCCTATCAACACTTCCGGAGACTACTGTTG |
| | | TTAGACGACGAGGCAGGTCCCCTAGAAGAAGAACTCCCTCGCCTCGC |
| | | AGACGAAGGTCTCAATCGCCGCGTCGCAGAAGATCTCAATCTCGGGA |
| | | ATCTCAATGTTAGTATTCCTTGGACTCATAAGGTGGGGAACTTTACTG |
| | | GGCTTTATTCTTCTACTGTACCTGTCTTTAATCCTCATTGGAAAACACC |
| | | ATCTTTTCCTAATATACATTTACACCAAGACATTATCAAAAAATGTGA |
| | | ACAGTTTGTAGGCCCACTCACAGTTAATGAGAAAAGAAGATTGCAAT |
| | | TGATTATGCCTGCCAGGTTTTATCCAAAGGTTACCAAATATTTACCAT |
| | | TGGATAAGGGTATTAAACCTTATTATCCAGAACATCTAGTTAATCATT |
| | | ACTTCCAAACTAGACACTATTTACACACTCTATGGAAGGCGGGTATAT |
| | | TATATAAGAGAGAAACAACACATAGCGCCTCATTTTGTGGGTCACCA |
| | | TATTCTTGGGAACAAGATCTACAGCATGGGGCAGAATCTTTCCACCA |
| | | GCAATCCTCTGGGATTCTTTCCCGACCACCAGTTGGATCCAGCCTTCA |
| | | GAGCAAACACCGCAAATCCAGATTGGGACTTCAATCCCAACAAGGAC |
| | | ACCTGGCCAGACGCCAACAAGGTAGGAGCTGGAGCATTCGGGCTGGG |
| | | TTTCACCCCACCGCACGGAGGCCTTTTGGGGTGGAGCCCTCAGGCTCA |
| | | GGGCATACTACAAACTTTGCCAGCAAATCCGCCTCCTGCCTCCACCAA |
| | | TCGCCAGTCAGGAAGGCAGCCTACCCCGCTGTCTCCACCTTTGAGAA |
| | | ACACTCATCCTCAGGCCATGCAGTGG |
| HCV | 121 | ATGAGCACGAATCCTAAACCTCAAAGAAAAACCAAACGTAACACCA |
| polyprotein | | ACCGTCGCCCACAGGACGTCAAGTTCCCGGGTGGCGGTCAGATCGTT |
| | | GGTGGAGTTTACTTGTTGCCGCGCAGGGGCCCTAGATTGGGTGTGCG |
| | | CGCGACGAGGAAGACTTCCGAGCGGTCGCAACCTCGAGGTAGACGTC |
| | | AGCCTATCCCCAAGGCACGTCGGCCCGAGGGCAGGACCTGGGCTCAG |
| | | CCCGGGTACCCTTGGCCCCTCTATGGCAATGAGGGTTGCGGGTGGGC |
| | | GGGATGGCTCCTGTCTCCCCGTGGCTCTCGGCCTAGCTGGGGCCCCAC |
| | | AGACCCCCGGCGTAGGTCGCGCAATTTGGGTAAGGTCATCGATACCC |
| | | TTACGTGCGGCTTCGCCGACCTCATGGGGTACATACCGCTCGTCGGCG |
| | | CCCCTCTTGGAGGCGCTGCCAGGGCCCTGGCGCATGGCGTCCGGGTT |
| | | CTGGAAGACGGCGTGAACTATGCAACAGGGAACCTTCCTGGTTGCTC |
| | | TTTCTCTATCTTCCTTCTGGCCCTGCTCTCTTGCCTGACTGTGCCCGCT |
| | | TCAGCCTACCAAGTGCGCAATTCCTCGGGGCTTTACCATGTCACCAAT |
| | | GATTGCCCTAACTCGAGTATTGTGTACGAGGCGGCCGATGCCATCCTG |
| | | CACACTCCGGGGTGTGTCCCTTGCGTTCGCGAGGGTAACGCCTCGAG |
| | | GTGTTGGGTGGCGGTGACCCCCACGGTGGCCACCAGGGACGGCAAAC |
| | | TCCCCACAACGCAGCTTCGACGTCATATCGATCTGCTTGTCGGGAGCG |
| | | CCACCCTCTGCTCGGCCCTCTACGTGGGGGACCTGTGCGGGTCTGTCT |
| | | TTCTTGTTGGTCAACTGTTTACCTTCTCTCCCAGGCGCCACTGGACGA |
| | | CGCAAGACTGCAATTGTTCTATCTATCCCGGCCATATAACGGGTCATC |
| | | GCATGGCATGGGATATGATGATGAACTGGTCCCCTACGGCAGCGTTG |
| | | GTGGTAGCTCAGCTGCTCCGGATCCCACAAGCCATCATGGACATGAT |
| | | CGCTGGTGCTCACTGGGGAGTCCTGGCGGGCATAGCGTATTTCTCCAT |
| | | GGTGGGGAACTGGGCGAAGGTCCTGGTAGTGCTGCTGCTATTTGCCG |
| | | GCGTCGACGCGGAAACCCACGTCACCGGGGGAAGTGCCGGCCGCACC |
| | | ACGGCTGGGCTTGTTGGTCTCCTTACACCAGGCGCCAAGCAGAACAT |
| | | CCAACTGATCAACACCAACGGCAGTTGGCACATCAATAGCACGGCCT |
| | | TGAACTGCAATGAAAGCCTTAACACCGGCTGGTTAGCAGGGCTCTTC |
| | | TATCAGCACAAATTCAACTCTTCAGGCTGTCCTGAGAGGTTGGCCAGC |
| | | TGCCGACGCCTTACCGATTTTGCCCAGGGCTGGGGTCCTATCAGTTAT |
| | | GCCAACGGAAGCGGCCTCGACGAACGCCCCTACTGCTGGCACTACCC |
| | | TCCAAGACCTTGTGGCATTGTGCCCGCAAAGAGCGTGTGTGGCCCGG |
| | | TATATTGCTTCACTCCCAGCCCCGTGGTGGTGGGAACGACCGACAGG |
| | | TCGGGCGCGCCTACCTACAGCTGGGGTGCAAATGATACGGATGTCTT |
| | | CGTCCTTAACAACACCAGGCCACCGCTGGGCAATTGGTTCGGTTGTAC |
| | | CTGGATGAACTCAACTGGATTCACCAAAGTGTGCGGAGCGCCCCCTT |
| | | GTGTCATCGGAGGGGTGGGCAACAACACCTTGCTCTGCCCCACTGAT |
| | | TGTTTCCGCAAGCATCCGGAAGCCACATACTCTCGGTGCGGCTCCGGT |
| | | CCCTGGATTACACCCAGGTGCATGGTCGACTACCCGTATAGGCTTTGG |
| | | CACTATCCTTGTACCATCAATTACACCATATTCAAAGTCAGGATGTAC |
| | | GTGGGAGGGGTCGAGCACAGGCTGGAAGCGGCCTGCAACTGGACGC |
| | | GGGGCGAACGCTGTGATCTGGAAGACAGGGACAGGTCCGAGCTCAG |
| | | CCCATTGCTGCTGTCCACCACACAGTGGCAGGTCCTTCCGTGTTCTTT |
| | | CACGACCCTGCCAGCCTTGTCCACCGGCCTCATCCACCTCCACCAGAA |
| | | CATTGTGGACGTGCAGTACTTGTACGGGGTAGGGTCAAGCATCGCGT |
| | | CCTGGGCCATTAAGTGGGAGTACGTCGTTCTCCTGTTCCTCCTGCTTG |
| | | CAGACGCGCGCGTCTGCTCCTGCTTGTGGATGATGTTACTCATATCCC |
| | | AAGCGGAGGCGGCTTTGGAGAACCTCGTAATACTCAATGCAGCATCC |
| | | CTGGCCGGGACGCACGGTCTTGTGTCCTTCCTCGTGTTCTTCTGCTTTG |
| | | CGTGGTATCTGAAGGGTAGGTGGGTGCCCGGAGCGGTCTACGCCTTC |
| | | TACGGGATGTGGCCTCTCCTCCTGCTCCTGCTGGCGTTGCCTCAGCGG |
| | | GCATACGCACTGGACACGGAGGTGGCCGCGTCGTGTGGCGGCGTTGT |
| | | TCTTGTCGGGTTAATGGCGCTGACTCTGTCGCCATATTACAAGCGCTA |
| | | CATCAGCTGGTGCATGTGGTGGCTTCAGTATTTTCTGACCAGAGTAGA |
| | | AGCGCAACTGCACGTGTGGGTTCCCCCCCTCAACGTCCGGGGGGGGC |
| | | GCGATGCCGTCATCTTACTCATGTGTGTTGTACACCCGACTCTGGTAT |
| | | TTGACATCACCAAACTACTCCTGGCCATCTTCGGACCCCTTTGGATTC |
| | | TTCAAGCCAGTTTGCTTAAAGTCCCCTACTTCGTGCGCGTTCAAGGCC |
| | | TTCTCCGGATCTGCGCGCTAGCGCGGAAGATAGCCGGAGGTCATTAC |
| | | GTGCAAATGGCCATCATCAAGTTAGGGGCGCTTACTGGCACCTATGT |
| | | GTATAACCATCTCACCCCTCTTCGAGACTGGGCGCACAACGGCCTGC |
| | | GAGATCTGGCCGTGGCTGTGGAACCAGTCGTCTTCTCCCGAATGGAG |
| | | ACCAAGCTCATCACGTGGGGGGCAGATACCGCCGCGTGCGGTGACAT |
| | | CATCAACGGCTTGCCCGTCTCTGCCCGTAGGGGCCAGGAGATACTGC |
| | | TTGGGCCAGCCGACGGAATGGTCTCCAAGGGGTGGAGGTTGCTGGCG |
| | | CCCATCACGGCGTACGCCCAGCAGACGAGAGGCCTCCTAGGGTGTAT |
| | | AATCACCAGCCTGACTGGCCGGGACAAAAACCAAGTGGAGGGTGAG |
| | | GTCCAGATCGTGTCAACTGCTACCCAAACCTTCCTGGCAACGTGCATC |
| | | AATGGGGTATGCTGGACTGTCTACCACGGGGCCGGAACGAGGACCAT |
| | | CGCATCACCCAAGGGTCCTGTCATCCAGATGTATACCAATGTGGACC |
| | | AAGACCTTGTGGGCTGGCCCGCTCCTCAAGGTTCCCGCTCATTGACAC |
| | | CCTGCACCTGCGGCTCCTCGGACCTTTACCTGGTCACGAGGCACGCCG |
| | | ATGTCATTCCCGTGCGCCGGCGAGGTGATAGCAGGGGTAGCCTGCTT |
| | | TCGCCCCGGCCCATTTCCTACTTGAAAGGCTCCTCGGGGGGTCCGCTG |
| | | TTGTGCCCCGCGGGACACGCCGTGGGCCTATTCAGGGCCGCGGTGTG |
| | | CACCCGTGGAGTGGCTAAGGCGGTGGACTTTATCCCTGTGGAGAACC |
| | | TAGAGACAACCATGAGATCCCCGGTGTTCACGGACAACTCCTCTCCA |
| | | CCAGCAGTGCCCCAGAGCTTCCAGGTGGCCCACCTGCATGCTCCCAC |
| | | CGGCAGCGGTAAGAGCACCAAGGTCCCGGCTGCGTACGCAGCCCAGG |
| | | GCTACAAGGTGTTGGTGCTCAACCCCTCTGTTGCTGCAACGCTGGGCT |
| | | TTGGTGCTTACATGTCCAAGGCCCATGGGGTTGATCCTAATATCAGGA |
| | | CCGGGGTGAGAACAATTACCACTGGCAGCCCCATCACGTACTCCACC |
| | | TACGGCAAGTTCCTTGCCGACGGCGGGTGCTCAGGAGGTGCTTATGA |
| | | CATAATAATTTGTGACGAGTGCCACTCCACGGATGCCACATCCATCTT |
| | | GGGCATCGGCACTGTCCTTGACCAAGCAGAGACTGCGGGGGCGAGAC |
| | | TGGTTGTGCTCGCCACTGCTACCCCTCCGGGCTCCGTCACTGTGTCCC |
| | | ATCCTAACATCGAGGAGGTTGCTCTGTCCACCACCGGAGAGATCCCTT |
| | | TTTACGGCAAGGCTATCCCCCTCGAGGTGATCAAGGGGGGAAGACAT |
| | | CTCATCTTCTGCCACTCAAAGAAGAAGTGCGACGAGCTCGCCGCGAA |
| | | GCTGGTCGCATTGGGCATCAATGCCGTGGCCTACTACCGCGGTCTTGA |
| | | CGTGTCTGTCATCCCGACCAGCGGCGATGTTGTCGTCGTGTCGACCGA |
| | | TGCTCTCATGACTGGCTTTACCGGCGACTTCGACTCTGTGATAGACTG |
| | | CAACACGTGTGTCACTCAGACAGTCGATTTCAGCCTTGACCCTACCTT |
| | | TACCATTGAGACAACCACGCTCCCCCAGGATGCTGTCTCCAGGACTC |
| | | AACGCCGGGGCAGGACTGGCAGGGGGAAGCCAGGCATCTACAGATT |
| | | TGTGGCACCGGGGGAGCGCCCCTCCGGCATGTTCGACTCGTCCGTCCT |
| | | CTGTGAGTGCTATGACGCGGGCTGTGCTTGGTATGAGCTCACGCCCGC |
| | | CGAGACTACAGTTAGGCTACGAGCGTACATGAACACCCCGGGGCTTC |
| | | CCGTGTGCCAGGACCATCTTGAATTTTGGGAGGGCGTCTTTACGGGCC |
| | | TCACTCATATAGATGCCCACTTTCTATCCCAGACAAAGCAGAGTGGG |
| | | GAGAACTTTCCTTACCTGGTAGCGTACCAAGCCACCGTGTGCGCTAG |
| | | GGCTCAAGCCCCTCCCCCATCGTGGGACCAGATGTGGAAGTGTTTGA |
| | | TCCGCCTTAAACCCACCCTCCATGGGCCAACACCCCTGCTATACAGAC |
| | | TGGGCGCTGTTCAGAATGAAGTCACCCTGACGCACCCAATCACCAAA |
| | | TACATCATGACATGCATGTCGGCCGACCTGGAGGTCGTCACGAGCAC |
| | | CTGGGTGCTCGTTGGCGGCGTCCTGGCTGCTCTGGCCGCGTATTGCCT |
| | | GTCAACAGGCTGCGTGGTCATAGTGGGCAGGATTGTCTTGTCCGGGA |
| | | AGCCGGCAATTATACCTGACAGGGAGGTTCTCTACCAGGAGTTCGAT |
| | | GAGATGGAAGAGTGCTCTCAGCACTTACCGTACATCGAGCAAGGGAT |
| | | GATGCTCGCTGAGCAGTTCAAGCAGAAGGCCCTCGGCCTCCTGCAGA |
| | | CCGCGTCCCGCCAAGCAGAGGTTATCACCCCTGCTGTCCAGACCAAC |
| | | TGGCAGAAACTCGAGGTCTTCTGGGCGAAGCACATGTGGAATTTCAT |
| | | CAGTGGGATACAATACTTGGCGGGCCTGTCAACGCTGCCTGGTAACC |
| | | CCGCCATTGCTTCATTGATGGCTTTTACAGCTGCCGTCACCAGCCCAC |
| | | TAACCACTGGCCAAACCCTCCTCTTCAACATATTGGGGGGGTGGGTG |
| | | GCTGCCCAGCTCGCCGCCCCCGGTGCCGCTACCGCCTTTGTGGGCGCT |
| | | GGCTTAGCTGGCGCCGCCATCGGCAGCGTTGGACTGGGGAAGGTCCT |
| | | CGTGGACATTCTTGCAGGGTATGGCGCGGGCGTGGCGGGAGCTCTTG |
| | | TAGCATTCAAGATCATGAGCGGTGAGGTCCCCTCCACGGAGGACCTG |
| | | GTCAATCTGCTGCCCGCCATCCTCTCGCCTGGAGCCCTTGTAGTCGGT |
| | | GTGGTCTGCGCAGCAATACTGCGCCGGCACGTTGGCCCGGGCGAGGG |
| | | GGCAGTGCAATGGATGAACCGGCTAATAGCCTTCGCCTCCCGGGGGA |
| | | ACCATGTTTCCCCCACGCACTACGTGCCGGAGAGCGATGCAGCCGCC |
| | | CGCGTCACTGCCATACTCAGCAGCCTCACTGTAACCCAGCTCCTGAGG |
| | | CGACTGCATCAGTGGATAAGCTCGGAGTGTACCACTCCATGCTCCGG |
| | | TTCCTGGCTAAGGGACATCTGGGACTGGATATGCGAGGTGCTGAGCG |
| | | ACTTTAAGACCTGGCTGAAAGCCAAGCTCATGCCACAACTGCCTGGG |
| | | ATTCCCTTTGTGTCCTGCCAGCGCGGGTATAGGGGGGTCTGGCGAGG |
| | | AGACGGCATTATGCACACTCGCTGCCACTGTGGAGCTGAGATCACTG |
| | | GACATGTCAAAAACGGGACGATGAGGATCGTCGGTCCTAGGACCTGC |
| | | AGGAACATGTGGAGTGGGACGTTCCCCATTAACGCCTACACCACGGG |
| | | CCCCTGTACTCCCCTTCCTGCGCCGAACTATAAGTTCGCGCTGTGGAG |
| | | GGTGTCTGCAGAGGAATACGTGGAGATAAGGCGGGTGGGGGACTTCC |
| | | ACTACGTATCGGGTATGACTACTGACAATCTTAAATGCCCGTGCCAG |
| | | ATCCCATCGCCCGAATTTTTCACAGAATTGGACGGGGTGCGCCTACAT |
| | | AGGTTTGCGCCCCCTTGCAAGCCCTTGCTGCGGGAGGAGGTATCATTC |
| | | AGAGTAGGACTCCACGAGTACCCGGTGGGGTCGCAATTACCTTGCGA |
| | | GCCCGAACCGGACGTAGCCGTGTTGACGTCCATGCTCACTGATCCCTC |
| | | CCATATAACAGCAGAGGCGGCCGGGAGAAGGTTGGCGAGAGGGTCA |
| | | CCCCCTTCTATGGCCAGCTCCTCGGCCAGCCAGCTGTCCGCTCCATCT |
| | | CTCAAGGCAACTTGCACCGCCAACCATGACTCCCCTGACGCCGAGCT |
| | | CATAGAGGCTAACCTCCTGTGGAGGCAGGAGATGGGCGGCAACATCA |
| | | CCAGGGTTGAGTCAGAGAACAAAGTGGTGATTCTGGACTCCTTCGAT |
| | | CCGCTTGTGGCAGAGGAGGATGAGCGGGAGGTCTCCGTACCCGCAGA |
| | | AATTCTGCGGAAGTCTCGGAGATTCGCCCGGGCCCTGCCCGTTTGGGC |
| | | GCGGCCGGACTACAACCCCCCGCTAGTAGAGACGTGGAAAAAGCCTG |
| | | ACTACGAACCACCTGTGGTCCATGGCTGCCCGCTACCACCTCCACGGT |
| | | CCCCTCCTGTGCCTCCGCCTCGGAAAAAGCGTACGGTGGTCCTCACCG |
| | | AATCAACCCTATCTACTGCCTTGGCCGAGCTTGCCACCAAAAGTTTTG |
| | | GCAGCTCCTCAACTTCCGGCATTACGGGCGACAATACGACAACATCC |
| | | TCTGAGCCCGCCCCTTCTGGCTGCCCCCCCGACTCCGACGTTGAGTCC |
| | | TATTCTTCCATGCCCCCCCTGGAGGGGGAGCCTGGGGATCCGGATCTC |
| | | AGCGACGGGTCATGGTCGACGGTCAGTAGTGGGGCCGACACGGAAG |
| | | ATGTCGTGTGCTGCTCAATGTCTTATTCCTGGACAGGCGCACTCGTCA |
| | | CCCCGTGCGCTGCGGAAGAACAAAAACTGCCCATCAACGCACTGAGC |
| | | AACTCGTTGCTACGCCATCACAATCTGGTGTATTCCACCACTTCACGC |
| | | AGTGCTTGCCAAAGGCAGAAGAAAGTCACATTTGACAGACTGCAAGT |
| | | TCTGGACAGCCATTACCAGGACGTGCTCAAGGAGGTCAAAGCAGCGG |
| | | CGTCAAAAGTGAAGGCTAACTTGCTATCCGTAGAGGAAGCTTGCAGC |
| | | CTGACGCCCCCACATTCAGCCAAATCCAAGTTTGGCTATGGGGCAAA |
| | | AGACGTCCGTTGCCATGCCAGAAAGGCCGTAGCCCACATCAACTCCG |
| | | TGTGGAAAGACCTTCTGGAAGACAGTGTAACACCAATAGACACTACC |
| | | ATCATGGCCAAGAACGAGGTTTTCTGCGTTCAGCCTGAGAAGGGGGG |
| | | TCGTAAGCCAGCTCGTCTCATCGTGTTCCCCGACCTGGGCGTGCGCGT |
| | | GTGCGAGAAGATGGCCCTGTACGACGTGGTTAGCAAGCTCCCCCTGG |
| | | CCGTGATGGGAAGCTCCTACGGATTCCAATACTCACCAGGACAGCGG |
| | | GTTGAATTCCTCGTGCAAGCGTGGAAGTCCAAGAAGACCCCGATGGG |
| | | GTTCTCGTATGATACCCGCTGTTTTGACTCCACAGTCACTGAGAGCGA |
| | | CATCCGTACGGAGGAGGCAATTTACCAATGTTGTGACCTGGACCCCC |
| | | AAGCCCGCGTGGCCATCAAGTCCCTCACTGAGAGGCTTTATGTTGGG |
| | | GGCCCTCTTACCAATTCAAGGGGGGAAAACTGCGGCTACCGCAGGTG |
| | | CCGCGCGAGCGGCGTACTGACAACTAGCTGTGGTAACACCCTCACTT |
| | | GCTACATCAAGGCCCGGGCAGCCTGTCGAGCCGCAGGGCTCCAGGAC |
| | | TGCACCATGCTCGTGTGTGGCGACGACTTAGTCGTTATCTGTGAAAGT |
| | | GCGGGGGTCCAGGAGGACGCGGCGAGCCTGAGAGCCTTCACGGAGG |
| | | CTATGACCAGGTACTCCGCCCCCCCCGGGGACCCCCCACAACCAGAA |
| | | TACGACTTGGAGCTTATAACATCATGCTCCTCCAACGTGTCAGTCGCC |
| | | CACGACGGCGCTGGAAAGAGGGTCTACTACCTTACCCGTGACCCTAC |
| | | AACCCCCCTCGCGAGAGCCGCGTGGGAGACAGCAAGACACACTCCAG |
| | | TCAATTCCTGGCTAGGCAACATAATCATGTTTGCCCCCACACTGTGGG |
| | | CGAGGATGATACTGATGACCCATTTCTTTAGCGTCCTCATAGCCAGGG |
| | | ATCAGCTTGAACAGGCTCTTAACTGTGAGATCTACGGAGCCTGCTACT |
| | | CCATAGAACCACTGGATCTACCTCCAATCATTCAAAGACTCCATGGCC |
| | | TCAGCGCATTTTCACTCCACAGTTACTCTCCAGGTGAAATCAATAGGG |
| | | TGGCCGCATGCCTCAGAAAACTTGGGGTCCCGCCCTTGCGAGCTTGG |
| | | AGACACCGGGCCCGGAGCGTCCGCGCTAGGCTTCTGTCCAGAGGAGG |
| | | CAGGGCTGCCATATGTGGCAAGTACCTCTTCAACTGGGCAGTAAGAA |
| | | CAAAGCTCAAACTCACTCCAATAGCGGCCGCTGGCCGGCTGGACTTG |
| | | TCCGGTTGGTTCACGGCTGGCTACAGCGGGGGAGACATTTATCACAG |
| | | CGTGTCTCATGCCCGGCCCCGCTGGTTCTGGTTTTGCCTACTCCTGCTC |
| | | GCTGCAGGGGTAGGCATCTACCTCCTCCCCAACCGATGA |
| HIV-1 gag | 122 | ATGGGTGCGAGAGCGTCAGTATTAAGCGGGGGAGAATTAGATCGATG |
| | | GGAAAAAATTCGGTTAAGGCCAGGGGGAAAGAAAAAATATAAATTA |
| | | AAACATATAGTATGGGCAAGCAGGGAGCTAGAACGATTCGCAGTTAA |
| | | TCCTGGCCTGTTAGAAACATCAGAAGGCTGTAGACAAATACTGGGAC |
| | | AGCTACAACCATCCCTTCAGACAGGATCAGAAGAACTTAGATCATTA |
| | | TATAATACAGTAGCAACCCTCTATTGTGTGCATCAAAGGATAGAGAT |
| | | AAAAGACACCAAGGAAGCTTTAGACAAGATAGAGGAAGAGCAAAAC |
| | | AAAAGTAAGAAAAAAGCACAGCAAGCAGCAGCTGACACAGGACACA |
| | | GCAATCAGGTCAGCCAAAATTACCCTATAGTGCAGAACATCCAGGGG |
| | | CAAATGGTACATCAGGCCATATCACCTAGAACTTTAAATGCATGGGT |
| | | AAAAGTAGTAGAAGAGAAGGCTTTCAGCCCAGAAGTGATACCCATGT |
| | | TTTCAGCATTATCAGAAGGAGCCACCCCACAAGATTTAAACACCATG |
| | | CTAAACACAGTGGGGGGACATCAAGCAGCCATGCAAATGTTAAAAG |
| | | AGACCATCAATGAGGAAGCTGCAGAATGGGATAGAGTGCATCCAGTG |
| | | CATGCAGGGCCTATTGCACCAGGCCAGATGAGAGAACCAAGGGGAA |
| | | GTGACATAGCAGGAACTACTAGTACCCTTCAGGAACAAATAGGATGG |
| | | ATGACAAATAATCCACCTATCCCAGTAGGAGAAATTTATAAAAGATG |
| | | GATAATCCTGGGATTAAATAAAATAGTAAGAATGTATAGCCCTACCA |
| | | GCATTCTGGACATAAGACAAGGACCAAAGGAACCCTTTAGAGACTAT |
| | | GTAGACCGGTTCTATAAAACTCTAAGAGCCGAGCAAGCTTCACAGGA |
| | | GGTAAAAAATTGGATGACAGAAACCTTGTTGGTCCAAAATGCGAACC |
| | | CAGATTGTAAGACTATTTTAAAAGCATTGGGACCAGCGGCTACACTA |
| | | GAAGAAATGATGACAGCATGTCAGGGAGTAGGAGGACCCGGCCATA |
| | | AGGCAAGAGTTTTGGCTGAAGCAATGAGCCAAGTAACAAATTCAGCT |
| | | ACCATAATGATGCAGAGAGGCAATTTTAGGAACCAAAGAAAGATTGT |
| | | TAAGTGTTTCAATTGTGGCAAAGAAGGGCACACAGCCAGAAATTGCA |
| | | GGGCCCCTAGGAAAAAGGGCTGTTGGAAATGTGGAAAGGAAGGACA |
| | | CCAAATGAAAGATTGTACTGAGAGACAGGCTAATTTTTTAGGGAAGA |
| | | TCTGGCCTTCCTACAAGGGAAGGCCAGGGAATTTTCTTCAGAGCAGA |
| | | CCAGAGCCAACAGCCCCACCAGAAGAGAGCTTCAGGTCTGGGGTAGA |
| | | GACAACAACTCCCCCTCAGAAGCAGGAGCCGATAGACAAGGAACTGT |
| | | ATCCTTTAACTTCCCTCAGGTCACTCTTTGGCAACGACCCCTCGTCAC |
| | | AATAA |
| HPV E2 | 123 | ATGGAGACTCTTTGCCAACGTTTAAATGTGTGTCAGGACAAAATACT |
| | | AACACATTATGAAAATGATAGTACAGACCTACGTGACCATATAGACT |
| | | ATTGGAAACACATGCGCCTAGAATGTGCTATTTATTACAAGGCCAGA |
| | | GAAATGGGATTTAAACATATTAACCACCAGGTGGTGCCAACACTGGC |
| | | TGTATCAAAGAATAAAGCATTACAAGCAATTGAACTGCAACTAACGT |
| | | TAGAAACAATATATAACTCACAATATAGTAATGAAAAGTGGACATTA |
| | | CAAGACGTTAGCCTTGAAGTGTATTTAACTGCACCAACAGGATGTAT |
| | | AAAAAAACATGGATATACAGTGGAAGTGCAGTTTGATGGAGACATAT |
| | | GCAATACAATGCATTATACAAACTGGACACATATATATATTTGTGAA |
| | | GAAGCATCAGTAACTGTGGTAGAGGGTCAAGTTGACTATTATGGTTT |
| | | ATATTATGTTCATGAAGGAATACGAACATATTTTGTGCAGTTTAAAGA |
| | | TGATGCAGAAAAATATAGTAAAAATAAAGTATGGGAAGTTCATGCGG |
| | | GTGGTCAGGTAATATTATGTCCTACATCTGTGTTTAGCAGCAACGAAG |
| | | TATCCTCTCCTGAAATTATTAGGCAGCACTTGGCCAACCACCCCGCCG |
| | | CGACCCATACCAAAGCCGTCGCCTTGGGCACCGAAGAAACACAGACG |
| | | ACTATCCAGCGACCAAGATCAGAGCCAGACACCGGAAACCCCTGCCA |
| | | CACCACTAAGTTGTTGCACAGAGACTCAGTGGACAGTGCTCCAATCC |
| | | TCACTGCATTTAACAGCTCACACAAAGGACGGATTAACTGTAATAGT |
| | | AACACTACACCCATAGTACATTTAAAAGGTGATGCTAATACTTTAAA |
| | | ATGTTTAAGATATAGATTTAAAAAGCATTGTACATTGTATACTGCAGT |
| | | GTCGTCTACATGGCATTGGACAGGACATAATGTAAAACATAAAAGTG |
| | | CAATTGTTACACTTACATATGATAGTGAATGGCAACGTGACCAATTTT |
| | | TGTCTCAAGTTAAAATACCAAAAACTATTACAGTGTCTACTGGATTTA |
| | | TGTCTATATGA |
| Malaria | 124 | ATGATGAGAAAATTAGCTATTTTATCTGTTTCTTCCTTTTTATTTGTTG |
| CSP | | AGGCCTTATTCCAGGAATACCAGTGCTATGGAAGTTCGTCAAACACA |
| | | AGGGTTCTAAATGAATTAAATTATGATAATGCAGGCACTAATTTATAT |
| | | AATGAATTAGAAATGAATTATTATGGGAAACAGGAAAATTGGTATAG |
| | | TCTTAAAAAAAATAGTAGATCACTTGGAGAAAATGATGATGGAAATA |
| | | ACGAAGACAACGAGAAATTAAGGAAACCAAAACATAAAAAATTAAA |
| | | GCAACCAGCGGATGGTAATCCTGATCCAAATGCAAACCCAAATGTAG |
| | | ATCCCAATGCCAACCCAAATGTAGATCCAAATGCAAACCCAAATGTA |
| | | GATCCAAATGCAAACCCAAATGCAAACCCAAATGCAAACCCAAATGC |
| | | AAACCCAAATGCAAACCCAAATGCAAACCCAAATGCAAACCCAAAT |
| | | GCAAACCCAAATGCAAACCCAAATGCAAACCCAAATGCAAACCCAA |
| | | ATGCAAACCCAAATGCAAACCCAAATGCAAACCCCAATGCAAATCCT |
| | | AATGCAAACCCAAATGCAAACCCAAACGTAGATCCTAATGCAAATCC |
| | | AAATGCAAACCCAAACGCAAACCCCAATGCAAATCCTAATGCAAACC |
| | | CCAATGCAAATCCTAATGCAAATCCTAATGCCAATCCAAATGCAAAT |
| | | CCAAATGCAAACCCAAACGCAAACCCCAATGCAAATCCTAATGCCAA |
| | | TCCAAATGCAAATCCAAATGCAAACCCAAATGCAAACCCAAATGCAA |
| | | ACCCCAATGCAAATCCTAATAAAAACAATCAAGGTAATGGACAAGGT |
| | | CACAATATGCCAAATGACCCAAACCGAAATGTAGATGAAAATGCTAA |
| | | TGCCAACAGTGCTGTAAAAAATAATAATAACGAAGAACCAAGTGATA |
| | | AGCACATAAAAGAATATTTAAACAAAATACAAAATTCTCTTTCAACT |
| | | GAATGGTCCCCATGTAGTGTAACTTGTGGAAATGGTATTCAAGTTAG |
| | | AATAAAGCCTGGCTCTGCTAATAAACCTAAAGACGAATTAGATTATG |
| | | CAAATGATATTGAAAAAAAAATTTGTAAAATGGAAAAATGTTCCAGT |
| | | GTGTTTAATGTCGTAAATAGTTCAATAGGATTAATAATGGTATTATCC |
| | | TTCTTGTTCCTTAATTAG |
| Tetanus TT | 125 | ATGCCCATCACCATCAACAACTTCAGGTACAGCGACCCCGTGAACAA |
| | | CGACACCATCATCATGATGGAGCCCCCCTACTGCAAGGGCCTGGACA |
| | | TCTACTACAAGGCCTTCAAGATCACCGACAGGATCTGGATCGTGCCC |
| | | GAGAGGTACGAGTTCGGCACCAAGCCCGAGGACTTCAACCCCCCCAG |
| | | CAGCCTGATCGAGGGCGCCAGCGAGTACTACGACCCCAACTACCTGA |
| | | GGACCGACAGCGACAAGGACAGGTTCCTGCAGACCATGGTGAAGCTG |
| | | TTCAACAGGATCAAGAACAACGTGGCCGGCGAGGCCCTGCTGGACAA |
| | | GATCATCAACGCCATCCCCTACCTGGGCAACAGCTACAGCCTGCTGG |
| | | ACAAGTTCGACACCAACAGCAACAGCGTGAGCTTCAACCTGCTGGAG |
| | | CAGGACCCCAGCGGCGCCACCACCAAGAGCGCCATGCTGACCAACCT |
| | | GATCATCTTCGGCCCCGGCCCCGTGCTGAACAAGAACGAGGTGAGGG |
| | | GCATCGTGCTGAGGGTGGACAACAAGAACTACTTCCCCTGCAGGGAC |
| | | GGCTTCGGCAGCATCATGCAGATGGCCTTCTGCCCCGAGTACGTGCCC |
| | | ACCTTCGACAACGTGATCGAGAACATCACCAGCCTGACCATCGGCAA |
| | | GAGCAAGTACTTCCAGGACCCCGCCCTGCTGCTGATGCACGAGCTGA |
| | | TCCACGTGCTGCACGGCCTGTACGGCATGCAGGTGAGCAGCCACGAG |
| | | ATCATCCCCAGCAAGCAGGAGATCTACATGCAGCACACCTACCCCAT |
| | | CAGCGCCGAGGAGCTGTTCACCTTCGGCGGCCAGGACGCCAACCTGA |
| | | TCAGCATCGACATCAAGAACGACCTGTACGAGAAGACCCTGAACGAC |
| | | TACAAGGCCATCGCCAACAAGCTGAGCCAGGTGACCAGCTGCAACGA |
| | | CCCCAACATCGACATCGACAGCTACAAGCAGATCTACCAGCAGAAGT |
| | | ACCAGTTCGACAAGGACAGCAACGGCCAGTACATCGTGAACGAGGA |
| | | CAAGTTCCAGATCCTGTACAACAGCATCATGTACGGCTTCACCGAGA |
| | | TCGAGCTGGGCAAGAAGTTCAACATCAAGACCAGGCTGAGCTACTTC |
| | | AGCATGAACCACGACCCCGTGAAGATCCCCAACCTGCTGGACGACAC |
| | | CATCTACAACGACACCGAGGGCTTCAACATCGAGAGCAAGGACCTGA |
| | | AGAGCGAGTACAAGGGCCAGAACATGAGGGTGAACACCAACGCCTT |
| | | CAGGAACGTGGACGGCAGCGGCCTGGTGAGCAAGCTGATCGGCCTGT |
| | | GCAAGAAGATCATCCCCCCCACCAACATCAGGGAGAACCTGTACAAC |
| | | AGGACCGCCAGCCTGACCGACCTGGGCGGCGAGCTGTGCATCAAGAT |
| | | CAAGAACGAGGACCTGACCTTCATCGCCGAGAAGAACAGCTTCAGCG |
| | | AGGAGCCCTTCCAGGACGAGATCGTGAGCTACAACACCAAGAACAA |
| | | GCCCCTGAACTTCAACTACAGCCTGGACAAGATCATCGTGGACTACA |
| | | ACCTGCAGAGCAAGATCACCCTGCCCAACGACAGGACCACCCCCGTG |
| | | ACCAAGGGCATCCCCTACGCCCCCGAGTACAAGAGCAACGCCGCCAG |
| | | CACCATCGAGATCCACAACATCGACGACAACACCATCTACCAGTACC |
| | | TGTACGCCCAGAAGAGCCCCACCACCCTGCAGAGGATCACCATGACC |
| | | AACAGCGTGGACGACGCCCTGATCAACAGCACCAAGATCTACAGCTA |
| | | CTTCCCCAGCGTGATCAGCAAGGTGAACCAGGGCGCCCAGGGCATCC |
| | | TGTTCCTGCAGTGGGTGAGGGACATCATCGACGACTTCACCAACGAG |
| | | AGCAGCCAGAAGACCACCATCGACAAGATCAGCGACGTGAGCACCA |
| | | TCGTGCCCTACATCGGCCCCGCCCTGAACATCGTGAAGCAGGGCTAC |
| | | GAGGGCAACTTCATCGGCGCCCTGGAGACCACCGGCGTGGTGCTGCT |
| | | GCTGGAGTACATCCCCGAGATCACCCTGCCCGTGATCGCCGCCCTGA |
| | | GCATCGCCGAGAGCAGCACCCAGAAGGAGAAGATCATCAAGACCAT |
| | | CGACAACTTCCTGGAGAAGAGGTACGAGAAGTGGATCGAGGTGTACA |
| | | AGCTGGTGAAGGCCAAGTGGCTGGGCACCGTGAACACCCAGTTCCAG |
| | | AAGAGGAGCTACCAGATGTACAGGAGCCTGGAGTACCAGGTGGACG |
| | | CCATCAAGAAGATCATCGACTACGAGTACAAGATCTACAGCGGCCCC |
| | | GACAAGGAGCAGATCGCCGACGAGATCAACAACCTGAAGAACAAGC |
| | | TGGAGGAGAAGGCCAACAAGGCCATGATCAACATCAACATCTTCATG |
| | | AGGGAGAGCAGCAGGAGCTTCCTGGTGAACCAGATGATCAACGAGG |
| | | CCAAGAAGCAGCTGCTGGAGTTCGACACCCAGAGCAAGAACATCCTG |
| | | ATGCAGTACATCAAGGCCAACAGCAAGTTCATCGGCATCACCGAGCT |
| | | GAAGAAGCTGGAGAGCAAGATCAACAAGGTGTTCAGCACCCCCATCC |
| | | CCTTCAGCTACAGCAAGAACCTGGACTGCTGGGTGGACAACGAGGAG |
| | | GACATCGACGTGATCCTGAAGAAGAGCACCATCCTGAACCTGGACAT |
| | | CAACAACGACATCATCAGCGACATCAGCGGCTTCAACAGCAGCGTGA |
| | | TCACCTACCCCGACGCCCAGCTGGTGCCCGGCATCAACGGCAAGGCC |
| | | ATCCACCTGGTGAACAACGAGAGCAGCGAGGTGATCGTGCACAAGGC |
| | | CATGGACATCGAGTACAACGACATGTTCAACAACTTCACCGTGAGCT |
| | | TCTGGCTGAGGGTGCCCAAGGTGAGCGCCAGCCACCTGGAGCAGTAC |
| | | GGCACCAACGAGTACAGCATCATCAGCAGCATGAAGAAGCACAGCCT |
| | | GAGCATCGGCAGCGGCTGGAGCGTGAGCCTGAAGGGCAACAACCTG |
| | | ATCTGGACCCTGAAGGACAGCGCCGGCGAGGTGAGGCAGATCACCTT |
| | | CAGGGACCTGCCCGACAAGTTCAACGCCTACCTGGCCAACAAGTGGG |
| | | TGTTCATCACCATCACCAACGACAGGCTGAGCAGCGCCAACCTGTAC |
| | | ATCAACGGCGTGCTGATGGGCAGCGCCGAGATCACCGGCCTGGGCGC |
| | | CATCAGGGAGGACAACAACATCACCCTGAAGCTGGACAGGTGCAAC |
| | | AACAACAACCAGTACGTGAGCATCGACAAGTTCAGGATCTTCTGCAA |
| | | GGCCCTGAACCCCAAGGAGATCGAGAAGCTGTACACCAGCTACCTGA |
| | | GCATCACCTTCCTGAGGGACTTCTGGGGCAACCCCCTGAGGTACGAC |
| | | ACCGAGTACTACCTGATCCCCGTGGCCAGCAGCAGCAAGGACGTGCA |
| | | GCTGAAGAACATCACCGACTACATGTACCTGACCAACGCCCCCAGCT |
| | | ACACCAACGGCAAGCTGAACATCTACTACAGGAGGCTGTACAACGGC |
| | | CTGAAGTTCATCATCAAGAGGTACACCCCCAACAACGAGATCGACAG |
| | | CTTCGTGAAGAGCGGCGACTTCATCAAGCTGTACGTGAGCTACAACA |
| | | ACAACGAGCACATCGTGGGCTACCCCAAGGACGGCAACGCCTTCAAC |
| | | AACCTGGACAGGATCCTGAGGGTGGGCTACAACGCCCCCGGCATCCC |
| | | CCTGTACAAGAAGATGGAGGCCGTGAAGCTGAGGGACCTGAAGACCT |
| | | ACAGCGTGCAGCTGAAGCTGTACGACGACAAGAACGCCAGCCTGGGC |
| | | CTGGTGGGCACCCACAACGGCCAGATCGGCAACGACCCCAACAGGG |
| | | ACATCCTGATCGCCAGCAACTGGTACTTCAACCACCTGAAGGACAAG |
| | | ATCCTGGGCTGCGACTGGTACTTCGTGCCCACCGACGAGGGCTGGAC |
| | | CAACGAC |
| Tuberculosis | 126 | GTGGCGAAGGTGAACATCAAGCCACTCGAGGACAAGATTCTCGTGCA |
| Mtb 10 kDa | | GGCCAACGAGGCCGAGACCACGACCGCGTCCGGTCTGGTCATTCCTG |
| chaperonin | | ACACCGCCAAGGAGAAGCCGCAGGAGGGCACCGTCGTTGCCGTCGGC |
| GroES | | CCTGGCCGGTGGGACGAGGACGGCGAGAAGCGGATCCCGCTGGACG |
| | | TTGCGGAGGGTGACACCGTCATCTACAGCAAGTACGGCGGCACCGAG |
| | | ATCAAGTACAACGGCGAGGAATACCTGATCCTGTCGGCACGCGACGT |
| | | GCTGGCCGTCGTTTCCAAGTAG |
| Tuberculosis | 127 | ATGTCATTTGTGGTCACGATCCCGGAGGCGCTAGCGGCGGTGGCGAC |
| Mtb PE | | CGATTTGGCGGGTATCGGGTCGACGATCGGCACCGCCAACGCGGCCG |
| family | | CCGCGGTCCCGACCACGACGGTGTTGGCCGCCGCCGCCGATGAGGTG |
| protein | | TCGGCGGCGATGGCGGCATTGTTCTCCGGACACGCCCAGGCCTATCA |
| | | GGCGCTGAGCGCCCAGGCGGCGCTGTTTCACGAGCAGTTCGTGCGGG |
| | | CGCTCACCGCCGGGGGGGGCTCGTATGCGGCCGCCGAGGCCGCCAGC |
| | | GCGGCCCCGCTAGAGGGTGTGCTCGACGTGATCAACGCCCCCGCCCT |
| | | GGCGCTGTTGGGGCGCCCACTGATCGGTAACGGAGCCAACGGGGCCC |
| | | CGGGGACCGGGGCAAACGGCGGCGACGGCGGAATCTTGATCGGCAA |
| | | CGGCGGGGCCGGCGGCTCCGGCGCGGCCGGCATGCCCGGGGGCAAC |
| | | GGCGGAGCCGCTGGCCTGTTCGGCAACGGCGGGGCCGGCGGCGCCGG |
| | | GGGGAACGTAGCGTCCGGCACCGCAGGGTTCGGCGGGGCCGGCGGG |
| | | GCCGGCGGGCTGCTCTACGGCGCCGGCGGGGCCGGCGGCGCCGGCGG |
| | | ACGCGCCGGTGGTGGGGTGGGCGGTATTGGTGGGGCCGGGGGGCCG |
| | | GCGGCAATGGCGGGCTGCTGTTCGGCGCCGGCGGGGCCGGCGGCGTC |
| | | GGCGGACTCGCGGCTGACGCCGGTGACGGCGGGGCCGGCGGAGACG |
| | | GCGGGTTGTTCTTCGGCGTGGGCGGTGCCGGCGGGGCCGGCGGCACC |
| | | GGCACTAATGTCACCGGCGGTGCCGGCGGGGCCGGCGGCAATGGCGG |
| | | GCTCCTGTTCGGCGCCGGCGGGGTGGGCGGTGTTGGCGGTGACGGTG |
| | | TGGCATTCCTGGGCACCGCCCCCGGCGGGCCCGGTGGTGCCGGCGGG |
| | | GCCGGTGGGCTGTTCGGCGTCGGTGGGGCCGGCGGCGCCGGCGGAAT |
| | | CGGATTGGTCGGGAACGGCGGTGCCGGGGGGTCCGGCGGGTCCGCCC |
| | | TGCTCTGGGGCGACGGCGGTGCCGGCGGCGCGGGTGGGGTCGGGTCC |
| | | ACTACCGGCGGTGCCGGCGGGGGGGGCGGCAACGCCGGCCTGCTGGT |
| | | AGGCGCCGGCGGGGCCGGCGGCGCCGGCGCACTCGGCGGTGGCGCT |
| | | ACCGGGGTGGGCGGCGCCGGCGGAAACGGCGGCACTGCGGGCCTGC |
| | | TGTTTGGTGCCGGCGGCGCCGGCGGATTCGGCTTCGGCGGTGCCGGG |
| | | GGCGCCGGTGGGCTCGGCGGCAAAGCCGGGCTGATCGGCGACGGCG |
| | | GTGACGGCGGCGCCGGAGGAAACGGCACCGGTGCCAAGGGCGGTGA |
| | | CGGCGGCGCTGGCGGCGGTGCCATCCTGGTCGGCAACGGCGGCAACG |
| | | GCGGCAACGCCGGGAGTGGCACACCTAACGGCAGCGCGGGCACCGG |
| | | CGGTGCCGGCGGGCTGTTGGGTAAGAACGGGATGAACGGGTTACCGT |
| | | AG |
| M. | 128 | ATGACAGACGTGAGCCGAAAGATTCGAGCTTGGGGACGCCGATTGAT |
| tuberculosis | | GATCGGCACGGCAGCGGCTGTAGTCCTTCCGGGCCTGGTGGGGCTTG |
| antigen 85B | | CCGGCGGAGCGGCAACCGCGGGCGCGTTCTCCCGGCCGGGGCTGCCG |
| precursor | | GTCGAGTACCTGCAGGTGCCGTCGCCGTCGATGGGCCGCGACATCAA |
| | | GGTTCAGTTCCAGAGCGGTGGGAACAACTCACCTGCGGTTTATCTGCT |
| | | CGACGGCCTGCGCGCCCAAGACGACTACAACGGCTGGGATATCAACA |
| | | CCCCGGCGTTCGAGTGGTACTACCAGTCGGGACTGTCGATAGTCATG |
| | | CCGGTCGGCGGGCAGTCCAGCTTCTACAGCGACTGGTACAGCCCGGC |
| | | CTGCGGTAAGGCTGGCTGCCAGACTTACAAGTGGGAAACCTTCCTGA |
| | | CCAGCGAGCTGCCGCAATGGTTGTCCGCCAACAGGGCCGTGAAGCCC |
| | | ACCGGCAGCGCTGCAATCGGCTTGTCGATGGCCGGCTCGTCGGCAAT |
| | | GATCTTGGCCGCCTACCACCCCCAGCAGTTCATCTACGCCGGCTCGCT |
| | | GTCGGCCCTGCTGGACCCCTCTCAGGGGATGGGGCCTAGCCTGATCG |
| | | GCCTCGCGATGGGTGACGCCGGCGGTTACAAGGCCGCAGACATGTGG |
| | | GGTCCCTCGAGTGACCCGGCATGGGAGCGCAACGACCCTACGCAGCA |
| | | GATCCCCAAGCTGGTCGCAAACAACACCCGGCTATGGGTTTATTGCG |
| | | GGAACGGCACCCCGAACGAGTTGGGCGGTGCCAACATACCCGCCGAG |
| | | TTCTTGGAGAACTTCGTTCGTAGCAGCAACCTGAAGTTCCAGGATGCG |
| | | TACAACGCCGCGGGGGGCACAACGCCGTGTTCAACTTCCCGCCCAA |
| | | CGGCACGCACAGCTGGGAGTACTGGGGCGCTCAGCTCAACGCCATGA |
| | | AGGGTGACCTGCAGAGTTCGTTAGGCGCCGGCTGA |
| Adenovirus | 129 | CCCCAGTGGAGCTACATGCACATCAGCGGCCAGGACGCCAGCGAGTA |
| 5 Hexon | | CCTGAGCCCCGGCCTGGTGCAGTTCGCCAGGGCCACCGAGACCTACT |
| | | TCAGCCTGAACAACAAGTTCAGGAACCCCACCGTGGCCCCCACCCAC |
| | | GACGTGACCACCGACAGGAGCCAGAGGCTGACCCTGAGGTTCATCCC |
| | | CGTGGACAGGGAGGACACCGCCTACAGCTACAAGGCCAGGTTCACCC |
| | | TGGCCGTGGGCGACAACAGGGTGCTGGACATGGCCAGCACCTACTTC |
| | | GACATCAGGGGCGTGCTGGACAGGGGCCCCACCTTCAAGCCCTACAG |
| | | CGGCACCGCCTACAACGCCCTGGCCCCCAAGGGCGCCCCCAACAGCT |
| | | GCGAGTGGGAGCAGACCGAGGACAGCGGCAGGGCCGTGGCCGAGGA |
| | | CGAGGAGGAGGAGGACGAGGACGAGGAGGAGGAGGAGGAGGAGCA |
| | | GAACGCCAGGGACCAGGCCACCAAGAAGACCCACGTGTACGCCCAG |
| | | GCCCCCCTGAGCGGCGAGACCATCACCAAGAGCGGCCTGCAGATCGG |
| | | CAGCGACAACGCCGAGACCCAGGCCAAGCCCGTGTACGCCGACCCCA |
| | | GCTACCAGCCCGAGCCCCAGATCGGCGAGAGCCAGTGGAACGAGGC |
| | | CGACGCCAACGCCGCCGGCGGCAGGGTGCTGAAGAAGACCACCCCC |
| | | ATGAAGCCCTGCTACGGCAGCTACGCCAGGCCCACCAACCCCTTCGG |
| | | CGGCCAGAGCGTGCTGGTGCCCGACGAGAAGGGCGTGCCCCTGCCCA |
| | | AGGTGGACCTGCAGTTCTTCAGCAACACCACCAGCCTGAACGACAGG |
| | | CAGGGCAACGCCACCAAGCCCAAGGTGGTGCTGTACAGCGAGGACGT |
| | | GAACATGGAGACCCCCGACACCCACCTGAGCTACAAGCCCGGCAAGG |
| | | GCGACGAGAACAGCAAGGCCATGCTGGGCCAGCAGAGCATGCCCAA |
| | | CAGGCCCAACTACATCGCCTTCAGGGACAACTTCATCGGCCTGATGT |
| | | ACTACAACAGCACCGGCAACATGGGCGTGCTGGCCGGCCAGGCCAGC |
| | | CAGCTGAACGCCGTGGTGGACCTGCAGGACAGGAACACCGAGCTGA |
| | | GCTACCAGCTGCTGCTGGACAGCATCGGCGACAGGACCAGGTACTTC |
| | | AGCATGTGGAACCAGGCCGTGGACAGCTACGACCCCGACGTGAGGAT |
| | | CATCGAGAACCACGGCACCGAGGACGAGCTGCCCAACTACTGCTTCC |
| | | CCCTGGGCGGCATCGGCGTGACCGACACCTACCAGGCCATCAAGGCC |
| | | AACGGCAACGGCAGCGGCGACAACGGCGACACCACCTGGACCAAGG |
| | | ACGAGACCTTCGCCACCAGGAACGAGATCGGCGTGGGCAACAACTTC |
| | | GCCATGGAGATCAACCTGAACGCCAACCTGTGGAGGAACTTCCTGTA |
| | | CAGCAACATCGCCCTGTACCTGCCCGACAAGCTGAAGTACAACCCCA |
| | | CCAACGTGGAGATCAGCGACAACCCCAACACCTACGACTACATGAAC |
| | | AAGAGGGTGGTGGCCCCCGGCCTGGTGGACTGCTACATCAACCTGGG |
| | | CGCCAGGTGGAGCCTGGACTACATGGACAACGTGAACCCCTTCAACC |
| | | ACCACAGGAACGCCGGCCTGAGGTACAGGAGCATGCTGCTGGGCAAC |
| | | GGCAGGTACGTGCCCTTCCACATCCAGGTGCCCCAGAAGTTCTTCGCC |
| | | ATCAAGAACCTGCTGCTGCTGCCCGGCAGCTACACCTACGAGTGGAA |
| | | CTTCAGGAAGGACGTGAACATGGTGCTGCAGAGCAGCCTGGGCAACG |
| | | ACCTGAGGGTGGACGGCGCCAGCATCAAGTTCGACAGCATCTGCCTG |
| | | TACGCCACCTTCTTCCCCATGGCCCACAACACCGCCAGCACCCTGGAG |
| | | GCCATGCTGAGG |
| SARS-CoV- | 130 | ATGGATTTGTTTATGAGAATCTTCACAATTGGAACTGTAACTTTGAAG |
| 2 ORF3a | | CAAGGTGAAATCAAGGATGCTACTCCTTCAGATTTTGTTCGCGCTACT |
| | | GCAACGATACCGATACAAGCCTCACTCCCTTTCGGATGGCTTATTGTT |
| | | GGCGTTGCACTTCTTGCTGTTTTTCAGAGCGCTTCCAAAATCATAACC |
| | | CTCAAAAAGAGATGGCAACTAGCACTCTCCAAGGGTGTTCACTTTGTT |
| | | TGCAACTTGCTGTTGTTGTTTGTAACAGTTTACTCACACCTTTTGCTCG |
| | | TTGCTGCTGGCCTTGAAGCCCCTTTTCTCTATCTTTATGCTTTAGTCTA |
| | | CTTCTTGCAGAGTATAAACTTTGTAAGAATAATAATGAGGCTTTGGCT |
| | | TTGCTGGAAATGCCGTTCCAAAAACCCATTACTTTATGATGCCAACTA |
| | | TTTTCTTTGCTGGCATACTAATTGTTACGACTATTGTATACCTTACAAT |
| | | AGTGTAACTTCTTCAATTGTCATTACTTCAGGTGATGGCACAACAAGT |
| | | CCTATTTCTGAACATGACTACCAGATTGGTGGTTATACTGAAAAATGG |
| | | GAATCTGGAGTAAAAGACTGTGTTGTATTACACAGTTACTTCACTTCA |
| | | GACTATTACCAGCTGTACTCAACTCAATTGAGTACAGACACTGGTGTT |
| | | GAACATGTTACCTTCTTCATCTACAATAAAATTGTTGATGAGCCTGAA |
| | | GAACATGTCCAAATTCACACAATCGACGGTTCATCCGGAGTTGTTAAT |
| | | CCAGTAATGGAACCAATTTATGATGAACCGACGACGACTACTAGCGT |
| | | GCCTTTGTAA |
| SARS-CoV | 131 | ATGTCTGATAATGGACCCCAATCAAACCAACGTAGTGCCCCCCGCAT |
| Nucleocapsid | | TACATTTGGTGGACCCACAGATTCAACTGACAATAACCAGAATGGAG |
| protein | | GACGCAATGGGGCAAGGCCAAAACAGCGCCGACCCCAAGGTTTACCC |
| | | AATAATACTGCGTCTTGGTTCACAGCTCTCACTCAGCATGGCAAGGA |
| | | GGAACTTAGATTCCCTCGAGGCCAGGGCGTTCCAATCAACACCAATA |
| | | GTGGTCCAGATGACCAAATTGGCTACTACCGAAGAGCTACCCGACGA |
| | | GTTCGTGGTGGTGACGGCAAAATGAAAGAGCTCAGCCCCAGATGGTA |
| | | CTTCTATTACCTAGGAACTGGCCCAGAAGCTTCACTTCCCTACGGCGC |
| | | TAACAAAGAAGGCATCGTATGGGTTGCAACTGAGGGAGCCTTGAATA |
| | | CACCCAAAGACCACATTGGCACCCGCAATCCTAATAACAATGCTGCC |
| | | ACCGTGCTACAACTTCCTCAAGGAACAACATTGCCAAAAGGCTTCTA |
| | | CGCAGAGGGAAGCAGAGGCGGCAGTCAAGCCTCTTCTCGCTCCTCAT |
| | | CACGTAGTCGCGGTAATTCAAGAAATTCAACTCCTGGCAGCAGTAGG |
| | | GGAAATTCTCCTGCTCGAATGGCTAGCGGAGGTGGTGAAACTGCCCT |
| | | CGCGCTATTGCTGCTAGACAGATTGAACCAGCTTGAGAGCAAAGTTT |
| | | CTGGTAAAGGCCAACAACAACAAGGCCAAACTGTCACTAAGAAATCT |
| | | GCTGCTGAGGCATCTAAAAAGCCTCGCCAAAAACGTACTGCCACAAA |
| | | ACAGTACAACGTCACTCAAGCATTTGGGAGACGTGGTCCAGAACAAA |
| | | CCCAAGGAAATTTCGGGGACCAAGACCTAATCAGACAAGGAACTGAT |
| | | TACAAACATTGGCCGCAAATTGCACAATTTGCTCCAAGTGCCTCTGCA |
| | | TTCTTTGGAATGTCACGCATTGGCATGGAAGTCACACCTTCGGGAACA |
| | | TGGCTGACTTATCATGGAGCCATTAAATTGGATGACAAAGATCCACA |
| | | ATTCAAAGACAACGTCATACTGCTGAACAAGCACATTGACGCATACA |
| | | AAACATTCCCACCAACAGAGCCTAAAAAGGACAAAAAGAAAAAGAC |
| | | TGATGAAGCTCAGCCTTTGCCGCAGAGACAAAAGAAGCAGCCCACTG |
| | | TGACTCTTCTTCCTGCGGCTGACATGGATGATTTCTCCAGACAACTTC |
| | | AAAATTCCATGAGTGGAGCTTCTGCTGATTCAACTCAGGCATAA |
| Dengue | 132 | GGCACCGGCAACATCGGCGAGACCCTGGGCGAGAAGTGGAAGAGCA |
| NS5 | | GGCTGAACGCCCTGGGCAAGAGCGAGTTCCAGATCTACAAGAAGAGC |
| | | GGCATCCAGGAGGTGGACAGGACCCTGGCCAAGGAGGGCATCAAGA |
| | | GGGGCGAGACCGACCACCACGCCGTGAGCAGGGGCAGCGCCAAGCT |
| | | GAGGTGGTTCGTGGAGAGGAACATGGTGACCCCCGAGGGCAAGGTG |
| | | GTGGACCTGGGCTGCGGCAGGGGCGGCTGGAGCTACTACTGCGGCGG |
| | | CCTGAAGAACGTGAGGGAGGTGAAGGGCCTGACCAAGGGCGGCCCC |
| | | GGCCACGAGGAGCCCATCCCCATGAGCACCTACGGCTGGAACCTGGT |
| | | GAGGCTGCAGAGCGGCGTGGACGTGTTCTTCATCCCCCCCGAGAAGT |
| | | GCGACACCCTGCTGTGCGACATCGGCGAGAGCAGCCCCAACCCCACC |
| | | GTGGAGGCCGGCAGGACCCTGAGGGTGCTGAACCTGGTGGAGAACTG |
| | | GCTGAACAACAACACCCAGTTCTGCATAAGGTGCTGAACCCCTACAT |
| | | GCCCAGCGTGATCGAGAAGATGGAGGCCCTGCAGAGGAAGTACGGC |
| | | GGCGCCCTGGTGAGGAACCCCCTGAGCAGGAACAGCACCCACGAGAT |
| | | GTACTGGGTGAGCAACGCCAGCGGCAACATCGTGAGCAGCGTGAACA |
| | | TGATCAGCAGGATGCTGATCAACAGGTTCACCATGAGGTACAAGAAG |
| | | GCCACCTACGAGCCCGACGTGGACCTGGGCAGCGGCACCAGGAACAT |
| | | CGGCATCGAGAGCGAGATCCCCAACCTGGACATCATCGGCAAGAGGA |
| | | TCGAGAAGATCAAGCAGGAGCACGAGACCAGCTGGCACTACGACCA |
| | | GGACCACCCCTACAAGACCTGGGCCTACCACGGCAGCTACGAGACCA |
| | | AGCAGACCGGCAGCGCCAGCAGCATGGTGAACGGCGTGGTGAGGCT |
| | | GCTGACCAAGCCCTGGGACGTGGTGCCCATGGTGACCCAGATGGCCA |
| | | TGACCGACACCACCCCCTTCGGCCAGCAGAGGGTGTTCAAGGAGAAG |
| | | GTGGACACCAGGACCCAGGAGCCCAAGGAGGGCACCAAGAAGCTGA |
| | | TGAAGATCACCGCCGAGTGGCTGTGGAAGGAGCTGGGCAAGAAGAA |
| | | GACCCCCAGGATGTGCACCAGGGAGGAGTTCACCAGGAAGGTGAGG |
| | | AGCAACGCCGCCCTGGGCGCCATCTTCACCGACGAGAACAAGTGGAA |
| | | GAGCGCCAGGGAGGCCGTGGAGGACAGCAGGTTCTGGGAGCTGGTG |
| | | GACAAGGAGAGGAACCTGCACCTGGAGGGCAAGTGCGAGACCTGCG |
| | | TGTACAACATGATGGGCAAGAGGGAGAAGAAGCTGGGCGAGTTCGG |
| | | CAAGGCCAAGGGCAGCAGGGCCATCTGGTACATGTGGCTGGGCGCCA |
| | | GGTTCCTGGAGTTCGAGGCCCTGGGCTTCCTGAACGAGGACCACTGG |
| | | TTCAGCAGGGAGAACAGCCTGAGCGGCGTGGAGGGCGAGGGCCTGC |
| | | ACAAGCTGGGCTACATCCTGAGGGACGTGAGCAAGAAGGAGGGCGG |
| | | CGCCATGTACGCCGACGACACCGCCGGCTGGGACACCAGGATCACCC |
| | | TGGAGGACCTGAAGAACGAGGAGATGGTGACCAACCACATGGAGGG |
| | | CGAGCACAAGAAGCTGGCCGAGGCCATCTTCAAGCTGACCTACCAGA |
| | | ACAAGGTGGTGAGGGTGCAGAGGCCCACCCCCAGGGGCACCGTGAT |
| | | GGACATCATCAGCAGGAGGGACCAGAGGGGCAGCGGCCAGGTGGGC |
| | | ACCTACGGCCTGAACACCTTCACCAACATGGAGGCCCAGCTGATCAG |
| | | GCAGATGGAGGGCGAGGGCGTGTTCAAGAGCATCCAGCACCTGACCA |
| | | TCACCGAGGAGATCGCCGTGCAGAACTGGCTGGCCAGGGTGGGCAGG |
| | | GAGAGGCTGAGCAGGATGGCCATCAGCGGCGACGACTGCGTGGTGA |
| | | AGCCCCTGGACGACAGGTTCGCCAGCGCCCTGACCGCCCTGAACGAC |
| | | ATGGGCAAGATCAGGAAGGACATCCAGCAGTGGGAGCCCAGCAGGG |
| | | GCTGGAACGACTGGACCCAGGTGCCCTTCTGCAGCCACCACTTCCAC |
| | | GAGCTGATCATGAAGGACGGCAGGGTGCTGGTGGTGCCCTGCAGGAA |
| | | CCAGGACGAGCTGATCGGCAGGGCCAGGATCAGCCAGGGCGCCGGC |
| | | TGGAGCCTGAGGGAGACCGCCTGCCTGGGCAAGAGCTACGCCCAGAT |
| | | GTGGAGCCTGATGTACTTCCACAGGAGGGACCTGAGGCTGGCCGCCA |
| | | ACGCCATCTGCAGCGCCGTGCCCAGCCACTGGGTGCCCACCAGCAGG |
| | | ACCACCTGGAGCATCCACGCCAAGCACGAGTGGATGACCACCGAGGA |
| | | CATGCTGACCGTGTGGAACAGGGTGTGGATCCAGGAGAACCCCTGGA |
| | | TGGAGGACAAGACCCCCGTGGAGAGCTGGGAGGAGATCCCCTACCTG |
| | | GGCAAGAGGGAGGACCAGTGGTGCGGCAGCCTGATCGGCCTGACCA |
| | | GCAGGGCCACCTGGGCCAAGAACATCCAGGCCGCCATCAACCAGGTG |
| | | AGGAGCCTGATCGGCAACGAGGAGTACACCGACTACATGCCCAGCAT |
| | | GAAGAGGTTCAGGAGGGAGGAGGAGGAGGCCGGCGTGCTGTGG |
| HBV | 133 | ATGCCCCTGAGCTACCAGCACTTCAGGAAGCTGCTGCTGCTGGACGA |
| polymerase | | GGAGGCCGGCCCCCTGGAGGAGGAGCTGCCCAGGCTGGCCGACGAG |
| | | GGCCTGAACAGGAGGGTGGCCGAGGACCTGAACCTGGGCAACCTGA |
| | | ACGTGAGCATCCCCTGGACCCACAAGGTGGGCAACTTCACCGGCCTG |
| | | TACAGCAGCACCGTGCCCTGCTTCAACCCCAAGTGGCAGACCCCCAG |
| | | CTTCCCCGACATCCACCTGCAGGAGGACATCGTGGACAGGTGCAAGC |
| | | AGTTCGTGGGCCCCCTGACCGTGAACGAGAACAGGAGGCTGAAGCTG |
| | | ATCATGCCCGCCAGGTTCTACCCCAACGTGACCAAGTACCTGCCCCTG |
| | | GACAAGGGCATCAAGCCCTACTACCCCGAGCACGTGGTGAACCACTA |
| | | CTTCCAGACCAGGCACTACCTGCACACCCTGTGGAAGGCCGGCATCC |
| | | TGTACAAGAGGGAGAGCACCAGGAGCGCCAGCTTCTGCGGCAGCCCC |
| | | TACAGCTGGGAGCAGGACCTGCAGCACGGCAGGCTGGTGTTCAAGAC |
| | | CAGCAAGAGGCACGGCGACAAGAGCTTCTGCCCCCAGAGCCCCGGCA |
| | | TCCTGCCCAGGAGCAGCGTGGGCCCCTGCATCCAGAGCCAGCTGAGG |
| | | AAGAGCAGGCTGGGCCCCCAGCCCGCCCAGGGCCAGCTGGCCGGCA |
| | | GGCAGCAGGGCGGCAGCGGCAGCATCAGGGCCAGGGTGCACCCCAG |
| | | CCCCTGGGGCACCGTGGGCGTGGAGCCCAGCGGCAGCGGCCACACCC |
| | | ACAACTGCGCCAGCAGCAGCAGCAGCTGCCTGCACCAGAGCGCCGTG |
| | | AGGAAGGCCGCCTACAGCCTGATCAGCACCAGCAAGGGCCACAGCA |
| | | GCAGCGGCCACGCCGTGGAGCTGCACCACTTCCCCCCCAACAGCAGC |
| | | AGGAGCCAGAGCCAGGGCCCCGTGCTGAGCTGCTGGTGGCTGCAGTT |
| | | CAGGAACAGCGAGCCCTGCAGCGAGTACTGCCTGTGCCACATCGTGA |
| | | ACCTGATCGAGGACTGGGGCCCCTGCACCGAGCACGGCGAGCACAGG |
| | | ATCAGGACCCCCAGGACCCCCGCCAGGGTGACCGGCGGCGTGTTCCT |
| | | GGTGGACAAGAACCCCCACAACACCACCGAGAGCAGGCTGGTGGTG |
| | | GACTTCAGCCAGTTCAGCAGGGGCGACACCAGGGTGAGCTGGCCCAA |
| | | GTTCGCCGTGCCCAACCTGCAGAGCCTGACCAACCTGCTGAGCAGCA |
| | | ACCTGAGCTGGCTGAGCCTGGACGTGAGCGCCGCCTTCTACCACCTG |
| | | CCCCTGCACCCCGCCGCCATGCCCCACCTGCTGGTGGGCAGCAGCGG |
| | | CCTGAGCAGGTACGTGGCCAGGCTGAGCAGCAACAGCAGGATCATCA |
| | | ACAACCAGCACAGGACCATGCAGAACCTGCACAACAGCTGCAGCAG |
| | | GAACCTGTACGTGAGCCTGATGCTGCTGTACAAGACCTACGGCAGGA |
| | | AGCTGCACCTGTACAGCCACCCCATCATCCTGGGCTTCAGGAAGATC |
| | | CCCATGGGCGTGGGCCTGAGCCCCTTCCTGCTGGCCCAGTTCACCAGC |
| | | GCCATCTGCAGCGTGGTGAGGAGGGCCTTCCCCCACTGCCTGGCCTTC |
| | | AGCTACATGGACGACGTGGTGCTGGGCGCCAAGAGCGTGCAGCACCT |
| | | GGAGAGCCTGTACGCCGCCGTGACCAACTTCCTGCTGAGCCTGGGCA |
| | | TCCACCTGAACCCCCACAAGACCAAGAGGTGGGGCTACAGCCTGAAC |
| | | TTCATGGGCTACGTGATCGGCTGCTGGGGCACCATGCCCCAGGAGCA |
| | | CATCGTGCAGAAGATCAAGATGTGCTTCAGGAAGCTGCCCGTGAACA |
| | | GGCCCATCGACTGGAAGGTGTGCCAGAGGATCGTGGGCCTGCTGGGC |
| | | TTCGCCGCCCCCTTCACCCAGTGCGGCTACCCCGCCCTGATGCCCCTG |
| | | TACGCCTGCATCCAGGCCAAGCAGGCCTTCACCTTCAGCCCCACCTAC |
| | | AAGGCCTTCCTGAGCAAGCAGTACCTGAACCTGTACCCCGTGGCCAG |
| | | GCAGAGGAGCGGCCTGTGCCAGGTGTTCGCCGACGCCACCCCCACCG |
| | | GCTGGGGCCTGGCCATCGGCCACCAGAGGATGAGGGGCACCTTCGTG |
| | | AGCCCCCTGCCCATCCACACCGCCGAGCTGCTGGCCGCCTGCTTCGCC |
| | | AGGAGCAGGAGCGGCGCCAAGCTGATCGGCACCGACAACAGCGTGG |
| | | TGCTGAGCAGGAAGTACACCAGCTTCCCCTGGCTGCTGGGCTGCGCC |
| | | GCCAACTGGATCCTGAGGGGCACCAGCTTCGTGTACGTGCCCAGCGC |
| | | CCTGAACCCCGCCGACGACCCCAGCAGGGGCAGGCTGGGCCTGTACA |
| | | GGCCCCTGCTGAGGCTGCTGTACAGGCCCACCACCGGCAGGACCAGC |
| | | CTGTACGCCGACAGCCCCAGCGTGCCCAGCCACCTGCCCGACAGGGT |
| | | GCACTTCGCCAGCCCCCTGCACGTGGCCTGGAGGCCCCCC |
| HCV NS5a | 134 | GACACCAGCTGGCTGAGGGACGTGTGGGACTGGGTGTGCACCGTGCT |
| | | GAGCGACTTCAGGGTGTGGCTGCAGGCCAAGCTGCTGCCCAGGCTGC |
| | | CCGGCATCCCCTTCTTCAGCTGCCAGACCGGCTACAGGGGCGTGTGG |
| | | GCCGGCGACGGCGTGTGCCACACCACCTGCACCTGCGGCGCCGTGAT |
| | | CGCCGGCCACGTGAAGAACGGCACCATGAAGATCACCGGCCCCAAG |
| | | ACCTGCAGCAACACCTGGCACGGCACCTTCCCCATCAACGCCACCAC |
| | | CACCGGCCCCAGCACCCCCAGGCCCGCCCCCAGCTACCAGAGGGCCC |
| | | TGTGGAGGGTGAGCGCCGAGGACTACGTGGAGGTGAGGAGGCTGGG |
| | | CGACAGGCACTACGTGGTGGGCGTGACCGCCGAGGGCCTGAAGTGCC |
| | | CCTGCCAGGTGCCCGCCCCCGAGTTCTTCACCGAGATCGACGGCGTG |
| | | AGGCTGCACAGGTACGCCCCCCCCTGCAAGCCCCTGCTGAGGGACGA |
| | | GGTGACCTTCAGCGTGGGCCTGAGCACCTACGCCATCGGCAGCCAGC |
| | | TGCCCTGCGAGCCCGAGCCCGACGTGACCGTGGTGACCAGCATGCTG |
| | | ACCGACCCCACCCACATCACCGCCGAGACCGCCGCCAGGAGGCTGAA |
| | | GAGGGGCAGCCCCCCCAGCCTGGCCAGCAGCAGCGCCAGCCAGCTGA |
| | | GCGCCCCCAGCCTGAAGGCCACCTGCACCACCAGCAAGGACCACCCC |
| | | GACATGGAGCTGATCGAGGCCAACCTGCTGTGGAGGCAGGAGATGG |
| | | GCGGCAACATCACCAGGGTGGAGAGCGAGAACAAGGTGGTGGTGCT |
| | | GGACAGCTTCGAGCCCCTGACCGCCGAGTACGACGAGAGGGAGATCA |
| | | GCGTGAGCGCCGAGTGCCACAGGCCCCCCAGGCACAAGTTCCCCCCC |
| | | GCCCTGCCCATCTGGGCCAGGCCCGACTACAACCCCCCCCTGATCCA |
| | | GGCCTGGCAGATGCCCGGCTACGAGCCCCCCGTGGTGAGCGGCTGCG |
| | | CCATCGCCCCCCCCAAGCCCGCCCCCATCCCCCCCCCCAGGAGGAAG |
| | | AGGCTGGTGAGGCTGGACGAGAGCACCGTGAGCCACGCCCTGGCCCA |
| | | GCTGGCCGACAAGGTGTTCGTGGAGAGCAGCAGCGACCCCGGCCCCA |
| | | GCAGCGACAGCGGCCTGAGCATCGCCAGCCCCGTGCCCCCCGCCCCC |
| | | ACCACCAGCGACGACGCCTGCAGCGAGGCCGAGAGCTACAGCAGCA |
| | | TGCCCCCCCTGGAGGGCGAGCCCGGCGACCCCGACCTGAGCAGCGGC |
| | | AGCTGGAGCACCGTGAGCGACCAGGACGACGTGGTGTGCTGC |
| Influenza A | 135 | ATGGCGTCCCAAGGCACCAAACGGTCTTATGAACAGATGGAAACTGA |
| NP | | TGGGGAACGCCAGAATGCAACTGAGATCAGAGCATCCGTCGGGAAG |
| | | ATGATTGATGGAATTGGACGATTCTACATCCAAATGTGCACCGAACTT |
| | | AAACTCAGTGATTATGAGGGGCGACTGATCCAGAACAGCTTAACAAT |
| | | AGAGAGAATGGTGCTCTCTGCTTTTGACGAGAGAAGGAATAAATATC |
| | | TGGAAGAACATCCCAGCGCGGGGAAGGATCCTAAGAAAACTGGAGG |
| | | ACCCATATACAAGAGAGTAGATGGAAAGTGGATGAGGGAACTCGTCC |
| | | TTTATGACAAAGAAGAAATAAGGCGAATCTGGCGCCAAGCCAATAAT |
| | | GGTGATGATGCAACAGCTGGGCTGACTCACATGATGATCTGGCATTC |
| | | CAATTTGAATGATACAACATACCAGAGGACAAGAGCTCTTGTTCGCA |
| | | CCGGAATGGATCCCAGGATGTGCTCTTTGATGCAGGGTTCGACTCTCC |
| | | CTAGGAGGTCTGGAGCTGCAGGCGCTGCAGTCAAAGGAGTTGGGACA |
| | | ATGGTGATGGAGTTGATCAGGATGATCAAACGTGGGATCAATGATCG |
| | | GAACTTCTGGAGAGGTGAGAATGGACGGAAAACAAGGAGTGCTTAC |
| | | GAGAGAATGTGCAACATTCTCAAAGGAAAATTTCAAACAGCTGCACA |
| | | AAGAGCAATGATGGATCAAGTGAGAGAAAGCCGGAACCCAGGAAAT |
| | | GCTGAGATCGAAGATCTAATCTTTCTGGCACGGTCTGCACTCATATTG |
| | | AGAGGGTCAGTTGCTCACAAATCTTGTCTGCCCGCCTGTGTGTATGGA |
| | | CCTGCCATAGCCAGTGGGTACAACTTCGAAAAAGAGGGATACTCTCT |
| | | AGTGGGAATAGACCCTTTCAAACTGCTTCAAAACAGCCAAGTATACA |
| | | GCCTAATCAGACCGAACGAGAATCCAGCACACAAGAGTCAGCTGGTG |
| | | TGGATGGCATGCAATTCTGCTGCATTTGAAGATCTAAGAGTATTAAGC |
| | | TTCATCAGAGGGACCAAAGTATCCCCAAGGGGGAAACTTTCCACTAG |
| | | AGGAGTACAAATTGCTTCAAATGAAAACATGGATACTATGGAATCAA |
| | | GTACTCTTGAACTAAGAAGCAGGTACTGGGCCATAAGGACCAGAAGT |
| | | GGAGGAAACACTAATCAACAGAGGGCCTCTGCAGGTCAAATCAGTGT |
| | | ACAACCTGCATTTTCTGTGCAAAGAAACCTCCCATTTGACAAACCAAC |
| | | CATCATGGCAGCATTCACTGGGAATACAGAGGGAAGAACATCAGACA |
| | | TGAGGGCAGAAATCATAAGGATGATGGAAGGTGCAAAACCAGAAGA |
| | | AATGTCCTTCCAGGGGGGGGGAGTCTTCGAGCTCTCGGACGAAAAGG |
| | | CAACGAACCCGATCGTGCCCTCTTTTGACATGAGTAATGAAGGATCTT |
| | | ATTTCTTCGGAGACAATGCAGAGGAGTACGACAATTAA |

In some embodiments, a MHC binding peptide comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 136-163.

TABLE 7
Example MHC binding peptide sequences

| Antigen | SEQ ID NO | Peptide sequence |
|---|---|---|
| Mycobacterium p25 | 136 | FQDAYNAAGGHNAVF |
| CNW59158.1 (M. tuberculosis antigen | | |
| 85B precursor CNW59158.1) | | |
| M. tuberculosis CFP-10 | 137 | EISTNIRQAGVQYSR |
| CFS32012.1 | | |
| SARS-CoV-2 Spike | 138 | TRFQTRFQTLLALHRSYLT |
| 7SBS_A | | |
| Influenza A HA | 139 | PKYVKQNTLKLAT |
| AYE19441.1 | | |
| Mtb ESAT-6 like protein | 140 | MSQIMYNYPAMMAHA |
| KCD52888.1 | | |
| Aspergillus fumigatus Crf1/p41 | 141 | HTYTIDWTKDAVTWS |
| AAC61261.1 | | |
| Pertussis toxin subunit 2 | 142 | YYSNVTATRLLSSTNS |
| WP_033468320.1 | | |
| HBV envelope | 143 | QAGFFLLTRILTIPQS |
| AGP09303.1 | | |
| HCV polyprotein | 144 | VYYLTRDPTTPLARAA |
| QTF98639.1 | | |
| HIV-1 gag | 145 | FRDYVDRFYKTLRAEQASQE |
| ABY76167.1 | | |
| HPV E2 | 146 | PIVQLQGDSNCLKCFR |
| ABC79060.1 | | |
| Malaria CSP | 147 | EYLNKIQNSLSTEWSPCSVT |
| CAB64182.1 | | |
| Tetanus TT | 148 | FNNFTVSFWLRVPKVSASHLE |
| WP_129031034.1 | | |
| Tuberculosis Mtb 10 kDa chaperonin | 149 | GEEYLILSARDVLAV |
| GroES MBV9319653.1 | | |
| Tuberculosis Mtb ESAT6 | 150 | MTEQQWNFAGIEAAA |
| KBS40701.1 | | |
| Tuberculosis Mtb PE family protein | 151 | MHVSFVMAYPEMLAA |
| CFI98308.1 | | |
| Adenovirus 5 Hexon | 152 | TDLGQNLLY |
| AAP31203.1 | | |
| Chlamydia trachomatis MOMP | 153 | RLNMFTPYI |
| P08780.1 | | |
| SARS-CoV-2 ORF3a | 154 | FTSDYYQLY |
| UAQ13861.1 | | |
| SARS-CoV Nucleocapsid protein | 155 | LLLDRLNQL |
| UBW56997.1 | | |
| SARS-CoV-2 ORF3a | 156 | LLYDANYFL |
| UAQ13861.1 | | |
| Dengue NS5 | 157 | KLAEAIFKL |
| QCH40793.1 | | |
| HBV polymerase | 158 | KYTSFPWLL |
| ABR22107.1 | | |
| HCV NS5a | 159 | VLSDFKTWL |
| ACF32936.1 | | |
| HIV-1 gag | 160 | RLRPGGKKK |
| ABY76167.1 | | |
| Influenza A NP | 161 | SPIVPSFDM |
| ABY81789.2 | | |
| Toxoplasma gondii H-2 Kb tgd057 | 162 | SVLAFRRL |
| PIL96569.1 | | |
| Tuberculosis ESAT-6 | 163 | AMASTEGNV |
| WP_055379083.1 | | |

In some embodiments, a composition herein encodes for or comprises two or more MHC binding peptides. For instance, the two or more MHC binding peptides is 2, 3, 4, 5, 6, 7, 8, 9, or 10 MHC binding peptides. Two MHC binding peptides may be the same or different. The two or more MHC binding peptides may be connected by a linker. The linker may be cleavable or non-cleavable. In some embodiments, the two or more MHC binding peptides are connected by a linker comprising a cleavage site. Non-limiting example cleavage sites include exopeptidase, endopeptidase, and exopeptidase cleavage sites. In some embodiments, the cleavage site is a proteasome cleavage site, a cysteine protease cleavage site (cathepsin B, F, H, L, S, Z, and AEP, for asparaginylendopeptidase), an aspartate protease cleavage site (cathepsin D, E), a serine protease cleavage site (cathepsin A, G), or a combination thereof. In some embodiments, the polynucleotide encoding the cleavage site comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to SEQ ID NO: 81.

Further non-limiting example cleavage sites are described elsewhere herein, including, but not limited to, as shown in Table 3. In some embodiments, the cleavage site comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 83-92. In some embodiments, the polynucleotide encoding the cleavage site comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of SEQ ID NOS: 73-82.

### Nucleic Acid Production Methods

In some embodiments, a nucleic acid construct (e.g., construct that will be transcribed into mRNA) is generated using nucleic acid construction methods, including but not limited to, gene synthesis, vector amplification, plasmid purification, plasmid linearization, and cDNA template synthesis. Once an antigen of interest is selected, a primary construct is designed. A first region of linked nucleotides encoding the antigen of interest may be constructed using an open reading frame (ORF) of a selected nucleic acid transcript. In some embodiments, the ORF comprises the wild type ORF, an isoform, variant of a fragment thereof. In some embodiments, an open reading frame (ORF) refers to a region of a nucleic acid molecule that is capable of encoding a polypeptide of interest. OFRs often begin with the start codon and end with a nonsense or termination codon or signal.

In some embodiments, the nucleic sequence is codon optimized. The codon optimization is a method to match codon frequencies in target and host organisms to ensure proper folding, customize transcriptional and translational control regions, insert or remove protein trafficking sequences, remove/add post translational modification sites in encoded protein (e.g. glycosylation sites), add, remove or shuffle protein domains, bias GC content to increase mRNA stability or reduce secondary structures, minimize tandem repeat codons or base runs that may impair gene construction or expression, insert or delete restriction sites; or modify ribosome binding sites and mRNA degradation sites. Examples of codon optimization tools, algorithms and services including, but not limited to, services from GeneArt (Life Technologies), DNA2.0 (Menlo Park Calif) and/or proprietary methods.

In some embodiments, mRNA is generated by the following processes, which include, but not limited to, in vitro transcription, cDNA template removal, mRNA capping, and tailing reactions. In some embodiments, mRNA construct undergoes a purification process to separate mRNA from at least one contaminant. In some embodiments, a contaminant is any substance that makes another unfit, impure, or inferior. The purification processes include, but not limited to mRNA clean-up, quality assurance, and quality control. mRNA clean-up may be performed by methods such as AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as strong anion exchange HPLC, weak anion exchange HPLC, reverse phase HPLC (RP-HPLC), and hydrophobic interaction HPLC (HIC-HPLC). A quality assurance and quality control may be performed using methods such as gel electrophoresis, UV absorbance, or analytical HPLC.

In some embodiments, mRNA is quantified using methods such as ultraviolet visible spectroscopy (UV/Vis). Examples of a UV/Vis spectrometer include but not limited to a NANODROP® spectrometer (ThermoFisher, Waltham, Mass.). The quantified mRNA may be analyzed in order to determine the size of the mRNA and to check whether the degradation of the mRNA has occurred. For instance, degradation of the mRNA may be checked using agarose gel electrophoresis or HPLC based purification methods. Examples of the HPLC based purification methods include, but not limited to strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Nucleic Acid Delivery

In some embodiments, a nucleic acid composition herein is delivered as a naked or unmodified nucleic acid. In other embodiments, the nucleic acid composition is delivered via a vehicle. In some embodiments, a nucleic acid composition herein is delivered as DNA. In some embodiments, a nucleic acid composition herein is delivered as RNA, e.g., mRNA.

In some embodiments, the nucleic acid is delivered to the subject via a vehicle. The vehicle may be a lipid nanoparticle or a virus-like particle.

In some embodiments, the nucleic acid is delivered via a lipid nanoparticle vehicle. Non-limiting lipid nanoparticles include, but are not limited to, 1,2-di-O-octadecenyl-3-trimethylammonium-propane (DOTMA), 1,2-dioleoyl-sn-glycero-3-phosphoethanolamine (DOSPA), 1,2-dioleoyl-3-trimethylammonium-propane (DOTAP), ethylphosphatidylcholine (ePC), (6Z,9Z,28Z,31Z)-heptatriaconta-6,9,28,31-tetraen-19-yl 4-(dimethylamino) butanoate (DLin-MC3-DMA; MC3), 1,1′-((2-(4-(2-((2-(bis(2-hydroxydodecyl)amino)ethyl) (2-hydroxydodecyl)amino)ethyl) piperazin-1-yl)ethyl)azanediyl)bis(dodecan-2-ol) (C12-200), ((4-hydroxybutyl)azanediyl)bis(hexane-6,1-diyl)bis(2-hexyldecanoate) (ALC-0315), 3,6-bis(4-(bis(2-hydroxydodecyl)amino)butyl)piperazine-2,5-dione (cKK-E12), heptadecan-9-yl 8-((2-hydroxyethyl)(6-oxo-6-(undecyloxy)hexyl)amino) octanoate (Lipid H (SM-102)), (((3,6-dioxopiperazine-2,5-diyl)bis(butane-4,1-diyl))bis(azanetriyl))tetrakis(ethane-2,1-diyl) (9Z,9′Z,9″Z,9″′Z,12Z,12′Z,12″Z,12″′Z)-tetrakis(octadeca-9,12-dienoate) (OF-Deg-Lin), ethyl 5,5-di((Z)-heptadec-8-en-1-yl)-1-(3-(pyrrolidin-1-yl)propyl)-2,5-dihydro-H-imidazole-2-carboxylate (A2-Iso5-2DC18), tetrakis(8-methylnonyl) 3,3′,3″,3″′-(((methylazanediyl)bis(propane-3,1 diyl))bis(azanetriyl))tetrapropionate (3060i10), bis(2-(dodecyldisulfanyl)ethyl) 3,3′-((3-methyl-9-oxo-10-oxa-13,14-dithia-3,6-diazahexacosyl)azanediyl)dipropionate (BAME-016B), N1,N3,N5-tris(3-(didodecylamino)propyl)benzene-1,3,5-tricarboxamide (TT3), decyl(2-(dioctylammonio)ethyl)phosphate (9A1P9), hexa(octan-3-yl) 9,9′,9″,9″′,9″″,9′″″-((((benzene-1,3,5-tricarbonyl)yris(azanediyl))tris(propane-3,1-diyl))tris(azanetriyl))hexanonanoate (FTT5), 1,2-distearoyl-sn-glycero-3-phosphocholine (DSPC), 1,2-dioleoyl-sn-glycero-3-phosphoethanolamine (DOPE), 1,2-dimyristoyl-rac-glycero-3-methoxypolyethylene glycol-2000 (PEG2000-DMG), 2-[(polyethylene glycol)-2000]—N,N-ditetradecylacetamide (ALC-0159), Cholesterol, 30-[N—(N′,N′-dimethylaminoethane)-carbamoyl]cholesterol (DC-Cholesterol), (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-TH-cyclopenta[a]phenanthren-3-ol ((3-sitosterol), and 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-TH-cyclopenta[a]phenanthren-3-yl)oxy)carbonyl)amino)-N,N-bis(2-hydroxyethyl)-N-methylethan-1-aminium bromide (BHEM-Cholesterol).

In some embodiments, the nucleic acid is delivered via a virus-like particle vehicle. Non-limiting virus-like particles include, but are not limited to, non-enveloped VLPs (single or multi-capsid protein VLPs) and enveloped VLPs.

### Methods of Inducing an Immune Response

Various embodiments provide for methods of inducing an immune response in a subject by administering to the subject a composition described herein. The immune response may comprise an antibody response and/or a cell-mediated immune response in the subject. For example, the subject is administered a composition comprising an antigen to stimulate production of antibodies that bind to the antigen. In another example, the subject is administered a composition comprising mRNA encoding an antigen to stimulate production of antibodies that bind to the antigen. In some embodiments, the antigen is expressed from the mRNA. Certain compositions comprise or encode a MHC binding peptide. In some embodiments, the composition stimulates the production of antibodies by stimulating the adaptive immune response after delivery of the composition to the subject. In some embodiments, the adaptive immune response of the subject comprises a stimulation of B lymphocytes to release polyclonal antibodies that specifically bind to the antigen. In some embodiments, the adaptive immune response of the subject comprises stimulating cell-mediated immune responses.

Also provided herein are methods for evaluating non-human or human subjects for antibody response to a composition herein. In some embodiments, the evaluating is before and/or after administration of the composition. A non-limiting method is provided in Example 3.

### Pharmaceutical Compositions, Administration and Dosage

In various embodiments, the compositions herein are formulated for delivery via any route of administration. “Route of administration” may refer to any administration pathway known in the art, including but not limited to intradermal, intramuscular, and/or subcutaneous administration. It is appreciated that actual dosage can vary depending on the route of administration, the delivery system used, the target cell, organ, or tissue, the subject, as well as the degree of effect sought. Size and weight of the tissue, organ, and/or patient can also affect dosing. Doses may further include additional agents, including but not limited to a carrier. Non-limiting examples of suitable carriers are known in the art: for example, water, saline, ethanol, glycerol, lactose, sucrose, dextran, agar, pectin, plant-derived oils, phosphate-buffered saline, and/or diluents.

In various embodiments, provided are pharmaceutical compositions including a pharmaceutically acceptable excipient along with a therapeutically effective amount of a nucleic acid and/or peptide described herein. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. The active ingredient can be mixed with excipients which are pharmaceutically acceptable and compatible with the active ingredient and in amounts suitable for use in therapeutic methods described herein. Such excipients may be solid, liquid, semisolid, or, in the case of an aerosol composition, gaseous. Suitable excipients are, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain auxiliary substances such as wetting or emulsifying agents, pH buffering agents and the like which enhance or maintain the effectiveness of the active ingredient, or increase the stability of the pharmaceutical product. In addition, if desired, the composition can contain auxiliary substances to modify the density of the pharmaceutical product. Therapeutic compositions as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine, and the like. Liquid compositions can contain liquid phases in addition to and in the exclusion of water, for example, glycerin, vegetable oils such as cottonseed oil, and water-oil emulsions. Physiologically tolerable carriers are well known in the art.

The pharmaceutical compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of nucleic acid (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration.

### Kits

Further provided is a kit to perform methods described herein. The kit is an assemblage of components, including at least one of the compositions described herein. Thus, in some embodiments, the kit comprises a nucleic acid and/or peptide composition described herein. The nucleic acid or peptide may be combined with, or complexed to, another component such as a vehicle for delivery, or may be unmodified for direct delivery.

Instructions for use of the components may be included in the kit. Optionally, the kit also contains other useful components, such as, diluents, buffers, pharmaceutically acceptable carriers, syringes, applicators, measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of a composition containing a nucleic acid herein. The packaging material generally has an external label which indicates the contents and/or purpose of the kit and/or its components.

### Non-Limiting Numbered Embodiments

1. A nucleic acid comprising (i) a first exogenous polynucleotide, and (ii) a 5′ untranslated region (5′ UTR) of a first flavivirus and/or a 3′ untranslated region (3′ UTR) of a second flavivirus.
2. The nucleic acid of embodiment 1, wherein the first flavivirus is a tick-borne flavivirus (TBFV), a mosquito-borne flavivirus (MBFV), an insect-specific flavivirus (ISFV), no-known vector flavivirus (NKFV), or a non-classified flavivirus (NCFV).
3. The nucleic acid of embodiment 1 or embodiment 2, wherein the first flavivirus is a dengue virus (DENV), West Nile virus (WNV), Japanese encephalitis virus (JEV), yellow fever virus (YFV), Zika virus (ZIKV), tick-born encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV).
4. The nucleic acid of embodiment 1, wherein the first flavivirus is a dengue virus (DENV).
5. The nucleic acid of embodiment 4, wherein the dengue virus is a dengue virus serotype 4 (DENV-4).
6. The nucleic acid of any one of embodiments 1-5, wherein the second flavivirus is a tick-borne flavivirus (TBFV), a mosquito-borne flavivirus (MBFV), an insect-specific flavivirus (ISFV), no-known vector flavivirus (NKFV), or a non-classified flavivirus (NCFV).
7. The nucleic acid of any one of embodiments 1-6, wherein the second flavivirus is a dengue virus (DENV), West Nile virus (WNV), Japanese encephalitis virus (JEV), yellow fever virus (YFV), Zika virus (ZIKV), tick-born encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV).
8. The nucleic acid of any one of embodiments 1-5, wherein the second flavivirus is a dengue virus (DENV).
9. The nucleic acid of embodiment 8, wherein the dengue virus is a dengue virus serotype 4 (DENV-4).
10. The nucleic acid of any one of embodiments 1-9, wherein the first flavivirus and the second flavivirus are the same flavivirus.
11. The nucleic acid of any one of embodiments 1-10, wherein the 5′ UTR comprises a sequence at least about 80% identical to any one of SEQ ID NOS: 1-36, or comprises a sequence at least 80% identical to at least 50, 60, 70, 80, 90, or 100 contiguous bases of a virus of Table 1.
12. The nucleic acid of any one of embodiments 1-10, wherein the 5′ UTR comprises a sequence derived from any one of SEQ ID NOS: 1-36, or of a virus of Table 1.
13. The nucleic acid of embodiment 11, wherein the 5′ UTR is at least 80% identical to SEQ ID NO: 5 or 36.
14. The nucleic acid of any one of embodiments 1-13, wherein the 3′ UTR comprises a sequence at least about 80% identical to any one of SEQ ID NOS: 37-70, or comprises a sequence at least 80% identical to at least 50, 60, 70, 80, 90, or 100 contiguous bases of a virus of Table 2.
15. The nucleic acid of any one of embodiments 1-13, wherein the 3′ UTR comprises a sequence derived from any one of SEQ ID NOS: 37-70, or of a virus of Table 2.
16. The nucleic acid of embodiment 14, wherein the 3′ UTR is at least 80% identical to SEQ ID NO: 40.
17. The nucleic acid of any one of embodiments 1-16, wherein the 5′ UTR comprises the stem loop A of the 5′ UTR of the first flavivirus.
18. The nucleic acid of any one of embodiments 1-17, wherein the 5′ UTR comprises the stem loop B of the 5′ UTR of the first flavivirus.
19. The nucleic acid of any one of embodiments 1-18, wherein the 5′ UTR comprises the 5′ ATG of the first flavivirus.
20. The nucleic acid of any one of embodiments 1-19, wherein the 5′ UTR comprises the capsid-coding region hairpin element (cHP) of the first flavivirus.
21. The nucleic acid of any one of embodiments 1-20, wherein the 5′ UTR comprises the 5′ conserved sequence of the first flavivirus.
22. The nucleic acid of any one of embodiments 1-21, wherein the 3′ UTR comprises at least one endonuclease resistance sequence of the second flavivirus.
23. The nucleic acid of any one of embodiments 1-22, wherein the 3′ UTR comprises the short hairpin structure of the second flavivirus.
24. The nucleic acid of any one of embodiments 1-23, wherein the 3′ UTR comprises the 3′ cyclization sequence of the second flavivirus.
25. The nucleic acid of any one of embodiments 1-24, wherein the 3′ UTR comprises the 3′ TAG, TAA, or TGA of the second flavivirus.
26. The nucleic acid of any one of embodiments 1-25, wherein the 5′ UTR does not comprise a 5′ cap modification.
27. The nucleic acid of any one of embodiments 1-25, wherein the 5′ UTR comprises a 5′ cap modification.
28. The nucleic acid of any one of embodiments 1-27, wherein the 5′ UTR has a length of about 80 bases to about 200 bases.
29. The nucleic acid of any one of embodiments 1-28, wherein the 3′ UTR has a length of about 200 to about 700 bases.
30. The nucleic acid of any one of embodiments 1-29, wherein the nucleic acid does not comprise a sequence encoding 10 or more contiguous amino acids of a structural protein of the first flavivirus or the second flavivirus.
31. The nucleic acid of any one of embodiments 1-30, wherein the nucleic acid does not comprise a sequence encoding 10 or more contiguous amino acids of any structural protein of the first flavivirus or the second flavivirus.
32. The nucleic acid of embodiment 30 or embodiment 31, wherein the structural protein is a capsid, membrane, or envelope protein of the first flavivirus or the second flavivirus.
33. The nucleic acid of any one of embodiments 1-32, wherein the nucleic acid does not comprise a sequence encoding 10 or more contiguous amino acids of a non-structural protein of the first flavivirus or the second flavivirus.
34. The nucleic acid of any one of embodiments 1-33, wherein the nucleic acid does not comprise a sequence encoding 10 or more contiguous amino acids of any non-structural protein of the first flavivirus or the second flavivirus.
35. The nucleic acid of any one of embodiments 1-34, wherein the nucleic acid does not comprise a sequence 3′ to the exogenous nucleotide sequence comprising at least 10 bases having at least 80% adenosine residues.
36. The nucleic acid of any one of embodiments 1-35, wherein the exogenous polynucleotide encodes a polypeptide.
37. The nucleic acid of embodiment 36, wherein the exogenous polynucleotide is translated into the polypeptide in healthy cells or during cellular stress responses.
38. The nucleic acid of any one of embodiments 1-37, wherein the nucleic acid is resistant to degradation by a RNAse.
39. The nucleic acid of embodiment 38, wherein the RNAse is XRN-1.
40. The nucleic acid of embodiment 38, wherein the RNAse comprises one or more of the extracellular RNAses selected from the group consisting of hRNAse1, hRNAse2, hRNAse3, hRNAse 4, hRNAse5, hRNAse6, hRNAse7, hRNAse8, hRNAse9, hRNAse10, hRNAse1l, hRNAse12, hRNAse13, bovine seminal RNAse, bovine milk RNAse, rodent RNAse, frog RNAse, RNAseT2, plant self-incompatibility RNAse, or bacterial RNAse.
41. The nucleic acid of any one of embodiments 1-40, wherein the nucleic acid has no or fewer than 10 base modifications.
42. The nucleic acid of any one of embodiments 1-41, wherein the nucleic acid has no or fewer than 10 backbone modifications.
43. The nucleic acid of any one of embodiments 1-42, wherein the nucleic acid has no or fewer than 10 sugar modifications.
44. The nucleic acid of any one of embodiments 1-43, wherein the nucleic acid is a deoxyribonucleic acid (DNA).
45. A ribonucleic acid (RNA) transcribed from the DNA of embodiment 44.
46. The RNA of embodiment 45, wherein the RNA is transcribed in vitro or in vivo.
47. The nucleic acid of any one of embodiments 1-43, wherein the nucleic acid is a ribonucleic acid (RNA).
48. The nucleic acid of any one of embodiments 45-47, wherein the RNA is a messenger RNA.
49. The nucleic acid of any one of embodiments 1-48, comprising a self-cleavage site.
50. The nucleic acid of any one of embodiments 1-49, comprising an internal ribosome entry site.
51. The nucleic acid of any one of embodiments 1-50, comprising a sequence encoding a peptide that induces ribosomal skipping during translation.
52. The nucleic acid of any one of embodiments 1-51, comprising a sequence encoding a peptide motif of DxExNPGP, where x is any amino acid.
53. The nucleic acid of any one of embodiments 1-52, comprising a sequence at least 80% identical to SEQ ID NO: 71.
54. The nucleic acid of any one of embodiments 1-53, comprising a sequence encoding a signal peptide.
55. The nucleic acid of embodiment 54, wherein the signal peptide is Gaussia luciferase, human albumin, human chymotrypsinogen, human interleukin-2, or human trypsinogen-2.
56. The nucleic acid of embodiment 54 or embodiment 55, wherein the signal peptide is at least 80% identical to any one of SEQ ID NOS: 107-112.
57. The nucleic acid of embodiment 54 or embodiment 55, wherein the signal peptide is at least 80% identical to SEQ ID NO: 107.
58. The nucleic acid of any one of embodiments 1-57, comprising a sequence encoding a cleavage site positioned between the 5′ UTR and the exogenous polynucleotide.
59. The nucleic acid of embodiment 58, wherein the cleavage site comprises an exopeptidase, endopeptidase and/or exopeptidase cleavage site.
60. The nucleic acid of embodiment 58 or embodiment 59, wherein the cleavage site is a proteasome cleavage site, a cysteine protease cleavage site, an aspartate protease cleavage site, a serine protease cleavage site, or a combination thereof.
61. The nucleic acid of any of embodiments 58-60, wherein the sequence encoding the cleavage site comprises a sequence at least 80% identical to any one of SEQ ID NOS: 73-82.
62. The nucleic acid of any of embodiments 58-60, wherein the sequence encoding the cleavage site comprises a sequence at least 80% identical to SEQ ID NO: 81.
63. The nucleic acid of any of embodiments 58-60, wherein the cleavage site comprises a sequence at least 80% identical to any one of SEQ ID NOS: 83-92.
64. The nucleic acid of any of embodiments 58-60, wherein the cleavage site comprises a sequence at least 80% identical to SEQ ID NO: 91.
65. The nucleic acid of any one of embodiments 1-64, wherein the exogenous polynucleotide encodes a pathogen-associated antigen.
66. The nucleic acid of embodiment 65, wherein the pathogen is a virus, bacteria, fungus, protozoa, or helminth.
67. The nucleic acid of embodiment 65 or embodiment 66, wherein the exogenous polynucleotide encodes a viral structural protein, a viral envelope protein, a viral capsid protein, or a viral nonstructural protein, or any combination thereof.
68. The nucleic acid of any one of embodiments 65-67, wherein the exogenous polynucleotide encodes an antigen from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus.
69. The nucleic acid of any one of embodiments 65-67, wherein the exogenous polynucleotide encodes an antigen from a bacteria selected from Helicobacter pylori, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii.
70. The nucleic acid of any one of embodiments 65-67, wherein the exogenous polynucleotide encodes an antigen from a fungi selected from Cryptococcus neoformans, Histoplasma capsulatum, Coccidioides immitis, Blastomyces dermatitidis, Chlamydia trachomatis, and Candida albicans.
71. The nucleic acid of any one of embodiments 65-67, wherein the exogenous polynucleotide encodes an antigen from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major.
72. The nucleic acid of any one of embodiments 1-71, wherein the exogenous polynucleotide comprises a sequence at least 80% identical to any one of SEQ ID NOS: 93-96.
73. The nucleic acid of any one of embodiments 1-72, wherein the exogenous polynucleotide encodes an antigen having a sequence at least 80% identical to any one of SEQ ID NOS: 97-100.
74. A method of inducing an immune response in a subject, the method comprising administering to the subject the nucleic acid of any one of embodiments 1-73.
75. A nucleic acid composition comprising a first sequence encoding a first antigen, and a second sequence encoding a MHC binding peptide.
76. The nucleic acid of embodiment 75, wherein the MHC binding peptide is a MHC class I and/or a MHC class II peptide.
77. The nucleic acid of embodiment 75 or embodiment 76, wherein the second sequence comprises a sequence at least 80% identical to any one of SEQ ID NOS: 113-135.
78. The nucleic acid of embodiment 77, wherein the second sequence comprises a sequence at least 80% identical to SEQ ID NO: 113.
79. The nucleic acid of embodiment 75 or embodiment 76, wherein the MHC binding peptide comprises a sequence at least 80% identical to any one of SEQ ID NOS: 136-163.
80. The nucleic acid of embodiment 79, wherein the MHC binding peptide comprises a sequence at least 80% identical to SEQ ID NO: 136.
81. The nucleic acid of embodiment 75 or embodiment 76, wherein the second sequence comprises a pathogen-associated sequence.
82. The nucleic acid of embodiment 81, wherein the pathogen is a virus, bacteria, fungus, protozoa, or helminth.
83. The nucleic acid of embodiment 81 or embodiment 82, wherein the second sequence is at least 80% identical to 10 or more nucleobases from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus.
84. The nucleic acid of embodiment 81 or embodiment 82, wherein the second sequence is at least 80% identical to 10 or more nucleobases from a bacteria selected from Helicobacter pylori, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii.
85. The nucleic acid of embodiment 81 or embodiment 82, wherein the second sequence is at least 80% identical to 10 or more nucleobases from a fungi selected from Cryptococcus neoformans, Histoplasma capsulatum, Coccidioides immitis, Blastomyces dermatitidis, Chlamydia trachomatis, and Candida albicans.
86. The nucleic acid of embodiment 81 or embodiment 81, wherein the second sequence is at least 80% identical to 10 or more nucleobases from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major.
87. The nucleic acid of any one of embodiments 75-86, wherein the MHC binding peptide has a length of 7-20 peptides.
88. The nucleic acid of any one of embodiments 75-87, comprising two or more sequences encoding a MHC binding peptide.
89. The nucleic acid of any one of embodiments 75-88, wherein the first sequence is at least 80% identical to 10 or more nucleobases from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus.
90. The nucleic acid of any one of embodiments 75-88, wherein the first sequence is at least 80% identical to 10 or more nucleobases from a bacteria selected from Helicobacter pyloris, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii.
91. The nucleic acid of any one of embodiments 75-88, wherein the first sequence is at least 80% identical to 10 or more nucleobases from a fungi selected from Cryptococcus neoformans, Histoplasma capsulatum, Coccidioides immitis, Blastomyces dermatitidis, Chlamydia trachomatis, and Candida albicans.
92. The nucleic acid of any one of embodiments 75-88, wherein the first sequence is at least 80% identical to 10 or more nucleobases from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major.
93. The nucleic acid of any one of embodiments 75-88, wherein the first antigen has a sequence at least 80% identical to any one of SEQ ID NOS: 97-100.
94. The nucleic acid of any one of embodiments 75-88, wherein the first sequence comprises a sequence at least 80% identical to any one of SEQ ID NOS: 93-96.
95. The nucleic acid of any one of embodiments 75-94, wherein the first sequence and the second sequence are present on two separate nucleic acid strands.
96. The nucleic acid of any one of embodiments 75-94, wherein the first sequence and the second sequence are connected.
97. The nucleic acid of any one of embodiments 75-96, comprising a sequence encoding a cleavage site.
98. The nucleic acid of embodiment 97, wherein the cleavage site comprises an exopeptidase, endopeptidase and/or exopeptidase cleavage site.
99. The nucleic acid of embodiment 97 or embodiment 98, wherein the cleavage site is a proteasome cleavage site, a cysteine protease cleavage site, an aspartate protease cleavage site, or a serine protease cleavage site.
100. The nucleic acid of any one of embodiments 97-99, wherein the sequence encoding the cleavage site comprises a sequence at least 80% identical to any one of SEQ ID NOS: 73-82.
101. The nucleic acid of any of embodiments 97-99, wherein the sequence encoding the cleavage site comprises a sequence at least 80% identical to SEQ ID NO: 81.
102. The nucleic acid of any of embodiments 97-99, wherein the cleavage site comprises a sequence at least 80% identical to any one of SEQ ID NOS: 83-92.
103. The nucleic acid of any of embodiments 97-99, wherein the cleavage site comprises a sequence at least 80% identical to SEQ ID NO: 91.
104. The nucleic acid of any one of embodiments 75-103, comprising a sequence encoding a signal peptide.
105. The nucleic acid of embodiment 104, wherein the signal peptide is Gaussia luciferase, human albumin, human chymotrypsinogen, human interleukin-2, or human trypsinogen-2.
106. The nucleic acid of embodiment 104 or embodiment 105, wherein the signal peptide is at least 80% identical to any one of SEQ ID NOS: 107-112.
107. The nucleic acid of embodiment 104 or embodiment 105, wherein the signal peptide is at least 80% identical to SEQ ID NO: 107.
108. The nucleic acid of any one of embodiments 75-107, wherein the nucleic acid is a deoxyribonucleic acid (DNA).
109. A ribonucleic acid (RNA) transcribed from the DNA of embodiment 108.
110. The RNA of embodiment 109, wherein the RNA is transcribed in vitro or in vivo.
111. The nucleic acid of any one of embodiments 75-107, wherein the nucleic acid is a ribonucleic acid (RNA).
112. The nucleic acid of any one of embodiments 109-111, wherein the RNA is a messenger RNA.
113. A peptide translated from the nucleic acid of any one of embodiments 109-112.
114. A method of inducing an immune response in a subject, the method comprising administering to the subject the nucleic acid of any one of embodiments 75-112 or the peptide of embodiment 113.
115. The method of embodiment 74 or embodiment 114, wherein the nucleic acid is delivered via a lipid nanoparticle, virus-like particle, or naked.
116. A nucleic acid comprising (i) a first exogenous polynucleotide, (ii) a 5′ untranslated region (5′ UTR) of a first flavivirus and/or a 3′ untranslated region (3′ UTR) of a second flavivirus, and (iii) a polynucleotide encoding a MHC binding peptide.
117. The nucleic acid of embodiment 116, wherein the first flavivirus is a tick-borne flavivirus (TBFV), a mosquito-bome flavivirus (MBFV), an insect-specific flavivirus (ISFV), no-known vector flavivirus (NKFV), or a non-classified flavivirus (NCFV).
118. The nucleic acid of embodiment 116 or embodiment 117, wherein the first flavivirus is a dengue virus (DENV), West Nile virus (WNV), Japanese encephalitis virus (JEV), yellow fever virus (YFV), Zika virus (ZIKV), tick-bom encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV).
119. The nucleic acid of embodiment 116, wherein the first flavivirus is a dengue virus (DENV).
120. The nucleic acid of embodiment 119, wherein the dengue virus is a dengue virus serotype 4 (DENV-4).
121. The nucleic acid of any one of embodiments 116-120, wherein the second flavivirus is a tick-bome flavivirus (TBFV), a mosquito-bome flavivirus (MBFV), an insect-specific flavivirus (ISFV), no-known vector flavivirus (NKFV), or a non-classified flavivirus (NCFV).
122. The nucleic acid of any one of embodiments 116-121, wherein the second flavivirus is a dengue virus (DENV), West Nile virus (WNV), Japanese encephalitis virus (JEV), yellow fever virus (YFV), Zika virus (ZIKV), tick-bom encephalitis virus (TBEV), Usutu virus (USUV), Apoi virus (APOIV), border disease virus (BDV), bovine viral diarrhea virus (BVDV), Bussuquara virus (BSQV), cell fusing agent virus (CFAV), classical swine fever virus (CSFV), Culex flavivirus (CxFV), Entebbe bat virus (ENTV), pestivirus giraffe-1, hepatitis C virus (HCV), hepatitis GB virus B (GBV-B), GB virus C/hepatitis G virus (GBV-C), Ilheus virus (ILHV), Kamiti river virus (KRV), Kokobera virus (KOKV), Langat virus (LGTV), Louping ill virus (LIV), Modoc virus (MODV), Montana myotis leukoencephalitis virus (MMLV), Murray Valley encephalitis virus (MVEV), Omsk hemorrhagic fever virus (OHFV), Powassan virus (POWV), Rio Bravo virus (RBV), Sepik virus (SEPV), Tamana bat virus (TABV), or Yokose virus (YOKV).
123. The nucleic acid of any one of the embodiments 116-120, wherein the second flavivirus is a dengue virus (DENV).
124. The nucleic acid of embodiment 123, wherein the dengue virus is a dengue virus serotype 4 (DENV-4).
125. The nucleic acid of any one of embodiments 116-124, wherein the first flavivirus and the second flavivirus are the same flavivirus.
126. The nucleic acid of any one of embodiments 116-125, wherein the 5′ UTR comprises a sequence at least about 80% identical to any one of SEQ ID NOS: 1-36 or comprises a sequence at least 80% identical to at least 50, 60, 70, 80, 90, or 100 contiguous bases of a virus of Table 1.
127. The nucleic acid of any one of embodiments 116-125, wherein the 5′ UTR comprises a sequence derived from any one of SEQ ID NOS: 1-36, or of a virus of Table 1.
128. The nucleic acid of embodiment 127, wherein the 5′ UTR is at least 80% identical to SEQ ID NO: 5 or 36.
129. The nucleic acid of any one of embodiments 116-128, wherein the 3′ UTR comprises a sequence at least about 80% identical to any one of SEQ ID NOS: 37-70, or comprises a sequence at least 80% identical to at least 50, 60, 70, 80, 90, or 100 contiguous bases of a virus of Table 2.
130. The nucleic acid of any one of embodiments 116-128, wherein the 3′ UTR comprises a sequence derived from any one of SEQ ID NOS: 37-70, or of a virus of Table 2.
131. The nucleic acid of embodiment 130, wherein the 3′ UTR is at least 80% identical to SEQ ID NO: 40.
132. The nucleic acid of any one of embodiments 116-131, wherein the 5′ UTR comprises the stem loop A of the 5′ UTR of the first flavivirus.
133. The nucleic acid of any one of embodiments 116-132, wherein the 5′ UTR comprises the stem loop B of the 5′ UTR of the first flavivirus.
134. The nucleic acid of any one of embodiments 116-133, wherein the 5′ UTR comprises the 5′ ATG of the first flavivirus.
135. The nucleic acid of any one of embodiments 116-134, wherein the 5′ UTR comprises the capsid-coding region hairpin element (cHP) of the first flavivirus.
136. The nucleic acid of any one of embodiments 116-135, wherein the 5′ UTR comprises the 5′ conserved sequence of the first flavivirus.
137. The nucleic acid of any one of embodiments 116-136, wherein the 3′ UTR comprises at least one endonuclease resistance sequence of the second flavivirus.
138. The nucleic acid of any one of embodiments 116-137, wherein the 3′ UTR comprises the short hairpin structure of the second flavivirus.
139. The nucleic acid of any one of embodiments 126-138, wherein the 3′ UTR comprises the 3′ cyclization sequence of the second flavivirus.
140. The nucleic acid of any one of embodiments 126-139, wherein the 3′ UTR comprises the 3′ TAG, TAA, or TGA of the second flavivirus.
141. The nucleic acid of any one of embodiments 116-140, wherein the 5′ UTR does not comprise a 5′ cap modification.
142. The nucleic acid of any one of embodiments 116-141, wherein the 5′ UTR comprises a 5′ cap modification.
143. The nucleic acid of any one of embodiments 116-142, wherein the 5′ UTR has a length of about 80 bases to about 200 bases.
144. The nucleic acid of any one of embodiments 116-143, wherein the 3′ UTR has a length of about 200 to about 700 bases.
145. The nucleic acid of any one of embodiments 116-144, wherein the nucleic acid does not comprise a sequence encoding 10 or more contiguous amino acids of a structural protein of the first flavivirus or the second flavivirus.
146. The nucleic acid of any one of embodiments 116-145, wherein the nucleic acid does not comprise a sequence encoding 10 or more contiguous amino acids of any structural protein of the first flavivirus or the second flavivirus.
147. The nucleic acid of embodiment 145 or embodiment 146, wherein the structural protein is a capsid, membrane, or envelope protein of the first flavivirus or the second flavivirus.
148. The nucleic acid of any one of embodiments 116-147, wherein the nucleic acid does not comprise a sequence encoding 10 or more contiguous amino acids of a non-structural protein of the first flavivirus or the second flavivirus.
149. The nucleic acid of any one of embodiments 116-148, wherein the nucleic acid does not comprise a sequence encoding 10 or more contiguous amino acids of any non-structural protein of the first flavivirus or the second flavivirus.
150. The nucleic acid of any one of embodiments 116-149, wherein the nucleic acid does not comprise a sequence 3′ to the exogenous nucleotide sequence comprising at least 10 bases having at least 80% adenosine residues.
151. The nucleic acid of any one of embodiments 116-150, wherein the exogenous polynucleotide encodes a polypeptide.
152. The nucleic acid of embodiment 151, wherein the exogenous polynucleotide is translated into the polypeptide in healthy cells or during cellular stress responses.
153. The nucleic acid of any one of embodiments 116-152, wherein the nucleic acid is resistant to degradation by a RNAse.
154. The nucleic acid of embodiment 153, wherein the RNAse is XRN-1.
155. The nucleic acid of embodiment 153, wherein the RNAse comprises one or more of the extracellular RNAses selected from the group consisting of hRNAse1, hRNAse2, hRNAse3, hRNAse 4, hRNAse5, hRNAse6, hRNAse7, hRNAse8, hRNAse9, hRNAse10, hRNAse1l, hRNAse12, hRNAse13, bovine seminal RNAse, bovine milk RNAse, rodent RNAse, frog RNAse, RNAseT2, plant self-incompatibility RNAse, or bacterial RNAse.
156. The nucleic acid of any one of embodiments 116-155, wherein the nucleic acid has no or fewer than 10 base modifications.
157. The nucleic acid of any one of embodiments 116-156, wherein the nucleic acid has no or fewer than 10 backbone modifications.
158. The nucleic acid of any one of embodiments 116-157, wherein the nucleic acid has no or fewer than 10 sugar modifications.
159. The nucleic acid of any one of embodiments 116-158, wherein the nucleic acid is a deoxyribonucleic acid (DNA).
160. A ribonucleic acid (RNA) transcribed from the DNA of embodiment 159.
161. The RNA of embodiment 160, wherein the RNA is transcribed in vitro or in vivo.
162. The nucleic acid of any one of embodiments 116-158, wherein the nucleic acid is a ribonucleic acid (RNA).
163. The nucleic acid of any one of embodiments 160-162, wherein the RNA is a messenger RNA.
164. The nucleic acid of any one of embodiments 116-163, comprising a self-cleavage site.
165. The nucleic acid of any one of embodiments 116-164, comprising an internal ribosome entry site.
166. The nucleic acid of any one of embodiments 116-165, comprising a sequence encoding a peptide that induces ribosomal skipping during translation.
167. The nucleic acid of any one of embodiments 116-166, comprising a sequence encoding a peptide motif of DxExNPGP, where x is any amino acid.
168. The nucleic acid of any one of embodiments 116-167, comprising a sequence at least 80% identical to SEQ ID NO: 71.
169. The nucleic acid of any one of embodiments 116-168, comprising a sequence encoding a signal peptide.
170. The nucleic acid of embodiment 169, wherein the signal peptide is Gaussia luciferase, human albumin, human chymotrypsinogen, human interleukin-2, or human trypsinogen-2.
171. The nucleic acid of embodiment 169 or embodiment 170, wherein the signal peptide is at least 80% identical to any one of SEQ ID NOS: 107-112.
172. The nucleic acid of embodiment 169 or embodiment 170, wherein the signal peptide is at least 80% identical to SEQ ID NO: 107.
173. The nucleic acid of any one of embodiments 116-172, comprising a sequence encoding a cleavage site.
174. The nucleic acid of embodiment 173, wherein the sequence encoding the cleavage site is positioned between the 5′ UTR and the exogenous polynucleotide.
175. The nucleic acid of embodiment 173 or embodiment 174, wherein the cleavage site comprises an exopeptidase, endopeptidase and/or exopeptidase cleavage site.
176. The nucleic acid of embodiment 173 or embodiment 174, wherein the cleavage site is a proteasome cleavage site, a cysteine protease cleavage site, an aspartate protease cleavage site, a seine protease cleavage site, or a combination thereof.
177. The nucleic acid of any of embodiments 173-176, wherein the sequence encoding the cleavage site comprises a sequence at least 80% identical to any one of SEQ ID NOS: 73-82.
178. The nucleic acid of any of embodiments 173-176, wherein the sequence encoding the cleavage site comprises a sequence at least 80% identical to SEQ ID NO: 81.
179. The nucleic acid of any of embodiments 173-176, wherein the cleavage site comprises a sequence at least 80% identical to any one of SEQ ID NOS: 83-92.
180. The nucleic acid of any of embodiments 173-176, wherein the cleavage site comprises a sequence at least 80% identical to SEQ ID NO: 91.
181. The nucleic acid of any one of embodiments 116-180, wherein the exogenous polynucleotide encodes a pathogen-associated antigen.
182. The nucleic acid of embodiment 181, wherein the pathogen is a virus, bacteria, fungus, protozoa, or helminth.
183. The nucleic acid of embodiment 181 or embodiment 182, wherein the exogenous polynucleotide encodes a viral structural protein, a viral envelope protein, a viral capsid protein, or a viral nonstructural protein, or any combination thereof.
184. The nucleic acid of any one of embodiments 181-183, wherein the exogenous polynucleotide encodes an antigen from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picomaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); Iridoviridae (e.g., African swine fever virus); Hepatitis C virus; Norwalk virus; and Astrovirus; optionally wherein the exogenous polynucleotide comprises a sequence at least 80% identical to 10 or more nucleobases from the virus.
185. The nucleic acid of any one of embodiments 181-183, wherein the exogenous polynucleotide encodes an antigen from a bacteria selected from Helicobacter pylori, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii; optionally wherein the exogenous polynucleotide comprises a sequence at least 80% identical to 10 or more nucleobases from the bacteria.
186. The nucleic acid of any one of embodiments 181-183, wherein the exogenous polynucleotide encodes an antigen from a fungi selected from Cryptococcus neoformans, Histoplasma capsulatum, Coccidioides immitis, Blastomyces dermatitidis, Chlamydia trachomatis, and Candida albicans; optionally wherein the exogenous polynucleotide comprises a sequence at least 80% identical to 10 or more nucleobases from the fungi.
187. The nucleic acid of any one of embodiments 181-183, wherein the exogenous polynucleotide encodes an antigen from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major; optionally wherein the exogenous polynucleotide comprises a sequence at least 80% identical to 10 or more nucleobases from the protozoa.
188. The nucleic acid of any one of embodiments 116-187, wherein the exogenous polynucleotide comprises a sequence at least 80% identical to any one of SEQ ID NOS: 93-96.
189. The nucleic acid of any one of embodiments 116-188, wherein the exogenous polynucleotide encodes an antigen having a sequence at least 80% identical to any one of SEQ ID NOS: 97-100.
190. The nucleic acid of any one of embodiments 116-189, wherein the first exogenous polynucleotide and the polynucleotide encoding the MHC binding peptide are present on two separate nucleic acid strands.
191. The nucleic acid of any one of embodiments 116-189, wherein the first exogenous polynucleotide and the polynucleotide encoding the MHC binding peptide are connected.
192. The nucleic acid of any one of embodiments 116-191, wherein the MHC binding peptide is a MHC class I and/or a MHC class II peptide.
193. The nucleic acid of any one of embodiments 116-192, wherein the polynucleotide encoding the MHC binding peptide comprises a sequence at least 80% identical to any one of SEQ ID NOS: 113-135.
194. The nucleic acid of embodiment 193, wherein the polynucleotide encoding the MHC binding peptide comprises a sequence at least 80% identical to SEQ ID NO: 113.
195. The nucleic acid of any one of embodiments 116-194, wherein the MHC binding peptide comprises a sequence at least 80% identical to any one of SEQ ID NOS: 136-163.
196. The nucleic acid of embodiment 195, wherein the MHC binding peptide comprises a sequence at least 80% identical to SEQ ID NO: 136.
197. The nucleic acid of any one of embodiments 116-192, wherein the polynucleotide encoding the MHC binding peptide comprises a pathogen-associated sequence.
198. The nucleic acid of embodiment 197, wherein the pathogen is a virus, bacteria, fungus, protozoa, or helminth.
199. The nucleic acid of embodiment 197 or embodiment 198, wherein the polynucleotide encoding the MHC binding peptide is at least 80% identical to 10 or more nucleobases from a virus selected from Coronaviridae (e.g., severe acute respiratory syndrome coronaviruses such as SARS-CoV-1, SARS-CoV-2, Middle East respiratory syndrome coronavirus (MERS-CoV)); Retroviridae (e.g., human immunodeficiency viruses, such as HIV-1); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), herpes viruses, Epstein-Barr virus); Poxviridae (variola viruses, vaccinia viruses, pox viruses); and Iridoviridae (e.g., African swine fever virus); Hepatitis C virus; Norwalk virus; and Astrovirus.
200. The nucleic acid of embodiment 197 or embodiment 198, wherein the polynucleotide encoding the MHC binding peptide is at least 80% identical to 10 or more nucleobases from a bacteria selected from Helicobacter pylori, Borrelia burgdorferi, Legionella pneumophila, Mycobacteria sps (e.g. M. tuberculosis, M. avium, M. intracellulare, M. kansasii, M. gordonae, M. bovis), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus moniliformis, Treponema pallidum, Treponema pertenue, Leptospira sp, and Actinomyces israelii.
201. The nucleic acid of embodiment 197 or embodiment 198, wherein the polynucleotide encoding the MHC binding peptide is at least 80% identical to 10 or more nucleobases from a fungi selected from Cryptococcus neoformans, Histoplasma capsulatum, Coccidioides immitis, Blastomyces dermatitidis, Chlamydia trachomatis, and Candida albicans.
202. The nucleic acid of embodiment 197 or embodiment 198, wherein the polynucleotide encoding the MHC binding peptide is at least 80% identical to 10 or more nucleobases from a protozoa selected from Plasmodium spp. (e.g., Plasmodium falciparum), Trypanosomes (e.g., Trypanosoma cruzi), Toxoplasma gondii, Leishmania spp (e.g., Leishmania braziliensis), Leishmania infantum, Leishmania amazonensis, and Leishmania Major.
203. The nucleic acid of any one of embodiments 116-202, wherein the MHC binding peptide has a length of 7-20 peptides.
204. The nucleic acid of any one of embodiments 116-203, comprising two or more sequences encoding a MHC binding peptide.
205. A peptide translated from the nucleic acid of any one of embodiments 116-204.
206. A method of inducing an immune response in a subject, the method comprising administering to the subject the nucleic acid of any one of embodiments 116-204 or the peptide of embodiment 205.
207. The method of embodiment 206, wherein the nucleic acid is delivered via a lipid nanoparticle, virus-like particle, or naked.

### Certain Definitions

Percent (%) sequence identity with respect to a reference polypeptide or polynucleotide sequence is the percentage of amino acid or nucleotide residues in a candidate sequence that are identical with the amino acid or nucleotide residues in the reference polypeptide or polynucleotide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid or polynucleotide sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid or polynucleotide sequence comparisons, the % amino acid or polynucleotide sequence identity of a given sequence A to, with, or against a given sequence B (which can alternatively be phrased as a given sequence A that has or comprises a certain % sequence identity to, with, or against a given sequence B) is calculated as follows: 100 times the fraction X/Y, where X is the number of residues scored as identical matches by the sequence alignment program ALIGN-2 in that program's alignment of A and B, and where Y is the total number of residues in B. It will be appreciated that where the length of sequence A is not equal to the length of sequence B, the % sequence identity of A to B will not equal the % sequence identity of B to A. Unless specifically stated otherwise, all % sequence identity values used herein are obtained as described in the immediately preceding paragraph using the ALIGN-2 computer program.

In some embodiments, the term “about” means within 10% of the stated amount. For instance, a peptide comprising about 80% identity to a reference peptide may comprise 72% to 88% identity to the reference peptide sequence.

### Examples

The following examples are illustrative of the embodiments described herein and are not to be interpreted as limiting the scope of this disclosure. To the extent that specific materials are mentioned, it is merely for purposes of illustration and is not intended to be limiting. One skilled in the art may develop equivalent means or reactants without the exercise of inventive capacity and without departing from the scope of this disclosure.

### Example 1: Preparation of mRNA vaccines

In a first example, the mRNA construct as encoded by the DNA of Table 8 is prepared. The sequence comprises, from 5′ to 3′: a dengue virus 5′ UTR (underline), internal ribosome entry site/cleavage site P2A (squiggly underline), signal peptide for the antigen (italics), cathepsin cleavage site (bold), MHC binding peptide p25 (thick underline), cathepsin cleavage site (bold), MHC binding peptide p25 (thick underline), cathepsin cleavage site (bold), MHC binding peptide p25 (thick underline), cathepsin cleavage site (bold), spike antigen from COVID-19 (not underlined or italicized), and a dengue virus 3′ UTR (underline). RNA is in vitro transcribed using a T7 or SP6 promoter, nucleotides used are both natural (A, C, U, G) or synthetic (including Cap analogues and modified nucleotides such as pseudouridine and n-methyl-pseudouridine). RNA is purified by affinity columns or precipitation. Following the purification, the RNA is sequenced by reverse-transcriptase-PCR or analyzed by gel electrophoresis to confirm that the RNA is of the proper size and that no degradation of the RNA has occurred. The RNA is encapsulated in the chosen delivery method.

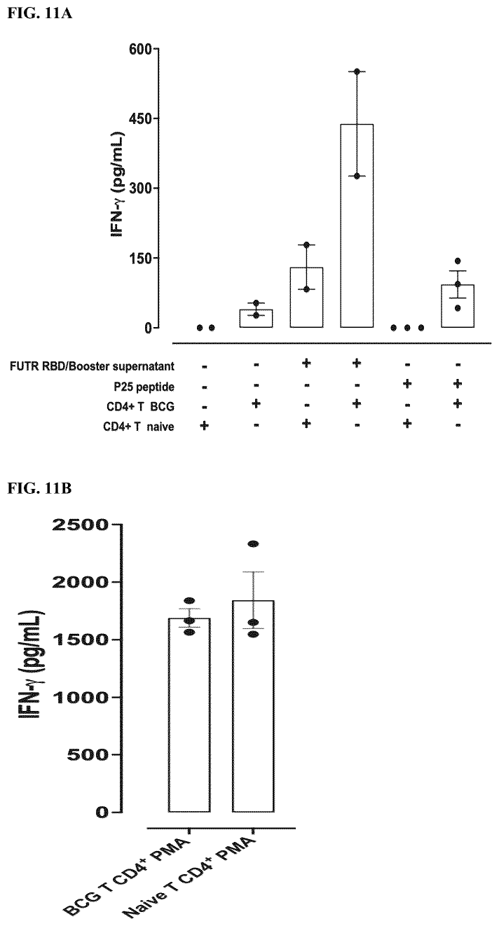

TABLE 8
Example DNA sequence encoding a mRNA vaccine construct

| SEQ ID NO | Sequence |
|---|---|
| 164 | 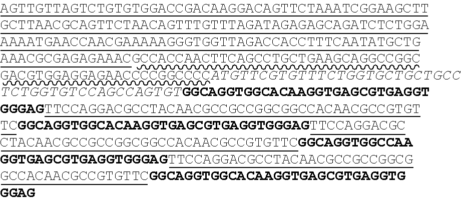 |
| | GTGAACCTGACCACCAGAACACAGCTGCCTCCAGCCTACACCAACAGCT |
| | TTACCAGAGGCGTGTACTACCCtGACAAGGTGTTCAGATCCAGtGTGCTG |
| | CACTCTACCCAGGACCTGTTCCTGCCTTTCTTCAGCAACGTGACCTGGTT |
| | CCACGCCATCCACGTGTCCGGCACCAATGGCACCAAGAGATTCGACAAC |
| | CCCGTGCTGCCCTTCAACGACGGGGTGTACTTTGCCAGCACCGAGAAGT |
| | CCAACATCATCAGAGGCTGGATCTTCGGCACCACACTGGACAGCAAGAC |
| | CCAGAGCCTGCTGATCGTGAACAACGCCACCAACGTGGTCATCAAAGTG |
| | TGCGAGTTCCAGTTCTGCAACGACCCCTTCCTGGGCGTCTACTACCACA |
| | AGAACAACAAGAGCTGGATGGAAAGCGAGTTCCGGGTGTACAGCAGCG |
| | CCAACAACTGCACCTTCGAGTACGTGTCCCAGCCTTTCCTGATGGACCT |
| | GGAAGGCAAGCAGGGCAACTTCAAGAACCTGCGCGAGTTCGTGTTCAA |
| | GAACATCGACGGCTACTTCAAGATCTACAGCAAGCACACCCCTATCAAC |
| | CTCGTGCGGGATCTGCCTCAGGGCTTCTCTGCTCTGGAACCCCTGGTGG |
| | ATCTGCCCATCGGCATCAACATCACCCGGTTTCAGACACTGCTGGCCCT |
| | GCACAGAAGCTACCTGACACCTGGCGATAGCAGCAGCGGATGGACAGC |
| | TGGTGCCGCCGCTTACTATGTGGGCTACCTGCAGCCTAGAACCTTCCTGC |
| | TGAAGTACAACGAGAACGGCACCATCACCGACGCCGTGGATTGTGCTCT |
| | GGCTCCTCTGAGCGAGACAAAGTGCACCCTGAAGTCCTTCACCGTGGAA |
| | AAGGGCATCTACCAGACCAGCAACTTCCGGGTGCAGCCCACCGAGTCCA |
| | TCGTGCGGTTCCCCAATATCACCAATCTGTGCCCCTTCGGCGAGGTGTTC |
| | AATGCCACCAGATTCGCCTCTGTGTACGCCTGGAACCGGAAGCGGATCA |
| | GCAATTGCGTGGCCGACTACTCCGTGCTGTACAACTCCGCCAGCTTCAG |
| | CACCTTCAAGTGCTACGGCGTGTCCCCTACCAAGCTGAACGACCTGTGC |
| | TTCACAAACGTGTACGCCGACAGCTTCGTGATCCGGGGAGATGAAGTGC |
| | GGCAGATTGCCCCTGGACAGACAGGCACTATCGCCGACTACAACTACAA |
| | GCTGCCCGACGACTTCACCGGCTGTGTGATTGCCTGGAACAGCAACAAC |
| | CTGGACTCCAAAGTCGGCGGCAACTACAATTACCTGTACCGGCTGTTCC |
| | GGAAGTCCAATCTGAAGCCCTTCGAGCGGGACATCTCCACCGAGATCTA |
| | TCAGGCCGGCGCACCCCTTGTAACGGCGTGAAAGGCTTCAACTGCTAC |
| | TTCCCACTGCAGTCCTACGGCTTTCAGCCCACGTATGGCGTGGGCTATCA |
| | GCCCTACAGAGTGGTGGTGCTGAGCTTCGAACTGCTGCATGCCCCTGCC |
| | ACAGTGTGCGGCCCTAAGAAAAGCACCAATCTCGTGAAGAACAAATGC |
| | GTGAACTTCAACTTCAACGGCCTGACCGGCACCGGCGTGCTGACAGAGA |
| | GCAACAAGAAGTTCCTGCCATTCCAGCAGTTTGGCCGGGACATCGCCGA |
| | TACCACAGACGCCGTTAGAGATCCCCAGACACTGGAAATCCTGGACATC |
| | ACCCCTTGCAGCTTCGGCGGAGTGTCTGTGATCACCCCTGGCACCAACA |
| | CCAGCAATCAGGTGGCAGTGCTGTACCAGGACGTGAACTGTACCGAAGT |
| | GCCCGTGGCCATTCACGCCGATCAGCTGACACCTACATGGCGGGTGTAC |
| | TCCACCGGCAGCAATGTGTTTCAGACCAGAGCCGGCTGTCTGATCGGAG |
| | CCGAGCACGTGAACAATAGCTACGAGTGCGACATCCCCATCGGCGCTGG |
| | CATCTGTGCCAGCTACCAGACACAGACAAACAGCCCCAGACGGGCCAG |
| | ATCTGTGGCCAGCCAGAGCATCATTGCCTACACAATGTCTCTGGGCGCC |
| | GAGAACAGCGTGGCCTACTCCAACAACTCTATCGCTATCCCCACCAACT |
| | TCACCATCAGCGTGACCACAGAGATCCTGCCTGTGTCCATGACCAAGAC |
| | CAGCGTGGACTGCACCATGTACATCTGCGGCGATTCCACCGAGTGCTCC |
| | AACCTGCTGCTGCAGTACGGCAGCTTCTGCACCCAGCTGAATAGAGCCC |
| | TGACAGGGATCGCCGTGGAACAGGACAAGAACACCCAAGAGGTGTTCG |
| | CCCAAGTGAAGCAGATCTACAAGACCCCTCCTATCAAGGACTTCGGCGG |
| | CTTCAATTTCAGCCAGATTCTGCCCGATCCTAGCAAGCCCAGCAAGCGG |
| | AGCTTCATCGAGGACCTGCTGTTCAACAAAGTGACACTGGCCGACGCCG |
| | GCTTCATCAAGCAGTATGGCGATTGTCTGGGCGACATTGCCGCCAGGGA |
| | TCTGATTTGCGCCCAGAAGTTTAACGGACTGACAGTGCTGCCTCCTCTGC |
| | TGACCGATGAGATGATCGCCCAGTACACATCTGCCCTGCTGGCCGGCAC |
| | AATCACAAGCGGCTGGACATTTGGAGCTGGCGCCGCTCTGCAGATCCCC |
| | TTTGCTATGCAGATGGCCTACCGGTTCAACGGCATCGGAGTGACCCAGA |
| | ATGTGCTGTACGAGAACCAGAAGCTGATCGCCAACCAGTTCAACAGCGC |
| | CATCGGCAAGATCCAGGACAGCCTGAGCAGCACAGCAAGCGCCCTGGG |
| | AAAGCTGCAGGACGTGGTCAACCAGAATGCCCAGGCACTGAACACCCT |
| | GGTCAAGCAGCTGTCCTCCAACTTCGGCGCCATCAGCTCTGTGCTGAAC |
| | GACATCCTGAGCAGACTGGACCCGCCGGAAGCCGAGGTGCAGATCGAC |
| | AGACTGATCACCGGAAGGCTGCAGTCCCTGCAGACCTACGTTACCCAGC |
| | AGCTGATCAGAGCCGCCGAGATTAGAGCCTCTGCCAATCTGGCCGCCAC |
| | CAAGATGTCTGAGTGTGTGCTGGGCCAGAGCAAGAGAGTGGACTTTTGC |
| | GGCAAGGGCTACCACCTGATGAGCTTCCCTCAGTCTGCCCCTCACGGCG |
| | TGGTGTTTCTGCACGTGACATACGTGCCCGCTCAAGAGAAGAATTTCAC |
| | CACCGCTCCAGCCATCTGCCACGACGGCAAAGCCCACTTTCCTAGAGAA |
| | GGCGTGTTCGTGTCCAACGGCACCCATTGGTTCGTGACCCAGCGGAACT |
| | TCTACGAGCCCCAGATCATCACCACCGACAACACCTTCGTGTCTGGCAA |
| | CTGCGACGTCGTGATCGGCATTGTGAACAATACCGTGTACGACCCTCTG |
| | CAGCCCGAGCTGGACAGCTTCAAAGAGGAACTGGATAAGTACTTTAAG |
| | AACCACACAAGCCCCGAtGTGGACCTGGGCGACATCAGCGGAATCAATG |
| | CCAGCGTCGTGAACATCCAGAAAGAGATCGACCGGCTGAACGAGGTGG |
| | CCAAGAATCTGAACGAGAGCCTGATCGACCTGCAAGAACTGGGGAAGT |
| | ACGAGCAGTACATCAAGTGGCCCTGGTACATCTGGCTGGGCTTTATCGC |
| | CGGACTGATTGCCATCGTGATGGTCACAATCATGCTGTGTTGCATGACC |
| | AGCTGCTGTAGCTGCCTGAAGGGCTGTTGTAGCTGTGGCAGCTGCTGCT |
| | AATAATTACCAACAACAAACACCAAAGGCTATTGAAGTCAGGCCACTTG |
| | TGCCACGGCTGGAGCAAACCGTGCTGCCTGTAGCTCCGCCAATAACGGG |
| | AGGCGTTATAATTCCCAGGGAGGCCATGCGCCACGGAAGCTGTACGCGT |
| | GGCATATTGGACTAGCGGTTAGAGGAGACCCCTCCCATCACCAACAAAA |
| | CGCAGCAAAAGGGGGCCCGAAGCCAGGAGGAAGCTGTACTCCTGGTGG |
| | AAGGACTAGAGGTTAGAGGAGACCCCCCCAACACAAAAACAGCATATT |
| | GACGCTGGGAAAGACCAGAGATCCTGCTGTCTCTACAACATCAATCCAG |
| | GCACAGAGCGCCGCAAGATGGATTGGTGTTGTTGATCCAACAGGTTCT |
| SEQ ID NOS: | DNA |
| 166-168 | AGTTGTTAGTCTGTGTGGACCGACAAGGACAGTTCTAAATCGGAAGCTT |
| FUTR-Renilla | GCTTAACGCAGTTCTAACAGTTTGTTTAGATAGAGAGCAGATCTCTGGA |
| | AAAATGAACCAACGAAAAAGGGTGGTTAGACCACCTTTCAATATGCTG |
| | AAACGCGAGAGAAACACTTCGAAAGTTTATGATCCAGAACAAAGGAAA |
| | CGGATGATAACTGGTCCGCAGTGGTGGGCCAGATGTAAACAAATGAAT |
| | GTTCTTGATTCATTTATTAATTATTATGATTCAGAAAAACATGCAGAAAA |
| | TGCTGTTATTTTTTTACATGGTAACGCGGCCTCTTCTTATTTATGGCGAC |
| | ATGTTGTGCCACATATTGAGCCAGTAGCGCGGTGTATTATACCAGATCT |
| | TATTGGTATGGGCAAATCAGGCAAATCTGGTAATGGTTCTTATAGGTTA |
| | CTTGATCATTACAAATATCTTACTGCATGGTTTGAACTTCTTAATTTACC |
| | AAAGAAGATCATTTTTGTCGGCCATGATTGGGGTGCTTGTTTGGCATTTC |
| | ATTATAGCTATGAGCATCAAGATAAGATCAAAGCAATAGTTCACGCTGA |
| | AAGTGTAGTAGATGTGATTGAATCATGGGATGAATGGCCTGATATTGAA |
| | GAAGATATTGCGTTGATCAAATCTGAAGAAGGAGAAAAAATGGTTTTG |
| | GAGAATAACTTCTTCGTGGAAACCATGTTGCCATCAAAAATCATGAGAA |
| | AGTTAGAACCAGAAGAATTTGCAGCATATCTTGAACCATTCAAAGAGAA |
| | AGGTGAAGTTCGTCGTCCAACATTATCATGGCCTCGTGAAATCCCGTTA |
| | GTAAAAGGTGGTAAACCTGACGTTGTACAAATTGTTAGGAATTATAATG |
| | CTTATCTACGTGCAAGTGATGATTTACCAAAAATGTTTATTGAATCGGAT |
| | CCAGGATTCTTTTCCAATGCTATTGTTGAAGGCGCCAAGAAGTTTCCTAA |
| | TACTGAATTTGTCAAAGTAAAAGGTCTTCATTTTTCGCAAGAAGATGCA |
| | CCTGATGAAATGGGAAAATATATCAAATCGTTCGTTGAGCGAGTTCTCA |
| | AAAATGAACAATAATTACCAACAACAAACACCAAAGGCTATTGAAGTC |
| | AGGCCACTTGTGCCACGGCTGGAGCAAACCGTGCTGCCTGTAGCTCCGC |
| | CAATAACGGGAGGCGTTATAATTCCCAGGGAGGCCATGCGCCACGGAA |
| | GCTGTACGCGTGGCATATTGGACTAGCGGTTAGAGGAGACCCCTCCCAT |
| | CACCAACAAAACGCAGCAAAAGGGGGCCCGAAGCCAGGAGGAAGCTGT |
| | ACTCCTGGTGGAAGGACTAGAGGTTAGAGGAGACCCCCCCAACACAAA |
| | AACAGCATATTGACGCTGGGAAAGACCAGAGATCCTGCTGTCTCTACAA |
| | CATCAATCCAGGCACAGAGCGCCGCAAGATGGATTGGTGTTGTTGATCC |
| | AACAGGTTCT |
| | RNA |
| | AGUUGUUAGUCUGUGUGGACCGACAAGGACAGUUCUAAAUCGGAAGC |
| | UUGCUUAACGCAGUUCUAACAGUUUGUUUAGAUAGAGAGCAGAUCUC |
| | UGGAAAAAUGAACCAACGAAAAAGGGUGGUUAGACCACCUUUCAAUA |
| | UGCUGAAACGCGAGAGAAACACUUCGAAAGUUUAUGAUCCAGAACAA |
| | AGGAAACGGAUGAUAACUGGUCCGCAGUGGUGGGCCAGAUGUAAACA |
| | AAUGAAUGUUCUUGAUUCAUUUAUUAAUUAUUAUGAUUCAGAAAAAC |
| | AUGCAGAAAAUGCUGUUAUUUUUUUACAUGGUAACGCGGCCUCUUCU |
| | UAUUUAUGGCGACAUGUUGUGCCACAUAUUGAGCCAGUAGCGCGGUG |
| | UAUUAUACCAGAUCUUAUUGGUAUGGGCAAAUCAGGCAAAUCUGGUA |
| | AUGGUUCUUAUAGGUUACUUGAUCAUUACAAAUAUCUUACUGCAUGG |
| | UUUGAACUUCUUAAUUUACCAAAGAAGAUCAUUUUUGUCGGCCAUGA |
| | UUGGGGUGCUUGUUUGGCAUUUCAUUAUAGCUAUGAGCAUCAAGAUA |
| | AGAUCAAAGCAAUAGUUCACGCUGAAAGUGUAGUAGAUGUGAUUGAA |
| | UCAUGGGAUGAAUGGCCUGAUAUUGAAGAAGAUAUUGCGUUGAUCAA |
| | AUCUGAAGAAGGAGAAAAAAUGGUUUUGGAGAAUAACUUCUUCGUGG |
| | AAACCAUGUUGCCAUCAAAAAUCAUGAGAAAGUUAGAACCAGAAGAA |
| | UUUGCAGCAUAUCUUGAACCAUUCAAAGAGAAAGGUGAAGUUCGUCG |
| | UCCAACAUUAUCAUGGCCUCGUGAAAUCCCGUUAGUAAAAGGUGGUA |
| | AACCUGACGUUGUACAAAUUGUUAGGAAUUAUAAUGCUUAUCUACGU |
| | GCAAGUGAUGAUUUACCAAAAAUGUUUAUUGAAUCGGAUCCAGGAUU |
| | CUUUUCCAAUGCUAUUGUUGAAGGCGCCAAGAAGUUUCCUAAUACUG |
| | AAUUUGUCAAAGUAAAAGGUCUUCAUUUUUCGCAAGAAGAUGCACCU |
| | GAUGAAAUGGGAAAAUAUAUCAAAUCGUUCGUUGAGCGAGUUCUCAA |
| | AAAUGAACAAUAAUUACCAACAACAAACACCAAAGGCUAUUGAAGUC |
| | AGGCCACUUGUGCCACGGCUGGAGCAAACCGUGCUGCCUGUAGCUCC |
| | GCCAAUAACGGGAGGCGUUAUAAUUCCCAGGGAGGCCAUGCGCCACG |
| | GAAGCUGUACGCGUGGCAUAUUGGACUAGCGGUUAGAGGAGACCCCU |
| | CCCAUCACCAACAAAACGCAGCAAAAGGGGGCCCGAAGCCAGGAGGA |
| | AGCUGUACUCCUGGUGGAAGGACUAGAGGUUAGAGGAGACCCCCCCA |
| | ACACAAAAACAGCAUAUUGACGCUGGGAAAGACCAGAGAUCCUGCUG |
| | UCUCUACAACAUCAAUCCAGGCACAGAGCGCCGCAAGAUGGAUUGGU |
| | GUUGUUGAUCCAACAGGUUCU |
| | Protein (Renilla) |
| | MNQRKRVVRPPFNMLKRERNTSKVYDPEQRKRMITGPQWWARCKQMNV |
| | LDSFINYYDSEKHAENAVIFLHGNAASSYLWRHVVPHIEPVARCIIPDLIGM |
| | GKSGKSGNGSYRLLDHYKYLTAWFELLNLPKKIIFVGHDWGACLAFHYSY |
| | EHQDKIKAIVHAESVVDVIESWDEWPDIEEDIALIKSEEGEKMVLENNFFVE |
| | TMLPSKIMRKLEPEEFAAYLEPFKEKGEVRRPTLSWPREIPLVKGGKPDVVQ |
| | IVRNYNAYLRASDDLPKMFIESDPGFFSNAIVEGAKKFPNTEFVKVKGLHFS |
| | QEDAPDEMGKYIKSFVERVLKNEQ |
| SEQ ID NO: | DNA |
| 169-171 | AGTTGTTAGTCTGTGTGGACCGACAAGGACAGTTCTAAATCGGAAGCTT |
| FUTR- | GCTTAACGCAGTTCTAACAGTTTGTTTAGATAGAGAGCAGATCTCTGGA |
| Renilla/ | AAAATGAACCAACGAAAAAGGGTGGTTAGACCACCTTTCAATATGCTG |
| Booster | AAACGCGAGAGAAACCTCGAGACTTCGAAAGTTTATGATCCAGAACAA |
| | AGGAAACGGATGATAACTGGTCCGCAGTGGTGGGCCAGATGTAAACAA |
| | ATGAATGTTCTTGATTCATTTATTAATTATTATGATTCAGAAAAACATGC |
| | AGAAAATGCTGTTATTTTTTTACATGGTAACGCGGCCTCTTCTTATTTAT |
| | GGCGACATGTTGTGCCACATATTGAGCCAGTAGCGCGGTGTATTATACC |
| | AGATCTTATTGGTATGGGCAAATCAGGCAAATCTGGTAATGGTTCTTAT |
| | AGGTTACTTGATCATTACAAATATCTTACTGCATGGTTTGAACTTCTTAA |
| | TTTACCAAAGAAGATCATTTTTGTCGGCCATGATTGGGGTGCTTGTTTGG |
| | CATTTCATTATAGCTATGAGCATCAAGATAAGATCAAAGCAATAGTTCA |
| | CGCTGAAAGTGTAGTAGATGTGATTGAATCATGGGATGAATGGCCTGAT |
| | ATTGAAGAAGATATTGCGTTGATCAAATCTGAAGAAGGAGAAAAAATG |
| | GTTTTGGAGAATAACTTCTTCGTGGAAACCATGTTGCCATCAAAAATCA |
| | TGAGAAAGTTAGAACCAGAAGAATTTGCAGCATATCTTGAACCATTCAA |
| | AGAGAAAGGTGAAGTTCGTCGTCCAACATTATCATGGCCTCGTGAAATC |
| | CCGTTAGTAAAAGGTGGTAAACCTGACGTTGTACAAATTGTTAGGAATT |
| | ATAATGCTTATCTACGTGCAAGTGATGATTTACCAAAAATGTTTATTGA |
| | ATCGGATCCAGGATTCTTTTCCAATGCTATTGTTGAAGGCGCCAAGAAG |
| | TTTCCTAATACTGAATTTGTCAAAGTAAAAGGTCTTCATTTTTCGCAAGA |
| | AGATGCACCTGATGAAATGGGAAAATATATCAAATCGTTCGTTGAGCGA |
| | GTTCTCAAAAATGAACAAGCTAGCGGCGGCGGCGGCAGCGGCGGCGGC |
| | GGCAGCGGCGGCGGCGGCAGCGGCAGGTGGCACAAGGTGAGCGTGA |
| | GGTGGGAGTTCCAGGACGCCTACAACGCCGCCGGCGGCCACAACGCCG |
| | TGTTCGGCAGGTGGCACAAGGTGAGCGTGAGGTGGGAGTTCCAGGA |
| | CGCCTACAACGCCGCCGGCGGCCACAACGCCGTGTTCGGCAGGTGGCA |
| | CAAGGTGAGCGTGAGGTGGGAGTTCCAGGACGCCTACAACGCCGCCG |
| | GCGGCCACAACGCCGTGTTCGGCAGGTGGCACAAGGTGAGCGTGAG |
| | GTGGGAGTAATAATTACCAACAACAAACACCAAAGGCTATTGAAGTCA |
| | GGCCACTTGTGCCACGGCTGGAGCAAACCGTGCTGCCTGTAGCTCCGCC |
| | AATAACGGGAGGCGTTATAATTCCCAGGGAGGCCATGCGCCACGGAAG |
| | CTGTACGCGTGGCATATTGGACTAGCGGTTAGAGGAGACCCCTCCCATC |
| | ACCAACAAAACGCAGCAAAAGGGGGCCCGAAGCCAGGAGGAAGCTGT |
| | ACTCCTGGTGGAAGGACTAGAGGTTAGAGGAGACCCCCCCAACACAAA |
| | AACAGCATATTGACGCTGGGAAAGACCAGAGATCCTGCTGTCTCTACAA |
| | CATCAATCCAGGCACAGAGCGCCGCAAGATGGATTGGTGTTGTTGATCC |
| | AACAGGTTCT |
| | RNA |
| | AGUUGUUAGUCUGUGUGGACCGACAAGGACAGUUCUAAAUCGGAAGC |
| | UUGCUUAACGCAGUUCUAACAGUUUGUUUAGAUAGAGAGCAGAUCUC |
| | UGGAAAAAUGAACCAACGAAAAAGGGUGGUUAGACCACCUUUCAAUA |
| | UGCUGAAACGCGAGAGAAACCUCGAGACUUCGAAAGUUUAUGAUCCA |
| | GAACAAAGGAAACGGAUGAUAACUGGUCCGCAGUGGUGGGCCAGAUG |
| | UAAACAAAUGAAUGUUCUUGAUUCAUUUAUUAAUUAUUAUGAUUCAG |
| | AAAAACAUGCAGAAAAUGCUGUUAUUUUUUUACAUGGUAACGCGGCC |
| | UCUUCUUAUUUAUGGCGACAUGUUGUGCCACAUAUUGAGCCAGUAGC |
| | GCGGUGUAUUAUACCAGAUCUUAUUGGUAUGGGCAAAUCAGGCAAAU |
| | CUGGUAAUGGUUCUUAUAGGUUACUUGAUCAUUACAAAUAUCUUACU |
| | GCAUGGUUUGAACUUCUUAAUUUACCAAAGAAGAUCAUUUUUGUCGG |
| | CCAUGAUUGGGGUGCUUGUUUGGCAUUUCAUUAUAGCUAUGAGCAUC |
| | AAGAUAAGAUCAAAGCAAUAGUUCACGCUGAAAGUGUAGUAGAUGUG |
| | AUUGAAUCAUGGGAUGAAUGGCCUGAUAUUGAAGAAGAUAUUGCGUU |
| | GAUCAAAUCUGAAGAAGGAGAAAAAAUGGUUUUGGAGAAUAACUUCU |
| | UCGUGGAAACCAUGUUGCCAUCAAAAAUCAUGAGAAAGUUAGAACCA |
| | GAAGAAUUUGCAGCAUAUCUUGAACCAUUCAAAGAGAAAGGUGAAGU |
| | UCGUCGUCCAACAUUAUCAUGGCCUCGUGAAAUCCCGUUAGUAAAAG |
| | GUGGUAAACCUGACGUUGUACAAAUUGUUAGGAAUUAUAAUGCUUAU |
| | CUACGUGCAAGUGAUGAUUUACCAAAAAUGUUUAUUGAAUCGGAUCC |
| | AGGAUUCUUUUCCAAUGCUAUUGUUGAAGGCGCCAAGAAGUUUCCUA |
| | AUACUGAAUUUGUCAAAGUAAAAGGUCUUCAUUUUUCGCAAGAAGAU |
| | GCACCUGAUGAAAUGGGAAAAUAUAUCAAAUCGUUCGUUGAGCGAGU |
| | UCUCAAAAAUGAACAAGCUAGCGGCGGCGGCGGCAGCGGCGGCGGCG |
| | GCAGCGGCGGCGGCGGCAGCGGCAGGUGGCACAAGGUGAGCGUGAGG |
| | UGGGAGUUCCAGGACGCCUACAACGCCGCCGGCGGCCACAACGCCGUG |
| | UUCGGCAGGUGGCACAAGGUGAGCGUGAGGUGGGAGUUCCAGGACGC |
| | CUACAACGCCGCCGGCGGCCACAACGCCGUGUUCGGCAGGUGGCACAA |
| | GGUGAGCGUGAGGUGGGAGUUCCAGGACGCCUACAACGCCGCCGGCG |
| | GCCACAACGCCGUGUUCGGCAGGUGGCACAAGGUGAGCGUGAGGUGG |
| | GAGUAAUAAUUACCAACAACAAACACCAAAGGCUAUUGAAGUCAGGC |
| | CACUUGUGCCACGGCUGGAGCAAACCGUGCUGCCUGUAGCUCCGCCAA |
| | UAACGGGAGGCGUUAUAAUUCCCAGGGAGGCCAUGCGCCACGGAAGC |
| | UGUACGCGUGGCAUAUUGGACUAGCGGUUAGAGGAGACCCCUCCCAU |
| | CACCAACAAAACGCAGCAAAAGGGGGCCCGAAGCCAGGAGGAAGCUG |
| | UACUCCUGGUGGAAGGACUAGAGGUUAGAGGAGACCCCCCCAACACA |
| | AAAACAGCAUAUUGACGCUGGGAAAGACCAGAGAUCCUGCUGUCUCU |
| | ACAACAUCAAUCCAGGCACAGAGCGCCGCAAGAUGGAUUGGUGUUGU |
| | UGAUCCAACAGGUUCU |
| | Protein (Renilla + Boosters) |
| | MNQRKRVVRPPFNMLKRERNLETSKVYDPEQRKRMITGPQWWARCKQM |
| | NVLDSFINYYDSEKHAENAVIFLHGNAASSYLWRHVVPHIEPVARCIIPDLIG |
| | MGKSGKSGNGSYRLLDHYKYLTAWFELLNLPKKIIFVGHDWGACLAFHYS |
| | YEHQDKIKAIVHAESVVDVIESWDEWPDIEEDIALIKSEEGEKMVLENNFFV |
| | ETMLPSKIMRKLEPEEFAAYLEPFKEKGEVRRPTLSWPREIPLVKGGKPDVV |
| | QIVRNYNAYLRASDDLPKMFIESDPGFFSNAIVEGAKKFPNTEFVKVKGLHF |
| | SQEDAPDEMGKYIKSFVERVLKNEQASGGGGSGGGGSGGGGSGRWHKVS |
| | VRWEFQDAYNAAGGHNAVFGRWHKVSVRWEFQDAYNAAGGHNAVFGR |
| | WHKVSVRWEFQDAYNAAGGHNAVFGRWHKVSVRWE |
| SEQ ID NO: | DNA |
| 172-174 | AGTTGTTAGTCTGTGTGGACCGACAAGGACAGTTCTAAATCGGAAGCTT |
| FUTR-RBD- | GCTTAACGCAGTTCTAACAGTTTGTTTAGATAGAGAGCAGATCTCTGGA |
| Booster | AAAATGAACCAACGAAAAAGGGTGGTTAGACCACCTTTCAATATGCTG |
| | AAACGCGAGAGAAACCTCGAGATGTTCGTGTTTCTGGTGCTGCTGCCTCT |
| | GGTGTCCAGCCAGCGGGTGCAGCCCACCGAATCCATCGTGCGGTTCCCC |
| | AATATCACCAATCTGTGCCCCTTCGGCGAGGTGTTCAATGCCACCAGAT |
| | TCGCCTCTGTGTACGCCTGGAACCGGAAGCGGATCAGCAATTGCGTGGC |
| | CGACTACTCCGTGCTGTACAACTCCGCCAGCTTCAGCACCTTCAAGTGCT |
| | ACGGCGTGTCCCCTACCAAGCTGAACGACCTGTGCTTCACAAACGTGTA |
| | CGCCGACAGCTTCGTGATCCGGGGAGATGAAGTGCGGCAGATTGCCCCT |
| | GGACAGACAGGCAAGATCGCCGACTACAACTACAAGCTGCCCGACGAC |
| | TTCACCGGCTGTGTGATTGCCTGGAACAGAACAACCTGGACTCCAAAG |
| | TCGGCGGCAACTACAATTACCTGTACCGGCTGTTCCGGAAGTCCAATCT |
| | GAAGCCCTTCGAGCGGGACATCTCCACCGAGATCTATCAGGCCGGCAGC |
| | ACCCCTTGTAACGGCGTGGAAGGCTTCAACTGCTACTTCCCACTGCAGT |
| | CCTACGGCTTTCAGCCCACAAATGGCGTGGGCTATCAGCCCTACAGAGT |
| | GGTGGTGCTGAGCTTCGAACTGCTGCATGCCCCTGCCACAGTGTGCGGC |
| | CCTAAGAAAAGCACCAATCTCGTGAAGAACAAATGCGTGAACTTCGCTA |
| | GCGGCGGCGGCGGCAGCGGCGGCGGCGGCAGCGGCGGCGGCGGCAGC |
| | GGCAGGTGGCACAAGGTGAGCGTGAGGTGGGAGTTCCAGGACGCCT |
| | ACAACGCCGCCGGCGGCCACAACGCCGTGTTCGGCAGGTGGCACAAG |
| | GTGAGCGTGAGGTGGGAGTTCCAGGACGCCTACAACGCCGCCGGCGG |
| | CCACAACGCCGTGTTCGGCAGGTGGCACAAGGTGAGCGTGAGGTGG |
| | GAGTTCCAGGACGCCTACAACGCCGCCGGCGGCCACAACGCCGTGTTC |
| | GGCAGGTGGCACAAGGTGAGCGTGAGGTGGGAGTAATAATTACCAA |
| | CAACAAACACCAAAGGCTATTGAAGTCAGGCCACTTGTGCCACGGCTGG |
| | AGCAAACCGTGCTGCCTGTAGCTCCGCCAATAACGGGAGGCGTTATAAT |
| | TCCCAGGGAGGCCATGCGCCACGGAAGCTGTACGCGTGGCATATTGGAC |
| | TAGCGGTTAGAGGAGACCCCTCCCATCACCAACAAAACGCAGCAAAAG |
| | GGGGCCCGAAGCCAGGAGGAAGCTGTACTCCTGGTGGAAGGACTAGAG |
| | GTTAGAGGAGACCCCCCCAACCAAAAACAGCATATTGACGCTGGGAA |
| | AGACCAGAGATCCTGCTGTCTCTACAACATCAATCCAGGCACAGAGCGC |
| | CGCAAGATGGATTGGTGTTGTTGATCCAACAGGTTCT |
| | RNA |
| | AGUUGUUAGUCUGUGUGGACCGACAAGGACAGUUCUAAAUCGGAAGC |
| | UUGCUUAACGCAGUUCUAACAGUUUGUUUAGAUAGAGAGCAGAUCUC |
| | UGGAAAAAUGAACCAACGAAAAAGGGUGGUUAGACCACCUUUCAAUA |
| | UGCUGAAACGCGAGAGAAACCUCGAGAUGUUCGUGUUUCUGGUGCUG |
| | CUGCCUCUGGUGUCCAGCCAGCGGGUGCAGCCCACCGAAUCCAUCGUG |
| | CGGUUCCCCAAUAUCACCAAUCUGUGCCCCUUCGGCGAGGUGUUCAA |
| | UGCCACCAGAUUCGCCUCUGUGUACGCCUGGAACCGGAAGCGGAUCA |
| | GCAAUUGCGUGGCCGACUACUCCGUGCUGUACAACUCCGCCAGCUUCA |
| | GCACCUUCAAGUGCUACGGCGUGUCCCCUACCAAGCUGAACGACCUGU |
| | GCUUCACAAACGUGUACGCCGACAGCUUCGUGAUCCGGGGAGAUGAA |
| | GUGCGGCAGAUUGCCCCUGGACAGACAGGCAAGAUCGCCGACUACAA |
| | CUACAAGCUGCCCGACGACUUCACCGGCUGUGUGAUUGCCUGGAACA |
| | GCAACAACCUGGACUCCAAAGUCGGCGGCAACUACAAUUACCUGUAC |
| | CGGCUGUUCCGGAAGUCCAAUCUGAAGCCCUUCGAGCGGGACAUCUC |
| | CACCGAGAUCUAUCAGGCCGGCAGCACCCCUUGUAACGGCGUGGAAG |
| | GCUUCAACUGCUACUUCCCACUGCAGUCCUACGGCUUUCAGCCCACAA |
| | AUGGCGUGGGCUAUCAGCCCUACAGAGUGGUGGUGCUGAGCUUCGAA |
| | CUGCUGCAUGCCCCUGCCACAGUGUGCGGCCCUAAGAAAAGCACCAAU |
| | CUCGUGAAGAACAAAUGCGUGAACUUCGCUAGCGGCGGCGGCGGCAG |
| | CGGCGGCGGCGGCAGCGGCGGCGGCGGCAGCGGCAGGUGGCACAAGG |
| | UGAGCGUGAGGUGGGAGUUCCAGGACGCCUACAACGCCGCCGGCGGC |
| | CACAACGCCGUGUUCGGCAGGUGGCACAAGGUGAGCGUGAGGUGGGA |
| | GUUCCAGGACGCCUACAACGCCGCCGGCGGCCACAACGCCGUGUUCGG |
| | CAGGUGGCACAAGGUGAGCGUGAGGUGGGAGUUCCAGGACGCCUACA |
| | ACGCCGCCGGCGGCCACAACGCCGUGUUCGGCAGGUGGCACAAGGUG |
| | AGCGUGAGGUGGGAGUAAUAAUUACCAACAACAAACACCAAAGGCUA |
| | UUGAAGUCAGGCCACUUGUGCCACGGCUGGAGCAAACCGUGCUGCCU |
| | GUAGCUCCGCCAAUAACGGGAGGCGUUAUAAUUCCCAGGGAGGCCAU |
| | GCGCCACGGAAGCUGUACGCGUGGCAUAUUGGACUAGCGGUUAGAGG |
| | AGACCCCUCCCAUCACCAACAAAACGCAGCAAAAGGGGGCCCGAAGCC |
| | AGGAGGAAGCUGUACUCCUGGUGGAAGGACUAGAGGUUAGAGGAGAC |
| | CCCCCCAACACAAAAACAGCAUAUUGACGCUGGGAAAGACCAGAGAU |
| | CCUGCUGUCUCUACAACAUCAAUCCAGGCACAGAGCGCCGCAAGAUG |
| | GAUUGGUGUUGUUGAUCCAACAGGUUCU |
| | Protein (RBD + Boosters) |
| | MNQRKRVVRPPFNMLKRERNLEMFVFLVLLPLVSSQRVQPTESIVRFPNITN |
| | LCPFGEVFNATRFASVYAWNRKRISNCVADYSVLYNSASFSTFKCYGVSPT |
| | KLNDLCFTNVYADSFVIRGDEVRQIAPGQTGKIADYNYKLPDDFTGCVIAW |
| | NSNNLDSKVGGNYNYLYRLFRKSNLKPFERDISTEIYQAGSTPCNGVEGFN |
| | CYFPLQSYGFQPTNGVGYQPYRVVVLSFELLHAPATVCGPKKSTNLVKNK |
| | CVNFASGGGGSGGGGSGGGGSGRWHKVSVRWEFQDAYNAAGGHNAVFG |
| | RWHKVSVRWEFQDAYNAAGGHNAVFGRWHKVSVRWEFQDAYNAAGGH |
| | NAVFGRWHKVSVREW |
| SEQ ID NO: 175-177 Commercial UTRs-Renilla | 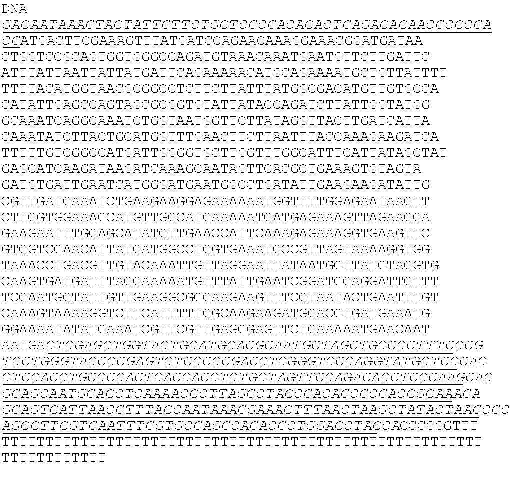 |
| | RNA |
| | GAGAAUAAACUAGUAUUCUUCUGGUCCCCACAGACUCAGAGAGAACC |
| | CGCCACCAUGACUUCGAAAGUUUAUGAUCCAGAACAAAGGAAACGGA |
| | UGAUAACUGGUCCGCAGUGGUGGGCCAGAUGUAAACAAAUGAAUGUU |
| | CUUGAUUCAUUUAUUAAUUAUUAUGAUUCAGAAAAACAUGCAGAAAA |
| | UGCUGUUAUUUUUUUACAUGGUAACGCGGCCUCUUCUUAUUUAUGGC |
| | GACAUGUUGUGCCACAUAUUGAGCCAGUAGCGCGGUGUAUUAUACCA |
| | GAUCUUAUUGGUAUGGGCAAAUCAGGCAAAUCUGGUAAUGGUUCUUA |
| | UAGGUUACUUGAUCAUUACAAAUAUCUUACUGCAUGGUUUGAACUUC |
| | UUAAUUUACCAAAGAAGAUCAUUUUUGUCGGCCAUGAUUGGGGUGCU |
| | UGUUUGGCAUUUCAUUAUAGCUAUGAGCAUCAAGAUAAGAUCAAAGC |
| | AAUAGUUCACGCUGAAAGUGUAGUAGAUGUGAUUGAAUCAUGGGAUG |
| | AAUGGCCUGAUAUUGAAGAAGAUAUUGCGUUGAUCAAAUCUGAAGAA |
| | GGAGAAAAAAUGGUUUUGGAGAAUAACUUCUUCGUGGAAACCAUGUU |
| | GCCAUCAAAAAUCAUGAGAAAGUUAGAACCAGAAGAAUUUGCAGCAU |
| | AUCUUGAACCAUUCAAAGAGAAAGGUGAAGUUCGUCGUCCAACAUUA |
| | UCAUGGCCUCGUGAAAUCCCGUUAGUAAAAGGUGGUAAACCUGACGU |
| | UGUACAAAUUGUUAGGAAUUAUAAUGCUUAUCUACGUGCAAGUGAUG |
| | AUUUACCAAAAAUGUUUAUUGAAUCGGAUCCAGGAUUCUUUUCCAAU |
| | GCUAUUGUUGAAGGCGCCAAGAAGUUUCCUAAUACUGAAUUUGUCAA |
| | AGUAAAAGGUCUUCAUUUUUCGCAAGAAGAUGCACCUGAUGAAAUGG |
| | GAAAAUAUAUCAAAUCGUUCGUUGAGCGAGUUCUCAAAAAUGAACAA |
| | UAAUGACUCGAGCUGGUACUGCAUGCACGCAAUGCUAGCUGCCCCUU |
| | UCCCGUCCUGGGUACCCCGAGUCUCCCCCGACCUCGGGUCCCAGGUAU |
| | GCUCCCACCUCCACCUGCCCCACUCACCACCUCUGCUAGUUCCAGACA |
| | CCUCCCAAGCACGCAGCAAUGCAGCUCAAAACGCUUAGCCUAGCCACA |
| | CCCCCACGGGAAACAGCAGUGAUUAACCUUUAGCAAUAAACGAAAGU |
| | UUAACUAAGCUAUACUAACCCCAGGGUUGGUCAAUUUCGUGCCAGCC |
| | ACACCCUGGAGCUAGCACCCGGGUUUUUUUUUUUUUUUUUUUUUUUU |
| | UUUUUUUUUUUUUUUUUUUUUUUUUUUUUUUUUUUUUUUUUUUUUU |
| | AUUU |
| | Protein (Renilla) |
| | MTSKVYDPEQRKRMITGPQWWARCKQMNVLDSFINYYDSEKHAENAVIFL |
| | HGNAASSYLWRHVVPHIEPVARCIIPDLIGMGKSGKSGNGSYRLLDHYKYL |
| | TAWFELLNLPKKIIFVGHDWGACLAFHYSYEHQDKIKAIVHAESVVDVIES |
| | WDEWPDIEEDIALIKSEEGEKMVLENNFFVETMLPSKIMRKLEPEEFAAYLE |
| | PFKEKGEVRRPTLSWPREIPLVKGGKPDVVQIVRNYNAYLRASDDLPKMFI |
| | ESDPGFFSNAIVEGAKKFPNTEFVKVKGLHFSQEDAPDEMGKYIKSFVERVL |
| | KNEQ |
| SEQ ID NO: 178-180 FUTR-SPIKE (FIG. 11) | 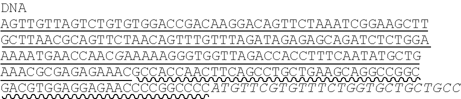 |
| | TCTGGTGTCCAGCCAGTGTCTCGAGGTGAACCTGACCACCAGAACACA |
| | GCTGCCTCCAGCCTACACCAACAGCTTTACCAGAGGCGTGTACTAC |
| | CCtGACAAGGTGTTCAGATCCAGtGTGCTGCACTCTACCCAGGACCT |
| | GTTCCTGCCTTTCTTCAGCAACGTGACCTGGTTCCACGCCATCCACG |
| | TGTCCGGCACCAATGGCACCAAGAGATTCGACAACCCCGTGCTGCC |
| | CTTCAACGACGGGGTGTACTTTGCCAGCACCGAGAAGTCCAACATC |
| | ATCAGAGGCTGGATCTTCGGCACCACACTGGACAGCAAGACCCAGA |
| | GCCTGCTGATCGTGAACAACGCCACCAACGTGGTCATCAAAGTGTG |
| | CGAGTTCCAGTTCTGCAACGACCCCTTCCTGGGCGTCTACTACCAC |
| | AAGAACAACAAGAGCTGGATGGAAAGCGAGTTCCGGGTGTACAGCA |
| | GCGCCAACAACTGCACCTTCGAGTACGTGTCCCAGCCTTTCCTGAT |
| | GGACCTGGAAGGCAAGCAGGGCAACTTCAAGAACCTGCGCGAGTTC |
| | GTGTTCAAGAACATCGACGGCTACTTCAAGATCTACAGCAAGCACA |
| | CCCCTATCAACCTCGTGCGGGATCTGCCTCAGGGCTTCTCTGCTCT |
| | GGAACCCCTGGTGGATCTGCCCATCGGCATCAACATCACCCGGTTT |
| | CAGACACTGCTGGCCCTGCACAGAAGCTACCTGACACCTGGCGATA |
| | GCAGCAGCGGATGGACAGCTGGTGCCGCCGCTTACTATGTGGGCTA |
| | CCTGCAGCCTAGAACCTTCCTGCTGAAGTACAACGAGAACGGCACC |
| | ATCACCGACGCCGTGGATTGTGCTCTGGCTCCTCTGAGCGAGACAA |
| | AGTGCACCCTGAAGTCCTTCACCGTGGAAAAGGGCATCTACCAGAC |
| | CAGCAACTTCCGGGTGCAGCCCACCGAGTCCATCGTGCGGTTCCCC |
| | AATATCACCAATCTGTGCCCCTTCGGCGAGGTGTTCAATGCCACCA |
| | GATTCGCCTCTGTGTACGCCTGGAACCGGAAGCGGATCAGCAATTG |
| | CGTGGCCGACTACTCCGTGCTGTACAACTCCGCCAGCTTCAGCACC |
| | TTCAAGTGCTACGGCGTGTCCCCTACCAAGCTGAACGACCTGTGCT |
| | TCACAAACGTGTACGCCGACAGCTTCGTGATCCGGGGAGATGAAGT |
| | GCGGCAGATTGCCCCTGGACAGACAGGCACTATCGCCGACTACAAC |
| | TACAAGCTGCCCGACGACTTCACCGGCTGTGTGATTGCCTGGAACA |
| | GCAACAACCTGGACTCCAAAGTCGGCGGCAACTACAATTACCTGTA |
| | CCGGCTGTTCCGGAAGTCCAATCTGAAGCCCTTCGAGCGGGACATC |
| | TCCACCGAGATCTATCAGGCCGGCAGCACCCCTTGTAACGGCGTGA |
| | AAGGCTTCAACTGCTACTTCCCACTGCAGTCCTACGGCTTTCAGCCC |
| | ACGTATGGCGTGGGCTATCAGCCCTACAGAGTGGTGGTGCTGAGCT |
| | TCGAACTGCTGCATGCCCCTGCCACAGTGTGCGGCCCTAAGAAAAG |
| | CACCAATCTCGTGAAGAACAAATGCGTGAACTTCAACTTCAACGGC |
| | CTGACCGGCACCGGCGTGCTGACAGAGAGCAACAAGAAGTTCCTGC |
| | CATTCCAGCAGTTTGGCCGGGACATCGCCGATACCACAGACGCCGT |
| | TAGAGATCCCCAGACACTGGAAATCCTGGACATCACCCCTTGCAGC |
| | TTCGGCGGAGTGTCTGTGATCACCCCTGGCACCAACACCAGCAATC |
| | AGGTGGCAGTGCTGTACCAGGACGTGAACTGTACCGAAGTGCCCGT |
| | GGCCATTCACGCCGATCAGCTGACACCTACATGGCGGGTGTACTCC |
| | ACCGGCAGCAATGTGTTTCAGACCAGAGCCGGCTGTCTGATCGGAG |
| | CCGAGCACGTGAACAATAGCTACGAGTGCGACATCCCCATCGGCGC |
| | TGGCATCTGTGCCAGCTACCAGACACAGACAAACAGCCCCAGACGG |
| | GCCAGATCTGTGGCCAGCCAGAGCATCATTGCCTACACAATGTCTC |
| | TGGGCGCCGAGAACAGCGTGGCCTACTCCAACAACTCTATCGCTAT |
| | CCCCACCAACTTCACCATCAGCGTGACCACAGAGATCCTGCCTGTG |
| | TCCATGACCAAGACCAGCGTGGACTGCACCATGTACATCTGCGGCG |
| | ATTCCACCGAGTGCTCCAACCTGCTGCTGCAGTACGGCAGCTTCTG |
| | CACCCAGCTGAATAGAGCCCTGACAGGGATCGCCGTGGAACAGGAC |
| | AAGAACACCCAAGAGGTGTTCGCCCAAGTGAAGCAGATCTACAAGA |
| | CCCCTCCTATCAAGGACTTCGGCGGCTTCAATTTCAGCCAGATTCTG |
| | CCCGATCCTAGCAAGCCCAGCAAGCGGAGCTTCATCGAGGACCTGC |
| | TGTTCAACAAAGTGACACTGGCCGACGCCGGCTTCATCAAGCAGTA |
| | TGGCGATTGTCTGGGCGACATTGCCGCCAGGGATCTGATTTGCGCC |
| | CAGAAGTTTAACGGACTGACAGTGCTGCCTCCTCTGCTGACCGATG |
| | AGATGATCGCCCAGTACACATCTGCCCTGCTGGCCGGCACAATCAC |
| | AAGCGGCTGGACATTTGGAGCTGGCGCCGCTCTGCAGATCCCCTTT |
| | GCTATGCAGATGGCCTACCGGTTCAACGGCATCGGAGTGACCCAGA |
| | ATGTGCTGTACGAGAACCAGAAGCTGATCGCCAACCAGTTCAACAG |
| | CGCCATCGGCAAGATCCAGGACAGCCTGAGCAGCACAGCAAGCGC |
| | CCTGGGAAAGCTGCAGGACGTGGTCAACCAGAATGCCCAGGCACTG |
| | AACACCCTGGTCAAGCAGCTGTCCTCCAACTTCGGCGCCATCAGCT |
| | CTGTGCTGAACGACATCCTGAGCAGACTGGACCCGCCGGAAGCCGA |
| | GGTGCAGATCGACAGACTGATCACCGGAAGGCTGCAGTCCCTGCAG |
| | ACCTACGTTACCCAGCAGCTGATCAGAGCCGCCGAGATTAGAGCCT |
| | CTGCCAATCTGGCCGCCACCAAGATGTCTGAGTGTGTGCTGGGCCA |
| | GAGCAAGAGAGTGGACTTTTGCGGCAAGGGCTACCACCTGATGAGC |
| | TTCCCTCAGTCTGCCCCTCACGGCGTGGTGTTTCTGCACGTGACAT |
| | ACGTGCCCGCTCAAGAGAAGAATTTCACCACCGCTCCAGCCATCTG |
| | CCACGACGGCAAAGCCCACTTTCCTAGAGAAGGCGTGTTCGTGTCC |
| | AACGGCACCCATTGGTTCGTGACCCAGCGGAACTTCTACGAGCCCC |
| | AGATCATCACCACCGACAACACCTTCGTGTCTGGCAACTGCGACGT |
| | CGTGATCGGCATTGTGAACAATACCGTGTACGACCCTCTGCAGCCC |
| | GAGCTGGACAGCTTCAAAGAGGAACTGGATAAGTACTTTAAGAACC |
| | ACACAAGCCCCGAtGTGGACCTGGGCGACATCAGCGGAATCAATGC |
| | CAGCGTCGTGAACATCCAGAAAGAGATCGACCGGCTGAACGAGGTG |
| | GCCAAGAATCTGAACGAGAGCCTGATCGACCTGCAAGAACTGGGGA |
| | AGTACGAGCAGTACATCAAGTGGCCCTGGTACATCTGGCTGGGCTT |
| | TATCGCCGGACTGATTGCCATCGTGATGGTCACAATCATGCTGTGT |
| | TGCATGACCAGCTGCTGTAGCTGCCTGAAGGGCTGTTGTAGCTGTG |
| | GCAGCTGCTGCTAATAAGCTAGCTTACCAACAACAAACACCAAAGGCT |
| | ATTGAAGTCAGGCCACTTGTGCCACGGCTGGAGCAAACCGTGCTGCCTG |
| | TAGCTCCGCCAATAACGGGAGGCGTTATAATTCCCAGGGAGGCCATGCG |
| | CCACGGAAGCTGTACGCGTGGCATATTGGACTAGCGGTTAGAGGAGAC |
| | CCCTCCCATCACCAACAAAACGCAGCAAAAGGGGGCCCGAAGCCAGGA |
| | GGAAGCTGTACTCCTGGTGGAAGGACTAGAGGTTAGAGGAGACCCCCC |
| | CAACACAAAAACAGCATATTGACGCTGGGAAAGACCAGAGATCCTGCT |
| | GTCTCTACAACATCAATCCAGGCACAGAGCGCCGCAAGATGGATTGGTG |
| | TTGTTGATCCAACAGGTTCT |
| | RNA |
| | AGUUGUUAGUCUGUGUGGACCGACAAGGACAGUUCUAAAUCGGAAGC |
| | UUGCUUAACGCAGUUCUAACAGUUUGUUUAGAUAGAGAGCAGAUCUC |
| | UGGAAAAAUGAACCAACGAAAAAGGGUGGUUAGACCACCUUUCAAUA |
| | UGCUGAAACGCGAGAGAAACGCCACCAACUUCAGCCUGCUGAAGCAG |
| | GCCGGCGACGUGGAGGAGAACCCCGGCCCCAUGUUCGUGUUUCUGGU |
| | GCUGCUGCCUCUGGUGUCCAGCCAGUGUCUCGAGGUGAACCUGACCA |
| | CCAGAACACAGCUGCCUCCAGCCUACACCAACAGCUUUACCAGAGGCG |
| | UGUACUACCCuGACAAGGUGUUCAGAUCCAGuGUGCUGCACUCUACCC |
| | AGGACCUGUUCCUGCCUUUCUUCAGCAACGUGACCUGGUUCCACGCCA |
| | UCCACGUGUCCGGCACCAAUGGCACCAAGAGAUUCGACAACCCCGUGC |
| | UGCCCUUCAACGACGGGGUGUACUUUGCCAGCACCGAGAAGUCCAAC |
| | AUCAUCAGAGGCUGGAUCUUCGGCACCACACUGGACAGCAAGACCCA |
| | GAGCCUGCUGAUCGUGAACAACGCCACCAACGUGGUCAUCAAAGUGU |
| | GCGAGUUCCAGUUCUGCAACGACCCCUUCCUGGGCGUCUACUACCACA |
| | AGAACAACAAGAGCUGGAUGGAAAGCGAGUUCCGGGUGUACAGCAGC |
| | GCCAACAACUGCACCUUCGAGUACGUGUCCCAGCCUUUCCUGAUGGAC |
| | CUGGAAGGCAAGCAGGGCAACUUCAAGAACCUGCGCGAGUUCGUGUU |
| | CAAGAACAUCGACGGCUACUUCAAGAUCUACAGCAAGCACACCCCUA |
| | UCAACCUCGUGCGGGAUCUGCCUCAGGGCUUCUCUGCUCUGGAACCCC |
| | UGGUGGAUCUGCCCAUCGGCAUCAACAUCACCCGGUUUCAGACACUG |
| | CUGGCCCUGCACAGAAGCUACCUGACACCUGGCGAUAGCAGCAGCGG |
| | AUGGACAGCUGGUGCCGCCGCUUACUAUGUGGGCUACCUGCAGCCUA |
| | GAACCUUCCUGCUGAAGUACAACGAGAACGGCACCAUCACCGACGCCG |
| | UGGAUUGUGCUCUGGCUCCUCUGAGCGAGACAAAGUGCACCCUGAAG |
| | UCCUUCACCGUGGAAAAGGGCAUCUACCAGACCAGCAACUUCCGGGU |
| | GCAGCCCACCGAGUCCAUCGUGCGGUUCCCCAAUAUCACCAAUCUGUG |
| | CCCCUUCGGCGAGGUGUUCAAUGCCACCAGAUUCGCCUCUGUGUACGC |
| | CUGGAACCGGAAGCGGAUCAGCAAUUGCGUGGCCGACUACUCCGUC |
| | UGUACAACUCCGCCAGCUUCAGCACCUUCAAGUGCUACGGCGUGUCCC |
| | CUACCAAGCUGAACGACCUGUGCUUCACAAACGUGUACGCCGACAGC |
| | UUCGUGAUCCGGGGAGAUGAAGUGCGGCAGAUUGCCCCUGGACAGAC |
| | AGGCACUAUCGCCGACUACAACUACAAGCUGCCCGACGACUUCACCGG |
| | CUGUGUGAUUGCCUGGAACAGCAACAACCUGGACUCCAAAGUCGGCG |
| | GCAACUACAAUUACCUGUACCGGCUGUUCCGGAAGUCCAAUCUGAAG |
| | CCCUUCGAGCGGGACAUCUCCACCGAGAUCUAUCAGGCCGGCAGCACC |
| | CCUUGUAACGGCGUGAAAGGCUUCAACUGCUACUUCCCACUGCAGUC |
| | CUACGGCUUUCAGCCCACGUAUGGCGUGGGCUAUCAGCCCUACAGAG |
| | UGGUGGUGCUGAGCUUCGAACUGCUGCAUGCCCCUGCCACAGUGUGC |
| | GGCCCUAAGAAAAGCACCAAUCUCGUGAAGAACAAAUGCGUGAACUU |
| | CAACUUCAACGGCCUGACCGGCACCGGCGUGCUGACAGAGAGCAACA |
| | AGAAGUUCCUGCCAUUCCAGCAGUUUGGCCGGGACAUCGCCGAUACC |
| | ACAGACGCCGUUAGAGAUCCCCAGACACUGGAAAUCCUGGACAUCAC |
| | CCCUUGCAGCUUCGGCGGAGUGUCUGUGAUCACCCCUGGCACCAACAC |
| | CAGCAAUCAGGUGGCAGUGCUGUACCAGGACGUGAACUGUACCGAAG |
| | UGCCCGUGGCCAUUCACGCCGAUCAGCUGACACCUACAUGGCGGGUG |
| | UACUCCACCGGCAGCAAUGUGUUUCAGACCAGAGCCGGCUGUCUGAU |
| | CGGAGCCGAGCACGUGAACAAUAGCUACGAGUGCGACAUCCCCAUCG |
| | GCGCUGGCAUCUGUGCCAGCUACCAGACACAGACAAACAGCCCCAGAC |
| | GGGCCAGAUCUGUGGCCAGCCAGAGCAUCAUUGCCUACACAAUGUCU |
| | CUGGGCGCCGAGAACAGCGUGGCCUACUCCAACAACUCUAUCGCUAUC |
| | CCCACCAACUUCACCAUCAGCGUGACCACAGAGAUCCUGCCUGUGUCC |
| | AUGACCAAGACCAGCGUGGACUGCACCAUGUACAUCGUCGGCGAUUC |
| | CACCGAGUGCUCCAACCUGCUGCUGCAGUACGGCAGCUUCUGCACCCA |
| | GCUGAAUAGAGCCCUGACAGGGAUCGCCGUGGAACAGGACAAGAACA |
| | CCCAAGAGGUGUUCGCCCAAGUGAAGCAGAUCUACAAGACCCUCCU |
| | AUCAAGGACUUCGGCGGCUUCAAUUUCAGCCAGAUUCUGCCCGAUCC |
| | UAGCAAGCCCAGCAAGCGGAGCUUCAUCGAGGACCUGCUGUUCAACA |
| | AAGUGACACUGGCCGACGCCGGCUUCAUCAAGCAGUAUGGCGAUUGU |
| | CUGGGCGACAUUGCCGCCAGGGAUCUGAUUUGCGCCCAGAAGUUUAA |
| | CGGACUGACAGUGCUGCCUCCUCUGCUGACCGAUGAGAUGAUCGCCC |
| | AGUACACAUCUGCCCUGCUGGCCGGCACAAUCACAAGCGGCUGGACA |
| | UUUGGAGCUGGCGCCGCUCUGCAGAUCCCCUUUGCUAUGCAGAUGGC |
| | CUACCGGUUCAACGGCAUCGGAGUGACCCAGAAUGUGCUGUACGAGA |
| | ACCAGAAGCUGAUCGCCAACCAGUUCAACAGCGCCAUCGGCAAGAUCC |
| | AGGACAGCCUGAGCAGCACAGCAAGCGCCCUGGGAAAGCUGCAGGAC |
| | GUGGUCAACCAGAAUGCCCAGGCACUGAACACCCUGGUCAAGCAGCU |
| | GUCCUCCAACUUCGGCGCCAUCAGCUCUGUGCUGAACGACAUCCUGAG |
| | CAGACUGGACCCGCCGGAAGCCGAGGUGCAGAUCGACAGACUGAUCA |
| | CCGGAAGGCUGCAGUCCCUGCAGACCUACGUUACCCAGCAGCUGAUCA |
| | GAGCCGCCGAGAUUAGAGCCUCUGCCAAUCUGGCCGCCACCAAGAUG |
| | UCUGAGUGUGUGCUGGGCCAGAGCAAGAGAGUGGACUUUUGCGGCAA |
| | GGGCUACCACCUGAUGAGCUUCCCUCAGUCUGCCCCUCACGGCGUGGU |
| | GUUUCUGCACGUGACAUACGUGCCCGCUCAAGAGAAGAAUUUCACCA |
| | CCGCUCCAGCCAUCUGCCACGACGGCAAAGCCCACUUUCCUAGAGAAG |
| | GCGUGUUCGUGUCCAACGGCACCCAUUGGUUCGUGACCCAGCGGAAC |
| | UUCUACGAGCCCCAGAUCAUCACCACCGACAACACCUUCGUGUCUGGC |
| | AACUGCGACGUCGUGAUCGGCAUUGUGAACAAUACCGUGUACGACCC |
| | UCUGCAGCCCGAGCUGGACAGCUUCAAAGAGGAACUGGAUAAGUACU |
| | UUAAGAACCACACAAGCCCCGAuGUGGACCUGGGCGACAUCAGCGGAA |
| | UCAAUGCCAGCGUCGUGAACAUCCAGAAAGAGAUCGACCGGCUGAAC |
| | GAGGUGGCCAAGAAUCUGAACGAGAGCCUGAUCGACCUGCAAGAACU |
| | GGGGAAGUACGAGCAGUACAUCAAGUGGCCCUGGUACAUCUGGCUGG |
| | GCUUUAUCGCCGGACUGAUUGCCAUCGUGAUGGUCACAAUCAUGCUG |
| | UGUUGCAUGACCAGCUGCUGUAGCUGCCUGAAGGGCUGUUGUAGCUG |
| | UGGCAGCUGCUGCUAAUAAGCUAGCUUACCAACAACAAACACCAAAG |
| | GCUAUUGAAGUCAGGCCACUUGUGCCACGGCUGGAGCAAACCGUGCU |
| | GCCUGUAGCUCCGCCAAUAACGGGAGGCGUUAUAAUUCCCAGGGAGG |
| | CCAUGCGCCACGGAAGCUGUACGCGUGGCAUAUUGGACUAGCGGUUA |
| | GAGGAGACCCCUCCCAUCACCAACAAAACGCAGCAAAAGGGGGCCCGA |
| | AGCCAGGAGGAAGCUGUACUCCUGGUGGAAGGACUAGAGGUUAGAGG |
| | AGACCCCCCCAACACAAAAACAGCAUAUUGACGCUGGGAAAGACCAG |
| | AGAUCCUGCUGUCUCUACAACAUCAAUCCAGGCACAGAGCGCCGCAA |
| | GAUGGAUUGGUGUUGUUGAUCCAACAGGUUCU |
| | Protein (Spike) |
| | MNQRKRVVRPPFNMLKRERNATNFSLLKQAGDVEENPGPMFVFLVLLPLV |
| | SSQCLEVNLTTRTQLPPAYTNSFTRGVYYPDKVFRSSVLHSTQDLFLPFFSN |
| | VTWFHAIHVSGTNGTKRFDNPVLPFNDGVYFASTEKSNIIRGWIFGTTLDSK |
| | TQSLLIVNNATNVVIKVCEFQFCNDPFLGVYYHKNNKSWMESEFRVYSSAN |
| | NCTFEYVSQPFLMDLEGKQGNFKNLREFVFKNIDGYFKIYSKHTPINLVRDL |
| | PQGFSALEPLVDLPIGINITRFQTLLALHRSYLTPGDSSSGWTAGAAAYYVG |
| | YLQPRTFLLKYNENGTITDAVDCALAPLSETKCTLKSFTVEKGIYQTSNFRV |
| | QPTESIVRFPNITNLCPFGEVFNATRFASVYAWNRKRISNCVADYSVLYNSA |
| | SFSTFKCYGVSPTKLNDLCFTNVYADSFVIRGDEVRQIAPGQTGTIADYNYK |
| | LPDDFTGCVIAWNSNNLDSKVGGNYNYLYRLFRKSNLKPFERDISTEIYQA |
| | GSTPCNGVKGFNCYFPLQSYGFQPTYGVGYQPYRVVVLSFELLHAPATVCG |
| | PKKSTNLVKNKCVNFNFNGLTGTGVLTESNKKFLPFQQFGRDIADTTDAVR |
| | DPQTLEILDITPCSFGGVSVITPGTNTSNQVAVLYQDVNCTEVPVAIHADQL |
| | TPTWRVYSTGSNVFQTRAGCLIGAEHVNNSYECDIPIGAGICASYQTQTNSP |
| | RRARSVASQSIIAYTMSLGAENSVAYSNNSIAIPTNFTISVTTEILPVSMTKTS |
| | VDCTMYICGDSTECSNLLLQYGSFCTQLNRALTGIAVEQDKNTQEVFAQVK |
| | QIYKTPPIKDFGGFNFSQILPDPSKPSKRSFIEDLLFNKVTLADAGFIKQYGDC |
| | LGDIAARDLICAQKFNGLTVLPPLLTDEMIAQYTSALLAGTITSGWTFGAGA |
| | ALQIPFAMQMAYRFNGIGVTQNVLYENQKLIANQFNSAIGKIQDSLSSTASA |
| | LGKLQDVVNQNAQALNTLVKQLSSNFGAISSVLNDILSRLDPPEAEVQIDRL |
| | ITGRLQSLQTYVTQQLIRAAEIRASANLAATKMSECVLGQSKRVDFCGKGY |
| | HLMSFPQSAPHGVVFLHVTYVPAQEKNFTTAPAICHDGKAHFPREGVFVSN |
| | GTHWFVTQRNFYEPQIITTDNTFVSGNCDVVIGIVNNTVYDPLQPELDSFKE |
| | ELDKYFKNHTSPDVDLGDISGINASVVNIQKEIDRLNEVAKNLNESLIDLQE |
| | LGKYEQYIKWPWYIWLGFIAGLIAIVMVTIMLCCMTSCCSCLKGCCSCGSC |
| | C |

FUTR UTRs (underline); P2A squiggly underline); signal peptide for the antigen
(italics); cathepsin cleavage site (bold); MHC binding peptide p25 (thick underline);
Linker (dot-dash underline); Commercial UTRs (italics + underline); Renilla protein
(not underlined or italicized); RBD protein (double underline); Spike protein
(bold + underlined).

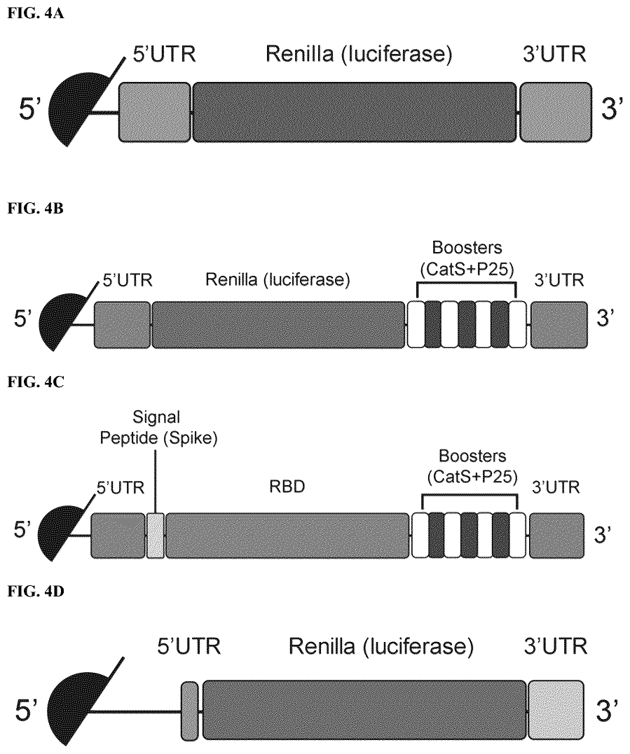

In a second example, mRNA constructs as shown in FIGS. 4A-4D and Table 8 were prepared. FUTR-Renilla includes 5 prime CAP, DV-4 UTRs, Renilla luciferase gene; FUTR Renilla-Boosters include 5 prime CAP, DV-4 UTRs, Renilla luciferase gene, Boosters (3× Cathepsin S cleavage site+mycobacterial MHC-II (p25) epitopes); and FUTR RBD-Boosters include 5 prime CAP, FUTR UTRs, signal peptide (Spike) SARS-CoV2 Receptor-Binding Domain-RBD gene, Boosters (3× Cathepsin S cleavage site+MHC-II (p25) epitopes). Commercial UTR construct includes 5 prime CAP, UTRs (see sequence in Table 8, SEQ ID NOS: 175-177), signal peptide, and Renilla luciferase gene. Poly-A tails were added in all constructs unless indicated in the figure.

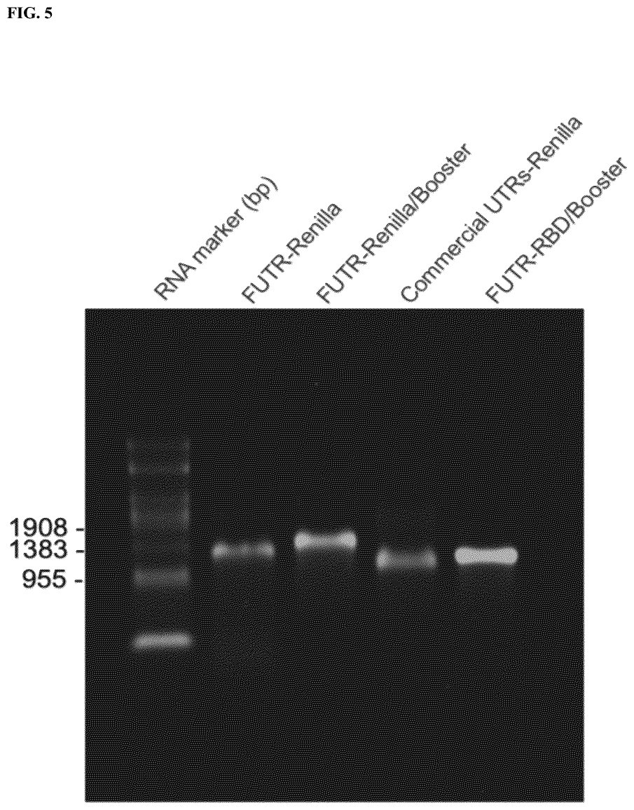

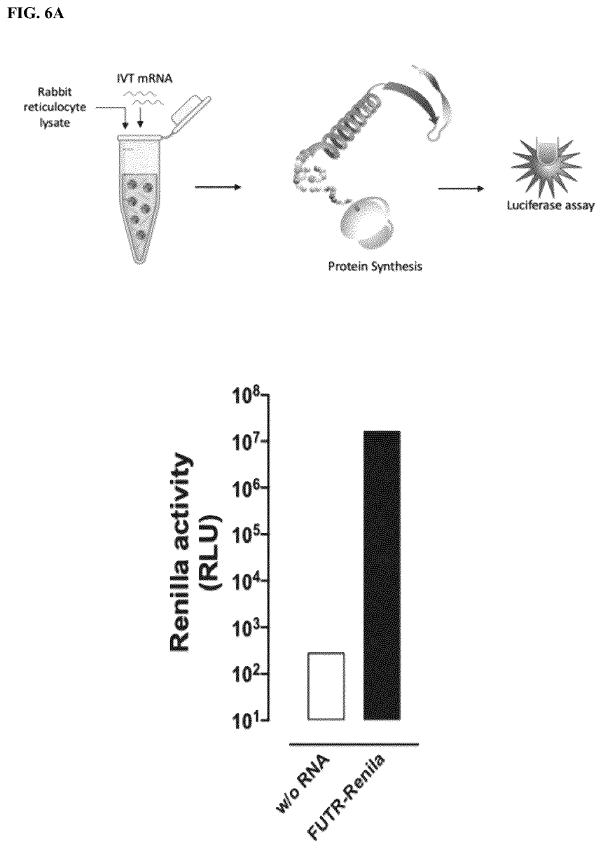

mRNA was in vitro transcribed using a T7XX promoter, nucleotides used are both natural (A, C, U, G) or synthetic (including Cap analogues and modified nucleotides such as pseudouridine and n-methyl-pseudouridine). RNA was purified by affinity columns or precipitation. Following the purification, the mRNA was analyzed by gel electrophoresis (FIG. 5). As shown in the figure, all of the example mRNA constructs were successfully transcribed. The results were reproducible.

### Example 2: Protein Expression in Cell Free and Mammalian Cell Systems

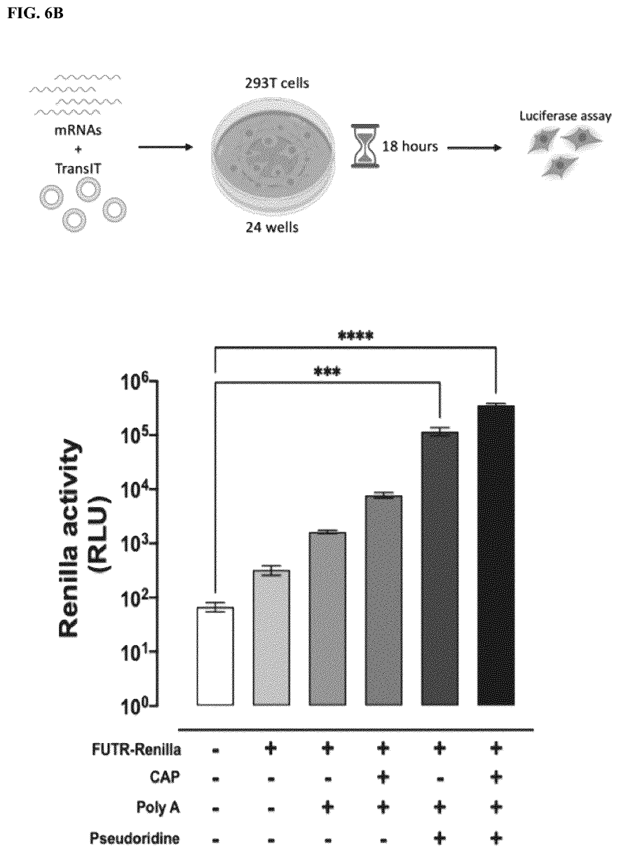

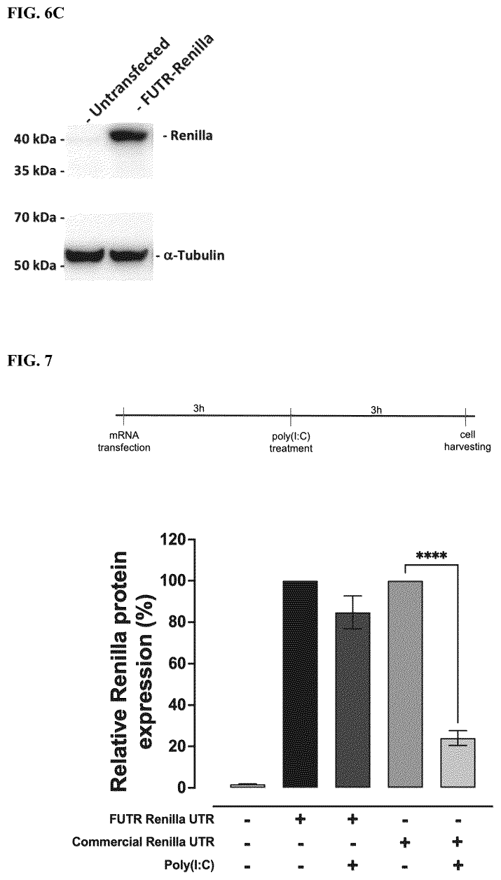

Cell free system: Renilla protein encoded in the in vitro transcribed mRNA construct shown in FIG. 4A (see also, Table 8) was translated in a rabbit reticulocyte lysate system (Promega). As shown in FIG. 6A, 2 μg of in vitro transcribed (IVT) FUTR-Renilla mRNA was incubated at 30° C. for 2 hours and quantified by measuring renilla activity (RLU).

Mammalian cell system: Renilla protein encoded in the in vitro transcribed mRNA construct shown in FIG. 4A (see also, Table 8) was translated in 293T cells. As shown in FIG. 6B, transfected with 0.5 μg of in vitro transcribed FUTR-Renilla mRNA was quantified by measuring renilla activity (RLU). The FUTR-Renilla mRNA construct was modified to include a 5′ cap (“CAP”), polyadenylation (“Poly A”), and/or substitution of uridine bases with pseudouridine (“Pseudouridine”). As shown in FIG. 6B, such modifications enhanced mRNA translation in mammalian cells by 1000 times over the unmodified FUTR-Renilla molecule.

Data in FIGS. 6A-6B are presented as mean±S.E.M. Statistical significance between groups was assessed by means of a one-way analysis of variance (ANOVA) followed by a post-hoc Dunnett test. The accepted level of significance for the tests was P<0.05. Data were plotted and analyzed using GraphPad Prism software.

The Renilla protein translated from the FUTR-Renilla mRNA was visualized by Western Blot (FIG. 6C). Supernatants from 293T cells transfected with FUTR-Renilla mRNA or untransfected 293T cells were used. 56.63 mg of protein from each sample was applied to an SDS-PAGE gel and transferred to an Nitrocellulose Transfer Membrane. Renilla protein was detected by Rabbit mAb anti-renilla [EPR17792]from Abcam (1:5000) and Tubulin protein was used as loader control, detected by Mouse mAb anti-a-tubulin [DM1A]from Millipore (1:5000). Respective anti-IgG antibody conjugated with HRP was used and SuperSignal™ West Pico PLUS Chemiluminescent Substrate ThermoFisher used for development.

### Example 3: Canonical and non-canonical antigen translation

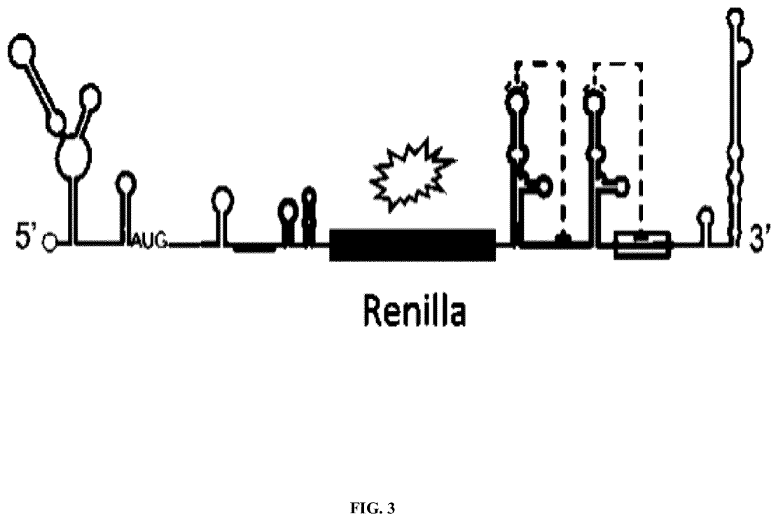

mRNA constructs are prepared from DNA comprising, from 5′ to 3′: a dengue virus 5′ UTR, a nucleic acid encoding a luminescent protein, and a dengue virus 3′ UTR (see, e.g., FIG. 3). mRNA is in vitro transcribed using a T7 or SP6 promoter, nucleotides used are both natural (A, C, U, G) or synthetic (including Cap analogues and modified nucleotides such as pseudouridine and n-methyl-pseudouridine). mRNA is generated with and without a Cap. Each mRNA is delivered to rabbit reticulocytes (RRL), and translation of the luminescent protein is measured in RLU to show that protein translation occurs in a Cap-1 (canonical) dependent or independent manner.

Protein translation following injection of exogenous mRNA encounters stress cellular microenvironments. In an example experiment, non-canonical translation mechanisms were tested for performance during cellular stress with both the FUTR-Renilla (FIG. 4A) and Commercial UTRs-Renilla (FIG. 4D). Human immunocompetent cells (A549) were transfected with 0.5 μg of each construct (FUTR-Renilla or Commercial UTRs-Renilla) using TransIT (Mirus), incubated for 3 hours and then stimulated with 10 μg/ml of poly(I:C) for 3 hours. Poly(I:C) is a double stranded RNA analogue that induces translational arrest via phosphorylation of eIF2a. This is a key mechanism of the immune system to control infections and other stressful situations. Renilla protein expression was evaluated by measuring renilla activity (RLU). Cells without poly(I:C) stimulation (100%) were used to calculate the impact of poly(I:C) transfection in renilla protein expression in A549 cells. FIG. 7 shows that FUTR-Renilla mRNA are significantly more resistant to stress than commercially available UTRs. Data are presented as mean±S.E.M. Statistical significance between groups was assessed by means of a one-way analysis of variance (ANOVA) followed by a post-hoc Tukey test. The accepted level of significance for the tests was P<0.05. Data were plotted and analyzed using GraphPad Prism software. The stress resistant mRNA may result in increased translation in stressed cellular conditions.

### Example 4: mRNA Stability: Comparative Resistance to RNAse

A first nucleic acid comprising an exogenous polynucleotide encoding an antigen, and a flavivirus 5′ UTR and/or flavivirus 3′ UTR is incubated with the RNase XRN-1. For example, the first nucleic acid is an mRNA transcribed from the construct of Example 1. Similarly, a second nucleic acid comprising the exogenous polynucleotide encoding the antigen, a non-flavivirus 5′ UTR, and a non-flavivirus 3′ UTR is incubated separately with the RNase XRN-1. For example, the second nucleic acid comprises a capped alpha globin 5′ and 3′ UTRs surrounding the stabilized form of SARS-CoV-2 spike protein. The second construct is polyadenylated and contains the same nucleotides, synthetic or natural of the first construct. The rate of degradation between the two nucleic acids is compared. Alternatively or in addition, depletion of XRN-1 from the cells is measured. The nucleic acid comprising the flavivirus 5′ UTR and/or flavivirus 3′ UTR is expected to have no or less degradation as compared to the nucleic acid lacking flavivirus UTRs.

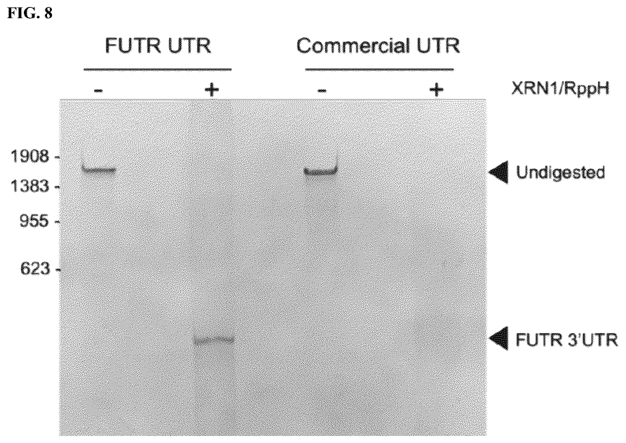

In an example experiment, the resistance of the FUTR-Renilla (FIG. 4A) and Commercial UTRs-Renilla (FIG. 4D) to the intracellular RNAase XRN-1 was tested. FUTR-Renilla mRNA and Commercial UTRs-Renilla mRNA (2 μg each) were incubated with 1.5 U of XRN1 (NEB, USA) and 15 U of RppH (NEB, USA) in 20 μl reaction mixture containing 1× NEB3 buffer and 1 u/μL RNAseout RNase Inhibitor (Invitrogen, USA). Incubation was performed for 150 min at 28° C. The reaction was stopped by adding 20 μL of Gel Loading Buffer II (Invitrogen, USA), heating for 10 min at 85° C. and placing it on ice. The entire volume was loaded into 10% polyacrylamide TBE-Urea gel and electrophoresis was performed for 180 min. 250 ng of undigested FUTR-Renilla and Commercial-UTRs mRNA was used as negative control. Gel was stained with SYBR-safe (Invitrogen, USA) and documented using dual LED blue/white light transilluminator (KASVI). As shown in FIG. 8, the FUTR-Renilla 3′ UTR remains intact, whereas the Commercial UTR was promptly degraded by XRN-1. The image is representative from three independent experiments that showed similar results.

### Example 5: Expression of Reporter Gene with Booster Fusion in Mammalian Cells

An mRNA construct was designed comprising a sequence encoding an immunodominant-based MHC-II peptide (FIGS. 4B, 4C). Without being bound by theory, this allows for bypassing the initial steps involved in the induction of immune responses, rescue TCR-specific memory CD4+ T cells and ultimately induce faster protective effects.

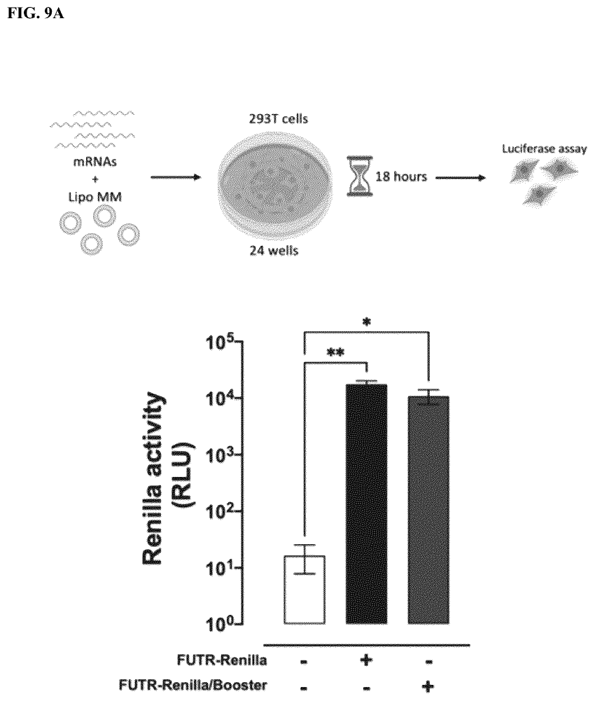

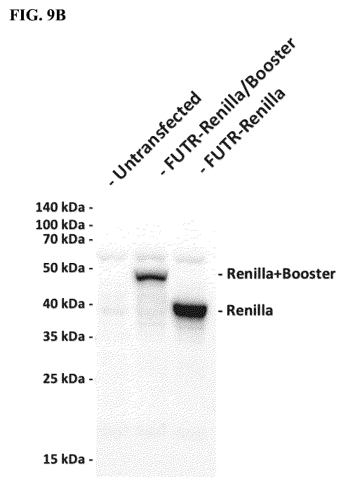

Briefly, renilla translation occurred in 293T cells transfected with 0.5 μg of in vitro transcribed FUTR-Renilla or FUTR-Renilla/Booster mRNA and quantified by measuring renilla activity (RLU) (FIG. 9A). Data are presented as mean±S.E.M. Statistical significance between groups was assessed by means of a one-way analysis of variance (ANOVA) followed by a post-hoc Dunnett test. The accepted level of significance for the tests was P<0.05. Data were plotted and analyzed using GraphPad Prism software. FIG. 9B shows the detection of Renilla and Renilla+Booster protein translated from FUTR-Renilla by Western Blot. Supernatant from HEK293T cells transfected with FUTR-Renilla/Booster, FUTR-Renilla or untransfected cells were used and 25 mL of each sample were applied to an SDS-PAGE gel and transferred to Nitrocellulose Transfer Membrane. Renilla protein was detected by Rabbit mAb anti-renilla from Abcam (1:5000). mAb anti-IgG rabbit HRP—Cell signaling was used as secondary antibody and SuperSignal™ West Pico PLUS Chemiluminescent Substrate ThermoFisher was used for development.

As shown in FIG. 9A, the addition of the boosters shows no major differences in mRNA translation, indicating that a functional polypeptide is also generated after incorporation of the boosters to the native mRNA renilla molecule. FIG. 9B, shows the expected increased molecular weight was observed in the FUTR-Renilla/Booster construct.

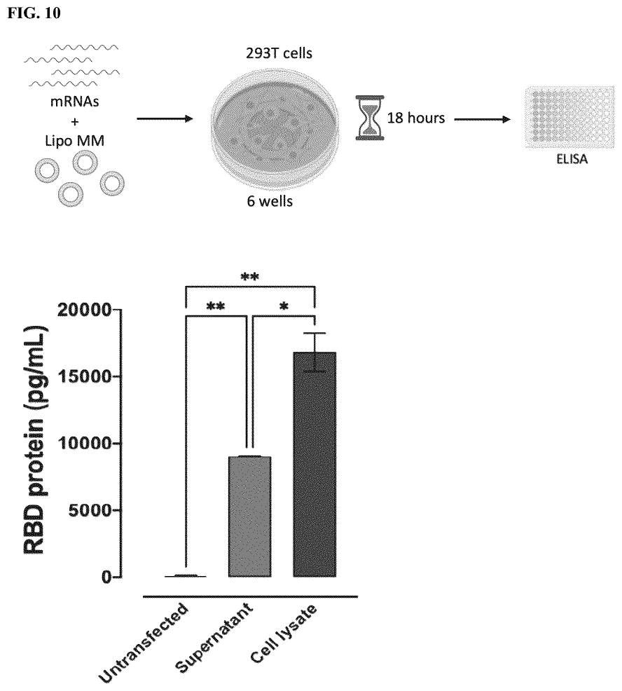

These results were confirmed with mRNA encoding a RBD from SARS-Cov-2 as an antigen and 3× BCG-derived p25 immunodominant MHC-II peptides as model boosters (FUTR-RBD/Booster) (FIG. 10). Briefly, 2.5 μg in vitro transcribed FUTR-RBD/Booster mRNA was transfected using Lipofectamine Messenger Max (Thermo Fisher) in 293T cells. SARS-CoV-2 Spike Detection ELISA Kit (Sino Biological) was used to measure RBD protein in cell culture supernatant or lysate. Wells were washed three times, then standard curve, cell lysate and supernatant of 293T transfected with FUTR RBD/Booster were added and incubated for 2 h. Next, wells were washed three times and incubated with detection antibody for 1 h. Wells were washed three times and substrate solution was provided for 6 min and reaction was stopped with an acid solution. Reading of O.D. was performed in a spectrophotometer at 450 nm. Results are means S.E.M. of data from triplicates. Experiment shown is representative of 3 performed. Statistical significance between groups was assessed by means of a One-way analysis of variance (ANOVA) followed by a post-hoc Tukey test. The accepted level of significance for the tests was P<0.05. Data were plotted and analyzed using GraphPad Prism software. The data indicate that the RBD-Booster protein is secreted by HEK293T cells.

### Example 6: FUTR-RBD/Booster Induces IFN-Gamma by Antigen-Primed CD4+ T Cells In Vitro

Example boosters were functionally assessed by in vitro recall assays with FUTR-RBD/Booster (FIG. 4C). In these assays, in vivo primed P25-specific CD4+ T cells generated following BCG immunization produce IFN-gamma only if these cells are activated by P25 peptide presented by antigen presenting cells in vitro. To test, either purified CD4+ T cells from control naïve or BCG-immunized C57BL/6 mice were co-cultured with antigen loaded bone marrow-derived dendritic cells (BMDCs). BMDCs were either loaded with supernatants from FUTR-RBD/Booster or mock-transfected HEK293T cells as produced in Example 5. As a control, DCs were treated with synthesized P25 peptides.

Briefly, supernatants from HEK293T cells as described in Example 5 were used to load bone marrow-derived dendritic cells (DCs) generated in vitro (described by Bafica A, Scanga CA et a]TLR9 regulates Th1 responses and cooperates with TLR2 in mediating optimal resistance to Mycobacterium tuberculosis. J Exp Med. 2005 Dec. 19; 202(12):1715-24. doi: 10.1084/jemn.20051782. PMID: 16365150; PMCID: PMC2212963). Supernatants-loaded DCs were then exposed to (1:2 ratio) CD4+ T cells purified from spleens of either naïve or BCG-immunized C57bl/6 mice for 72h. IFN-gamma was assayed by a commercial ELISA kit. As positive controls, cells were exposed to synthesized P25 peptide or b) PMA. The means±SEM of measurements from duplicate or triplicate wells are presented.

FIG. 11A shows significant increased IFN-gamma production by CD4+ T BCG when compared with CD4+ T naïve cells, suggesting DCs cleave FUTR-RBD/Booster at the Cathepsin S catalytic sites (FIG. 4, pink boxes) and properly present P25 peptides via MHC-II. Similar results were found when DCs were loaded with synthesized P25 peptides (FIG. 11A, last two groups). Of note, as a control, both naïve and BCG-immunized CD4+ T cell groups had the ability to produce high amounts of IFN-gamma when cells were treated with PMA, an unspecific stimulus (FIG. 11B), confirming that IFN-gamma produced by BCG CD4+ T cells are dependent upon P25 peptide presentation.

### Brief Summary of Examples 1-6

The data presented herein show at least that:

Example mRNA constructs (FIG. 4A-4C, Table 8) produce stable functional proteins.

Example UTRs described herein promote translation of exogenous polynucleotides during stress conditions.

The addition of molecular boosters to an mRNA composition does not impair protein function nor cellular secretion.

Example boosters described herein are correctly cleaved and presented to primed CD4+ T cells.

### Example 7: Antigen translation in vivo

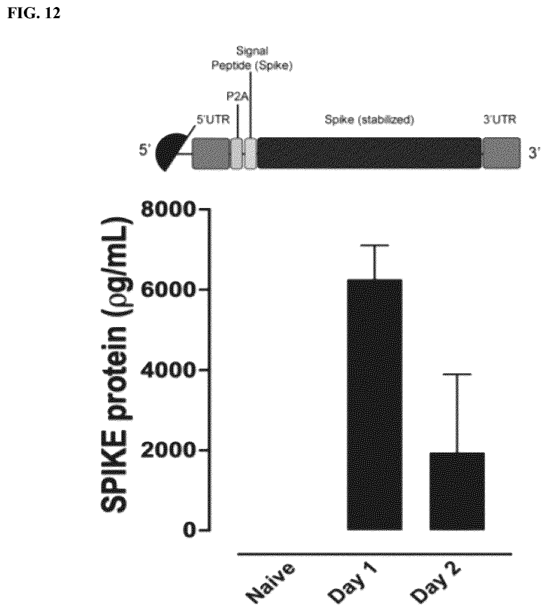

Groups of C57BL/6 mice were immunized with 20 μg of naked FUTR-SPIKE (without PolyA tail) (FIG. 12, top) complexed with 10 μg of protamine in Ringer's lactate solution by intramuscular route (i.m.). Uninjected naïve mice were used as controls. Spike protein levels were measured in serum (1:20) from days 1 and 2 by the SARS-CoV-2 Spike Detection ELISA Kit (Sino Biological) (FIG. 12, bottom). Results are means±S.E.M of data from 2 mice each group. Data were plotted using GraphPad Prism software. The results show that spike protein was detected in sera from the mice, and thus the mRNA composition comprising example DV UTRs is translated in vivo.

### Example 8: Induction of an Immune Response with a Vaccine Comprising a MHC Binding Peptide

Groups of mice are immunized with a mRNA vaccine disclosed herein, e.g., as described in Example 1 or 2, or a control vaccine, where the vaccine is constructed with or without a booster. At different time points, specific immune responses are evaluated in sera and spleen from immunized animals. qPCR and western blot are used to confirm the antigen, e.g., Spike gene, and its protein product, in sera and spleen from immunized animals. Specifically, immunoglobulin G (IgG), anti-Spike antibodies (ELISA and pseudotyped virus sera neutralization assays) as well as CD4+/CD+8 T cell activation (flow cytometry) are measured in immunized and control mice.

1. A nucleic acid composition comprising a 5′ untranslated region (5′ UTR) of a first flavivirus, a 3′ untranslated region (3′ UTR) of a second flavivirus, a first polynucleotide encoding a first peptide that is exogenous to the first flavivirus and/or the second flavivirus, and a polynucleotide encoding a major histocompatibility complex (MHC) binding peptide.

2. A method of expressing the first peptide in a cell, the method comprising delivering to the cell the nucleic acid composition of claim 1.

3. A method of inducing an immune response in a subject, the method comprising administering to the subject the nucleic acid composition of claim 1.

4. The nucleic acid composition of claim 1, wherein the 5′ UTR is a 5′ UTR of a dengue virus (DENV), West Nile virus (WNV), Japanese encephalitis virus (JEV), yellow fever virus (YFV), Zika virus (ZIKV), or tick-born encephalitis virus (TBEV); and the 3′ UTR is a 3′ UTR of a dengue virus (DENV), West Nile virus (WNV), Japanese encephalitis virus (JEV), yellow fever virus (YFV), Zika virus (ZIKV), or tick-born encephalitis virus (TBEV); and/or wherein the first flavivirus is the same as the second flavivirus; and/or wherein the 5′ UTR is at least 90% identical to a sequence of Table 1, and the 3′ UTR is at least 90% identical to a sequence of Table 2.

5. (canceled)

6. (canceled)

7. The nucleic acid composition of claim 1, wherein the MHC binding peptide comprises a sequence at least 90% identical to any one of SEQ ID NOS: 136-163, and/or a sequence at least 90% identical to 10 or more nucleobases of a pathogen.

8. (canceled)

9. (canceled)

10. (canceled)

11. (canceled)

12. (canceled)

13. The nucleic acid composition of claim 1, wherein the nucleic acid composition is more resistant to RNAse degradation as compared to a control composition comprising a non-flavivirus 5′ UTR, a non-flavivirus 3′ UTR, and the polynucleotide encoding the first peptide.

14. (canceled)

15. (canceled)

16. The nucleic acid composition of claim 1 wherein the nucleic acid composition does not comprise a sequence encoding 10 or more contiguous amino acids of a structural protein of the first flavivirus or the second flavivirus, and/or the nucleic acid composition does not comprise a sequence encoding 10 or more contiguous amino acids of a non-structural protein of the first flavivirus or the second flavivirus.

17. (canceled)

18. The nucleic acid composition of claim 1 wherein the first peptide is a pathogen-associated antigen.

19. A nucleic acid composition comprising a 5′ untranslated region (5′ UTR) of a first flavivirus, a 3′ untranslated region (3′ UTR) of a second flavivirus, and a polynucleotide encoding a peptide, wherein the polynucleotide encoding the peptide is exogenous to the first flavivirus and/or the second flavivirus.

20. A method of inducing an immune response in a subject, the method comprising administering to the subject the nucleic acid composition of claim 19.

22. The method of claim 20, wherein the peptide is expressed from the nucleic acid composition more than the peptide expressed from a control composition comprising a non-flavivirus 5′ UTR, a non-flavivirus 3′ UTR, and the polynucleotide encoding the peptide.

23. A method of expressing the peptide in a cell, the method comprising delivering to the cell the nucleic acid composition of claim 19.

24. The m nucleic acid composition of claim 19, wherein the nucleic acid composition is more resistant to RNAse degradation as compared to a control composition comprising a non-flavivirus 5′ UTR, a non-flavivirus 3′ UTR, and the polynucleotide encoding the peptide.

25. (canceled)

26. (canceled)

27. (canceled)

28. (canceled)

29. (canceled)

30. (canceled)

31. (canceled)

32. (canceled)

33. The nucleic acid composition of claim 19, wherein the peptide is a pathogen-associated antigen.

34. A nucleic acid composition comprising a polynucleotide encoding a first peptide and a polynucleotide encoding a major histocompatibility complex (MHC) binding peptide.

35. A method of inducing an immune response in a subject, the method comprising administering to the subject the nucleic acid composition of claim 34.

36. (canceled)

37. (canceled)

38. (canceled)

39. (canceled)

40. The nucleic acid composition of claim 34, wherein the MHC binding peptide comprises a sequence at least 90% identical to any one of SEQ ID NOS: 136-163 and/or a sequence at least 90% identical to 10 or more nucleobases of a pathogen.

41. (canceled)

42. The nucleic acid composition of claim 34, wherein the first peptide is a pathogen-associated antigen.

43. A method of expressing the first peptide in a cell, the method comprising delivering to the cell the nucleic acid composition of claim 34.


SEQUENCE LISTING (180 sequences)

SEQ ID NO: 1 - DNA, 94 bp, Dengue virus
  source 1..94: mol_type = genomic DNA
agttgttagtctacgtggaccgacaagaacagtttcgaatcggaagcttgcttaacgtagttctaacagttttttattagagagcagatctctg

SEQ ID NO: 2 - DNA, 96 bp, Dengue virus
  source 1..96: mol_type = genomic DNA
agttgttagtctacgtggaccgacaaagacagattctttgagggagctaagctcaacgtagttctaacagttttttaattagagagcagatctctg

SEQ ID NO: 3 - DNA, 94 bp, Dengue virus
  source 1..94: mol_type = genomic DNA
agttgtttatctacgtggaccgacaagaacagtttcgactcggaagcttgcttaacgtagtgctgacagttttttattagagagcagatctctg

SEQ ID NO: 4 - DNA, 101 bp, Dengue virus
  source 1..101: mol_type = genomic DNA
agttgttagtctgtgtggaccgacaaggacagttccaaatcggaagcttgcttaacacagttctaacagtttatttagatagagagcagatctctggaaaa

SEQ ID NO: 5 - DNA, 101 bp, Dengue virus
  source 1..101: mol_type = genomic DNA
agttgttagtctgtgtggaccgacaaggacagttctaaatcggaagcttgcttaacgcagttctaacagtttgtttagatagagagcagatctctggaaaa

SEQ ID NO: 6 - DNA, 96 bp, West Nile virus
  source 1..96: mol_type = genomic DNA
agtagttcgcctgtgtgagctgacaaacttagtagtgtttgtgaggattaacaacaattaacacagtgcgagctgtttcttagcacgaagatctcg

SEQ ID NO: 7 - DNA, 95 bp, Japanese encephalitis virus
  source 1..95: mol_type = genomic DNA
agaagtttatctgtgtgaacttcttggcttagtattgttgagaagaatcgagagattagtgcagtttaaacagttttttagaacggaagataacc

SEQ ID NO: 8 - DNA, 118 bp, Yellow fever virus
  source 1..118: mol_type = genomic DNA
agtaaatcctgtgtgctaattgaggtgcattggtctgcaaatcgagttgctaggcaataaacacatttggattaattttaatcgttcgttgagcgattagcagagaactgaccagaac

SEQ ID NO: 9 - DNA, 106 bp, Yellow fever virus
  source 1..106: mol_type = genomic DNA
gtgctaattgaggtgcattggtctgcaaatcgagttgctaggcaataaacacatttggattaattttaatcgttcgttgagcgattagcagagaactgaccagaac

SEQ ID NO: 10 - DNA, 95 bp, Zika virus
  source 1..95: mol_type = genomic DNA
gtgtgaatcagactgcgacagttcgagtttgaagcgaaagctagcaacagtatcaacaggttttattttggatttggaaacgagagtttctggtc

SEQ ID NO: 11 - DNA, 132 bp, Tick-borne encephalitis virus
  source 1..132: mol_type = genomic DNA
agattttcttgcacgtgcatgcgtttgcttcggatagcattagcagcggcaggttcggaagagacattgtctcgtttctactagtcgtgaacgtgttgagaaaaagacagcttaggagaacaagagctgggg

SEQ ID NO: 12 - DNA, 96 bp, Usutu virus
  source 1..96: mol_type = genomic DNA
agtcgttcgtctgcgtgagctctactacttagtattgtttttggaggatcgtgagattaacacagtgccggcagtttctttgagcgttgattttca

SEQ ID NO: 13 - DNA, 372 bp, Border disease virus
  source 1..372: mol_type = genomic DNA
gtatacgggagtagctcatgcccgtatacaaaattggatattccaaaactcgattgggttagggagccctcctagcgacggccgaaccgtgttaaccatacacgtagtaggactagcagacgggaggactagccatcgtggtgagatccctgagcagtctaaatcctgagtacaggatagtcgtcagtagttcaacgcaggcacggttctgccttgagatgctacgtggacgagggcatgcccaagacttgctttaatctcggcgggggtcgccgaggtgaaaacacctaacggtgttggggttacagcctgatagggtgctgcagaggcccacgaataggctagtataaaaatctctgctgtacatggcac

SEQ ID NO: 14 - DNA, 385 bp, Bovine viral diarrhea virus
  source 1..385: mol_type = genomic DNA
gtatacgagaattagaaaaggcactcgtatacgtattgggcaattaaaaataataattaggcctagggaacaaatccctctcagcgaaggccgaaaagaggctagccatgcccttagtaggactagcataatgaggggggtagcaacagtggtgagttcgttggatggcttaagccctgagtacagggtagtcgtcagtggttcgacgccttggaataaaggtctcgagatgccacgtggacgagggcatgcccaaagcacatcttaacctgagcgggggtcgcccaggtaaaagcagttttaaccgactgttacgaatacagcctgatagggtgctgcagaggcccactgtattgctactaaaaatctctgctgtacatggcac

SEQ ID NO: 15 - DNA, 104 bp, Bussuquara virus
  source 1..104: mol_type = genomic DNA
agtatttcttctgcgtgagaccattgcgacagttcgtaccggtgagttttgacttaacgcagtgagaaaagttttcgaggaaagacgagaagcgaattctctga

SEQ ID NO: 16 - DNA, 103 bp, Cell fusing agent virus
  source 1..103: mol_type = genomic DNA
acttcggcttagctacaccacagttttggttacgcttatattttcaaagcttaagttgtttttaatttttgccgagagaccgtgaggttgaacccggcaagga

SEQ ID NO: 17 - DNA, 373 bp, Classical swine fever virus
  source 1..373: mol_type = genomic DNA
gtatacgaggttagttcattctcgtatgcatgattggacaaatcaaaatttcaatttggttcagggcctccctccagcgacggccgaactgggctagccatgcccacagtaggactagcaaacggagggactagccgtagtggcgagctccctgggtggtctaagtcctgagtacaggacagtcgtcagtagttcgacgtgagcagaagcccacctcgagatgctatgtggacgagggcatgcccaagacacaccttaaccctagcgggggtcgctagggtgaaatcacaccacgtgatgggagtacgacctgatagggcgctgcagaggcccactattaggctagtataaaaatctctgctgtacatggcac

SEQ ID NO: 18 - DNA, 91 bp, Culex flavivirus
  source 1..91: mol_type = genomic DNA
agtttttaaaaacttcggcttggttacaccgcagattggttacacctacacaaggcttgagttgtttataatagtcgtttttctcgcagaa

SEQ ID NO: 19 - DNA, 119 bp, Entebbe bat virus
  source 1..119: mol_type = genomic DNA
agtaaattttgcgtgctagtcgcttggcgttagtccgtgaagtgagtttttggatacattgtaccagagattaacacgttgaaattatttctgaaaacagaaaatcagaatcagacgcg

SEQ ID NO: 20 - DNA, 382 bp, Pestivirus giraffe-1
  source 1..382: mol_type = genomic DNA
gtatacgagtttagctcaatcctcgtatacaatattgggcgtcaccaaatatagatttggcataggcaacaccccgatgcgaaggccgaaaagggctaaccatgcccttagtaggactagcaaaaaatcggggactagcccaggtggtgagcttcctggatgaccgaagccctgagtacagggcagtcgtcaacagttcaacacgcagaataggtttgcgtcttgatatgctgtgtggacgagggcatgcccacggtacatcttaacctatccgggggtcggataggcgaaagtccagtattggactgggagtacagcctgatagggtgttgcagagacccatctgataggctagtataaaaaactctgctgtacatggcac

SEQ ID NO: 21 - DNA, 341 bp, Hepatitis C virus
  source 1..341: mol_type = genomic DNA
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcacc

SEQ ID NO: 22 - DNA, 445 bp, Hepatitis GB virus B
  source 1..445: mol_type = genomic DNA
accacaaacactccagtttgttacactccgctaggaatgctcctggagcaccccccctagcagggcgtgggggatttcccctgcccgtctgcagaagggtggagccaaccaccttagtatgtaggcggcgggactcatgacgctcgcgtgatgacaagcgccaagcttgacttggatggccctgatgggcgttcatgggttcggtggtggtggcgctttaggcagcctccacgcccaccacctcccagatagagcggcggcactgtagggaagaccggggaccggtcactaccaaggacgcagacctctttttgagtatcacgcctccggaagtagttgggcaagcccacctatatgtgttgggatggttggggttagccatccataccgtactgcctgatagggtccttgcgaggggatctgggagtctcgtagaccgtagcac

SEQ ID NO: 23 - DNA, 458 bp, GB virus C/Hepatitis G virus
  source 1..458: mol_type = genomic DNA
acgtgggggagttgatcccccccccccggcactgggtgcaagccccagaaaccgacgcctatctaagtagacgcaatgactcggcgccgactcggcgaccggccaaaaggtggtggatgggtgatgacagggttggtaggtcgtaaatcccggtcaccttggtagccactataggtgggtcttaagagaaggttaagattcctcttgtgcctgcggcgagaccgcgcacggtccacaggtgttggccctaccggtgggaataagggcccgacgtcaggctcgtcgttaaaccgagcccgttacccacctgggcaaacgacgcccacgtacggtccacgtcgcccttcaatgtctctcttgaccaataggcgtagccggcgagttgacaaggaccagtgggggccgggggcttggagagggactccaagtcccgcccttcccggtgggccgggaaatgc

SEQ ID NO: 24 - DNA, 92 bp, Ilheus virus
  source 1..92: mol_type = genomic DNA
agaaattcacctgtgtgaatttcactaaccgttttagtggagagaacttttgtttaacacagtctgaatagttttttagcaagggatttccc

SEQ ID NO: 25 - DNA, 96 bp, Kamiti River virus
  source 1..96: mol_type = genomic DNA
agtttttgaaaacttctgtgaatgtttatatccttagtcggatcgagctaaattttaaatcaaaggagttgttcggaaaagtgaccttggttcgtt

SEQ ID NO: 26 - DNA, 83 bp, Kokobera virus
  source 1..83: mol_type = genomic DNA
agatgttcacctgtgtgaactaaccagacagatcgaagttaggtgattacataacacagtgtgaacaagttttttgaacagca

SEQ ID NO: 27 - DNA, 130 bp, Langat virus
  source 1..130: mol_type = genomic DNA
agattttcttgcgcgtgcatgcgtgtgcttcagacagcccaggcagcgactgtgattgtggatattctttctgcaagttttgtcgtgaacgtgttgagaaaaagacagcttaggagaacaagagctggga

SEQ ID NO: 28 - DNA, 129 bp, Louping ill virus
  source 1..129: mol_type = genomic DNA
agattttcttgcacgtgcgatagcttcggacagctttggcagcggcaggtttgaaagagacatttttttttctttcatcagccgtgaacgtgttgagaaaaagacagcttaggagaacaagagctgggg

SEQ ID NO: 29 - DNA, 109 bp, Modoc virus
  source 1..109: mol_type = genomic DNA
agttgatcctgccagcggtgggtcgctactgtttcgcgaaccagtcgttttgacagttggttgggatcaaatttgttctgtgcgcgtcacgccactttttgtggcggga

SEQ ID NO: 30 - DNA, 108 bp, Montana myotis leukoencephalitis virus
  source 1..108: mol_type = genomic DNA
agttggttttgccggctacaacgatcctccgtaggaagcgttggtgtcttggacattgccgagttgaaaccttggtttccggctggaaaccacgtcgctcttcgtcaa

SEQ ID NO: 31 - DNA, 95 bp, Murray Valley encephalitis virus
  source 1..95: mol_type = genomic DNA
agacgttcatctgcgtgagcttccgatctcagtattgtttggaaggatcattgattaacgcggtttgaacagttttttggagcttttgatttcaa

SEQ ID NO: 32 - DNA, 132 bp, Omsk hemorrhagic fever virus
  source 1..132: mol_type = genomic DNA
agattttcttgcacgtgcgtgcgcttgcttcagacagcaatagcagcggcagggttggtggaaggaattgcccgcatcagccagtcgtgaacgtgttgagaaaaagacagcttaggagaacaagagctgggg

SEQ ID NO: 33 - DNA, 111 bp, Powassan virus
  source 1..111: mol_type = genomic DNA
agattttcttgcacgtgtgtgcgggtgctttagtcagtgtccgcagcgttctgttgaacgtgagtgtgttgagaaaaagacagcttaggagaacaagagctgggagtggtt

SEQ ID NO: 34 - DNA, 116 bp, Sepik virus
  source 1..116: mol_type = genomic DNA
agtatattctgcgtgctaatcgttcaacgttagtccgtggagtgagcttctgttaagttgttaacacgtttgaataatttctactgaaagggtagagaaaaggagttttgcttctc

SEQ ID NO: 35 - DNA, 150 bp, Yokose virus
  source 1..150: mol_type = genomic DNA
agtaaattttgcgtgctagtcgctgagcgtcagaccgcaaagtgagtttttagtgatctaaagtgaggagttattcttactgtcatcaaacactacaaataaacacgttgaaattatttccggaagaacaactgtccggaatcaaagacg

SEQ ID NO: 36 - DNA, 161 bp, Dengue virus
  source 1..161: mol_type = genomic DNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaac

SEQ ID NO: 37 - DNA, 462 bp, Dengue virus
  source 1..462: mol_type = genomic DNA
gtcaacacactcatgaaataaaggaaaatagaagatcaaacaaagtgagaagtcaggccagattaagccatagtacggaaagagctatgctgcctgtgagccccgtccaaggacgtaaaatgaagtcaggccgaaagccacggattgagcaagccgtgctgcctgtggctccatcgtggggatgtaaaaacccgggaggctgcaacccatggaagctgtacgcatggggtagcagactagtggttagaggagacccctccctagacataacgcagcagcggggcccaacaccaggggaagctgtaccttggtggtaaggactagaggttagaggagaccccccgcacaacaacaaacagcatattgacgctgggagagaccagagatcctgctgtctctacagcatcattccaggcacagaacgccagaaaatggaatggtgctgttgaatcaacaggttct

SEQ ID NO: 38 - DNA, 453 bp, Dengue virus
  source 1..453: mol_type = genomic DNA
aaggcgaaactaacatgaaacaaggctgaaagtcaggtcggattaagccatagtacgggaaaaactatgctacctgtgagccccgtccaaggacgtaaaaagaagtcaggccatcacaaaaatgccacagcttgagcaaactgtgcagcctgtagctccacctgaggaggtgtaaaaaacccgggaggccacaaaccatggaagctgtacgcatggcgtagtggactagcggttagaggagacccctcccttacaaatcgcagcaacaacgggggcccaaggtgagatgaagctgtagtctcactggaaggactagaggttagaggagacccccccaaaacaaaaaacagcatattgacgctgggaaagaccagagatcctgctgtctcctcagcatcattccaggcacagaacgccagaaaatggaatggtgctgttgaatcaacaggttct

SEQ ID NO: 39 - DNA, 441 bp, Dengue virus
  source 1..441: mol_type = genomic DNA
acacaggaagtgaaaaagaggcaaactgtcaggccactttaagccacagtacggaagaagctgtgcagcctgtgagccccgtccaaggacgttaaaagaagaagtcaggcccaaaagccacggtttgagcaaaccgtgctgcctgtagctccgtcgtggggacgtaaaaacctgggaggctgcaaactgtggaagctgtacgcacggtgtagcagactagcggttagaggagacccctcccatgacacaacgcagcagcggggcccgagcactgagggaagctgtacctctttgcaaaggactagaggttagaggagaccccccgcaaacaaaaacagcatattgacgctgggagagaccagagatcctgctgtctcctcagcatcattccaggcacagaacgccagaaaatggaatggtgctgttgaatcaacaggttct

SEQ ID NO: 40 - DNA, 384 bp, Dengue virus
  source 1..384: mol_type = genomic DNA
ttaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 41 - DNA, 649 bp, West Nile virus
  source 1..649: mol_type = genomic DNA
ataacaaagctgtattgagtagttgtatagttgtagtgtttttagtaatttgaattatgattaattatttaggcttaagatagtattatagttagtttagtgtaaataggatttattgagaatggaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttagtgtgccactctgcggagggtgcagtctgcgatagtgccccaggtggactgggttaacaaaggcaaaacatcgccccacgcggccataaccctggctatggtgttaaccagggagaagggactagaggttagaggagaccccgcgtcaaaaagtgcacggcccaacttggctaaagctgtaagccaagggaaggactagaggttagaggagaccccgtgccaaaaacaccaaaagaaacagcatattgacacctgggatagactaggggatcttctgctctgcacaaccagccacacggcacagtgcgccgatataggtggctggtggtgctagaacacaggatct

SEQ ID NO: 42 - DNA, 583 bp, Japanese encephalitis virus
  source 1..583: mol_type = genomic DNA
tttgatttaaggtagaaaaataaaccatgtaaataatgtaaatgagaaaatgtatgtatatggagtcaggccagcaaaagctgccaccggatactgggtagacggtgctgcctgcgtctcagtcccaggaggactgggttaacaaatctgacaacagaaagtgagaaagccctcggaaccgtctcggaagtaggtccctgctcaccggaagttgaaagaccaacgtcaggccacaagtttgtgccactccgcttgggagtgcggcctgcgcagccccaggaggactgggttaccaaagccgttgaggcccccacggcccaagccttgtctaggatgcaatagacgaggtgtaaggactagaggttagaggagaccccgtggaaacaacaacatgcggcccaagccccctcgaagctgtagaggaggtggaaggactagaggttagaggagaccccgcatttgcatcaaacagcatattgacacctgggaatagactgggagatcttctgctctatctcaacatcagctactaggcacagagcgccgaagtatgtagctggtggtgaggaagaacacaggatct

SEQ ID NO: 43 - DNA, 508 bp, Yellow fever virus
  source 1..508: mol_type = genomic DNA
aacaccatctaacaggaataaccgggatacaaaccacgggtggagaaccggactccccacaacctgaaaccgggatataaaccacggctggagaaccggactccgcacttaaaatgaaacagaaaccgggataaaaactacggatggagaaccggactccacacattgagacagaagaagttgtcagcccagaaccccacacgagttttgccactgctaagctgtgaggcagtgcaggctgggacagccgacctccaggttgcgaaaaacctggtttctgggacctcccaccccagagtaaaaagaacggagcctccgctaccaccctcccacgtggtggtagaaagacggggtctagaggttagaggagaccctccagggaacaaatagtgggaccatattgacgccagggaaagaccggagtggttctctgcttttcctccagaggtctgtgagcacagtttgctcaagaataagcagacctttggatgacaaacacaaaaccact

SEQ ID NO: 44 - DNA, 480 bp, Yellow fever virus
  source 1..480: mol_type = genomic DNA
aacaccatctaataggaataaccgggatacaaaccacgggtggagaaccggactccccacaacttgaaaccgggatataaaccacggctggagaaccggactccgcacttaaaatgaaacagaaaccgggataaaaactacggatggagaaccggactccacacattgagacagaagaagttgtcagcccagaactccacacgagttttgccactgctaagctgtgaggcagtgcaggctgggacagccgacctccaggttgcgaaaaacctggtttctgggacctcccaccccagagtaaaaagaacggagcctccgctaccaccctcccacgtggtggtagaaagacggggtctagaggttagaggagaccctccagggaacaaatagtgggaccatattgacgccagggaaagaccggagtggttctctgcttttcctccaggggtctgtgagcacagtttgctcaagaataagcag

SEQ ID NO: 45 - DNA, 417 bp, Zika virus
  source 1..417: mol_type = genomic DNA
gcaccaatcttaatgttgtcaggcctgctagtcagccacagcttggggaaagctgtgcagcctgtgacccccccaggagaagctgggaaaccaagcctatagtcaggccgggaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccctggaaaacgcaaaacagcatattgacgctgggaaagaccagagactccatgagtttccaccacgctggccgccaggcacagatcgccgaatagcggcggccggtgtggggaaa

SEQ ID NO: 46 - DNA, 730 bp, Tick-borne encephalitis virus
  source 1..730: mol_type = genomic DNA
aaccaaagtgtgacagagcaaaacctggagggctcgtaaaatattgtccagaatcaaaaaccacagcaagcaaaacacagaaacagagctcggactggagagctcttaaaacaaaaaagccagaattgagctgaacctggagggctcattaaacattgtccagacaaaacaaaacagacatgatcacaagcaaaggaaagaggctgagcaaaggtcctgaatgaccagaccggtcttaccgcgggctgggaaggggggccagaatgcgaggccacagaccatggaatgctgcggcagcgcgcgagagcgacggggaaatggtcgcacccgacgcaccatccatgaagcaacacttcgtgagacccccccggccagtggagggggaagctggtcaggggtgaaagcacccccagagtgcactatggcaacacgccagtgagagtggcgacgggaaaatggtcgatcccgacgtagggcactctgtaaaactttgtgagaccccctgcatcatgacaaggcctaacatgatgcacgaaagggaggcccccggaagcgagcttccgggaggagggaagggagaaattggcagctcccttcaggatttttcctcctcctatactaaattccccctcaatagagggggggcggttcttgttctccctgagccaccatcacccagacacagatagtctgacaaggaggtgatgtgtgactcggaaaaacacccgct

SEQ ID NO: 47 - DNA, 665 bp, Usutu virus
  source 1..665: mol_type = genomic DNA
ataagtgtttagggttttgcaatttaattaaatatgcaatgtaatttagttgtaaatatttgattgtgtagctttatttagcattgttttaggatagtagaagttaaggttttatttagttattttatttaattgaatttgatagtcaggccagggcaacctgccaccggaagttgagtagacggtgctgcctgcgactcaaccccaggcggactgggttaacaaagctgaccgctgatgatgggaaagcccctcagaaccgtttcggagagggaccctgcctattggaagcgtccagcccgtgtcaggccgcaaagcgccacttcgccaaggagtgcagcctgtacggccccaggaggactgggttaccaaagccgaaaggcccccacggcccaagcgaacagacggtgatgcgaactgttcgtggaaggactagaggttagaggagaccccgtggaacttaggtgcggcccaagccgtttccgaagctgtaggaacggtggaaggactagaggttagaggagaccccgcatcataagcatcaaaaaaacagcatattgacacctgggaattagactaggagatcttctgctctattccaacatcaaccacaaggcacagagcgccgaaaattgtggctggtggggaactagaccacaggatct

SEQ ID NO: 48 - DNA, 273 bp, Border disease virus
  source 1..273: mol_type = genomic DNA
accatagctgagcatttcatgacaacacgccaagggccactaaattgtatatataactgtgtaaatatttacctatttatttactgttatttatttaatagagacagtgatatttatttaatagcttatctatttatttatttgatgggatgtagatggcaactaactacctcataggaccacactacactcatttttaaaactacagcactttagctggaagggaaaagcctgaagtccagagttggattaaggaaaaaccctaacagcccc

SEQ ID NO: 49 - DNA, 221 bp, Bovine viral diarrhea virus
  source 1..221: mol_type = genomic DNA
gacaaaatgtatatattgtaaataaattaatccatgtacatagtgtatataaatatagttgggaccgtccacctcaagaagacgacacgcccaacacgcacagctaaacagtagtcaagattatctacctcaagataacactacatttaatgcacacagcactttagctgtatgaggatacgcccgacgtctatagttggactagggaagacctctaacag

SEQ ID NO: 50 - DNA, 421 bp, Bussuquara virus
  source 1..421: mol_type = genomic DNA
gctaagataaaagagaaaaagagggtttgagtcaggccagaaatgccaccggataaaggtagacggtgctgcctgcaacctttctgcggaaggaataaccgcagtcaataaaaccaaaaagagggagttgagaaccctttgggccgcccaggcctgggattgaaccgttgatcccaggcgaagggactagaggttagaggagacccagcctttctcaccaacccaaggcccaaccttgctgaacctttaggcaggtaaaaggactagaggttagaggagaccccttggcaaaacagttaacgcaccaaaagaaacagcatattgacacctgggatagaccggagaatttgctgcctcgcaacacctcccacccggcacagaacgccgacatggtgggaggggtcgtaagacaccagattct

SEQ ID NO: 51 - DNA, 553 bp, Cell fusing agent virus
  source 1..553: mol_type = genomic DNA
acgaaatcgaatagagccgtgaggaaccagcatcctcccggccacaggagcagggcatgaaaatgtcgggcatgacgaacccgctcccccgagtcccctggcaacagggtgtgttcccttatggagcacgttcgagcagggcacattagtgtcgggcgtgacgcacccgctcccctcagtcccctgtgcaacagggagggcacttgtaacccccgtaggagggtgcccgcttccgtcctacaaaaacctctgatcataggtacctgatctaagatggtggtggcggcccatcttatcatttagctagctgatggtcttaagcatccctcccatggaatgggtaagagaagcctgcaaacaaaactggatggcaccagtgctcttacaaaatggcagccaaagcgatccagagctttcaaaactggacggggcaacagggagaaatcccggggtagcgaacctcctccgttaatgtgaaaaagtatggggaaagaactcatcttaacctcccaccgttagggagttttgattatcttttctataccatagatgc

SEQ ID NO: 52 - DNA, 231 bp, Classical swine fever virus
  source 1..231: mol_type = genomic DNA
gcgcgggtaacccgggatctggacccgccagtagaaccctgttgtagataacactaatttttttttatttatttagatattactatttatttatttatttatttattgaatgagtaagaactggtacaaactacctcaagttaccacactacactcatttttaacagcactttagctggaaggaaaattcctgacgtccacagttggactaaggtaatttcctaacggccc

SEQ ID NO: 53 - DNA, 654 bp, Culex flavivirus
  source 1..654: mol_type = genomic DNA
gaatcacgcgaatcgtagagaaccacatctctagaaaaggttaacgttgcgaagcaacgggaaccccgtaaggaaggacaaggctgtccttgagtactaacgacactccggccccagttcccagagccagggttttagctccacggtgctggaagtcaccctcgcagccatggctgcacgacgcgcgcaaggaaggacatggctgtccttgggtacgaacgacaccccgcccccagttctcaaggttagagttataacctcagggtgttggaagacatccaggccatagtagggccatcgcaagggaggattttcctcgggtactgaccataccccgaccccagtccgataggtcatggaatgaccccatggtgctgagagggcatccaaacaagctgagcatcttggattctgctcccgtaaggaaagcgcaagctttgagcattgacaacgctccggccccagtcccccaggttatgggagaataaccccgacgtgctggaagggcacgaatcaccgcaaggtgagggcgcacaggatagaatccaggtgactgacgccacctcccgaaatgtgtatagtaacagagcatgcctgcagcagcaggtctccaccgttaggagacttgttgcgggcaagctcttgttcacgtct

SEQ ID NO: 54 - DNA, 155 bp, Entebbe bat virus
  source 1..155: mol_type = genomic DNA
atgaaaatcttggaataaagtcaggccgcagcgtctaaaaccggagcctccgctgggaaaccagtcgacggggactagaggttagaggagaccccccgcgcccataaccaacataaaacagcatattgacacctgggaaaagaccggagactctg

SEQ ID NO: 55 - DNA, 250 bp, Pestivirus giraffe-1
  source 1..250: mol_type = genomic DNA
gcagtaagcagctcccaatgtaacataatgtaaataaatgtgactttatgtaaatgcaaggcagtaagcagctcccaatgtaacataatgtaaataaatgtaactttatgtaaatgcaagtagagtagttagagttctaaggacatactacatagagacaacaactacctcatttttaaaaacagcactttagctggaaggggatattccgacgtccactgttggtctaggaaaaaaccctgaaggcccc

SEQ ID NO: 56 - DNA, 269 bp, Hepatitis C virus
  source 1..269: mol_type = genomic DNA
aggttggggtaaacactccggcctcttaggccatttcctgtttttttttttttttttttttttttttttttttttttttttttttttttttctttttttttttttttttccttttttttttttttttttttctttccttcttttttcctttcttttccttccttctttaatggtggctccatcttagccctagtcacggctagctgtgaaaggtccgtgagccgcatgactgcagagagtgctgatactggcctctctgcagatcatgt

SEQ ID NO: 57 - DNA, 359 bp, Hepatitis GB virus B
  source 1..359: mol_type = genomic DNA
acccccaaattcaaaattaactaacagtttttttttttttttttttttttagggcagcggcaacaggggagaccccgggcttaacgaccccgccgatgtgagtttggcgaccatggtggatcagaaccgtttcgggtgaagccatggtctgaaggggatgacgtcccttctggctcatccacaaaaaccgtctcgggtgggtgaggagtcctggctgtgtgggaagcagtcagtataattcccgtcgtgtgtggtgacgcctcacgacgtatttgtccgctgtgcagagcgtagtaccaagggctgcaccccggtttttgttccaagcggagggcaacccccgcttggaattaaaaact

SEQ ID NO: 58 - DNA, 312 bp, GB virus C/Hepatitis G virus
  source 1..312: mol_type = genomic DNA
actaaattcatctgttgcggcaaggtctggtgactgatcatcaccggaggaggttcccgccctccccgccccaggggtctccccgctgggtaaaaagggcccggccttgggaggcatggtggttactaaccccctggcagggtcaaagcctgatggtgctaatgcactgccacttcggtggcgggtcgctaccttatagcgtaatccgtgactacgggctgctcgcagagccctccccggatggggcacagtgcactgtgatctgaaggggtgcaccccgggaagagctcggcccgaaggccggsttctact

SEQ ID NO: 59 - DNA, 388 bp, Ilheus virus
  source 1..388: mol_type = genomic DNA
acccaaaagaccaaaaaaggacaattgtgtcaggccatggaaacatgccacccaaagcttgtagagggtgcagcctgcgccaagccccaggaggactgggttaccaaagccgttaggcccccacggcccatttcaggagacagcgcgactcctggaggaaggactagaggttagaggagacccgtggaacatcgctgaggcccaaaccagcccgaagctgtaggactggtggaaggactagaggttagtggagacccctcagcaccaagcgcgaaacaaacagcatattgacgcctgggaaagaccgggagatcctctgctttccatcaccagccactaggcacagatcgccgcaagtagtggctggtggtgaaaaacacatggatct

SEQ ID NO: 60 - DNA, 1208 bp, Kamiti River virus
  source 1..1208: mol_type = genomic DNA
tgagacaaaggtccttgagtccaagttcctatccaagaaggaacaccctccccctaacccccccctccaaaagtccccatcccttccccctctcctttctggagtttgcatctgtctctatcccaagccctcagtggtttaagacagggggtatttggaactgatttccataacccctcatgcgcgacttttagagcagggcacgaaagtgtcgggcatgacgcacccgctcccccgagtcccctgaaaatagggtgggcaatgcactcctgagtaggacgggagcccagaatcctacaaaaccctcgccatgggaactggcatgacacaggagtggtgacctgtctcatacatgacaccttgaaaccccacccgtgacagcatgggctggcctctaaccctctgggtaatgctcgtacatggcagcaatcctggttctcgcaactccagtcgaatcttcgagtacacgggaacaaggatcagcaatgtttttacgacatcaccaagacgggtggaatgtccaaccccccggtagcatccgtgccaaaatggtggctctcgcaactccggtggaatcttcgatcccatcggagtgagagtcagtaatttttcgcggtgcctcccggaccgtggaatgccggcccggacgtctaggtaggaacgtaggcgtttcggattgtggttgaccgctgggtggtgctcatatttgaagcatctctcagagtctcttaccacaacctgaaatgtctgagatagaagtggcggcctatctcattgaaaacgccatttgagcagggcacgaaagtgtcgggcctgacgcacccgctcccccgagtcccctggaaacagggtgggcctcgaaaaatccaccgtaggaaggagcccaatcctacaagaaccctctggtcataggcacctgacctgggataagagtggcgccttatctcatatttagctagctggtggactcaagcacccccccccatggaatggggtaagagaggcctgtaaacatcgctggatggctccagcactcttataaattggccgccaagcgatccggagctttcaaaaccggacggagcaacagggaatttcccggggacgcgtaccccctccgtaatgtgaaaaagtatggggaaaagaacccagctaaatctcccaccgatagggagtttggactatcttttctataccataaatgcgct

SEQ ID NO: 61 - DNA, 558 bp, Kokobera virus
  source 1..558: mol_type = genomic DNA
atgaagagaatgaagtgagttattttgttgtgatagtcaggcctgaaaagccacctgatccggtgaaggtgctgcctgcatccggcctggagtgatgctccagtgtcgtggaacaacaaccgatggagccaagcccggaggggatccggcccccgacttccggaggttgccacaccttgtaaatatgtacatacagagtcagatccgaaaggccaccagtttggtgcagaactggtgctatctgtgaacactcccaggaggactgggtaaacaaagccattagggaccatcacggcccgagggggagaagaacgcgaactcccccaaaggactagaggttagaggagacccgtgattagggagatgagggagcccatctcagggaaagctgtaaccctgggggaaggactagaggttagaggagaccctcccacaaagaagcgcaaacacaaaacagcatattgacacctgggaaagactaggggatttgctgctctggacttccggctctcggcacagaacgccgttgaggagccggaggcccaaaacaccagatct

SEQ ID NO: 62 - DNA, 568 bp, Langat virus
  source 1..568: mol_type = genomic DNA
agccagacacaaggagtccaacctggagggctcttgaaaaactcgtccagaaaccaaacaaatgagcaagtcaacaggagatgataactcgtacgagctgatctccaacacacaagaaaaatggtgggatgcggcaacgcacgaggctcgtgacggggaaatgatcgctcccgacgcacccctccattggagacaacttcgtgagatcccccaggtgtttaggggcacacgcctgaggtaagcaagccccagggcgcattccggcagcacaccagtgagagtggtgacgggaaactggtcactcccgacggagctgcgccttgtgaaactttgtgagaccccttgcgtccagagaaggccgaactgggcgttataaggaggcccccagggggaaacccctgggaggagggaagagagaaattggcaactctcttcaggatatttcctcctcctataccaaattccccctcgtcagagggggggcggttcttgttctccctgagccaccatcacctagacacagatagtctgaaaaggaggtgatgcgtgtctcggaaaaacacccgct

SEQ ID NO: 63 - DNA, 497 bp, Louping ill virus
  source 1..497: mol_type = genomic DNA
gcctagcttgtgacagagcaaaacttgaagagctcgcaaggaaaccatggaatgatgcggcacggcgcgacagcgacggggaaatggtcgcacccgacgcaccatccatgaggcagcaattcgtgagacccccctggccaggaaaggggaaaacaggccaggggtgaaaacacccccagagtgcaccacggcaacacgccagtgagagtggcgacggggagatggtcgatcccgacgtagggcactctgcaagattttgcgagaccccccgccccatgacaaggccgaacatggagcattaaagggaggcccccggaagcatgcttccgggaggagggaagagagaaattggcagctctcttcagggtttttcctcctcctataccaaatttccccctcgacagagggggggcggttcttgttctccctgagccaccatcacccagacacagatagtctgacaaggaggtgatgtgtgactcggaaaaacacccgct

SEQ ID NO: 64 - DNA, 366 bp, Modoc virus
  source 1..366: mol_type = genomic DNA
acaatgaaataattaaatgaaagagtgttgagggcaaccagtgggctagccacatgggtatgacgcacccaccctctgcattcttgtaaatactttggccagtcattgtaaataggttagggagccgggcccaacccagctagggatagcctttctggggtaaggactagaggttagtggagacccccggcttttgaagttagggcaacacagggagtggttcaattggccagaaccgctctggcgtttgcctcctgttattttccaaattcccgttaccgggggtggggtgattagccatggtcgcacagatcaagctcagattgcttacatgtaatctgtgtggtcatgaatatgacctccgct

SEQ ID NO: 65 - DNA, 460 bp, Montana myotis leukoencephalitis virus
  source 1..460: mol_type = genomic DNA
tagatccagcaacacctaaaatgtacatagaaaacaactaatggaaaaaatgcgagtgagggcaactctgggattagctcaatgggtgtgacgaccctacccttccgcatttgtaaataattgagccagtcatttccgtagggaagagagttattcgctcctctcgagattgagcggcctgctccttggagcatgagatgggaggcccgaagcaaagctgaaaggactagcggttagaggagaccccttccatctctggtatcaaatttcatggagtttactccatggtggctagaacccatagcgggggtgaaccacattggctaaggttcaccagcttttgctcccgcgtttttcaaattgcctcatcttgaatggggggcggcgtggatatatactccagccagaaaagactcagattgtctcatgactttctgactggcgtacatagccatccgct

SEQ ID NO: 66 - DNA, 614 bp, Murray Valley encephalitis virus
  source 1..614: mol_type = genomic DNA
ataacattgatagaaaattttgtaaatatttaatgtaatatagtataggtaaaattttttgaaattaagtaaaattaagtagcaagacttgatagtcaggccagccggttaggctgccaccgaaggttggtagacggtgctgcctgcgaccaaccccaggaggactgggttaccaaagctgattctccacggttggaaagcctcccagaaccgtctcggaagaggagtccctgccaacaatggagatgaagcccgtgtcagatcgcgaaagcgccacttcgccgaggagtgcaatctgtgaggccccaggaggactgggtaaacaaagccgtaaggcccccgcagcccgggccgggaggaggtgatgcaaaccccggcgaaggactagaggttagaggagaccctgcggaagaaatgagtggcccaagctcgccgaagctgtaaggcgggtggacggactagaggttagaggagaccccactctcaaaagcatcaaacaacagcatattgacacctgggaaaagactaggagatcttctgctctattccaacatcagtcacaaggcaccgagcgccgaacactgtgactgatgggggagaagaccacaggatct

SEQ ID NO: 67 - DNA, 410 bp, Omsk hemorrhagic fever virus
  source 1..410: mol_type = genomic DNA
ccacagacaaccatagagcaaaagcaccatttcgtgagacccccctgccagttgaagggggaagctggccggtggtagaaaaccccccaacagggtgccaaacggcaacacgccagtgagagtggcgacgggaacatggtcgctcccgacgtagggcactctatccaattttgtgagaccccccgcaccatggaaggccaaacatggtgcatgaagggaaaggcccccggaagcttgcttccgggaggagggaagagagaaattggcagctctcttcaggaaatttcctcctcctataccaaattccccctcatctgagggggggcggttcttgttctccctgagccaccatcacccagacacaggcagtctaacaaggaggtgatgtgtgactcggaacaacacccgct

SEQ ID NO: 68 - DNA, 480 bp, Powassan virus
  source 1..480: mol_type = genomic DNA
actagcatgactgaacagtcaaaagaaccctaacacaggggatggtgtggcagcgcacaacgacatcgtgacgggagtgggtcgcccccgacgcaccatcctcttgggaaaaattttcgtgagaccctcacggctggcaaagggcaccagtcgtgtagtaagaaggccctggcccagtgcggcagcacactcagtgacgggaaagtggtcgctcccgacgtaactgggtaaaaacgaactttgtgagaccaaaaggcctcctggaaggctcaccaggagttaggccgtttaggagcccccgagcataactcgggaggagggaggaagaaaattggcaatcttcctcgggatttttccgcctcctatactaaatttcccccaggaaactgggggggcggttcttgttctccctgagccaccaccatccaggcacagatagcctgacaaggagatggtgtgtgactcggaaaaacacccgct

SEQ ID NO: 69 - DNA, 459 bp, Sepik virus
  source 1..459: mol_type = genomic DNA
acagactgacacaaaataagtgaccagaatgggactaaaccacctactatatgtaaaaccgggataaaaaccacggagaggaccggacctctcactatgtaaaaccggtatacaaaccaaaacagacaggaccggacctgcctgatgtcagcccgtcataatgacgccatggctaagctgtgaggccatgctggctgggatagccgcgaccacccgcgtaatggggttcctggattgctcgatccggggtaaaaaatttttagggagcctccgcctgctgcgtccgcgcgcagcaggaaagaaggggtctagaggttagaggagaccctcccgagcactatagcggaccatattgacgcctgggaaagaccggagacactccttgattctcacctttctcacccttaagcacagattgcttgaatgcagggtggggaagttgggaaccaactagtgtct

SEQ ID NO: 70 - DNA, 429 bp, Yokose virus
  source 1..429: mol_type = genomic DNA
gagcaataaaaaattttaaagacaaaagtgtcaggccaagattgagaaaatcttgccacagcttggcagactgtgcagcctgcagccctagagggagactgaccaactccctttagtagaaaaggtcagggaagaacttgaggatgggtgtggcctcaagatctcttctcaaaaaacggactgaacaccacacctagatgaagatagtaggggagcctccgccaatggtggctttacatattgagctactgcattggtcgatggggactagcggttagaggagaccctctcctacgcatggattttgcaatatgttgacatcagggaaagaccgggtgtttgtcggttccggagagctccggaggccagggcgccgtttgcccgtagtttataactggccttcggggatcgaaggagttgccaaacact

SEQ ID NO: 71 - DNA, 57 bp, synthetic construct
  source 1..57: mol_type = other DNA
gccaccaacttcagcctgctgaagcaggccggcgacgtggaggagaaccccggcccc

SEQ ID NO: 72 - protein, 19 aa, synthetic construct
ATNFSLLKQAGDVEENPGP

SEQ ID NO: 73 - DNA, 30 bp, synthetic construct
  source 1..30: mol_type = other DNA
gacagggtgtacatccaccccttccacctg

SEQ ID NO: 74 - DNA, 24 bp, synthetic construct
  source 1..24: mol_type = other DNA
atcctggcccaggtggtgggcgac

SEQ ID NO: 75 - DNA, 24 bp, synthetic construct
  source 1..24: mol_type = other DNA
gagaggaacctgctgagcgtggcc

SEQ ID NO: 76 - DNA, 24 bp, synthetic construct
  source 1..24: mol_type = other DNA
atcaggagcttcgtggagaccaag

SEQ ID NO: 77 - DNA, 24 bp, synthetic construct
  source 1..24: mol_type = other DNA
agcgccaagcccgtgagccagatg

SEQ ID NO: 78 - DNA, 27 bp, synthetic construct
  source 1..27: mol_type = other DNA
caggaggccttcgacatcagcaagaag

SEQ ID NO: 79 - DNA, 24 bp, synthetic construct
  source 1..24: mol_type = other DNA
aaccagggcaggatcgagcccgac

SEQ ID NO: 80 - DNA, 24 bp, synthetic construct
  source 1..24: mol_type = other DNA
gtgctggtggagaggagcgccgcc

SEQ ID NO: 81 - DNA, 33 bp, synthetic construct
  source 1..33: mol_type = other DNA
ggcaggtggcacaaggtgagcgtgaggtgggag

SEQ ID NO: 82 - DNA, 24 bp, synthetic construct
  source 1..24: mol_type = other DNA
gcctacaagaacgtggtgggcgcc

SEQ ID NO: 83 - protein, 10 aa, synthetic construct
DRVYIHPFHL

SEQ ID NO: 84 - protein, 8 aa, synthetic construct
ILAQVVGD

SEQ ID NO: 85 - protein, 8 aa, synthetic construct
ERNLLSVA

SEQ ID NO: 86 - protein, 8 aa, synthetic construct
IRSFVETK

SEQ ID NO: 87 - protein, 8 aa, synthetic construct
SAKPVSQM

SEQ ID NO: 88 - protein, 9 aa, synthetic construct
QEAFDISKK

SEQ ID NO: 89 - protein, 8 aa, synthetic construct
NQGRIEPD

SEQ ID NO: 90 - protein, 8 aa, synthetic construct
VLVERSAA

SEQ ID NO: 91 - protein, 11 aa, synthetic construct
GRWHKVSVRWE

SEQ ID NO: 92 - protein, 8 aa, synthetic construct
AYKNVVGA

SEQ ID NO: 93 - DNA, 3720 bp, synthetic construct
  source 1..3720: mol_type = other DNA
gtgaacctgaccaccagaacacagctgcctccagcctacaccaacagctttaccagaggcgtgtactaccctgacaaggtgttcagatccagtgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggctcctctgagcgagacaaagtgcaccctgaagtccttcaccgtggaaaagggcatctaccagaccagcaacttccgggtgcagcccaccgagtccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcactatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaaaggcttcaactgctacttcccactgcagtcctacggctttcagcccacgtatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggacatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacccgccggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgatgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgctaa

SEQ ID NO: 94 - DNA, 2499 bp, synthetic construct
  source 1..2499: mol_type = other DNA
atgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtga

SEQ ID NO: 95 - DNA, 1518 bp, synthetic construct
  source 1..1518: mol_type = other DNA
atgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaa

SEQ ID NO: 96 - DNA, 1707 bp, synthetic construct
  source 1..1707: mol_type = other DNA
atggagaaaatagtgcttcttcttgcaatagtcagtcttgtcaaaagtgatcagatttgcattggttaccatgcaaacaactcgacagagcaggttgacacaataatggaaaagaacgttactgttacacatgcccaagacatactggaaaagacacacaatgggaagctctgcgatctaaatggagtgaagcctctcattttgagagattgtagtgtagctggatggctcctcggaaaccctatgtgtgacgaattcatcaatgtgccggaatggtcttacatagtggagaaggccagtccagccaatgacctctgttacccaggggatttcaacgactatgaagaactgaaacacctattgagcagaacaaaccattttgagaaaattcagatcatccccaaaagttcttggtccaatcatgatgcctcatcaggggtgagctcagcatgtccataccatgggaggtcctcctttttcagaaatgtggtatggcttatcaaaaagaacagtgcatacccaacaataaagaggagctacaataataccaaccaagaagatcttttagtactgtgggggattcaccatcctaatgatgcggcagagcagacaaagctctatcaaaacccaaccacttacatttccgttggaacatcaacactgaaccagagattggttccagaaatagctactagacccaaagtaaacgggcaaagtggaagaatggagttcttctggacaattttaaagccgaatgatgccatcaatttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccccctcaccatcggggaatgccccaaatatgtgaaatcaaacagattagtccttgcgactggactcagaaatacccctcagagagagagaagaagaaaaaagagaggactatttggagctatagcaggttttatagagggaggatggcagggaatggtagatggttggtatgggtaccaccatagcaatgagcaggggagtggatacgctgcagacaaagaatccactcaaaaggcaatagatggagtcaccaataaggtcaactcgatcattgacaaaatgaacactcagtttgaggccgttggaagggaatttaataacttggaaaggaggatagagaatttaaacaagcagatggaagacggattcctagatgtctggacttataatgctgaacttctggttctcatggaaaatgagagaactctagactttcatgactcaaatgtcaagaacctttatgacaaggtccgactacagcttagggataatgcaaaggagctgggtaatggttgtttcgagttctatcacaaatgtgataatgaatgtatggaaagtgtaaaaaacggaacgtatgactacccgcagtattcagaagaagcaagactaaacagagaggaaataagtggagtaaaattggaatcaatgggaacttaccaaatactgtcaatttattcaacagtggcgagttccctagcactggcaatcatggtagctggtctatctttatggatgtgctccaatggatcgttacaatgcagaatttgcatttaa

SEQ ID NO: 97 - protein, 1239 aa, synthetic construct
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALAPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 98 - protein, 832 aa, synthetic construct
MPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPKVTKYLPLDKGIKPYYPEHLVNHYFQTRHYLHTLWKAGILYKRETTHSASFCGSPYSWEQDLQHGAESFHQQSSGILSRPPVGSSLQSKHRKSRLGLQSQQGHLARRQQGRSWSIRAGFHPTARRPFGVEPSGSGHTTNFASKSASCLHQSPVRKAAYPAVSTFEKHSSSGHAVEFHNLPPNSARSQSERPVFPCWWLQFRNSKPCSDYCLSLIVNLLEDWGPCAEHGEHHIRIPRTPSRVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRILNNQHGTMPDLHDYCSRNLYVSLLLLYQTFGRKLHLYSHPIILGFRKIPMGVGLSPFLLAQFTSAICSVVRRAFPHCLAFSYMDDVVLGAKSVQHLESLFTAVTNFLLSLGIHLNPNKTKRWGYSLNFMGYVIGCYGSLPQEHIIQKIKECFRKLPINRPIDWKVCQRIVGLLGFAAPFTQCGYPALMPLYACIQSKQAFTFSPTYKAFLCKQYLNLYPVARQRPGLCQVFADATPTGWGLVMGHQRMRGTFSAPLPIHTAELLAACFARSRSGANIIGTDNSVVLSRKYTSFPWLLGCAANWILRGTSFVYVPSALNPADDPSRGRLGLSRPLLRLPFRPTTGRTSLYADSPSVPSHLPDRVHFASPLHVAWRPP

SEQ ID NO: 99 - protein, 505 aa, synthetic construct
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGTVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 100 - protein, 568 aa, synthetic construct
MEKIVLLLAIVSLVKSDQICIGYHANNSTEQVDTIMEKNVTVTHAQDILEKTHNGKLCDLNGVKPLILRDCSVAGWLLGNPMCDEFINVPEWSYIVEKASPANDLCYPGDFNDYEELKHLLSRTNHFEKIQIIPKSSWSNHDASSGVSSACPYHGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIIDKMNTQFEAVGREFNNLERRIENLNKQMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 101 - DNA, 45 bp, synthetic construct
  source 1..45: mol_type = other DNA
atgttcgtgtttctggtgctgctgcctctggtgtccagccagtgt

SEQ ID NO: 102 - DNA, 51 bp, synthetic construct
  source 1..51: mol_type = other DNA
atgggcgtgaaggtgctgttcgccctgatctgcatcgccgtggccgaggcc

SEQ ID NO: 103 - DNA, 54 bp, synthetic construct
  source 1..54: mol_type = other DNA
atgaagtgggtgaccttcatcagcctgctgttcctgttcagcagcgcctacagc

SEQ ID NO: 104 - DNA, 54 bp, synthetic construct
  source 1..54: mol_type = other DNA
atggccttcctgtggctgctgagctgctgggccctgctgggcaccaccttcggc

SEQ ID NO: 105 - DNA, 42 bp, synthetic construct
  source 1..42: mol_type = other DNA
atgcagctgctgagctgcatcgccctgatcctggccctggtg

SEQ ID NO: 106 - DNA, 45 bp, synthetic construct
  source 1..45: mol_type = other DNA
atgaacctgctgctgatcctgaccttcgtggccgccgccgtggcc

SEQ ID NO: 107 - protein, 15 aa, synthetic construct
MFVFLVLLPLVSSQC

SEQ ID NO: 108 - protein, 17 aa, synthetic construct
MGVKVLFALICIAVAEA

SEQ ID NO: 109 - protein, 18 aa, synthetic construct
MKWVTFISLLFLFSSAYS

SEQ ID NO: 110 - protein, 18 aa, synthetic construct
MAFLWLLSCWALLGTTFG

SEQ ID NO: 111 - protein, 14 aa, synthetic construct
MQLLSCIALILALV

SEQ ID NO: 112 - protein, 15 aa, synthetic construct
MNLLLILTFVAAAVA

SEQ ID NO: 113 - DNA, 45 bp, synthetic construct
  source 1..45: mol_type = other DNA
ttccaggacgcctacaacgccgccggcggccacaacgccgtgttc

SEQ ID NO: 114 - DNA, 303 bp, synthetic construct
  source 1..303: mol_type = other DNA
atggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttctga

SEQ ID NO: 115 - DNA, 3818 bp, synthetic construct
  source 1..3818: mol_type = other DNA
atgttcgtgttcctggtgctgctgcccctggtgagcagccagtgcgtgaacctgaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccgcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacaaggtggaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 116 - DNA, 1707 bp, synthetic construct; identical to SEQ ID NO: 96
  source 1..1707: mol_type = other DNA

SEQ ID NO: 117 - DNA, 288 bp, synthetic construct
  source 1..288: mol_type = other DNA
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag

SEQ ID NO: 118 - DNA, 1303 bp, synthetic construct
  source 1..1303: mol_type = other DNA
atgtatttcaagtacacagcagcagccctagctgcggtgctccctctttgctctgcacagacttggtcaaagtgcaatccccttgagagtgagtgttttcataccgacatatgatatacatcagcttatctaacgattgttttgcagagacctgcccgcccaacaagggtcttgctgcatccacttacaccgccgacttcacctcagcttcagctttggatcaatgggaagtcactgcaggcaaagttcccgttggcccacagggcgccgagttcactgtcgctaagcaaggcgacgcacctaccattgacaccgacttctacttcttcttcggaaaggccgaagtggtgatgaaggccgctcctggcacaggtgttgttagcagcatcgtcctggagtcggatgatctggatgaggttgactgggtaagcctgcttgtctatcatgtgttcgtcttgagccggacttaacgaaagcgcaggaagtattgggcggtgacaccactcaggttcagacaaactactttggcaaaggagacaccaccacatatgaccgaggcacttacgtgcccgttgccactcctcaggagactttccacacctacaccatcgactggaccaaggatgccgttacctggtctattgacggtgcggtcgtgcgtacgctcacgtacaacgatgccaagggtggcactcgcttccctcagactcctatgcgcctgagacttggcagctgggccggcggcgaccccagcaaccccaagggcaccatcgagtgggccggtggcttgaccgactacagcgcgggaccgtacaccatgtacgtcaagtccgtccgtatcgagaacgccaaccccgccgagtcctacacctactcggacaactctggctcttggcagagcatcaagttcgacggctccgtcgatatctcctccagctcttccgtgacctcctccaccaccagcaccgccagctccgccagctctacctcgagcaagaccccttccacctccaccctggccacttccaccaaggcgactcccaccccgtctggaaccagctccggctctaactcgagctccagcgcggaacctactaccaccggcggcaccggcagcagcaacaccggctctggctccggctccggctctggctctggctctagctctagcacgggctcctccactagcgccggagcctccgccacccccgagctctcccagggcgccgccggctccatcaagggctcggtcaccgcctgcgctctggtgttcggcgccgtcgctgccgtgttggcattctaa

SEQ ID NO: 119 - DNA, 681 bp, synthetic construct
  source 1..681: mol_type = other DNA
atgccgatcgaccgcaagacgctctgccatctcctgtccgttctgccgttggccctcctcggatctcacgtggcgcgggcctccacgccaggcatcgtcattccgccgcaggaacagattacccagcacggcggcccctatggacgctgcgcgaacaagacccgtgccctgaccgtggcggaattgcgcggcagcggcgatctgcaggagtacctgcgtcatgtgacgcgcggctggtcaatatttgcgctctacgatggcacctatctcggcggcgaatatggcggcgtgatcaaggacggaacacccggcggcgcattcgacctgaaaacgacgttctgcatcatgaccacgcgcaatacgggtcaacccgcaacggatcacttctacagcaacgtcaccgccactcgcctgctctccagcaccaacagcaggctatgcgcggtcttcgtcagaagcgggcaaccggtcattggcgcctgcaccagcccgtatgacggcaagtactggagcatgtacagccggctgcggaaaatgctttacctgatctacgtggccggcatctccgtacgcgtccatgtcagcaaggaagaacagtattacgactacgaagacgcaacgttcgagacttacgcccttaccggcatctccatctgcaatccgggatcatccttatgctga

SEQ ID NO: 120 - DNA, 3182 bp, synthetic construct
  source 1..3182: mol_type = other DNA
aattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtgg

SEQ ID NO: 121 - DNA, 9036 bp, synthetic construct
  source 1..9036: mol_type = other DNA
atgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaagtgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttcctcctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccttctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctacatcagctggtgcatgtggtggcttcagtattttctgaccagagtagaagcgcaactgcacgtgtgggttccccccctcaacgtccggggggggcgcgatgccgtcatcttactcatgtgtgttgtacacccgactctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgcacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaggcggtggactttatccctgtggagaacctagagacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccctttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctacagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttctatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggattgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccaagcagaggttatcacccctgctgtccagaccaactggcagaaactcgaggtcttctgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctaccgcctttgtgggcgctggcttagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacataggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggccagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacccgcagaaattctgcggaagtctcggagattcgcccgggccctgcccgtttgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatga

SEQ ID NO: 122 - DNA, 1503 bp, synthetic construct
  source 1..1503: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataa

SEQ ID NO: 123 - DNA, 1098 bp, synthetic construct
  source 1..1098: mol_type = other DNA
atggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatga

SEQ ID NO: 124 - DNA, 1194 bp, synthetic construct
  source 1..1194: mol_type = other DNA
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattag

SEQ ID NO: 125 - DNA, 3945 bp, synthetic construct
  source 1..3945: mol_type = other DNA
atgcccatcaccatcaacaacttcaggtacagcgaccccgtgaacaacgacaccatcatcatgatggagcccccctactgcaagggcctggacatctactacaaggccttcaagatcaccgacaggatctggatcgtgcccgagaggtacgagttcggcaccaagcccgaggacttcaacccccccagcagcctgatcgagggcgccagcgagtactacgaccccaactacctgaggaccgacagcgacaaggacaggttcctgcagaccatggtgaagctgttcaacaggatcaagaacaacgtggccggcgaggccctgctggacaagatcatcaacgccatcccctacctgggcaacagctacagcctgctggacaagttcgacaccaacagcaacagcgtgagcttcaacctgctggagcaggaccccagcggcgccaccaccaagagcgccatgctgaccaacctgatcatcttcggccccggccccgtgctgaacaagaacgaggtgaggggcatcgtgctgagggtggacaacaagaactacttcccctgcagggacggcttcggcagcatcatgcagatggccttctgccccgagtacgtgcccaccttcgacaacgtgatcgagaacatcaccagcctgaccatcggcaagagcaagtacttccaggaccccgccctgctgctgatgcacgagctgatccacgtgctgcacggcctgtacggcatgcaggtgagcagccacgagatcatccccagcaagcaggagatctacatgcagcacacctaccccatcagcgccgaggagctgttcaccttcggcggccaggacgccaacctgatcagcatcgacatcaagaacgacctgtacgagaagaccctgaacgactacaaggccatcgccaacaagctgagccaggtgaccagctgcaacgaccccaacatcgacatcgacagctacaagcagatctaccagcagaagtaccagttcgacaaggacagcaacggccagtacatcgtgaacgaggacaagttccagatcctgtacaacagcatcatgtacggcttcaccgagatcgagctgggcaagaagttcaacatcaagaccaggctgagctacttcagcatgaaccacgaccccgtgaagatccccaacctgctggacgacaccatctacaacgacaccgagggcttcaacatcgagagcaaggacctgaagagcgagtacaagggccagaacatgagggtgaacaccaacgccttcaggaacgtggacggcagcggcctggtgagcaagctgatcggcctgtgcaagaagatcatcccccccaccaacatcagggagaacctgtacaacaggaccgccagcctgaccgacctgggcggcgagctgtgcatcaagatcaagaacgaggacctgaccttcatcgccgagaagaacagcttcagcgaggagcccttccaggacgagatcgtgagctacaacaccaagaacaagcccctgaacttcaactacagcctggacaagatcatcgtggactacaacctgcagagcaagatcaccctgcccaacgacaggaccacccccgtgaccaagggcatcccctacgcccccgagtacaagagcaacgccgccagcaccatcgagatccacaacatcgacgacaacaccatctaccagtacctgtacgcccagaagagccccaccaccctgcagaggatcaccatgaccaacagcgtggacgacgccctgatcaacagcaccaagatctacagctacttccccagcgtgatcagcaaggtgaaccagggcgcccagggcatcctgttcctgcagtgggtgagggacatcatcgacgacttcaccaacgagagcagccagaagaccaccatcgacaagatcagcgacgtgagcaccatcgtgccctacatcggccccgccctgaacatcgtgaagcagggctacgagggcaacttcatcggcgccctggagaccaccggcgtggtgctgctgctggagtacatccccgagatcaccctgcccgtgatcgccgccctgagcatcgccgagagcagcacccagaaggagaagatcatcaagaccatcgacaacttcctggagaagaggtacgagaagtggatcgaggtgtacaagctggtgaaggccaagtggctgggcaccgtgaacacccagttccagaagaggagctaccagatgtacaggagcctggagtaccaggtggacgccatcaagaagatcatcgactacgagtacaagatctacagcggccccgacaaggagcagatcgccgacgagatcaacaacctgaagaacaagctggaggagaaggccaacaaggccatgatcaacatcaacatcttcatgagggagagcagcaggagcttcctggtgaaccagatgatcaacgaggccaagaagcagctgctggagttcgacacccagagcaagaacatcctgatgcagtacatcaaggccaacagcaagttcatcggcatcaccgagctgaagaagctggagagcaagatcaacaaggtgttcagcacccccatccccttcagctacagcaagaacctggactgctgggtggacaacgaggaggacatcgacgtgatcctgaagaagagcaccatcctgaacctggacatcaacaacgacatcatcagcgacatcagcggcttcaacagcagcgtgatcacctaccccgacgcccagctggtgcccggcatcaacggcaaggccatccacctggtgaacaacgagagcagcgaggtgatcgtgcacaaggccatggacatcgagtacaacgacatgttcaacaacttcaccgtgagcttctggctgagggtgcccaaggtgagcgccagccacctggagcagtacggcaccaacgagtacagcatcatcagcagcatgaagaagcacagcctgagcatcggcagcggctggagcgtgagcctgaagggcaacaacctgatctggaccctgaaggacagcgccggcgaggtgaggcagatcaccttcagggacctgcccgacaagttcaacgcctacctggccaacaagtgggtgttcatcaccatcaccaacgacaggctgagcagcgccaacctgtacatcaacggcgtgctgatgggcagcgccgagatcaccggcctgggcgccatcagggaggacaacaacatcaccctgaagctggacaggtgcaacaacaacaaccagtacgtgagcatcgacaagttcaggatcttctgcaaggccctgaaccccaaggagatcgagaagctgtacaccagctacctgagcatcaccttcctgagggacttctggggcaaccccctgaggtacgacaccgagtactacctgatccccgtggccagcagcagcaaggacgtgcagctgaagaacatcaccgactacatgtacctgaccaacgcccccagctacaccaacggcaagctgaacatctactacaggaggctgtacaacggcctgaagttcatcatcaagaggtacacccccaacaacgagatcgacagcttcgtgaagagcggcgacttcatcaagctgtacgtgagctacaacaacaacgagcacatcgtgggctaccccaaggacggcaacgccttcaacaacctggacaggatcctgagggtgggctacaacgcccccggcatccccctgtacaagaagatggaggccgtgaagctgagggacctgaagacctacagcgtgcagctgaagctgtacgacgacaagaacgccagcctgggcctggtgggcacccacaacggccagatcggcaacgaccccaacagggacatcctgatcgccagcaactggtacttcaaccacctgaaggacaagatcctgggctgcgactggtacttcgtgcccaccgacgagggctggaccaacgac

SEQ ID NO: 126 - DNA, 303 bp, synthetic construct
  source 1..303: mol_type = other DNA
gtggcgaaggtgaacatcaagccactcgaggacaagattctcgtgcaggccaacgaggccgagaccacgaccgcgtccggtctggtcattcctgacaccgccaaggagaagccgcaggagggcaccgtcgttgccgtcggccctggccggtgggacgaggacggcgagaagcggatcccgctggacgttgcggagggtgacaccgtcatctacagcaagtacggcggcaccgagatcaagtacaacggcgaggaatacctgatcctgtcggcacgcgacgtgctggccgtcgtttccaagtag

SEQ ID NO: 127 - DNA, 1497 bp, synthetic construct
  source 1..1497: mol_type = other DNA
atgtcatttgtggtcacgatcccggaggcgctagcggcggtggcgaccgatttggcgggtatcgggtcgacgatcggcaccgccaacgcggccgccgcggtcccgaccacgacggtgttggccgccgccgccgatgaggtgtcggcggcgatggcggcattgttctccggacacgcccaggcctatcaggcgctgagcgcccaggcggcgctgtttcacgagcagttcgtgcgggcgctcaccgccggggcgggctcgtatgcggccgccgaggccgccagcgcggccccgctagagggtgtgctcgacgtgatcaacgcccccgccctggcgctgttggggcgcccactgatcggtaacggagccaacggggccccggggaccggggcaaacggcggcgacggcggaatcttgatcggcaacggcggggccggcggctccggcgcggccggcatgcccgggggcaacggcggagccgctggcctgttcggcaacggcggggccggcggcgccggggggaacgtagcgtccggcaccgcagggttcggcggggccggcggggccggcgggctgctctacggcgccggcggggccggcggcgccggcggacgcgccggtggtggggtgggcggtattggtggggccggcggggccggcggcaatggcgggctgctgttcggcgccggcggggccggcggcgtcggcggactcgcggctgacgccggtgacggcggggccggcggagacggcgggttgttcttcggcgtgggcggtgccggcggggccggcggcaccggcactaatgtcaccggcggtgccggcggggccggcggcaatggcgggctcctgttcggcgccggcggggtgggcggtgttggcggtgacggtgtggcattcctgggcaccgcccccggcgggcccggtggtgccggcggggccggtgggctgttcggcgtcggtggggccggcggcgccggcggaatcggattggtcgggaacggcggtgccggggggtccggcgggtccgccctgctctggggcgacggcggtgccggcggcgcgggtggggtcgggtccactaccggcggtgccggcggggcgggcggcaacgccggcctgctggtaggcgccggcggggccggcggcgccggcgcactcggcggtggcgctaccggggtgggcggcgccggcggaaacggcggcactgcgggcctgctgtttggtgccggcggcgccggcggattcggcttcggcggtgccgggggcgccggtgggctcggcggcaaagccgggctgatcggcgacggcggtgacggcggcgccggaggaaacggcaccggtgccaagggcggtgacggcggcgctggcggcggtgccatcctggtcggcaacggcggcaacggcggcaacgccgggagtggcacacctaacggcagcgcgggcaccggcggtgccggcgggctgttgggtaagaacgggatgaacgggttaccgtag

SEQ ID NO: 128 - DNA, 978 bp, synthetic construct
  source 1..978: mol_type = other DNA
atgacagacgtgagccgaaagattcgagcttggggacgccgattgatgatcggcacggcagcggctgtagtccttccgggcctggtggggcttgccggcggagcggcaaccgcgggcgcgttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctga

SEQ ID NO: 129 - DNA, 1932 bp, synthetic construct
  source 1..1932: mol_type = other DNA
ccccagtggagctacatgcacatcagcggccaggacgccagcgagtacctgagccccggcctggtgcagttcgccagggccaccgagacctacttcagcctgaacaacaagttcaggaaccccaccgtggcccccacccacgacgtgaccaccgacaggagccagaggctgaccctgaggttcatccccgtggacagggaggacaccgcctacagctacaaggccaggttcaccctggccgtgggcgacaacagggtgctggacatggccagcacctacttcgacatcaggggcgtgctggacaggggccccaccttcaagccctacagcggcaccgcctacaacgccctggcccccaagggcgcccccaacagctgcgagtgggagcagaccgaggacagcggcagggccgtggccgaggacgaggaggaggaggacgaggacgaggaggaggaggaggaggagcagaacgccagggaccaggccaccaagaagacccacgtgtacgcccaggcccccctgagcggcgagaccatcaccaagagcggcctgcagatcggcagcgacaacgccgagacccaggccaagcccgtgtacgccgaccccagctaccagcccgagccccagatcggcgagagccagtggaacgaggccgacgccaacgccgccggcggcagggtgctgaagaagaccacccccatgaagccctgctacggcagctacgccaggcccaccaaccccttcggcggccagagcgtgctggtgcccgacgagaagggcgtgcccctgcccaaggtggacctgcagttcttcagcaacaccaccagcctgaacgacaggcagggcaacgccaccaagcccaaggtggtgctgtacagcgaggacgtgaacatggagacccccgacacccacctgagctacaagcccggcaagggcgacgagaacagcaaggccatgctgggccagcagagcatgcccaacaggcccaactacatcgccttcagggacaacttcatcggcctgatgtactacaacagcaccggcaacatgggcgtgctggccggccaggccagccagctgaacgccgtggtggacctgcaggacaggaacaccgagctgagctaccagctgctgctggacagcatcggcgacaggaccaggtacttcagcatgtggaaccaggccgtggacagctacgaccccgacgtgaggatcatcgagaaccacggcaccgaggacgagctgcccaactactgcttccccctgggcggcatcggcgtgaccgacacctaccaggccatcaaggccaacggcaacggcagcggcgacaacggcgacaccacctggaccaaggacgagaccttcgccaccaggaacgagatcggcgtgggcaacaacttcgccatggagatcaacctgaacgccaacctgtggaggaacttcctgtacagcaacatcgccctgtacctgcccgacaagctgaagtacaaccccaccaacgtggagatcagcgacaaccccaacacctacgactacatgaacaagagggtggtggcccccggcctggtggactgctacatcaacctgggcgccaggtggagcctggactacatggacaacgtgaaccccttcaaccaccacaggaacgccggcctgaggtacaggagcatgctgctgggcaacggcaggtacgtgcccttccacatccaggtgccccagaagttcttcgccatcaagaacctgctgctgctgcccggcagctacacctacgagtggaacttcaggaaggacgtgaacatggtgctgcagagcagcctgggcaacgacctgagggtggacggcgccagcatcaagttcgacagcatctgcctgtacgccaccttcttccccatggcccacaacaccgccagcaccctggaggccatgctgagg

SEQ ID NO: 130 - DNA, 828 bp, synthetic construct
  source 1..828: mol_type = other DNA
atggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaa

SEQ ID NO: 131 - DNA, 1269 bp, synthetic construct
  source 1..1269: mol_type = other DNA
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 132 - DNA, 2699 bp, synthetic construct
  source 1..2699: mol_type = other DNA
ggcaccggcaacatcggcgagaccctgggcgagaagtggaagagcaggctgaacgccctgggcaagagcgagttccagatctacaagaagagcggcatccaggaggtggacaggaccctggccaaggagggcatcaagaggggcgagaccgaccaccacgccgtgagcaggggcagcgccaagctgaggtggttcgtggagaggaacatggtgacccccgagggcaaggtggtggacctgggctgcggcaggggcggctggagctactactgcggcggcctgaagaacgtgagggaggtgaagggcctgaccaagggcggccccggccacgaggagcccatccccatgagcacctacggctggaacctggtgaggctgcagagcggcgtggacgtgttcttcatcccccccgagaagtgcgacaccctgctgtgcgacatcggcgagagcagccccaaccccaccgtggaggccggcaggaccctgagggtgctgaacctggtggagaactggctgaacaacaacacccagttctgcataaggtgctgaacccctacatgcccagcgtgatcgagaagatggaggccctgcagaggaagtacggcggcgccctggtgaggaaccccctgagcaggaacagcacccacgagatgtactgggtgagcaacgccagcggcaacatcgtgagcagcgtgaacatgatcagcaggatgctgatcaacaggttcaccatgaggtacaagaaggccacctacgagcccgacgtggacctgggcagcggcaccaggaacatcggcatcgagagcgagatccccaacctggacatcatcggcaagaggatcgagaagatcaagcaggagcacgagaccagctggcactacgaccaggaccacccctacaagacctgggcctaccacggcagctacgagaccaagcagaccggcagcgccagcagcatggtgaacggcgtggtgaggctgctgaccaagccctgggacgtggtgcccatggtgacccagatggccatgaccgacaccacccccttcggccagcagagggtgttcaaggagaaggtggacaccaggacccaggagcccaaggagggcaccaagaagctgatgaagatcaccgccgagtggctgtggaaggagctgggcaagaagaagacccccaggatgtgcaccagggaggagttcaccaggaaggtgaggagcaacgccgccctgggcgccatcttcaccgacgagaacaagtggaagagcgccagggaggccgtggaggacagcaggttctgggagctggtggacaaggagaggaacctgcacctggagggcaagtgcgagacctgcgtgtacaacatgatgggcaagagggagaagaagctgggcgagttcggcaaggccaagggcagcagggccatctggtacatgtggctgggcgccaggttcctggagttcgaggccctgggcttcctgaacgaggaccactggttcagcagggagaacagcctgagcggcgtggagggcgagggcctgcacaagctgggctacatcctgagggacgtgagcaagaaggagggcggcgccatgtacgccgacgacaccgccggctgggacaccaggatcaccctggaggacctgaagaacgaggagatggtgaccaaccacatggagggcgagcacaagaagctggccgaggccatcttcaagctgacctaccagaacaaggtggtgagggtgcagaggcccacccccaggggcaccgtgatggacatcatcagcaggagggaccagaggggcagcggccaggtgggcacctacggcctgaacaccttcaccaacatggaggcccagctgatcaggcagatggagggcgagggcgtgttcaagagcatccagcacctgaccatcaccgaggagatcgccgtgcagaactggctggccagggtgggcagggagaggctgagcaggatggccatcagcggcgacgactgcgtggtgaagcccctggacgacaggttcgccagcgccctgaccgccctgaacgacatgggcaagatcaggaaggacatccagcagtgggagcccagcaggggctggaacgactggacccaggtgcccttctgcagccaccacttccacgagctgatcatgaaggacggcagggtgctggtggtgccctgcaggaaccaggacgagctgatcggcagggccaggatcagccagggcgccggctggagcctgagggagaccgcctgcctgggcaagagctacgcccagatgtggagcctgatgtacttccacaggagggacctgaggctggccgccaacgccatctgcagcgccgtgcccagccactgggtgcccaccagcaggaccacctggagcatccacgccaagcacgagtggatgaccaccgaggacatgctgaccgtgtggaacagggtgtggatccaggagaacccctggatggaggacaagacccccgtggagagctgggaggagatcccctacctgggcaagagggaggaccagtggtgcggcagcctgatcggcctgaccagcagggccacctgggccaagaacatccaggccgccatcaaccaggtgaggagcctgatcggcaacgaggagtacaccgactacatgcccagcatgaagaggttcaggagggaggaggaggaggccggcgtgctgtgg

SEQ ID NO: 133 - DNA, 2529 bp, synthetic construct
  source 1..2529: mol_type = other DNA
atgcccctgagctaccagcacttcaggaagctgctgctgctggacgaggaggccggccccctggaggaggagctgcccaggctggccgacgagggcctgaacaggagggtggccgaggacctgaacctgggcaacctgaacgtgagcatcccctggacccacaaggtgggcaacttcaccggcctgtacagcagcaccgtgccctgcttcaaccccaagtggcagacccccagcttccccgacatccacctgcaggaggacatcgtggacaggtgcaagcagttcgtgggccccctgaccgtgaacgagaacaggaggctgaagctgatcatgcccgccaggttctaccccaacgtgaccaagtacctgcccctggacaagggcatcaagccctactaccccgagcacgtggtgaaccactacttccagaccaggcactacctgcacaccctgtggaaggccggcatcctgtacaagagggagagcaccaggagcgccagcttctgcggcagcccctacagctgggagcaggacctgcagcacggcaggctggtgttcaagaccagcaagaggcacggcgacaagagcttctgcccccagagccccggcatcctgcccaggagcagcgtgggcccctgcatccagagccagctgaggaagagcaggctgggcccccagcccgcccagggccagctggccggcaggcagcagggcggcagcggcagcatcagggccagggtgcaccccagcccctggggcaccgtgggcgtggagcccagcggcagcggccacacccacaactgcgccagcagcagcagcagctgcctgcaccagagcgccgtgaggaaggccgcctacagcctgatcagcaccagcaagggccacagcagcagcggccacgccgtggagctgcaccacttcccccccaacagcagcaggagccagagccagggccccgtgctgagctgctggtggctgcagttcaggaacagcgagccctgcagcgagtactgcctgtgccacatcgtgaacctgatcgaggactggggcccctgcaccgagcacggcgagcacaggatcaggacccccaggacccccgccagggtgaccggcggcgtgttcctggtggacaagaacccccacaacaccaccgagagcaggctggtggtggacttcagccagttcagcaggggcgacaccagggtgagctggcccaagttcgccgtgcccaacctgcagagcctgaccaacctgctgagcagcaacctgagctggctgagcctggacgtgagcgccgccttctaccacctgcccctgcaccccgccgccatgccccacctgctggtgggcagcagcggcctgagcaggtacgtggccaggctgagcagcaacagcaggatcatcaacaaccagcacaggaccatgcagaacctgcacaacagctgcagcaggaacctgtacgtgagcctgatgctgctgtacaagacctacggcaggaagctgcacctgtacagccaccccatcatcctgggcttcaggaagatccccatgggcgtgggcctgagccccttcctgctggcccagttcaccagcgccatctgcagcgtggtgaggagggccttcccccactgcctggccttcagctacatggacgacgtggtgctgggcgccaagagcgtgcagcacctggagagcctgtacgccgccgtgaccaacttcctgctgagcctgggcatccacctgaacccccacaagaccaagaggtggggctacagcctgaacttcatgggctacgtgatcggctgctggggcaccatgccccaggagcacatcgtgcagaagatcaagatgtgcttcaggaagctgcccgtgaacaggcccatcgactggaaggtgtgccagaggatcgtgggcctgctgggcttcgccgcccccttcacccagtgcggctaccccgccctgatgcccctgtacgcctgcatccaggccaagcaggccttcaccttcagccccacctacaaggccttcctgagcaagcagtacctgaacctgtaccccgtggccaggcagaggagcggcctgtgccaggtgttcgccgacgccacccccaccggctggggcctggccatcggccaccagaggatgaggggcaccttcgtgagccccctgcccatccacaccgccgagctgctggccgcctgcttcgccaggagcaggagcggcgccaagctgatcggcaccgacaacagcgtggtgctgagcaggaagtacaccagcttcccctggctgctgggctgcgccgccaactggatcctgaggggcaccagcttcgtgtacgtgcccagcgccctgaaccccgccgacgaccccagcaggggcaggctgggcctgtacaggcccctgctgaggctgctgtacaggcccaccaccggcaggaccagcctgtacgccgacagccccagcgtgcccagccacctgcccgacagggtgcacttcgccagccccctgcacgtggcctggaggcccccc

SEQ ID NO: 134 - DNA, 1353 bp, synthetic construct
  source 1..1353: mol_type = other DNA
gacaccagctggctgagggacgtgtgggactgggtgtgcaccgtgctgagcgacttcagggtgtggctgcaggccaagctgctgcccaggctgcccggcatccccttcttcagctgccagaccggctacaggggcgtgtgggccggcgacggcgtgtgccacaccacctgcacctgcggcgccgtgatcgccggccacgtgaagaacggcaccatgaagatcaccggccccaagacctgcagcaacacctggcacggcaccttccccatcaacgccaccaccaccggccccagcacccccaggcccgcccccagctaccagagggccctgtggagggtgagcgccgaggactacgtggaggtgaggaggctgggcgacaggcactacgtggtgggcgtgaccgccgagggcctgaagtgcccctgccaggtgcccgcccccgagttcttcaccgagatcgacggcgtgaggctgcacaggtacgcccccccctgcaagcccctgctgagggacgaggtgaccttcagcgtgggcctgagcacctacgccatcggcagccagctgccctgcgagcccgagcccgacgtgaccgtggtgaccagcatgctgaccgaccccacccacatcaccgccgagaccgccgccaggaggctgaagaggggcagcccccccagcctggccagcagcagcgccagccagctgagcgcccccagcctgaaggccacctgcaccaccagcaaggaccaccccgacatggagctgatcgaggccaacctgctgtggaggcaggagatgggcggcaacatcaccagggtggagagcgagaacaaggtggtggtgctggacagcttcgagcccctgaccgccgagtacgacgagagggagatcagcgtgagcgccgagtgccacaggccccccaggcacaagttcccccccgccctgcccatctgggccaggcccgactacaacccccccctgatccaggcctggcagatgcccggctacgagccccccgtggtgagcggctgcgccatcgccccccccaagcccgcccccatccccccccccaggaggaagaggctggtgaggctggacgagagcaccgtgagccacgccctggcccagctggccgacaaggtgttcgtggagagcagcagcgaccccggccccagcagcgacagcggcctgagcatcgccagccccgtgccccccgcccccaccaccagcgacgacgcctgcagcgaggccgagagctacagcagcatgccccccctggagggcgagcccggcgaccccgacctgagcagcggcagctggagcaccgtgagcgaccaggacgacgtggtgtgctgc

SEQ ID NO: 135 - DNA, 1497 bp, synthetic construct
  source 1..1497: mol_type = other DNA
atggcgtcccaaggcaccaaacggtcttatgaacagatggaaactgatggggaacgccagaatgcaactgagatcagagcatccgtcgggaagatgattgatggaattggacgattctacatccaaatgtgcaccgaacttaaactcagtgattatgaggggcgactgatccagaacagcttaacaatagagagaatggtgctctctgcttttgacgagagaaggaataaatatctggaagaacatcccagcgcggggaaggatcctaagaaaactggaggacccatatacaagagagtagatggaaagtggatgagggaactcgtcctttatgacaaagaagaaataaggcgaatctggcgccaagccaataatggtgatgatgcaacagctgggctgactcacatgatgatctggcattccaatttgaatgatacaacataccagaggacaagagctcttgttcgcaccggaatggatcccaggatgtgctctttgatgcagggttcgactctccctaggaggtctggagctgcaggcgctgcagtcaaaggagttgggacaatggtgatggagttgatcaggatgatcaaacgtgggatcaatgatcggaacttctggagaggtgagaatggacggaaaacaaggagtgcttacgagagaatgtgcaacattctcaaaggaaaatttcaaacagctgcacaaagagcaatgatggatcaagtgagagaaagccggaacccaggaaatgctgagatcgaagatctaatctttctggcacggtctgcactcatattgagagggtcagttgctcacaaatcttgtctgcccgcctgtgtgtatggacctgccatagccagtgggtacaacttcgaaaaagagggatactctctagtgggaatagaccctttcaaactgcttcaaaacagccaagtatacagcctaatcagaccgaacgagaatccagcacacaagagtcagctggtgtggatggcatgcaattctgctgcatttgaagatctaagagtattaagcttcatcagagggaccaaagtatccccaagggggaaactttccactagaggagtacaaattgcttcaaatgaaaacatggatactatggaatcaagtactcttgaactaagaagcaggtactgggccataaggaccagaagtggaggaaacactaatcaacagagggcctctgcaggtcaaatcagtgtacaacctgcattttctgtgcaaagaaacctcccatttgacaaaccaaccatcatggcagcattcactgggaatacagagggaagaacatcagacatgagggcagaaatcataaggatgatggaaggtgcaaaaccagaagaaatgtccttccaggggcggggagtcttcgagctctcggacgaaaaggcaacgaacccgatcgtgccctcttttgacatgagtaatgaaggatcttatttcttcggagacaatgcagaggagtacgacaattaa

SEQ ID NO: 136 - protein, 15 aa, synthetic construct
FQDAYNAAGGHNAVF

SEQ ID NO: 137 - protein, 15 aa, synthetic construct
EISTNIRQAGVQYSR

SEQ ID NO: 138 - protein, 19 aa, synthetic construct
TRFQTRFQTLLALHRSYLT

SEQ ID NO: 139 - protein, 13 aa, synthetic construct
PKYVKQNTLKLAT

SEQ ID NO: 140 - protein, 15 aa, synthetic construct
MSQIMYNYPAMMAHA

SEQ ID NO: 141 - protein, 15 aa, synthetic construct
HTYTIDWTKDAVTWS

SEQ ID NO: 142 - protein, 16 aa, synthetic construct
YYSNVTATRLLSSTNS

SEQ ID NO: 143 - protein, 16 aa, synthetic construct
QAGFFLLTRILTIPQS

SEQ ID NO: 144 - protein, 16 aa, synthetic construct
VYYLTRDPTTPLARAA

SEQ ID NO: 145 - protein, 20 aa, synthetic construct
FRDYVDRFYKTLRAEQASQE

SEQ ID NO: 146 - protein, 16 aa, synthetic construct
PIVQLQGDSNCLKCFR

SEQ ID NO: 147 - protein, 20 aa, synthetic construct
EYLNKIQNSLSTEWSPCSVT

SEQ ID NO: 148 - protein, 21 aa, synthetic construct
FNNFTVSFWLRVPKVSASHLE

SEQ ID NO: 149 - protein, 15 aa, synthetic construct
GEEYLILSARDVLAV

SEQ ID NO: 150 - protein, 15 aa, synthetic construct
MTEQQWNFAGIEAAA

SEQ ID NO: 151 - protein, 15 aa, synthetic construct
MHVSFVMAYPEMLAA

SEQ ID NO: 152 - protein, 9 aa, synthetic construct
TDLGQNLLY

SEQ ID NO: 153 - protein, 9 aa, synthetic construct
RLNMFTPYI

SEQ ID NO: 154 - protein, 9 aa, synthetic construct
FTSDYYQLY

SEQ ID NO: 155 - protein, 9 aa, synthetic construct
LLLDRLNQL

SEQ ID NO: 156 - protein, 9 aa, synthetic construct
LLYDANYFL

SEQ ID NO: 157 - protein, 9 aa, synthetic construct
KLAEAIFKL

SEQ ID NO: 158 - protein, 9 aa, synthetic construct
KYTSFPWLL

SEQ ID NO: 159 - protein, 9 aa, synthetic construct
VLSDFKTWL

SEQ ID NO: 160 - protein, 9 aa, synthetic construct
RLRPGGKKK

SEQ ID NO: 161 - protein, 9 aa, synthetic construct
SPIVPSFDM

SEQ ID NO: 162 - protein, 8 aa, synthetic construct
SVLAFRRL

SEQ ID NO: 163 - protein, 9 aa, synthetic construct
AMASTEGNV

SEQ ID NO: 164 - DNA, 4637 bp, synthetic construct
  source 1..4637: mol_type = other DNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaacgccaccaacttcagcctgctgaagcaggccggcgacgtggaggagaaccccggccccatgttcgtgtttctggtgctgctgcctctggtgtccagccagtgtggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggaggtgaacctgaccaccagaacacagctgcctccagcctacaccaacagctttaccagaggcgtgtactaccctgacaaggtgttcagatccagtgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggctcctctgagcgagacaaagtgcaccctgaagtccttcaccgtggaaaagggcatctaccagaccagcaacttccgggtgcagcccaccgagtccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcactatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaaaggcttcaactgctacttcccactgcagtcctacggctttcagcccacgtatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggacatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacccgccggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgatgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgctaataattaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 165 - protein, 8 aa, synthetic construct
DXEXNPGP

SEQ ID NO: 166 - DNA, 1478 bp, synthetic construct
  source 1..1478: mol_type = other DNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaacacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 167 - RNA, 1478 nt, synthetic construct
  source 1..1478: mol_type = other RNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaacacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 168 - protein, 330 aa, synthetic construct
MNQRKRVVRPPFNMLKRERNTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 169 - DNA, 1805 bp, synthetic construct
  source 1..1805: mol_type = other DNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaacctcgagacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaagctagcggcggcggcggcagcggcggcggcggcagcggcggcggcggcagcggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggagtaataattaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 170 - RNA, 1805 nt, synthetic construct
  source 1..1805: mol_type = other RNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaacctcgagacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaagctagcggcggcggcggcagcggcggcggcggcagcggcggcggcggcagcggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggagtaataattaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 171 - protein, 438 aa, synthetic construct
MNQRKRVVRPPFNMLKRERNLETSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQASGGGGSGGGGSGGGGSGRWHKVSVRWEFQDAYNAAGGHNAVFGRWHKVSVRWEFQDAYNAAGGHNAVFGRWHKVSVRWEFQDAYNAAGGHNAVFGRWHKVSVRWE

SEQ ID NO: 172 - DNA, 1586 bp, synthetic construct
  source 1..1586: mol_type = other DNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaacctcgagatgttcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcgctagcggcggcggcggcagcggcggcggcggcagcggcggcggcggcagcggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggagtaataattaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 173 - RNA, 1586 nt, synthetic construct
  source 1..1586: mol_type = other RNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaacctcgagatgttcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcgctagcggcggcggcggcagcggcggcggcggcagcggcggcggcggcagcggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggagttccaggacgcctacaacgccgccggcggccacaacgccgtgttcggcaggtggcacaaggtgagcgtgaggtgggagtaataattaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 174 - protein, 365 aa, synthetic construct
MNQRKRVVRPPFNMLKRERNLEMFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFASGGGGSGGGGSGGGGSGRWHKVSVRWEFQDAYNAAGGHNAVFGRWHKVSVRWEFQDAYNAAGGHNAVFGRWHKVSVRWEFQDAYNAAGGHNAVFGRWHKVSVRWE

SEQ ID NO: 175 - DNA, 1365 bp, synthetic construct
  source 1..1365: mol_type = other DNA
gagaataaactagtattcttctggtccccacagactcagagagaacccgccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcacccgggtttttttttttttttttttttttttttttttttttttttttttttttttttttttttttttttttttttt

SEQ ID NO: 176 - RNA, 1369 nt, synthetic construct
  source 1..1369: mol_type = other RNA
gagaataaactagtattcttctggtccccacagactcagagagaacccgccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcacccgggttttttttttttttttttttttttttttttttttttttttttttttttttttttttttttttttttttttattt

SEQ ID NO: 177 - protein, 311 aa, synthetic construct
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 178 - DNA, 4382 bp, synthetic construct
  source 1..4382: mol_type = other DNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaacgccaccaacttcagcctgctgaagcaggccggcgacgtggaggagaaccccggccccatgttcgtgtttctggtgctgctgcctctggtgtccagccagtgtctcgaggtgaacctgaccaccagaacacagctgcctccagcctacaccaacagctttaccagaggcgtgtactaccctgacaaggtgttcagatccagtgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggctcctctgagcgagacaaagtgcaccctgaagtccttcaccgtggaaaagggcatctaccagaccagcaacttccgggtgcagcccaccgagtccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcactatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaaaggcttcaactgctacttcccactgcagtcctacggctttcagcccacgtatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggacatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacccgccggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgatgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgctaataagctagcttaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 179 - RNA, 4382 nt, synthetic construct
  source 1..4382: mol_type = other RNA
agttgttagtctgtgtggaccgacaaggacagttctaaatcggaagcttgcttaacgcagttctaacagtttgtttagatagagagcagatctctggaaaaatgaaccaacgaaaaagggtggttagaccacctttcaatatgctgaaacgcgagagaaacgccaccaacttcagcctgctgaagcaggccggcgacgtggaggagaaccccggccccatgttcgtgtttctggtgctgctgcctctggtgtccagccagtgtctcgaggtgaacctgaccaccagaacacagctgcctccagcctacaccaacagctttaccagaggcgtgtactaccctgacaaggtgttcagatccagtgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggctcctctgagcgagacaaagtgcaccctgaagtccttcaccgtggaaaagggcatctaccagaccagcaacttccgggtgcagcccaccgagtccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcactatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaaaggcttcaactgctacttcccactgcagtcctacggctttcagcccacgtatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggacatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacccgccggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgatgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgctaataagctagcttaccaacaacaaacaccaaaggctattgaagtcaggccacttgtgccacggctggagcaaaccgtgctgcctgtagctccgccaataacgggaggcgttataattcccagggaggccatgcgccacggaagctgtacgcgtggcatattggactagcggttagaggagacccctcccatcaccaacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 180 - protein, 1295 aa, synthetic construct
MNQRKRVVRPPFNMLKRERNATNFSLLKQAGDVEENPGPMFVFLVLLPLVSSQCLEVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALAPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC